(19) United States
(12) Patent Application Publication
(10) Pub. No.: US 2024/0139345 A1
(43) Pub. Date: May 2, 2024

(54) NUCLEIC ACID VECTORS AND METHODS OF USE

(71) Applicant: Intergalactic Therapeutics, Inc., Cambridge, MA (US)

(72) Inventors: Jin HUH, Cambridge, MA (US); Jodi KENNEDY, Cambridge, MA (US); Raj MEHTA, Cambridge, MA (US); Gayathri RAMASWAMY, Cambridge, MA (US); Robert FARRA, Cambridge, MA (US); Cathleen GONZALES, Cambridge, MA (US)

(21) Appl. No.: 18/278,235

(22) PCT Filed: Feb. 23, 2022
(86) PCT No.: PCT/US2022/017575

§ 371 Date: Aug. 22, 2023

Related U.S. Application Data
(60) Provisional application No. 63/152,540, filed on Feb. 23, 2021.
(60) Provisional application No. 63/152,575, filed on Feb. 23, 2021.

(51) Int. Cl. A61K 48/00; A61P 35/00; C07K 14/52; C07K 14/53; C07K 14/535; C07K 14/54; C12N 15/85

(52) U.S. Cl. CPC A61K 48/0066; A61K 48/0016; A61K 48/0083; A61P 35/00; C07K 14/521; C07K 14/53; C07K 14/535; C07K 14/5434; C12N 15/85; C12N 2800/107; C12N 2830/50

(57) ABSTRACT

The present invention involves nucleic acid vectors (e.g., circular DNA vectors) and compositions thereof. Also provided herein are methods of administering nucleic acid vectors (e.g., circular DNA vectors) and compositions thereof, e.g., in combination with pulsed electric field therapy.

### FIELD OF THE INVENTION

In general, the invention features compostions and methods involving nucleic acid vectors (e.g., circular DNA vectors), e.g., for treatment of cancer.

### BACKGROUND

Cancer occurs when normal physiological responses to stimuli (e.g., responses to insult or injury, initiation of self-repair, or mounting innate and acquired defenses) become abnormal or excessive, leading to additional disease or pathology (e.g., tumor growth and/or metastasis causing tissue damage and systemic failure). Pathogenesis of cancer has been extensively studied, leading to numerous hypotheses suggesting a wide variety of treatment approaches with varying rates of success. Nevertheless, cancer remains one of the deadliest threats to human health. Globally, cancer is the second leading cause of death, accounting for about one in six deaths, according to the World Health Organization, and the American Cancer Society estimates that cancer causes over 1,500 deaths per day in the United States alone.

Gene therapy holds promise as an effective treatment of cancer, but its full potential is yet unrealized. Thus, there is a need in the field for improved gene therapies capable of providing a robust and sustained anti-tumor effect.

### SUMMARY OF THE INVENTION

The present invention provides compositions and methods for treating cancer (e.g., cancers characterized by the presence of solid tumors) by administering nucleic acid vectors (e.g., nonviral nucleic acid vectors, e.g., circular DNA vectors) carrying heterologous genes to modulate the tumor microenvironment. In some embodiments, the modulatory genes are a combination of immunomodulatory genes that work in concert to mount an anti-tumor immune response. Such modulatory genes include various combinations of a dendric cell chemoattractant-encoding gene (e.g., XCL1, XCL2, CCL4, or CCL5), a dendric cell growth factor or activator-encoding gene (e.g., FLT3L, GM-CSF, CD40, or CD40L), and a lymphocyte signaling protein-encoding genes (e.g., IL-12, IL-15, CXCL9, or CXCL10). In certain embodiments described herein, the nucleic acid vectors encode self-replicating RNA molecules in which an RNA replicase is operably linked to one or more modulatory genes to mitigate dilution as tumor cells proliferate. Administration of nucleic acid vectors (e.g., circular DNA vectors, e.g., circular DNA vectors encoding self-replicating RNA molecules) can be accompanied by transmission of an electric field (e.g., a pulsed electric field) into a tumor microenvironment to enhance electrotransfer into target cells.

In one aspect, the invention provides a nucleic acid vector (e.g., a DNA vector, e.g., a circular DNA vector) comprising a dendritic cell chemoattractant-encoding gene (e.g., an XCL1-encoding gene, an XCL2-encoding gene, a CCL5-encoding gene, or a CCL4-encoding gene), a dendritic cell growth factor or activator-encoding gene (e.g., a FLT3L-encoding gene (e.g., a sFLT3L-encoding gene), a GM-CSF-encoding gene, or a CD40- or CD40L-encoding gene), and a lymphocyte signaling protein-encoding gene (e.g., an IL-12-encoding gene, an IL-15-encoding gene, a CXCL9-encoding gene, or a CXCL10-encoding gene). In some embodiments, the nucleic acid vector (e.g., DNA vector, e.g., circular DNA vector) comprises a first promoter, a second promoter, and a third promoter driving expression of the dendritic cell chemoattractant-encoding gene (e.g., the XCL1-encoding gene, the XCL2-encoding gene, the CCL5-encoding gene, or the CCL4-encoding gene), the dendritic cell growth factor or activator-encoding gene (e.g., the FLT3L-encoding gene, the GM-CSF-encoding gene, or the CD40- or CD40L-encoding gene), and the lymphocyte signaling protein-encoding gene (e.g., the IL-12-encoding gene, the IL-15-encoding gene, the CXCL9-encoding gene, or the CXCL10-encoding gene), respectively. In some embodiments, one or more of the first promoter, the second promoter, and the third promoter is a human cytomegalovirus (CMV) promoter. In some embodiments, the first promoter is a CMV promoter, the second promoter is a CMV promoter, and the third promoter is a CMV promoter. In some embodiments, one or more of the first promoter, the second promoter, and the third promoter is a CAG promoter. In some embodiments, the first promoter is a CAG promoter, the second promoter is a CAG promoter, and the third promoter is a CAG promoter. In some embodiments, the nucleic acid vector (e.g., DNA vector, e.g., circular DNA vector) comprises a single promoter driving expression of the dendritic cell chemoattractant-encoding gene (e.g., the XCL1-encoding gene, the XCL2-encoding gene, the CCL5-encoding gene, or the CCL4encoding gene), the dendritic cell growth factor or activator-encoding gene (e.g., the FLT3L-encoding gene, the GM-CSF-encoding gene, or the CD40- or CD40L-encoding gene), and the lymphocyte signaling protein-encoding gene (e.g., the IL-12-encoding gene, the IL-15-encoding gene, the CXCL9-encoding gene, or the CXCL10-encoding gene). In particular embodiments, the nucleic acid vector is a non-viral nucleic acid vector. In some embodiments, the promoter is a CMV promoter. In other embodiments, the promoter is a CAG promoter.

In some of any of the preceding embodiments, the nucleic acid vector is a circular DNA vector that lacks a bacterial origin of replication and/or a drug resistance gene (e.g., lacks both a bacterial origin of replication and/or a drug resistance gene). In some embodiments, the nucleic acid vector (e.g., DNA vector, e.g., circular DNA vector) is 2.5 kb to 20 kb in length. In some embodiments, the nucleic acid (e.g., DNA vector, e.g., circular DNA vector) vector is 3.5 kb to 10 kb in length.

In some embodiments, the nucleic acid vector (e.g., circular DNA vector) includes, in a 5′ to 3′ direction, the dendritic cell growth factor or activator-encoding gene, the lymphocyte signaling protein-encoding gene, and the dendritic cell chemoattractant-encoding gene. In some embodiments, the circular DNA vector is a synthetic circular DNA vector lacking a recombination site (e.g., a synthetic circular DNA vector lacking a recombination site and a bacterial backbone).

In another aspect, the invention features a circular DNA vector that lacks a bacterial origin of replication and/or a drug resistance gene (e.g., lacks both a bacterial origin of replication and/or a drug resistance gene). The circular DNA vector has the following elements arranged (e.g., operably linked) in a 5′ to 3 direction: (a) a promoter (e.g., a CMV or a CAG promoter); (b) a self-replicating RNA molecule-encoding sequence comprising (i) a replicase-encoding sequence that transcribes RNA from the self-replicating RNA molecule and (ii) one or more heterologous protein-encoding sequences (e.g., genes); and (c) a polyadenylation sequence. In some embodiments, the one or more heterologous protein-encoding sequences (e.g., genes) include one or more immunomodulatory protein-encoding genes. In some embodiments, the one or more (e.g., one, two, three, or more) immunomodulatory protein-encoding genes are selected from a dendritic cell chemoattractant-encoding gene (e.g., an XCL1-encoding gene, an XCL2-encoding gene, a CCL5-encoding gene, or a CCL4-encoding gene), a dendritic cell growth factor or activator-encoding gene (e.g., a FLT3L-encoding gene (e.g., a sFLT3L-encoding gene), a GM-CSF-encoding gene, or a CD40 or CD40L-encoding gene), and a lymphocyte signaling protein-encoding gene (e.g., an IL-12-encoding gene, an IL-15-encoding gene, a CXCL9-encoding gene, or a CXCL10-encoding gene). In some embodiments, the one or more immunomodulatory protein-encoding genes include all three of a dendritic cell chemoattractant-encoding gene (e.g., an XCL1-encoding gene, an XCL2-encoding gene, a CCL5-encoding gene, or a CCL4-encoding gene), a dendritic cell growth factor or activator-encoding gene (e.g., a FLT3L-encoding gene (e.g., a sFLT3L-encoding gene), a GM-CSF-encoding gene, or a CD40- or CD40L-encoding gene), and a lymphocyte signaling protein-encoding gene (e.g., an IL-12-encoding gene, an IL-15-encoding gene, a CXCL9-encoding gene, or a CXCL10-encoding gene). In some embodiments of any of the preceding aspects, the replicase is an alphavirus replicase (e.g., a VEE replicase or a variant thereof (e.g., a replicase having one or more (one, two, three, or all four) amino acid sequences of SEQ ID NOs: 2, 4, 6, and/or 8, or a variant thereof having at least 95%, at least 96%, at least 97%, at least 98%, at least 99%, or 100% sequence identity to SEQ ID NOs: 2, 4, 6, and/or 8)).

In another aspect, the invention features a pharmaceutical composition including any of the nucleic acid vectors or circular DNA vectors of the previous aspects and a pharmaceutically acceptable carrier (e.g., as a liquid or dry (e.g., lyophilized) composition).

In another aspect, provided herein are methods of treating a cancer in an individual in need thereof (e.g., a human cancer patient). In some embodiments, the method includes administering the nucleic acid vector (e.g., non-viral nucleic acid vector), the circular DNA vector, or the pharmaceutical composition of any one of the preceding aspects to the individual in an effective amount to treat the cancer. In some embodiments, the nucleic acid vector, the circular DNA vector, or the pharmaceutical composition is administered intratumorally. In other embodiments, the nucleic acid vector, the circular DNA vector, or the pharmaceutical composition is administered systemically. In some embodiments, the nucleic acid vector, the circular DNA vector, or the pharmaceutical composition is administered in combination with transmission of an electric field to the tumor microenvironment (e.g., a pulsed electric field therapy, e.g., a pulsed electric field therapy administered using an intratumorally positioned electrode). In some embodiments, the method includes transmitting the electric field into the tumor microenvironment, wherein the electric field promotes transfer of the nucleic acid vector (e.g., the circular DNA vector) into a cell (e.g., a tumor cell), thereby delivering the heterologous modulatory gene to the cell (e.g., tumor cell) to treat the cancer. Additionally, or alternatively, the nucleic acid vector, the circular DNA vector, or the pharmaceutical composition can be administered in combination with an additional anti-cancer therapy (e.g., a radiation therapy (e.g., a cytotoxic radiotherapy), a photodynamic therapy, a hyperthermic therapy, an oncolytic therapy (e.g., administration of an oncolytic virus), or an anti-cancer agent (e.g., a chemotherapeutic agent, a checkpoint inhibitor, a cytotoxic agent, a growth inhibitory agent, an anti-angiogenic agent, a cytokine, a cytokine antagonist, an antibody-drug conjugate, a cancer vaccine, or a combination thereof)).

In another aspect, the invention provides a method treating a cancer in an individual (e.g., a human cancer patient) who has been, or will be, administered the nucleic acid vector (e.g., non-viral nucleic acid vector), the circular DNA vector, or the pharmaceutical composition of any embodiment of the present invention by transmitting an electric field to the tumor microenvironment (e.g., by transmitting a pulsed electric field to the tumor microenvironment, e.g., using an intratumorally positioned electrode).

In another aspect, the invention features a method of modulating a tumor microenvironment in an individual in need thereof (e.g., a human cancer patient, e.g., a human cancer patient having a solid tumor). In some embodiments, the method includes administering the nucleic acid vector (e.g., non-viral nucleic acid vector), the circular DNA vector, or the pharmaceutical composition of any one of the preceding aspects to the individual in an effective amount to modulate the tumor microenvironment. In some embodiments, the nucleic acid vector, the circular DNA vector, or the pharmaceutical composition is administered intratumorally. In other embodiments, the nucleic acid vector, the circular DNA vector, or the pharmaceutical composition is administered systemically. In some embodiments, the nucleic acid vector, the circular DNA vector, or the pharmaceutical composition is administered in combination with transmission of an electric field to the tumor microenvironment (e.g., a pulsed electric field therapy, e.g., a pulsed electric field therapy administered using an intratumorally positioned electrode). In some embodiments, the method includes modulating the tumor microenvironment by transmitting the electric field into the tumor microenvironment, wherein the electric field promotes transfer of the nucleic acid vector (e.g., the circular DNA vector) into a cell (e.g., a tumor cell), thereby delivering the heterologous modulatory gene to the cell (e.g., tumor cell) to modulate the tumor microenvironment in the individual.

Additionally, or alternatively, the nucleic acid vector, the circular DNA vector, or the pharmaceutical composition can be administered in combination with an additional anti-cancer therapy (e.g., a radiation therapy (e.g., a cytotoxic radiotherapy), a photodynamic therapy, a hyperthermic therapy, an oncolytic therapy (e.g., administration of an oncolytic virus), or an anti-cancer agent (e.g., a chemotherapeutic agent, a checkpoint inhibitor, a cytotoxic agent, a growth inhibitory agent, an anti-anglogenic agent, a cytokine, a cytokine antagonist, an antibody-drug conjugate, a cancer vaccine, or a combination thereof)).

In another aspect, the invention provides a method of modulating a tumor microenvironment in an individual (e.g., a human cancer patient) who has been, or will be, administered the nucleic acid vector (e.g., non-viral nucleic acid vector), the circular DNA vector, or the pharmaceutical composition of any embodiment of the present invention by transmitting an electric field to the tumor microenvironment (e.g., by transmitting a pulsed electric field to the tumor microenvironment, e.g., using an intratumorally positioned electrode), thereby delivering the heterologous modulatory gene to the cell (e.g., tumor cell) to modulate the tumor microenvironment in the individual.

In another aspect, the invention features inducing expression of a modulatory protein in a tumor microenvironment in an individual in need thereof (e.g., a human cancer patient, e.g., a human cancer patient having a solid tumor). In some embodiments, the method includes administering the nucleic acid vector (e.g., non-viral nucleic acid vector), the circular DNA vector, or the pharmaceutical composition of any one of the preceding aspects to the individual in an effective amount to induce expression of the modulatory protein encoded thereby. In some embodiments, the nucleic acid vector, the circular DNA vector, or the pharmaceutical composition is administered intratumorally. In other embodiments, the nucleic acid vector, the circular DNA vector, or the pharmaceutical composition is administered systemically. In some embodiments, the nucleic acid vector, the circular DNA vector, or the pharmaceutical composition is administered in combination with transmission of an electric field to the tumor microenvironment (e.g., a pulsed electric field therapy, e.g., a pulsed electric field therapy administered using an intratumorally positioned electrode). In some embodiments, the method includes modulating the tumor microenvironment by transmitting the electric field into the tumor microenvironment, wherein the electric field promotes transfer of the nucleic acid vector (e.g., the circular DNA vector) into a cell (e.g., a tumor cell), thereby delivering the heterologous modulatory gene to the cell (e.g., tumor cell) to induce its expression. Additionally, or alternatively, the nucleic acid vector, the circular DNA vector, or the pharmaceutical composition can be administered in combination with an additional anti-cancer therapy (e.g., a radiation therapy (e.g., a cytotoxic radiotherapy), a photodynamic therapy, a hyperthermic therapy, an oncolytic therapy (e.g., administration of an oncolytic virus), or an anti-cancer agent (e.g., a chemotherapeutic agent, a checkpoint inhibitor, a cytotoxic agent, a growth inhibitory agent, an anti-angiogenic agent, a cytokine, a cytokine antagonist, an antibody-drug conjugate, a cancer vaccine, or a combination thereof)).

In another aspect, the invention provides a method of inducing expression of a modulatory protein in a tumor microenvironment in an individual (e.g., a human cancer patient) who has been, or will be, administered the nucleic acid vector (e.g., non-viral nucleic acid vector), the circular DNA vector, or the pharmaceutical composition of any embodiment of the present invention by transmitting an electric field to the tumor microenvironment (e.g., by transmitting a pulsed electric field to the tumor microenvironment, e.g., using an intratumorally positioned electrode), thereby delivering the heterologous modulatory gene to the cell (e.g., tumor cell) to induce expression of the modulatory protein encoded by the vector in the tumor microenvironment.

In another aspect, provided herein are methods of treating a cancer in an individual in need thereof (e.g., a human cancer patient) by administering a non-viral nucleic acid vector comprising a dendritic cell chemoattractant-encoding gene in an effective amount to treat the cancer. In some embodiments, the non-viral nucleic acid vector is administered intratumorally. In other embodiments, the non-viral nucleic acid vector is administered systemically. In some embodiments, the non-viral nucleic acid vector is administered in combination with transmission of an electric field to the tumor microenvironment (e.g., a pulsed electric field therapy, e.g., a pulsed electric field therapy administered using an intratumorally positioned electrode). In some embodiments, the method includes transmitting the electric field into the tumor microenvironment, wherein the electric field promotes transfer of the non-viral nucleic acid vector, thereby delivering the dendritic cell chemoattractant-encoding gene to the cell (e.g., tumor cell) to treat the cancer. Additionally, or alternatively, the non-viral nucleic acid vector can be administered in combination with an additional anti-cancer therapy (e.g., a radiation therapy (e.g., a cytotoxic radiotherapy), a photodynamic therapy, a hyperthermic therapy, an oncolytic therapy (e.g., administration of an oncolytic virus), or an anti-cancer agent (e.g., a chemotherapeutic agent, a checkpoint inhibitor, a cytotoxic agent, a growth inhibitory agent, an anti-angiogenic agent, a cytokine, a cytokine antagonist, an antibody-drug conjugate, a cancer vaccine, or a combination thereof)).

In another aspect, the invention provides a method treating a cancer in an individual (e.g., a human cancer patient) who has been, or will be, administered the non-viral nucleic acid vector having a dendritic cell chemoattractant-encoding gene by transmitting an electric field to the tumor microenvironment (e.g., by transmitting a pulsed electric field to the tumor microenvironment, e.g., using an intratumorally positioned electrode).

In another aspect, the invention features a method of modulating a tumor microenvironment in an individual in need thereof (e.g., a human cancer patient, e.g., a human cancer patient having a solid tumor) by administering a non-viral nucleic acid vector having a dendritic cell chemoattractant-encoding gene to the individual in an effective amount to modulate the tumor microenvironment. In some embodiments, the non-viral nucleic acid vector is administered intratumorally. In other embodiments, the non-viral nucleic acid vector is administered systemically. In some embodiments, the non-viral nucleic acid vector is administered in combination with transmission of an electric field to the tumor microenvironment (e.g., a pulsed electric field therapy, e.g., a pulsed electric field therapy administered using an intratumorally positioned electrode). In some embodiments, the method includes modulating the tumor microenvironment by transmitting the electric field into the tumor microenvironment, wherein the electric field promotes transfer of the non-viral nucleic acid vector into a cell (e.g., a tumor cell), thereby delivering the dendritic cell chemoattractant-encoding gene to the cell (e.g., tumor cell) to modulate the tumor microenvironment in the individual. Additionally, or alternatively, the non-viral nucleic acid vector can be administered in combination with an additional anti-cancer therapy (e.g., a radiation therapy (e.g., a cytotoxic radiotherapy), a photodynamic therapy, a hyperthermic therapy, an oncolytic therapy (e.g., administration of an oncolytic virus), or an anti-cancer agent (e.g., a chemotherapeutic agent, a checkpoint inhibitor, a cytotoxic agent, a growth inhibitory agent, an anti-angiogenic agent, a cytokine, a cytokine antagonist, an antibody-drug conjugate, a cancer vaccine, or a combination thereof)).

In another aspect, the invention provides a method of modulating a tumor microenvironment in an individual (e.g., a human cancer patient) who has been, or will be, administered a non-viral nucleic acid vector having a dendritic cell chemoattractant-encoding gene by transmitting an electric field to the tumor microenvironment (e.g., by transmitting a pulsed electric field to the tumor microenvironment, e.g., using an intratumorally positioned electrode), thereby delivering the dendritic cell chemoattractant-encoding gene to the cell (e.g., tumor cell) to modulate the tumor microenvironment in the individual.

In another aspect, the invention features inducing expression of a modulatory protein in a tumor microenvironment in an individual in need thereof (e.g., a human cancer patient, e.g., a human cancer patient having a solid tumor) by administering a non-viral nucleic acid vector having a dendritic cell chemoattractant-encoding gene to the individual in an effective amount to induce expression of the dendritic cell chemoattractant-encoding gene. In some embodiments, the non-viral nucleic acid vector is administered intratumorally. In other embodiments, the non-viral nucleic acid vector is administered systemically. In some embodiments, the non-viral nucleic acid vector is administered in combination with transmission of an electric field to the tumor microenvironment (e.g., a pulsed electric field therapy, e.g., a pulsed electric field therapy administered using an intratumorally positioned electrode). In some embodiments, the method includes modulating the tumor microenvironment by transmitting the electric field into the tumor microenvironment, wherein the electric field promotes transfer of the non-viral nucleic acid vector into a cell (e.g., a tumor cell), thereby delivering the dendritic cell chemoattractant-encoding gene to the cell (e.g., tumor cell) to induce its expression. Additionally, or alternatively, the non-viral nucleic acid vector can be administered in combination with an additional anti-cancer therapy (e.g., a radiation therapy (e.g., a cytotoxic radiotherapy), a photodynamic therapy, a hyperthermic therapy, an oncolytic therapy (e.g., administration of an oncolytic virus), or an anti-cancer agent (e.g., a chemotherapeutic agent, a checkpoint inhibitor, a cytotoxic agent, a growth inhibitory agent, an anti-angiogenic agent, a cytokine, a cytokine antagonist, an antibody-drug conjugate, a cancer vaccine, or a combination thereof)).

In another aspect, the invention provides a method of inducing expression of a dendritic cell chemoattractant in a tumor microenvironment in an individual (e.g., a human cancer patient) who has been, or will be, administered a non-viral nucleic acid vector having a dendritic cell chemoattractant-encoding gene by transmitting an electric field to the tumor microenvironment (e.g., by transmitting a pulsed electric field to the tumor microenvironment, e.g., using an intratumorally positioned electrode), thereby delivering the dendritic cell chemoattractant-encoding gene to the cell (e.g., tumor cell) to induce expression of the dendritic cell chemoattractant in the tumor microenvironment.

In another aspect, the invention provides a method of expressing a protein in a tumor microenvironment in an individual in need thereof. In some embodiments, the method includes: (a) administering a synthetic circular DNA vector encoding the protein; and (b) transmitting an electric field into the tumor microenvironment, wherein the electric field promotes transfer of the synthetic circular DNA vector into a tumor cell, thereby delivering the protein-encoding sequence to the tumor cell to be expressed in the tumor microenvironment in the individual. In some embodiments, step (b) comprises transmitting a pulsed electric field. In some embodiments, the pulsed electric field is transmitted through an intratumorally positioned electrode. In some embodiments, the synthetic circular DNA vector is administered intratumorally. In some embodiments, the synthetic circular DNA vector is administered systemically.

In some embodiments, the synthetic circular DNA vector is administered in combination with an additional anti-cancer therapy. In some embodiments, the protein is selected from the group consisting of a dendritic cell chemoattractant-encoding gene, a dendritic cell growth factor or activator-encoding gene, and a lymphocyte signaling protein-encoding gene.

In some embodiments, the synthetic circular DNA vector encodes a dendritic cell chemoattractant-encoding gene, a dendritic cell growth factor or activator-encoding gene, and a lymphocyte signaling protein-encoding gene. In some embodiments, the synthetic circular DNA vector does not comprise an origin of replication, a drug resistance gene, or a recombination site. In some embodiments, the synthetic circular DNA vector does not comprise a bacterial backbone.

### BRIEF DESCRIPTION OF THE DRAWINGS

FIG. 1A is a schematic diagram showing an exemplary DNA vector of the invention having a single transcription unit. The DNA vector contains a 5′ CAG promoter (P_{CAG}), an sFlt3L-encoding gene, a first furin-P2A sequence, an IL-12-encoding gene, a second furin-P2A sequence, an XCL1-encoding gene, and a 3′ polyA tail. The circularized DNA vector has a size of about 3.5 to 5 kb.

FIG. 1B is an image of a gel showing bands representing a synthetic circular DNA vector of FIG. 1A, which has a size of 4537 bp. The asterisk shows the position of the supercoiled monomeric form, which is 78% of the sample shown here. Lane 1 shows a sample loaded at 200 ng, whereas lane 2 shows the same sample loaded at 800 ng. The ladder is a supercoiled ladder (New England Biosciences).

FIG. 2A is a schematic diagram showing an exemplary multi-transcription unit DNA vector of the invention. The DNA vector contains a first transcription unit having a first P_{CAG}, an sFlt3L-encoding gene, and a first 3′ poly-A tail; a second transcription unit having a second P_{CAG}, an IL-12-encoding gene, and a second 3′ poly-A tail; and a third transcription unit having a third P_{CAG}, an XCL1-encoding gene, and a third 3′ poly-A tail. The circularized DNA vector has a size of about 5 to 10 kb.

FIG. 2B is an image of a gel showing bands representing a synthetic circular DNA vector of FIG. 2A (SEQ ID NO: 37), which has a size of 8035 bp. The asterisk shows the position of the supercoiled monomeric form, which is 85% of the sample shown here. Lane 1 shows a sample loaded at 200 ng, whereas lane 2 shows the same sample loaded at 800 ng. The ladder is a supercoiled ladder (New England Biosciences).

FIGS. 3A-3C are graphs showing protein expression of immunomodulatory proteins from the 8035-bp multi-transcription unit synthetic circular DNA vector shown in FIG. 2A (c3-Tx), normalized to media and measured by ELISA. FIG. 3A shows expression of sFlt3L; FIG. 3B shows expression of IL-12p70; and FIG. 3C shows expression of XCL1.

FIG. 4 is a graph showing expression of functional IL-12p70 by c3-Tx by SEAP-QuantiBlue assay.

FIGS. 5A-5C are flow cytometry plots showing differentiation of bone marrow derived dendritic cells (BMDCs) into conventional DCs (cDCs) and plasmacytoid DCs (pDCs) under various conditions. FIG. 5A shows differentiation in 20 ng/mL sFLT3L expressed by cells transfected with c3-Tx (in conditioned media); FIG. 5B shows differentiation in 20 ng/mL recombinant sFLT3L; and FIG. 5C shows differentiation in recombinant soluble GMCSF (rsGMCSF).

FIGS. 6A-6C are bar graphs showing differentiation of BMDCs into cDCs and pDCs under various conditions. FIG. 6A shows differentiation in 20 ng/mL sFLT3L expressed by cells transfected with c3-Tx (in conditioned media); FIG. 6B shows differentiation in 20 ng/mL recombinant sFLT3L; and FIG. 6C shows differentiation in recombinant soluble GMCSF (rsGMCSF).

FIGS. 7A-7E are in-life images showing fLuc luminescence in tumor-bearing mice under various treatment conditions over time. FIGS. 7A and 7B show PBS control mice at 3 days post-treatment and 17 days post-treatment, respectively. FIGS. 7C and 7D show mice treated with plasmid DNA vectors encoding fLuc (p-fLuc) at 3 days post-treatment and 17 days post-treatment, respectively. FIGS. 7E and 7F show mice treated with synthetic circular DNA vectors encoding fLuc (c3-fLuc) at 3 days post-treatment and 17 days post-treatment, respectively.

FIG. 8 is a graph showing fLuc radiance relative to PBS controls for the experiment of FIGS. 7A-7F, including additional timepoints at days 7, 10, and 14.

FIG. 9 is a graph showing tumor growth over time in mice treated with c3-Tx by electrotransfer, compared to PBS control mice.

FIG. 10 is a graph showing immune cell infiltration in tumors administered with a single dose of c3-Tx or PBS and administered electrical energy. Each datapoint represents the percentage of cells in tumor that express CD45 in c3-Tx-treated mice or PBS control mice.

FIG. 11 is a graph showing survival over time of mice treated two doses of c3-Tx compared with mice injected with PBS or c3-fLuc.

FIG. 12 is a graph showing average tumor volume over time of mice treated with two doses of c3-Tx compared with mice injected with PBS or c3-fLuc.

FIG. 13 is a graph showing GFP expression in tumors injected with mRNA GFP followed by electrotransfer, compared to tumors injected with synthetic circular DNA vector (c3-GFP) followed by electrotransfer and tumors injected with PBS followed by electrotransfer.

FIG. 14A is a schematic diagram showing a tricistronic self-replicating RNA molecule encoding sFLT3L, IL-12, and XCL1, which has a length of 10,429 bp.

FIG. 14B is a schematic diagram showing a plasmid encoding a tricistronic self-replicating RNA molecule encoding sFLT3L, IL-12, and XCL1, which has a length of 13,978.

FIGS. 15A-15C are graphs showing protein expression of immunomodulatory proteins from the tricistronic self-replicating RNA molecule and the plasmid encoding the tricistronic self-replicating RNA molecule, normalized to media and measured by ELISA. FIG. 15A shows expression of sFlt3L; FIG. 15B shows expression of IL-12p70; and FIG. 15C shows expression of XCL1.

FIG. 16 is a graph showing expression of functional IL-12p70 by the tricistronic self-replicating RNA molecule and the plasmid encoding the tricistronic self-replicating RNA molecule by SEAP-QuantiBlue assay.

FIG. 17 is a bar graph showing differentiation of BMDCs into cDCs and pDCs under various conditions, including treatment with conditioned media from the tricistronic self-replicating RNA molecule and the plasmid encoding the tricistronic self-replicating RNA molecule, compared to treatment with rFLT3L and rGMCSF.

FIGS. 18A and 18B are a schematic diagram showing an exemplary method of synthesizing a circular DNA vector encoding a self-replicating RNA molecule of the invention. The relative organization of each element is depicted. FIG. 18A is a schematic diagram showing individual DNA fragments useful as starting materials. A 5′ sequence having a CAG promoter (P_{CAG}), a nonstructural protein (replicase)-encoding sequence, and a subgenomic promoter (SGP) is divided into three portions. A soluble Flt3 ligand (sFlt3L)-encoding gene, an IL-12-encoding gene, and an XCL1-encoding gene are operatively connected trough furin-P2A sequences. A 3′ polyadenylation signal sequence (polyA) is provided. FIG. 18B is a schematic diagram showing a circular DNA plasmid that is produced upon golden gate assembly of the DNA fragments shown in FIG. 18A. The circular plasmid has a size of about 13 kb. The relative organization of each element is depicted. The DNA vector contains a P_{CAG} to 5′ UTR, a nonstructural protein (replicase)-encoding sequence, an SGP, an sFlt3L-encoding gene, a first furin-P2A sequence, an IL-12-encoding gene, a second furin-P2A sequence, an XCL1-encoding gene, a ribozyme, and a polyA tail.

FIGS. 19A-19E are a schematic diagram showing an exemplary method of synthesizing a self-replicating RNA molecule of the invention. The relative organization of each element is depicted. FIG. 19A is a schematic diagram showing individual DNA fragments useful as starting materials. A 5′ sequence having a T7 promoter (Pr), a nonstructural protein (replicase)-encoding sequence, and a subgenomic promoter (SGP) is divided into three portions. An XCL1-encoding gene, a soluble Flt3 ligand (sFlt3L)-encoding gene, and an IL-12-encoding gene are operatively connected through furin-P2A sequences. A 3′ polyadenylation sequence (AAAA) is provided. FIG. 19B is a schematic diagram showing a circular DNA plasmid that is produced upon golden gate assembly of the DNA fragments shown in FIG. 19A. The circular plasmid has a size of about 13.5 kb. FIG. 19C is a schematic diagram showing a Energized DNA molecule that is produced upon digestion with I-Scel. The linearized DNA molecule has a size of about 10.5 kb. FIG. 19D is a schematic diagram showing a self-replicating RNA molecule that is produced upon in-vitro transcription of the linearized DNA molecule of FIG. 19C. FIG. 19E is a schematic diagram showing a 5′ capped self-replicating RNA molecule having an elongated polyadenylation sequence at its 3′ end.

### DETAILED DESCRIPTION

The present invention involves compositions and methods for treating cancer by administering nucleic acid vectors (e.g., circular DNA vectors) carrying modulatory genes to modulate the tumor microenvironment. The present invention is based, at least in part, on the discovery that nucleic acid vectors (e.g., circular DNA vectors) of the invention can be effective in generating an anti-tumor immune response through delivery of multiple immunomodulatory genes. Compositions and methods of the present invention can effectively modulate the tumor microenvironment through recruitment and/or activation of dendritic cells, which can direct anti-tumor immunity by presenting tumor antigen to lymphocytes (e.g., T cells). In some instances, the nucleic acid vectors (e.g., circular DNA vectors) are administered to an individual having a cancer upon transmitting an electric field to the tumor microenvironment (e.g., by transmitting a pulsed electric field through an intratumorally positioned electrode). In particular embodiments of the invention, the compositions and methods of the invention can mitigate dilution of modulatory gene expression as a result of rapid tumor cell proliferation by providing self-replicating RNA molecules to a tumor microenvironment.

### I. Definitions

Unless defined otherwise, technical and scientific terms used herein have the same meaning as commonly understood by one of ordinary skill in the art to which this invention belongs and by reference to published texts, which provide one skilled in the art with a general guide to many of the terms used in the present application. In the event of any conflicting definitions between those set forth herein and those of a referenced publication, the definition provided herein shall control.

As used herein, the term “circular DNA vector” refers to a DNA molecule in a circular form. Such circular form is typically capable of being amplified into concatamers by rolling circle amplification. A linear double-stranded nucleic acid having conjoined strands at its termini (e.g., covalently conjugated backbones, e.g., by hairpin loops or other structures) is not a circular vector, as used herein. The term “circular DNA vector” Is used interchangeable herein with the term “covalently closed and circular DNA vector.” A skilled artisan will understand that such circular vectors include vectors that are covalently closed with supercoiling and complex DNA topology, as is described herein. In particular embodiments, a circular DNA vector is supercoiled. In certain instances, a circular DNA vector lacks a bacterial origin of replication (e.g., In instances in which the circular DNA vector encodes a self-replicating RNA molecule, the circular DNA vector lacks a bacterial origin of replication and encodes an RNA origin of replication).

As used herein, a “cell-free method” of producing a circular DNA vector refers to a method that does not rely on containment of any of the DNA within a host cell, such as a bacterial (e.g., E. coli) host cell, to facilitate any step of the method, from providing the template DNA vector (e.g., plasmid DNA vector) through producing the therapeutic circular DNA vector). For example, a cell-free method occurs within one or more synthetic containers (e.g., glass or plastic tubes, bioreactors, vessels, tanks, or other suitable containers) within appropriate solutions (e.g., buffered solutions), to which enzymes and other agents may be added to facilitate DNA amplification, modification, and isolation.

As used herein, the terms “recombination site” and “site-specific recombination recognition site” each refers to a nucleic acid sequence that is a product of site-specific recombination, which includes a first sequence that corresponds to a portion of a first recombinase attachment site and a second sequence that corresponds to a portion of a second recombinase attachment site. One example of a hybrid recombination site is attR, which is a product of site-specific recombination and includes a first sequence that corresponds to a portion of attP and a second sequence that corresponds to a portion of attB. Alternatively, recombination sites can be generated from Cre/Lox recombination. Thus, a vector generated from Cre/Lox recombination (e.g., a vector including a LoxP site) includes a recombination site, as used herein. Other site-specific recombination events that generate recombination sites involve, e.g., lambda integrase, FLP recombinase, and Kw recombinase. Nucleic acid sequences that result from non-site-specific recombination events (e.g., ITR-mediated intermolecular recombination) are not recombination sites, as defined herein.

As used herein, the term “self-replicating RNA molecule” refers to a self-replicating genetic element comprising an RNA that replicates from one origin of replication. The terms “self-replicating RNA,” “replicon RNA,” and “self-amplifying replicon RNA” are used interchangeably herein.

As used herein, the term “protein” refers to a plurality of amino acids attached to one another through peptide bonds (i.e., as a primary structure), including multimeric (e.g., dimeric, trimeric, etc.) proteins that are non-covalently associated (e.g., proteins having quaternary structure). Thus, the term “protein” encompasses peptides, native proteins, recombinant proteins, and fragments thereof. In some embodiments, a protein has a primary structure and no secondary, tertiary, or quaternary structure in physiological conditions. In some embodiments, a protein has a primary and secondary structure and no tertiary or quaternary structure in physiological conditions. In particular embodiments, a protein has a primary structure, a secondary structure, and a tertiary structure, but no quaternary structure in physiological conditions (e.g., a monomeric protein having one or more folded alpha-helices and/or beta sheets). In some embodiments, any of the proteins described herein have a length of at least 25 amino acids (e.g., 50 to 1,000 amino acids).

As used herein, the term “modulatory sequence” refers to a nucleic acid sequence that encodes one or more proteins that engage and modulate a cell by binding and signaling through a receptor expressed thereby (e.g., a surface receptor). Modulatory sequences may be monocistronic or polycistronic (e.g., bicistronic or tricistronic).

As used herein, the term “immunomodulatory sequence” refers to a nucleic acid sequence that encodes one or more proteins that engage and modulate an immune cell (e.g., an innate immune cell or an adaptive immune cell), e.g., by binding and signaling through a receptor (e.g., a surface receptor) on the immune cell. Immunomodulatory sequences may be monocistronic or polycistronic (e.g., bicistronic or tricistronic).

As used herein, the term “polycistronic” refers to a nucleic acid sequence (e.g., a DNA vector or RNA sequence) that includes more than one protein-encoding gene downstream of a single promoter (i.e., one promoter drives expression of more than one protein). Polycistronic sequences include bicistronic sequences and tricistronic sequences. In some instances, a polycistronic sequence includes two, three, four, or more immunomodulatory protein-encoding genes. Methods of synthesizing polycistronic nucleic acid sequences are known in the art. In some instances, multiple protein-encoding genes are separated by termination signals and/or cleavage sites (e.g., furin-P2A sites).

The terms “first,” “second,” and “third,” as used herein to identify promoters in a nucleic acid vector, do not describe the positioning of the promoters on a vector, i.e., a first, second, and third promoter may be arranged 5′ to 3′ orientation, but need not be. For instance, a nucleic acid vector that comprises a first, second, and third promoter driving expression of XCL1, FLT3L, and IL-12, respectively, covers each of the following 5′ to 3′ orientations: Promoter (P)-XCL1-P-FLT3L-P-IL-12; P-FLT3L-P-IL-12-P-XCL1; P-IL-12-P-XCL1-P-FLT3L; P-XCL1-P-IL-12-P-FLT3L; P-FLT3L-P-XCL1-P-IL-12; and P-IL-12-P-FLT3L-P-XCL1. A first, second, and third promoter may be the same promoter (e.g., CAG).

As used herein, the term “dendritic cell chemoattractant” refers to a protein that attracts (e.g., promotes infiltration of) a dendritic cell (e.g., a conventional dendritic cell (e.g., cDC1 or cDC2) or a plasmacytoid DC (e.g., pDC)) upon binding to a receptor expressed thereon (e.g., by binding to a dendritic cell surface receptor). Dendritic call chemoattractants can promote infiltration of dendritic cells to a region of tissue by forming a concentration gradient across which dendritic cells travel toward high concentration of the chemoattractant. In some embodiments, the dendritic cell chemoattractant is an inflammatory dendritic cell chemoattractant. Exemplary dendritic cell chemoattractants include XCL1, XCL2, CCL5, and CCL4.

As used herein, “XCL1” refers to any native XCL1 (a member of the C-chemokine subfamily, also known as lymphotactin, Ltn, ATAC, SCYC-α, or SCYC-1) from any vertebrate source, including mammals such as primates (e.g., human and cynomolgus monkeys) and rodents (e.g., mice and rats), unless otherwise indicated, as well as functionally equivalent or improved variants (e.g., natural or synthetic variants), e.g., mutants, muteins, analogs, subunits, receptor complexes, isotypes, spice variants, and fragments thereof. Functionally equivalent and improved variants can be determined on the basis XCR1 signaling (e.g., through XCL1 binding to DC-expressed XCR1). In some embodiments, a functionally equivalent or improved variant exhibits improved stability (e.g., a mutein that confers structural (e.g., folding) stability of XCL1). XCL1 encompasses full-length, unprocessed XCL1, as well as any form of XCL1 that results from native processing in the cell. An exemplary human XCL1 sequence is provided as National Center for Biotechnology Information (NCBI) Gene ID: 6375. In some instances, the XCL1 is encoded by a heterologous gene having at least 95% sequence identity to SEQ ID NO: 9 or 9A (e.g., at least 96%, at least 97%, at least 98%, at least 99% or 100% sequence identity to SEQ ID NO: 9 or 9A). In some instances, the XCL1 encoded by the heterologous gene has at least 95% sequence identity to SEQ ID NO: 10 (e.g., at least 96%, at least 97%, at least 98%, at least 99%, or 100% sequence identity to SEQ ID NO: 10).

As used herein, “XCL2” refers to any native XCL2 (a member of the C-chemokine subfamily, also known as SCM1-β or SCYC-2) from any vertebrate source, including mammals such as primates (e.g., human and cynomolgus monkeys) and rodents (e.g., mice and rats), unless otherwise indicated, as well as functionally equivalent or improved variants (e.g., natural or synthetic variants), e.g., mutants, muteins, analogs, subunits, receptor complexes, isotypes, splice variants, and fragments thereof. Functionally equivalent and improved variants can be determined on the basis XCR2 signaling (e.g., through XCL2 binding to DC-expressed XCR2). In some embodiments, a functionally equivalent or improved variant exhibits improved stability (e.g., a mutein that confers structural (e.g., folding) stability of XCL2). XCL2 encompasses full-length, unprocessed XCL2, as well as any form of XCL2 that results from native processing in the cell. An exemplary human XCL2 sequence is provided as National Center for Biotechnology Information (NCBI) Gene ID: 6846. In some instances, the XCL2 is encoded by a heterologous gene having at least 95% sequence identity to SEQ ID NO: 11 or 11A (e.g., at least 96%, at least 97%, at least 98%, at least 99% or 100% sequence identity to SEQ ID NO: 11 or 11A). In some instances, the XCL1 encoded by the heterologous gene has at least 95% sequence identity to SEQ ID NO: 12 (e.g., at least 96%, at least 97%, at least 98%, at least 99%, or 100% sequence identity to SEQ ID NO: 12).

As used herein, “CCL5” refers to any native CCL5 (also known as regulated on activation, normal T cell expressed and secreted (RANTES)) from any vertebrate source, including mammals such as primates (e.g., human and cynomolgus monkeys) and rodents (e.g., mice and rats), unless otherwise indicated, as well as functionally equivalent or improved variants (e.g., natural or synthetic variants), e.g., mutants, muteins, analogs, subunits, receptor complexes, isotypes, splice variants, and fragments thereof. Functionally equivalent and improved variants can be determined on the basis of CCR5 signaling and/or known downstream effects thereof (e.g., CCR5-associated migration and/or tumor infiltration by dendritic cells, eosinophils, basophils, monocytes, effector memory T cells, B cells, or NK cells). In some embodiments, a functionally equivalent or improved variant exhibits improved stability. CCL5 encompasses full-length, unprocessed CCL5, as well as any form of CCL5 that results from native processing in the cell. An exemplary human CCL5 protein sequence is provided as UniProt Accession No. P13501. In some instances, the CCL5 is encoded by a heterologous gene having at least 95% sequence identity to SEQ ID NO: 13 or 13A (e.g., at least 96%, at least 97%, at least 98%, at least 99% or 100% sequence identity to SEQ ID NO: 13 or 13A). In some instances, the CCL5 encoded by the heterologous gene has at least 95% sequence identity to SEQ ID NO: 14 (e.g., at least 96%, at least 97%, at least 98%, at least 99%, or 100% sequence identity to SEQ ID NO: 14).

As used herein, “CCL4” refers to any native CCL4 (also known as macrophage inflammatory protein-1p (MIP-1p)) from any vertebrate source, including mammals such as primates (e.g., human and cynomolgus monkeys) and rodents (e.g., mice and rats), unless otherwise indicated, as well as functionally equivalent or improved variants (e.g., natural or synthetic variants), e.g., mutants, muteins, analogs, subunits, receptor complexes, isotypes, splice variants, and fragments thereof. Functionally equivalent and improved variants can be determined on the basis of CCR5 receptor signaling and/or known downstream effects thereof (e.g., CCR5-associated migration and/or tumor infiltration by dendritic cells, eosinophils, basophils, monocytes, effector memory T cells, B cells, or NK cells). In some embodiments, a functionally equivalent or improved variant exhibits improved stability. CCL4 encompasses full-length, unprocessed CCL4, as well as any form of CCL4 that results from native processing in the cell. An exemplary human CCL4 protein sequence is provided as UniProt Accession No. P13236. In some instances, the CCL4 is encoded by a heterologous gene having at least 95% sequence identity to SEQ ID NO: 15 or 15A (e.g., at least 96%, at least 97%, at least 98%, at least 99% or 100% sequence identity to SEQ ID NO: 15 or 15A). In some instances, the CCL4 encoded by the heterologous gene has at least 95% sequence identity to SEQ ID NO: 16 (e.g., at least 96%, at least 97%, at least 98%, at least 99%, or 100% sequence identity to SEQ ID NO: 16).

As used herein, the term “dendritic cell growth factor or activator” refers to a protein that promotes differentiation, activation, and/or mobilization of a dendritic cell (e.g., a conventional dendritic cell (e.g., cDC1 or cDC2) or a plasmacytoid DC (e.g., pDC)) upon binding to a receptor expressed thereon (e.g., by binding to a dendritic cell surface receptor). Dendritic cell growth factors and activators include FLT3L, GM-CSF, CD40, and CD40L. As used herein, FLT3L-encoding genes (e.g., sFLT3L-encoding genes), GM-CSF-encoding genes, and CD40- or CD40L-encoding genes are dendritic cell growth factor or activator-encoding genes.

As used herein, “Fms-related receptor tyrosine kinase 3 Ligand (FLT3L)” refers to any native FLT3L from any vertebrate source, including mammals such as primates (e.g., human and cynomolgus monkeys) and rodents (e.g., mice and rats), unless otherwise indicated, as well as functionally equivalent or improved variants (e.g., natural or synthetic variants), e.g., mutants, muteins, analogs, subunits, receptor complexes, isotypes, splice variants, and fragments thereof. FLT3L encompasses soluble FLT3L (sFLT3L). Functionally equivalent and improved variants can be determined on the basis of tyrosine kinase III receptor signaling and/or known downstream effects thereof (e.g., FLT3L-associated hematopoietic regulation). In some embodiments, a functionally equivalent or improved variant exhibits improved stability. FLT3L encompasses full-length, unprocessed FLT3L, as well as any form of FLT3L that results from native processing in the cell. An exemplary human FLT3L protein sequence is provided as UniProt Accession No. P49771. In some instances, the sFLT3L is encoded by a heterologous gene having at least 95% sequence identity to SEQ ID NO: 17 or 17A (e.g., at least 96%, at least 97%, at least 98%, at least 99% or 100% sequence identity to SEQ ID NO: 17 or 17A). In some instances, the FLT3L encoded by the heterologous gene has at least 95% sequence identity to SEQ ID NO: 18 (e.g., at least 96%, at least 97%, at least 98%, at least 99%, or 100% sequence identity to SEQ ID NO: 18).

As used herein, “granulocyte-macrophage colony-stimulating factor (GM-CSF)” refers to any native GM-CSF (also known CSF2) from any vertebrate source, including mammals such as primates (e.g., human and cynomolgus monkeys) and rodents (e.g., mice and rats), unless otherwise indicated, as well as functionally equivalent or improved variants (e.g., natural or synthetic variants), e.g., mutants, muteins, analogs, subunits, receptor complexes, isotypes, splice variants, and fragments thereof. Functionally equivalent and improved variants can be determined on the basis of CSF2R receptor signaling and/or known downstream effects thereof (e.g., JAK-2 recruitment and activation, STATS phosphorylation, pim-1 and/or CIS transcription, PI3K signaling, and/or JAK/STAT-Bcl-2 signaling). In some embodiments, a functionally equivalent or improved variant exhibits improved stability. GM-CSF encompasses full-length, unprocessed GM-CSF, as well as any form of GM-CSF that results from native processing in the cell. An exemplary human GM-CSF protein sequence is provided as UniProt Accession No. P04141. In some instances, the GM-CSF is encoded by a heterologous gene having at least 95% sequence identity to SEQ ID NO: 19 or 19A (e.g., at least 96%, at least 97%, at least 98%, at least 99% or 100% sequence identity to SEQ ID NO: 19 or 19A). In some instances, the GM-CSF encoded by the heterologous gene has at least 95% sequence identity to SEQ ID NO: 20 (e.g., at least 96%, at least 97%, at least 98%, at least 99%, or 100% sequence identity to SEQ ID NO: 20).

As used herein, “duster of differentiation 40 ligand (CD40L)” refers to refers to any native CD40L from any vertebrate source, including mammals such as primates (e.g., human and cynomolgus monkeys) and rodents (e.g., mice and rats), unless otherwise indicated, as well as functionally equivalent or improved variants (e.g., natural or synthetic variants), e.g., mutants, muteins, analogs, subunits, receptor complexes, isotypes, splice variants, and fragments thereof. Functionally equivalent and improved variants can be determined on the basis of CD40/TNFRSF5 signaling and/or known downstream effects thereof (e.g., T cell costimulation in conjunction with T cell receptor (TCR) binding, IL-4 and/or IL-10 production, NFκB activation, MAPK8 activation in T cells, and or PAK2 activation in T cells). In some embodiments, a functionally equivalent or improved variant exhibits improved stability. CD40L encompasses full-length, unprocessed CD40L, as well as any form of CD40L that results from native processing in the cell. An exemplary human CD40L protein sequence is provided as Uniprot Accession No. P29965. In some instances, the CD40L is encoded by a heterologous gene having at least 95% sequence identity to SEQ ID NO: 21 or 21A (e.g., at least 96%, at least 97%, at least 98%, at least 99% or 100% sequence identity to SEQ ID NO: 21 or 21A). In some instances, the CD40L encoded by the heterologous gene has at least 95% sequence identity to SEQ ID NO: 22 (e.g., at least 96%, at least 97%, at least 98%, at least 99%, or 100% sequence identity to SEQ ID NO: 22).

As used herein, the term “lymphocyte signaling protein” refers to a protein that signals to a lymphocyte upon binding to a receptor expressed thereon (e.g., by binding to a lymphocyte surface receptor). In some instances, a lymphocyte signaling protein triggers activation and/or infiltration (e.g., tumor infiltration) of lymphocytes, such as T calls and/or NK cells. In some instances, the lymphocyte signaling protein is a soluble lymphocyte signaling protein. Lymphocyte signaling proteins include cytokines and chemokines.

The term “cytokine” refers to a soluble protein that acts on an immune cell (e.g., an innate or an adaptive immune cell) as an intercellular mediator (e.g., as a paracrine signal). Exemplary cytokines include interleukins (ILs) such as IL-1, IL-1a, IL-2, IL-3, IL-4, IL-5, IL-6, IL-7, IL-8, IL-9, IL-11, IL-12, IL-13, IL-14, IL-15, IL-16, IL-17, IL-18, IL-21, and IL-36-γ; tumor necrosis factors such as TNF-α or TNF-β; interferons (IFNs), such as IFN-γ and IFN-α; and other protein factors including leukemia inhibitory factor (LIF) and kit ligand (KL). In some instances, a cytokine triggers activation of lymphocytes, such as T cells and/or NK cells.

As used herein, the term “chemokine” refers to a soluble protein released by a cell that acts on an immune cell as to selectively induce chemotaxis and activation (e.g., tumor infiltration and activation). Chemokines may also trigger angiogenesis, inflammation, wound healing, and tumorgenesis. In some instances, chemokines are inflammatory. Exemplary chemokines include CXCL9 and CXCL10. Other non-limiting examples of chemokines useful as part of the present invention include CCL14, CCL19, CCL20, CCL21, CCL25, CCL27, CXCL12, CXCL13, CXCL-8, CCL2, CCL3, CCL4, CCL5, CCL11, and CXCL10.

As used herein, “interleukin-12 (IL-12),” as used herein, refers to any native IL-12 (e.g., IL-12 p35/p40 heterodimer; IL-12 p70) from any vertebrate source, including mammals such as primates (e.g., human and cynomolgus monkeys) and rodents (e.g., mice and rats), unless otherwise indicated, as well as functionally equivalent or improved variants (e.g., natural or synthetic variants), e.g., mutants, muteins, analogs, subunits, receptor complexes, isotypes, splice variants, and fragments thereof. Functionally equivalent and improved variants can be determined on the basis of specific proinflammatory and/or Th1-skewing effects known in the art (e.g., induction of IFN-γ, e.g., from T cell and/or NK calls). In some embodiments, a functionally equivalent or improved variant exhibits improved stability. IL-12 encompasses full-length, unprocessed IL-12, as well as any form of IL-12 that results from native processing in the cell. IL-12 encompasses synthetic fusions of IL-12 subunits p35 and p40 (e.g., through a linker). In some instances, the IL-12 Is encoded by a heterologous gene having at least 95% sequence identity to SEQ ID NO: 23 or 23A (e.g., at least 96%, at least 97%, at least 98%, at least 99% or 100% sequence identity to SEQ ID NO: 23 or 23A). In some instances, the IL-12 encoded by the heterologous gene has at least 95% sequence identity to SEQ ID NO: 24 (e.g., at least 96%, at least 97%, at least 98%, at least 99%, or 100% sequence identity to SEQ ID NO: 24).

As used herein, “interleukin-15 (IL-15),” as used herein, refers to any native IL-15 from any vertebrate source, including mammals such as primates (e.g., human and cynomolgus monkeys) and rodents (e.g., mice and rats), unless otherwise indicated, as well as functionally equivalent or improved variants (e.g., natural or synthetic variants), e.g., mutants, muteins, analogs, subunits, receptor complexes, isotypes, splice variants, and fragments thereof. Functionally equivalent and improved variants can be determined on the basis of IL-15R signaling pathway activation (e.g., JAK kinase activation, STAT3 activation, STATS activation, and/or STAT6 activation; and/or T cell and NK cell activation). In some embodiments, a functionally equivalent or improved variant exhibits improved stability. IL-15 encompasses full-length, unprocessed IL-15, as well as any form of IL-15 that results from native processing in the cell. IL-15 encompasses synthetic fusions of IL-15 and its receptor. In some instances, the IL-15 is encoded by a heterologous gene having at least 95% sequence identity to SEQ ID NO: 25 or 25A (e.g., at least 98%, at least 97%, at least 98%, at least 99% or 100% sequence identity to SEQ ID NO: 25 or 25A). In some instances, the IL-15 encoded by the heterologous gene has at least 95% sequence identity to SEQ ID NO: 26 (e.g., at least 96%, at least 97%, at least 98%, at least 99%, or 100% sequence identity to SEQ ID NO: 26).

As used herein, “chemokine (C—X—C motif) ligand 9 (CXCL9)” refers to any native CXCL9 (also known as monokine induced by gamma interferon (MIG)) from any vertebrate source, including mammals such as primates (e.g., human and cynomolgus monkeys) and rodents (e.g., mice and rats), unless otherwise indicated, as well as functionally equivalent or improved variants (e.g., natural or synthetic variants), e.g., mutants, muteins, analogs, subunits, receptor complexes, isotypes, spice variants, and fragments thereof. Functionally equivalent and improved variants can be determined on the basis of CXCR3 receptor interactions. In some embodiments, a functionally equivalent or improved variant exhibits improved stability. CXCL9 encompasses full-length, unprocessed CXCL9, as well as any form of CXCL9 that results from native processing in the cell. An exemplary human CXCL9 protein sequence is provided as UniProt Accession No. 007325. In some instances, the CXCL9 is encoded by a heterologous gene having at least 95% sequence identity to SEQ ID NO: 27 or 27A (e.g., at least 96%, at least 97%, at least 98%, at least 99% or 100% sequence identity to SEQ ID NO: 27 or 27A). In some instances, the CXCL9 encoded by the heterologous gene has at least 95% sequence identity to SEQ ID NO: 28 (e.g., at least 96%, at least 97%, at least 98%, at least 99%, or 100% sequence identity to SEQ ID NO: 28).

As used herein, “chemokine (C—X—C motif) ligand 10 (CXCL10)” refers to any native CXCL10 (also known as monokine induced by gamma interferon (MIG)) from any vertebrate source, including mammals such as primates (e.g., human and cynomolgus monkeys) and rodents (e.g., mice and rats), unless otherwise indicated, as well as functionally equivalent or improved variants (e.g., natural or synthetic variants), e.g., mutants, muteins, analogs, subunits, receptor complexes, isotypes, spice variants, and fragments thereof. Functionally equivalent and improved variants can be determined on the basis of CXCR3 receptor interactions. In some embodiments, a functionally equivalent or improved variant exhibits improved stability. CXCL10 encompasses full-length, unprocessed CXCL10, as well as any form of CXCL10 that results from native processing in the cell. An exemplary human CXCL10 protein sequence is provided as Uniprot Accession No. P02778. In some instances, the CXCL10 Is encoded by a heterologous gene having at least 95% sequence identity to SEQ ID NO: 29 or 29A (e.g., at least 96%, at least 97%, at least 98%, at least 99% or 100% sequence identity to SEQ ID NO: 29 or 29A). In some instances, the CXCL10 encoded by the heterologous gene has at least 95% sequence identity to SEQ ID NO: 30 (e.g., at least 96%, at least 97%, at least 98%, at least 99%, or 100% sequence identity to SEQ ID NO: 30).

As used herein, the term “therapeutic sequence” refers to the portion of a DNA molecule (e.g., a plasmid DNA vector or a concatemer thereof) that contains any genetic material required for transcription in a target cell of one or more therapeutic moieties, which may include one or more coding sequences, promoters, terminators, introns, and/or other regulatory elements. A therapeutic moiety can be a therapeutic protein (e.g., a replacement protein (e.g., a protein that replaces a defective protein in the target cell) or an endogenous protein (e.g., an immunomodulatory protein, such as a cytokine)) and/or a therapeutic nucleic acid (e.g., one or more microRNAs). In DNA vectors having more than one transcription unit, the therapeutic sequence contains the plurality of transcription units. A therapeutic sequence may include one or more genes (e.g., heterologous genes or transgenes) to be administered for a therapeutic purpose.

The term “heterologous gene” refers to a transgene to be administered (e.g., as part of a DNA vector or self-replicating RNA molecule). A heterologous gene can be a mammalian gene that is endogenously expressed by the individual receiving the heterologous gene (e.g., a heterologous modulatory (e.g., immunomodulatory) protein-encoding gene). As used herein, a “variant” of a heterologous gene, a replicase, or a fragment thereof, differs in at least one amino acid residue from the reference amino acid sequence, such as a naturally occurring amino acid sequence or an amino acid sequence. In this context, the difference in at least one amino acid residue may consist, for example, in a mutation of an amino acid residue to another amino acid, a deletion or an insertion. A variant may be a homolog, Isoform, or transcript variant of a therapeutic protein or a fragment thereof as defined herein, wherein the homolog, isoform or transcript variant is characterized by a degree of identity or homology, respectively, as defined herein.

In some instances, a variant of a heterologous gene, a replicase, or a fragment thereof, includes at least one amino acid substitution (e.g., 1-100 amino acid substitutions, 1-50 amino acid substitutions, 1-20 amino acid substitutions, 1-10 amino acid substitutions, e.g., 1 amino acid substitution, 2 amino acid substitutions, 3 amino acid substitutions, 4 amino acid substitutions, 5 amino acid substitutions, 6 amino acid substitutions, 7 amino acid substitutions, 8 amino acid substitutions, 9 amino acid substitutions, or 10 amino acid substitutions). Substitutions in which amino acids from the same class are exchanged for one another are called conservative substitutions. In particular, these are amino acids having aliphatic side chains, positively or negatively charged side chains, aromatic groups in the side chains or amino acids, the side chains of which can form hydrogen bridges, e.g., side chains which have a hydroxyl function. By conservative constitution, e.g., an amino acid having a polar side chain may be replaced by another amino acid having a corresponding polar side chain, or, for example, an amino acid characterized by a hydrophobic side chain may be substituted by another amino acid having a corresponding hydrophobic side chain (e.g., serine (threonine) by threonine (serine) or leucine (isoleucine) by isoleucine (leucine)). In certain embodiments, a variant of a protein or a fragment thereof may be encoded by the RNA according to the invention, wherein at least one amino acid residue of the amino acid sequence includes at least one conservative substitution compared to a reference sequence, such as the respective naturally occurring sequence.

In some instances, Insertions, deletions, and/or non-conservative substitutions are also encompassed by the term variant. e.g., at those positions that do not cause a substantial modification of the three-dimensional structure of the protein. Modifications to a three-dimensional structure by insertion(s) or deletion(s) can readily be determined by a person of skill in the art, e.g., using CD spectra (circular dichroism spectra).

In order to determine the percentage to which two sequences (e.g., nucleic acid sequences, e.g., DNA, RNA, or amino acid sequences) are identical, the sequences can be aligned in order to be subsequently compared to one another. For this purpose, gaps can be inserted into the sequence of the first sequence and the component at the corresponding position of the second sequence can be compared. If a position in the first sequence is occupied by the same component as is the case at a corresponding position in the second sequence, the two sequences are identical at this position. The percentage, to which two sequences are identical, is a function of the number of identical positions divided by the total number of positions. The percentage to which two sequences are identical can be determined using a mathematical algorithm. A preferred, but not limiting, example of a mathematical algorithm, which can be used is the algorithm of Kadin et al. (1993), PNAS USA, 90:5873-5877 or Altschul et al. (1997), Nucleic Acids Res., 25:3389-3402. Such an algorithm can be integrated, for example, in the BLAST program. Sequences, which are identical to the sequences of the present invention to a certain extent, can be identified by this program. In any embodiments described herein, a sequence may have at least 80%, 85%, 90%, 95%, 96%, 97%, 98%, 99%, or 100% sequence identity to a particular SEQ ID NO: recited thereto (e.g., any one of SEQ ID NOs: 1-36). One of skill in the art understands that, by virtue of the genetic code, a DNA sequence as described herein allows deduction of a corresponding RNA sequences and likewise, an RNA sequence allows the deduction of a corresponding DNA sequence. All sequences described herein are listed in Table 1.

As used herein, the term ‘operably linked’ or “operatively linked” refers to an arrangement of elements, wherein the components so described are configured so as to perform their usual function. A nucleic acid is “operably linked” or “operatively linked” when it is placed into a functional relationship with another nucleic acid sequence. For example, a promoter is operably linked to one or more heterologous genes if it affects the transcription of the one or more heterologous genes. Further, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence and the promoter sequence can still be considered “operably inked” to the coding sequence.

As used herein, the term “isolated” means artificially produced and not integrated into a native host genome. For example, an isolated nucleic acid vector includes nucleic acid vectors that are encapsulated in a lipid envelope (e.g., a liposome) or a polymer matrix. In some embodiments, the term “Isolated” refers to a DNA vector that is: (i) amplified in vitro (e.g., in a cell-free environment), for example, by rolling-circle amplification or polymerase chain reaction (PCR); (ii) recombinantly produced by molecular cloning; (iii) purified, as by restriction endonuclease cleavage and gel electrophoretic fractionation, or column chromatography; or (iv) synthesized by, for example, chemical synthesis. An isolated nucleic acid vector is one which is readily manipulable by recombinant DNA techniques well-known in the art. Thus, a nucleotide sequence contained in a vector in which 5′ and 3′ restriction sites are known or for which polymerase chain reaction (PCR) primer sequences have been disclosed is considered isolated, but a nucleic acid sequence existing in Its native state in Its natural host is not. An isolated nucleic acid vector may be substantially purified, but need not be. An isolated self-replicating molecule is one which is readily manipulable by recombinant techniques well-known in the art. An isolated self-replicating RNA molecule may be substantially purified, but it need not be. For example, isolated self-replicating RNA molecules include self-replicating RNA molecules that are encapsulated in a lipid envelope (e.g., a liposome) or a polymer matrix.

As used herein, the term “naked” refers to a nucleic acid molecule (e.g., a circular DNA vector) that is not encapsulated in a lipid envelope (e.g., a liposome) or a polymer matrix and is not physically associated with (e.g., covalently or non-covalently bound to) a solid structure (e.g., a particulate structure) upon administration to the individual. In some instances of the present invention, a pharmaceutical composition includes a naked circular DNA vector (e.g., a naked synthetic circular DNA vector).

The term “pharmaceutical composition” refers to an agent which is in a form such that the biological activity of one or more active ingredients contained therein is effective and does not contain additional ingredients that are unacceptably toxic to the patient to which the formulation is administered. A pharmaceutical composition may include a pharmaceutically acceptable carrier (e.g., In liquid or In dry (e.g. lyophilized) form).

The phrase “pharmaceutically acceptable” indicates that the substance or composition must be compatible chemically and/or toxicologically, with the other ingredients comprising a formulation, and/or the mammal being treated therewith.

The term “carrier” refers to a diluent, adjuvant, excipient, or vehicle with which a vector or composition of the invention is administered. Examples of suitable pharmaceutical carriers are described in “Remington's Pharmaceutical Sciences,” Academic Press., 23^{rd} edition, 2020.

As used herein, a “vector” refers to a nucleic acid molecule capable of carrying a sequence of interest (e.g., a heterologous gene or a therapeutic sequence) to which is it linked into a target cell in which the heterologous gene can then be replicated, processed, and/or expressed in the target cell. After a target cell or host cell processes the sequence of interest (e.g., genome) of the vector, the sequence of interest (e.g., genome) is not considered a vector. One type of vector is a “plasmid”, which refers to a circular double stranded DNA loop containing a bacterial backbone into which additional DNA segments may be ligated. Another type of vector is a phage vector. Another type of vector is a viral vector, wherein additional DNA segments may be ligated into the viral genome. Certain vectors are capable of autonomous replication in a host call info which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply. “recombinant vectors” or “expression vectors”). In general, expression vectors of utility in recombinant DNA techniques are often in the form of plasmids.

As used herein, a “target cell” refers to a cell that expresses a modulatory protein encoded by a heterologous gene. Target cells include tumor cells (i.e., cancer cells) and tumor-resident cells (e.g., healthy cells present in the tumor microenvironment (e.g., tumor-resident antigen-presenting cells, such as dendritic cells, and tumor infiltrating lymphocytes, such as T cells)).

As used herein, the term “individual” includes any mammal in need of the methods of treatment or prophylaxis described herein (e.g., a mammal having cancer). In some embodiments, the individual is a human. In other embodiments, the individual is a non-human mammal (e.g., a non-human primate (e.g., a monkey), a mouse, a pig, a rabbit, a cat, or a dog). The subject may be male or female. In one embodiment, the subject has a cancer, e.g., a cancer characterized by the presence of one or more sold tumors.

“Cancer” refers to abnormal proliferation of malignant cancer cells and includes cancers characterized by the presence of solid tumors such as sarcoma, skin cancer, melanoma, bladder cancer, brain cancer, breast cancer, uterine cancer, ovarian cancer, prostate cancer, lung cancer, colorectal cancer, cervical cancer, liver cancer, head and neck cancer, esophageal cancer, pancreatic cancer, kidney cancer, adrenal cancer, gastric cancer, testicular cancer, gallbladder cancer and biliary tract cancer, thyroid cancer, thymic cancer, bone cancer, and brain cancer. Cancer cells (e.g., tumor cells) in cancer patients may be immunologically distinct from normal somatic cells in the subject (e.g., cancer tumors may be immunogenic). For example, cancer cells can elicit a systemic immune response in cancer patients against one or more antigens expressed by the cancer cells. The antigen that elicits the immune response may be a tumor antigen or may be shared by normal cols. Patients with cancer may exhibit sufficient test results to diagnose cancer according to at least one identifiable indication, symptom or clinical standard known in the art. Examples of such clinical standards are described in medical textbooks, such as, for example, Harrison's Principles of Internal Medicine (Longo DL, Fauci AS, Kasper DL, Hauser SL, Jameson J, Loscalzo J. eds. 18e. New York, NY McGraw-Hill; 2012). In some cases, the diagnosis of cancer in an individual can include the identification of a particular cell type (e.g., cancer cell) in a sample of body fluids or tissue obtained from the individual.

As used herein, a “solid tumor” is any cancer of body tissue other than the blood, bone marrow, or lymphatic system. Solid tumors can be further divided into those of epithelial cell origin and those of non-epithelial con origin. Examples of epithelial cons or solid tumors include tumors of the head and neck, the gastrointestinal tract, colon, breast, prostate, lungs, kidneys, liver, pancreas, ovary, oral cavity, stomach, duodenum, small intestine, large intestine, anus, gallbladder, labia, nasopharynx, skin, uterus, male reproductive organs, urine organs, bladder, and sin. Solid tumors of non-epithelial origin include sarcomas, brain tumors, and bone tumors.

As used herein, “tumor microenvironment” refers to the collection of cells and extracellular material (e.g., matrix proteins, vesicles, and soluble factors (e.g., cytokines)) within a sold tumor.

As used herein, an “effective amount” or “effective dose” of a nucleic acid vector or composition thereof refers to an amount sufficient to achieve a desired biological and/or pharmacological effect, e.g., when administered to the individual according to a selected administration form, route, and/or schedule. As will be appreciated by those of ordinary skill in this art, the absolute amount of a particular composition that is effective can vary depending on such factors as the desired biological or pharmacological endpoint, the agent to be delivered, the target tissue, etc. Those of ordinary skill in the art will further understand that an “effective amount” can be contacted with calls or administered to a subject in a single dose or through use of multiple doses. An effective amount of a composition to treat a cancer may slow or stop disease progression (e.g., tumor growth and/or metastasis) Increase partial or complete response, increase overall survival (e.g., relative to a reference population, e.g., an untreated or placebo population).

As used herein, “treatment” (and grammatical variations thereof such as “treat” or “treating”) refers to clinical intervention in an attempt to alter the natural course of the individual being treated, which can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, nucleic acid vectors (e.g., circular DNA vectors) of the invention are used to delay development of a disease or to slow the progression of a disease.

In particular, the treatment may comprise reducing or inhibiting cancer growth, including complete cancer remission and/or inhibition of cancer metastasis. Cancer growth generally refers to any one of a number of indices that point to changes in cancer in a more developed form. Thus, indices for measuring inhibition of cancer growth may be reduced in cancer cell viability, in tumor volume or in form (e.g., as determined using computed tomography (CT), ultrasonography, or other imaging methods). Delayed tumor growth, disruption of the tumor vasculature, improved performance in delayed hypersensitivity skin tests, increased cytolytic T-lymphocyte activity and a decrease in tumor specific antigen levels. Reducing immune suppression in a cancerous tumor in a subject can improve the ability of the subject to resist cancer growth, in particular, the growth of a cancer already present in the subject and/or to reduce the propensity for cancer growth in the subject.

By “reduce or inhibit” is meant the ability to cause an overall decrease preferably of 20% or greater, more preferably of 50% or greater, and most preferably of 75%, 85%, 90%, 95%, or greater. Reduce or Inhibit can refer to the symptoms of the disorder being treated, the presence or size of metastases, the size of the primary tumor.

As used herein, “modulating,” or “to modulate,” a tumor microenvironment means to cause a measurable change in any tumor biomarker known to correlate with, or influence (i.e., directly or indirectly) the balance between tumor progression and tumor clearance. For example, a tumor microenvironment is modulated by a treatment if the treatment increases the relative expression of a biomarker associated with tumor clearance, e.g., an immunogenic protein in the tumor microenvironment (e.g., a pro-inflammatory cytokine or a dendritic cell activating protein, or a protein expressed on the surface of a cell, such as an activated tumor infiltrating lymphocyte) and/or an immunogenic cell profile in the tumor microenvironment (e.g., an increased presence of tumor-Infiltrating lymphocytes). In some embodiments, a tumor microenvironment is said to be modulated by a treatment if the treatment decreases the relative expression of a biomarker associated with tumor progression (e.g., a biomarker of T cell exhaustion, e.g., PD-1 or PD-L1 expression in a tumor microenvironment). Biomarkers associated with tumor clearance or tumor progression can be determined by any suitable means, e.g., Western blot, immunohistochemistry (IHC), polymerase chain reaction (PCR), singe-cell sequencing, flow cytometry, meso-scale discovery (MSD), or other conventional assays, e.g., performed on a biopsy of the tumor microenvironment or blood (e.g., circulating cells, plasma, or serum). In some embodiments, a measurable change is any statistically significant change, e.g., in expression level of an immunogenic protein or other biomarker associated with tumor progression. For example, a measurable change in expression level may be a change of at least 10%, at least 20%, at least 30%, at least 50%, at least 75%, or at least 90% change in expression level of an immunogenic protein or other biomarker associated with tumor progression relative to a reference expression level (e.g., expression in a reference cell, tissue, or subject without treatment or prior to treatment). In some instances, a tumor microenvironment is determined to have been modulated by a treatment if the treatment extends progression-free survival, results in an objective response, including a partial response or a complete response. Increases overall survival time, and/or improves one or more symptoms of the cancer.

As used herein, the term “biomarker” refers to a protein, DNA, RNA, carbohydrate, or glycolipid-based molecular marker, the expression or presence of which in an individual's, subject's, or patient's sample can be detected by standard methods (or methods disclosed herein) and is useful for monitoring the responsiveness or sensitivity of a tumor environment to a circular DNA vector, or composition thereof. Expression of such a biomarker may be determined to be higher or lower in a sample obtained from an individual treated according to a method of the present invention relative to a reference level (including, e.g., the median expression level of the biomarker in a sample from a group/population of patients, e.g., patients having a solid tumor the median expression level of the biomarker in a sample from a group/population of patients, e.g., patients having solid tumors; or the level in a sample previously obtained from the individual at a prior time. Individuals having an expression level that is greater than or less than the reference expression level of the biomarker can be characterized as individuals having tumor microenvironments that have been modulated by treatment. For example, such individuals who exhibit gene expression levels at the most extreme 50%, 45%, 40%, 35%, 30%, 25%, 20%, 15%, 10%, or 5% relative to (i.e., higher or lower than) the reference level (such as the median level, noted above), can be identified as individuals having tumor microenvironments that have been modulated by treatment.

The terms “level of expression” or “expression level” are used interchangeably and generally refer to the amount of a polynucleotide or an amino acid product or protein in a biological sample (e.g., a tumor sample). ‘Expression’ generally refers to the process by which gene-encoded information is converted into the structures present and operating in the call. Therefore, according to the invention, “expression” of a gene may refer to transcription into a polynucleotide, translation into a protein, or post-translational modification of the protein. Fragments of the transcribed polynucleotide, the translated protein, or the post-translationally modified protein shall also be regarded as expressed whether they originate from a transcript generated by alternative spicing or a degraded transcript, or from a post-translational processing of the protein, e.g., by proteolysis. “Expressed genes” Include those that are transcribed into a polynucleotide as mRNA and then translated into a protein, and also those that are transcribed into RNA but not translated into a protein (for example, transfer and ribosomal RNAs).

As used herein, the term “expression persistence” refers to the duration of time during which a sequence of interest (e.g., a heterologous gene or a therapeutic sequence), or a functional portion thereof (e.g., one or more coding sequences of a therapeutic DNA vector), is expressible in the cell in which it was transfected (“intra-cellular persistence”) or any progeny of the cell in which it was transfected (“trans-generational persistence”). A sequence of interest (e.g., a heterologous gene or a therapeutic sequence), or functional portion thereof, may be expressible if it is not silenced, e.g., by DNA methylation and/or histone methylation and compaction. Expression persistence can be assessed by detecting or quantifying (i) mRNA transcribed from the sequence of interest (e.g., a heterologous gene or a therapeutic sequence) in the target cell or progeny thereof (e.g., through qPCR, RNA-seq, or any other suitable method) and (ii) protein translated from the sequence of interest (e.g., a heterologous gene or a therapeutic sequence) in the target cell or progeny thereof (e.g., through Western blot, ELISA, or any other suitable method). In some instances, expression persistence is assessed by detecting or quantifying therapeutic DNA in the target cell or progeny thereof (e.g., the presence of therapeutic circular DNA vector in the target cell, e.g., through episomal DNA copy number analysis) In conjunction with either or both of (i) mRNA transcribed from the sequence of interest (e.g., a heterologous gene or a therapeutic sequence) in the target cell or progeny thereof and (H) protein translated from the sequence of interest (e.g., a heterologous gene or a therapeutic sequence) In the target cell or progeny thereof. Expression persistence of a sequence of interest (e.g., a heterologous gene or a therapeutic sequence), or a functional portion thereof, can be quantified relative to a reference vector, such as a control vector produced in bacteria (e.g., a circular vector produced in bacteria or having one or more bacterial signatures not present in the vector of the invention (e.g., a plasmid)), using any gene expression characterization method known in the art. Expression persistence can be quantified at any given time point following administration of the vector. For example, in some embodiments, expression of a therapeutic circular DNA vector of the invention persists for at least two weeks after administration if it is detectable in the target cell, or progeny thereof, two weeks after administration of the therapeutic circular DNA vector. In some embodiments, expression of a gene “persists” in a target cell if it is detectable in the target cell at one week, two weeks, three weeks, four weeks, six weeks, two months, three months, four months, five months, six months, seven months, eight months, nine months, ten months, eleven months, one year, or longer after administration. In some embodiments, expression of a sequence of interest (e.g., a heterologous gene or a therapeutic sequence) is said to persist for a given period after administration if any detectable fraction of the original expression level remains (e.g., at least 1%, at least 5%, at least 10%, at least 20%, at least 30%, at least 40%, at least 50%, at least 70%, or at least 100% of the original expression level) after the given period of time (e.g., one week, two weeks, three weeks, four weeks, six weeks, two months, three months, four months, five months, six months, seven months, eight months, nine months, ten months, eleven months, one year, or longer after administration).

The terms “sample” and “biological sample” are used interchangeably to refer to any biological sample obtained from an individual including body tissue (e.g., tumor tissue), body fluids, cells, or other sources. Body fluids are, e.g., lymph, sera, whole fresh blood, peripheral blood mononuclear cells, frozen whole blood, plasma (including fresh or frozen), urine, saliva, semen, synovial fluid and spinal fluid. Samples also include breast tissue, renal tissue, colonic tissue, brain tissue, muscle tissue, synovial tissue, skin, hair follicle, bone marrow, and tumor tissue. Methods for obtaining tissue biopsies and body fluids from mammals are well known in the art.

As used herein, “administering” is meant a method of giving a dosage of a nucleic acid vector of the invention or a composition thereof (e.g., a pharmaceutical composition, e.g., a pharmaceutical composition including a nucleic acid vector) to an individual. The compositions utilized in the methods described herein can be administered, for example, intratumorally, peritumorally, intravenously, subcutaneously, intradermally, percutaneously, intramuscularly, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subconjunctivally, intravesicularlly, mucosally, intrapericardially, intraumbilically, intraocularly, intraorbitally, orally, topically, transdermally, periocularly, conjunctivally, subtenonly, intracamerally, subretinally, retrobulbarly, intracanaecularly, by inhalation, by injection, by implantation, by infusion, by continuous infusion, by localized perfusion bathing target cells directly, by catheter, by lavage, in cremes, or in lipid compositions. The compositions utilized in the methods described herein can be administered systemically or locally (e.g., Intratumorally or peritumorally). The method of administration can vary depending on various factors (e.g., the compound or composition being administered and the severity of the condition, disease, or disorder being treated).

To be “administered in combination with” refers to administration of multiple therapeutic components as part of the same therapeutic regimen. A nucleic acid vector (e.g., circular DNA vector) of the invention can be administered in combination with a pulsed electric field therapy, e.g., as part of the same outpatient procedure or over the course of multiple days. Additionally, or alternatively, a nucleic acid vector (e.g., circular DNA vector) of the invention can be administered in combination with another therapeutic agent (e.g., as part of the same pharmaceutical composition or as separate pharmaceutical compositions, at the same time or at different times).

As used herein, “electrotransfer” refers to movement of a molecule (e.g., a nucleic acid vector, e.g., a circular DNA vector) across a membrane of a target cell (e.g., from outside to inside the target cell) that is caused by transmission of an electric field (e.g., a pulsed electric field) to the microenvironment in which the cell resides. Electrotransfer may occur as a result of electrophoresis, i.e., movement of a molecule (e.g., a nucleic acid vector, e.g., a circular DNA vector) along an electric field (e.g., in the direction of current), based on a charge of the molecule. Electrophoresis can induce electrotransfer, for example, by moving a molecule (e.g., a nucleic acid vector, e.g., a circular DNA vector) into proximity of a cell membrane to allow a biotransport process (e.g., endocytosis including pinocytosis or phagocytosis) or passive transport (e.g., diffusion or lipid partitioning) to carry the molecule into the cell. Additionally, or alternatively, electrotransfer may occur as a result of electroporation, i.e., generation of pores in the target cell caused by transmission of an electric field (e.g., a pulsed electric field), wherein the size, shape, and duration of the pores are suitable to accommodate movement of a molecule (e.g., a nucleic acid vector, e.g., a circular DNA vector) from outside the target cell to inside the target cell. Thus, in some instances, electrotransfer occurs as a result of a combination of electrophoresis and electroporation.

A “chemotherapeutic agent” is a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylmelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); combretastatin; folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); decarbazine; mannomustine; mitobronitol; mitolactol; pipobroman; gacytosine; arabinoside (“Ara-C”); thiotepa; taxoid, e.g., paclitaxel (TAXOL®, Bristol-Myers Squibb Oncology, Princeton. N.J.), albumin-engineered nanoparticle formulation of paclitaxel (ABRAXANE®), and docetaxel (TAXOTERE®, Rhome-Poulene Rorer, Antony, France); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylomithine (DMFO); retinoids such as retinoic acid. Including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant call proliferation, such as, for example, PKC-alpha, Raf, H-Ras, and epidermal growth factor receptor (EGF-R) (e.g., erlotinib (Tarceva™)); and VEGF-A that reduce cell proliferation; vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 Inhibitor (e.g. celecoxib or etoricoxib), proteosome inhibitor (e.g. PS341); bortezomib (VELCADE®); CCI-779; tipifamib (R11577); sorafenib, ABT510; Bcl-2 Inhibitor such as oblimersen sodium (GENASENSE®); pixantrone; EGFR inhibitors; tyrosine kinase inhibitors; serine-threonine kinase inhibitors such as rapamycin (sirolimus, RAPAMUNE®); famesyitransferase inhibitors such as lonafamib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN®) combined with 5-FU and leucovorin, and pharmaceutically acceptable salts, acids or derivatives of any of the above; as well as combinations of two or more of the above.

Chemotherapeutic agents as defined herein include “anti-hormonal agents” or“endocrine therapeutics” which act to regulate, reduce, block, or inhibit the effects of hormones that can promote the growth of cancer. They may be hormones themselves, including, but not limited to: anti-estrogens with mixed agonist/antagonist profile, including, tamoxifen (NOLVADEX®), 4-hydroxytamoxifen, toremifene (FARESTON®), idoxifene, droloxifene, raloxifene (EVISTA®), trioxifene, keoxifene, and selective estrogen receptor modulators (SERMs) such as SERM3; pure anti-estrogens without agonist properties, such as fulvestrant (FASLODEX®), and EM800 (such agents may block estrogen receptor (ER) dimerization, inhibit DNA binding, increase ER turnover, and/or suppress ER levels); aromatase inhibitors, including steroidal aromatase inhibitors such as formestane and exemestane (AROMASIN®), and nonsteroidal aromatase inhibitors such as anastrazole (ARIMIDEX®), letrozole (FEMARA®) and aminoglutethimide, and other aromatase inhibitors include vorozole (RIVISOR®), megestrol acetate (MEGASE®), fadrozole, and 4(5)-imidazoles; luteinizing hormone-releasing hormone agonists, including leuprolide (LUPRON® and ELIGARD®), goserelin, buserelin, and triptorelin; sex steroids, including progestines such as megestrol acetate and medroxyprogesterone acetate, estrogens such as diethylstilbestrol and premarin, and androgens/retinoids such as fluoxymesterone, all transretinoic acid and fenretinide; onapristone; anti-progesterones; estrogen receptor down-regulators (ERDs); anti-androgens such as flutamide, nilutamide and bicalutamide; and pharmaceutically acceptable salts, acids or derivatives of any of the above; as well as combinations of two or more of the above.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁵⁰, Re¹⁸⁶, Re¹⁸⁸, Sm¹⁵³, Bi²¹², P³², Pb²¹², and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed herein.

A“growth inhibitory agent” when used herein refers to a compound or composition which inhibits growth of a cell either in vitro or in vivo. Thus, the growth inhibitory agent may be one which significantly reduces the percentage of cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase if inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn et al. eds., The Molecular Basis of Cancer, Chapter 1, entitled “Cell cycle regulation, oncogenes, and antineoplastic drugs” by Murakami et al. (W.B. Saunders, Philadelphia, 1995). e.g., p. 13. Taxanes (pacitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Pacitaxel and docetaxel promote the assembly of microtubules from tubulin dimers and stabilize microtubules by preventing depolymerization, which results in the inhibition of mitosis in cells.

The terms “a” and “an” mean “one or more of.” For example, “a gene” is understood to represent one or more such genes. As such, the terms “a” and “an,” “one or more of a (or an),” and “at least one of a (or an)” are used interchangeably herein.

As used herein, the term “about” refers to a value within ±10% variability from the reference value, unless otherwise specified.

### II. Therapeutic Compositions

The present invention involves nucleic acid vectors (e.g., circular DNA vectors, e.g., naked circular DNA vectors; or self-replicating RNA molecules), and pharmaceutical compositions thereof, which are useful in methods of treating cancer, e.g., through modulation of the tumor microenvironment (e.g., immunomodulation of the tumor microenvironment). Such nucleic acid vectors include circular DNA vectors (e.g., circular DNA vectors having multiple transcription units, each encoding an immunodulatory gene; circular DNA vectors having a single transcription unit encoding multiple immunomodulatory genes; and circular DNA vectors encoding a self-replicating RNA molecule encoding a replicase and one or more immunomodulatory genes). In some instances, the circular DNA vector (e.g., naked circular DNA vector) is a synthetic circular DNA vector, which refers to a circular DNA vector produced using cell-free processes in which their production from templates does not involve bacterial cells.

### Nucleic Acid Vectors

Provided herein are nucleic acid vectors (e.g., DNA vectors, e.g., circular DNA vectors; or self-replicating RNA molecules) having modulatory sequences, wherein each modulatory sequence encodes multiple modulatory genes. In embodiments involving self-replicating RNA molecules, modulatory sequences can be operably linked to a replicase. Modulatory sequences can be immunomodulatory, i.e., capable of modulating immune cells, e.g., by encoding an immunomodulatory protein that binds to the surface of an immune cell to mobilize and/or activate the immune cell. Modulatory sequences (e.g., immunomodulatory sequences) can be monocistronic or polycistronic (i.e., bicistronic, tricistronic, etc.). Immunomodulatory sequences include one or more immunomodulatory protein-encoding genes, which encode peptides and proteins that can bind to various immune cells, e.g., a dendritic cell chemoattractant-encoding gene, a dendritic cell growth-factor encoding gene, and a lymphocyte signaling protein-encoding gene.

In some instances, the immunomodulatory protein binds to and signals through an antigen-presenting cell (e.g., a cross-presenting antigen-presenting cell, e.g., a professional antigen-presenting cell, such as a dendritic cell (e.g., a conventional dendritic cell (e.g., cDC1 or cDC2) or a plasmacytoid DC (e.g., pDC))). Such antigen-presenting cell-binding immunomodulatory proteins include dendritic cell chemoattractants, such as XCL1, XCL2, CCL5, or CCL4. Thus, in some instances of the present invention, the immunomodulatory sequence includes an XCL1-encoding gene (e.g., a gene encoding an amino acid sequence having at least 95% identity to, at least 96% identity to, at least 97% identity to, at least 98% identity to, at least 99% identity to, or 100% identity to SEQ ID NO: 10; and/or a gene having at least 95% identity to, at least 96% identity to, at least 97% identity to, at least 98% identity to, at least 99% identity to, or 100% identity to SEQ ID NO: 9 or 9A), an XCL2-encoding gene (e.g., a gene encoding an amino acid sequence having at least 95% identity to, at least 96% identity to, at least 97% identity to, at least 98% identity to, at least 99% identity to, or 100% Identity to SEQ ID NO: 12; and/or a gene having at least 95% identity to, at least 98% identity to, at least 97% identity to, at least 98% Identity to, at least 99% Identity to, or 100% Identity to SEQ ID NO: 11 or 11A), a CCL5-encoding gene (e.g., a gene encoding an amino acid sequence having at least 95% identity to, at least 96% identity to, at least 97% identity to, at least 98% identity to, at least 99% identity to, or 100% identity to SEQ ID NO: 14; and/or a gene having at least 95% identity to, at least 96% identity to, at least 97% Identity to, at least 98% Identity to, at least 99% identity to, or 100% identity to SEQ ID NO: 13 or 13A), or a CCL4-encoding gene (e.g., a gene encoding an amino acid sequence having at least 95% identity to, at least 96% identity to, at least 97% identity to, at least 98% Identity to, at least 99% identity to, or 100% Identity to SEQ ID NO: 16; and/or a gene having at least 96% identity to, at least 96% Identity to, at least 97% Identity to, at least 98% Identity to, at least 99% Identity to, or 100% Identity to SEQ ID NO: 15 or 15A). Antigen-presenting cell-binding immunomodulatory proteins also include dendritic cell growth factors and activators, such as FLT3L (e.g., soluble FLT3L (sFLT3L)), GM-CSF, CD40, or CD40L). Thus, in some instances of the present invention, the immunomodulatory sequence includes a FLT3L-encoding gene (e.g., a sFLT3L-encoding gene (e.g., a gene encoding an amino acid sequence having at least 95% identity to, at least 96% identity to, at least 97% identity to, at least 98% identity to, at least 99% identity to, or 100% Identity to SEQ ID NO: 18; and/or a gene having at least 95% identity to, at least 96% Identity to, at least 97% identity to, at least 98% identity to, at least 99% identity to, or 100% identity to SEQ ID NO: 17 or 17A)). In other instances, the immunomodulatory sequence includes a GM-CSF-encoding gene (e.g., a gene encoding an amino acid sequence having at least 95% Identity to, at least 96% identity to, at least 97% identity to, at least 98% identity to, at least 99% Identity to, or 100% identity to SEQ ID NO: 20; and/or a gene having at least 95% identity to, at least 96% identity to, at least 97% identity to, at least 98% identity to, at least 99% identity to, or 100% identity to SEQ ID NO: 19 or 19A). In other instances, the immunomodulatory sequence includes a CD40L-encoding gene (e.g., a gene encoding an amino acid sequence having at least 95% identity to, at least 96% identity to, at least 97% identity to, at least 98% identity to, at least 99% identity to, or 100% identity to SEQ ID NO: 22; and/or a gene having at least 95% identity to, at least 96% identity to, at least 97% identity to, at least 98% identity to, at least 99% Identity to, or 100% identity to SEQ ID NO: 21 or 21A). In some instances, the nucleic acid vector features a single promoter driving expression of the multiple genes in the immunomodulatory sequence (e.g., a single promoter driving expression of a dendritic cell chemoattractant-encoding gene, a dendritic cell growth factor or activator-encoding gene, and/or a lymphocyte signaling protein-encoding gene). In other embodiments, the nucleic acid vector includes a promoter that drives each gene in the immunomodulatory sequence (e.g., a first, second, and third promoter driving expression of a dendritic cell chemoattractant-encoding gene, a dendritic cell growth factor or activator-encoding gene, and a lymphocyte signaling protein-encoding gene, respectively).

In some instances, the immunomodulatory sequence includes a dendritic cell chemoattractant-encoding gene (e.g., an XCL1-encoding gene, an XCL2-encoding gene, a CCL5-encoding gene, or a CCL4-encoding gene) and a dendritic cell growth factor or activator-encoding gene (e.g., a FLT3L-encoding gene (e.g., a sFLT3L-encoding gene), a GMCSF-encoding gene, or a CD40L-encoding gene). In certain embodiments, such immunomodulatory sequences include no more than two genes. In some embodiments, the immunomodulatory sequence has multiple transcription units and includes a first promoter 5′ to the dendritic cell chemoattractant-encoding gene and a second promoter 5′ to the dendritic cell growth factor or activator-encoding gene. In other embodiments, the immunomodulatory sequence is bicistronic and includes a single promoter 5′ to the dendritic cell chemoattractant-encoding gene and the dendritic cell growth factor or activator-encoding gene.

Immunomodulatory proteins can also bind to and signal through lymphocytes (e.g., T cells, NK cells, or B cells). In some instances, self-replicating RNA molecules of the invention include immunomodulatory sequences having a lymphocyte signaling protein-encoding gene. Lymphocyte signaling proteins include cytokines and cytokines and may activate or stimulate the lymphocyte upon binding its receptor. Thus, in some embodiments, immunomodulatory sequences include a lymphocyte signaling protein-encoding gene, such as a cytokine or a chemokine. Cytokines include interleukins (ILs) such as IL-1, IL-1a, IL-2, IL-3, IL-4, IL-5, IL-6, IL-7, IL-8, IL-9, IL-11, IL-12, IL-13, IL-14, IL-15, IL-16, IL-17, IL-18, IL-21, and IL-36-γ; tumor necrosis factors such as TNF-α or TNF-β; Interferons (IFNs), such as IFN-β, IFN-γ, and IFN-α; and other protein factors including leukemia inhibitory factor (LIF) and kit ligand (KL). In particular embodiments, the cytokine encoded by the immunomodulatory sequence is IL-12 (e.g., a cytokine having an amino acid sequence having at least 95% identity to, at least 96% identity to, at least 97% identity to, at least 98% identity to, at least 99% identity to, or 100% Identity to SEQ ID NO: 24; and/or an immunomodulatory sequence having at least 95% Identity to, at least 96% Identity to, at least 97% Identity to, at least 98% Identity to, at least 99% Identity to, or 100% Identity to SEQ ID NO: 23 or 23A), IL-15 (e.g., a cytokine having an amino acid sequence having at least 95% Identity to, at least 96% identity to, at least 97% identity to, at least 98% Identity to, at least 99% Identity to, or 100% identity to SEQ ID NO: 26; and/or an immunomodulatory sequence having at least 95% Identity to, at least 96% Identity to, at least 97% Identity to, at least 98% identity to, at least 99% identity to, or 100% identity to SEQ ID NO: 25 or 25A), IFN-β (e.g., a cytokine having an amino acid sequence having at least 95% identity to, at least 96% identity to, at least 97% Identity to, at least 98% identity to, at least 99% identity to, or 100% identity to SEQ ID NO: 32; and/or an immunomodulatory sequence having at least 95% Identity to, at least 96% identity to, at least 97% Identity to, at least 98% Identity to, at least 99% Identity to, or 100% identity to SEQ ID NO: 31 or 31A), or IL-36-γ (e.g., a cytokine having an amino acid sequence having at least 95% Identity to, at least 96% Identity to, at least 97% Identity to, at least 98% Identity to, at least 99% Identity to, or 100% identity to SEQ ID NO: 36; and/or an immunomodulatory sequence having at least 95% Identity to, at least 96% Identity to, at least 97% Identity to, at least 98% Identity to, at least 99% identity to, or 100% identity to SEQ ID NO: 35 or 35A). Chemokines include CXCL9, CXCL10, CCL14, CCL19, CCL20, CCL21, CCL25, CCL27, CXCL12, CXCL13. CXCL-8, CCL2, CCL3, CCL4, CCL5, and CCL11. In some embodiments, the chemokine encoded by the immunomodulatory sequence is CXCL9 (e.g., a cytokine having an amino acid sequence having at least 95% identity to, at least 96% Identity to, at least 97% identity to, at least 98% identity to, at least 99% Identity to, or 100% Identity to SEQ ID NO: 28; and/or an immunomodulatory sequence having at least 95% Identity to, at least 96% identity to, at least 97% Identity to, at least 98% Identity to, at least 99% Identity to, or 100% Identity to SEQ ID NO: 27 or 27A) or CXCL10 (e.g., a cytokine having an amino acid sequence having at least 95% identity to, at least 96% identity to, at least 97% identity to, at least 98% identity to, at least 99% identity to, or 100% identity to SEQ ID NO: 30; and/or an immunomodulatory sequence having at least 95% Identity to, at least 96% identity to, at least 97% Identity to, at least 98% identity to, at least 99% identity to, or 100% identity to SEQ ID NO: 29 or 29A).

In some instances, the immunomodulatory sequence includes a dendritic cell chemoattractant-encoding gene (e.g., an XCL1-encoding gene, an XCL2-encoding gene, a CCL5-encoding gene, or a CCL4-encoding gene) and a lymphocyte signaling protein-encoding gene (e.g., a lymphocyte-activating cytokine-encoding gene (e.g., an IL-12-encoding gene or an IL-15-encoding gene) or a chemokine-encoding gene (e.g., a CXCL9-encoding gene or a CXCL10-encoding gene)). In other instances, the immunomodulatory sequence includes a dendritic cell growth factor or activator-encoding gene (e.g., a FLT3L-encoding gene (e.g., a sFLT3L-encoding gene), a GM-CSF-encoding gene, or a CD40L-encoding gene) and a lymphocyte signaling protein-encoding gene (e.g., a lymphocyte-activating cytokine-encoding gene (e.g., an IL-12-encoding gene or an IL-15-encoding gene) or a chemokine-encoding gene (e.g., a CXCL9-encoding gene or a CXCL10-encoding gene). In certain embodiments, such immunomodulatory sequences include no more than two genes. In some embodiments, the immunomodulatory sequence has multiple transcription units and includes a first promoter 5′ to the dendritic cell chemoattractant-encoding gene and a second promoter 5′ to the lymphocyte signaling protein-encoding gene. In other embodiments, the immunomodulatory sequence is bicistronic and includes a single promoter 5′ to the dendritic cell chemoattractant-encoding gene and the lymphocyte signaling protein-encoding gene.

In some instances, the immunomodulatory sequence includes a dendritic cell chemoattractant-encoding gene (e.g., an XCL1-encoding gene, an XCL2-encoding gene, a CCL5-encoding gene, or a CCL4-encoding gene), a dendritic cell growth factor or activator-encoding gene (e.g., a FLT3L-encoding gene (e.g., a sFLT3L-encoding gene), a GMCSF-encoding gene, or a CD40L-encoding gene), and a lymphocyte signaling protein-encoding gene (e.g., a lymphocyte-activating cytokine-encoding gene (e.g., an IL-12-encoding gene or an IL-15-encoding gene) or a chemokine-encoding gene (e.g., a CXCL9-encoding gene or a CXCL10-encoding gene). In some embodiments, the immunomodulatory sequence has multiple transcription units and includes a first promoter 5′ to the dendritic cell chemoattractant-encoding gene, a second promoter 5′ to the dendritic cell growth factor or activator-encoding gene, and a third promoter 5′ to the lymphocyte signaling protein-encoding gene. In other embodiments, the immunomodulatory sequence is tricistronic and includes a single promoter 5′ to the dendritic cell chemoattractant-encoding gene, the dendritic cell growth factor or activator-encoding gene, and the lymphocyte signaling protein-encoding gene.

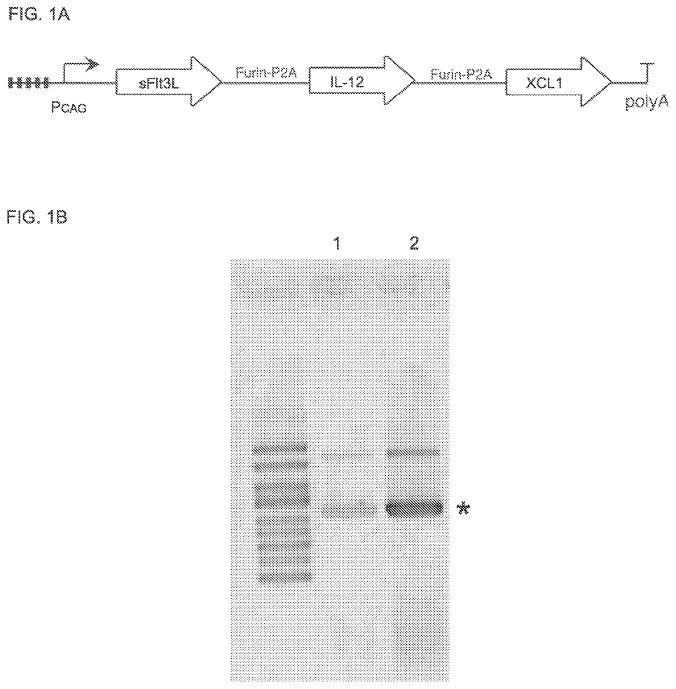

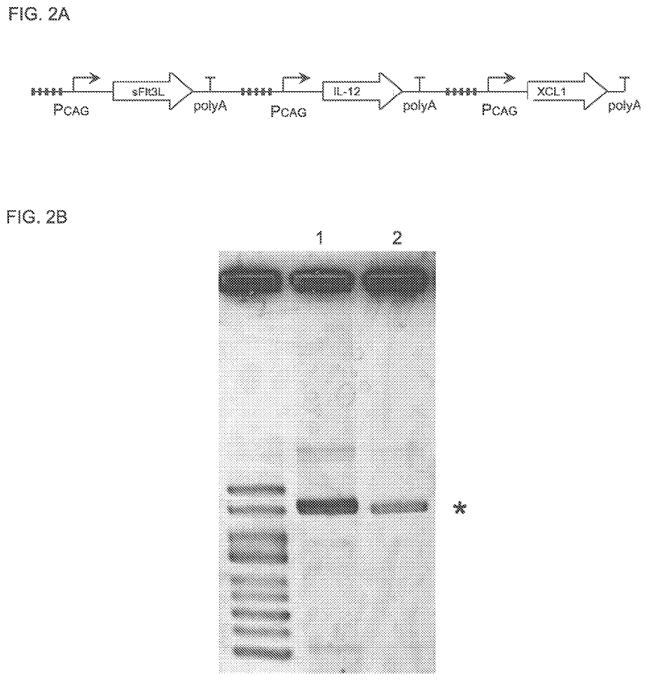

For example, an immunomodulatory sequence (e.g., an immunomodulatory sequence that has three transcription units, as exemplified in FIG. 2, or a tricistronic immunomodulatory sequence, as exemplified in FIG. 1) may include a dendritic col chemoattractant-encoding gene (e.g., an XCL1-encoding gene, an XCL2-encoding gene, a CCL5-encoding gene, or a CCL4-encoding gene), a dendritic cell growth factor or activator-encoding gene (e.g., a FLT3L-encoding gene (e.g., a sFLT3L-encoding gene), a GM-CSF-encoding gene, or a CD40L-encoding gene), and an IL-12-encoding gene. Alternatively, an immunomodulatory sequence (e.g., an immunomodulatory sequence that has three transcription units, or a tricistronic immunomodulatory sequence) may include a dendritic cell chemoattractant-encoding gene (e.g., an XCL1-encoding gene, an XCL2-encoding gene, a CCL5-encoding gene, or a CCL4-encoding gene), a dendritic cell growth factor or activator-encoding gene (e.g., a FLT3L-encoding gene (e.g., a sFLT3L-encoding gene), a GM-CSF-encoding gene, or a CD40L-encoding gene), and an IL-15-encoding gene. Alternatively, an immunomodulatory sequence (e.g., an immunomodulatory sequence that has three transcription units, or a tricistronic immunomodulatory sequence) may include a dendritic cell chemoattractant-encoding gene (e.g., an XCL1-encoding gene, an XCL2-encoding gene, a CCL5-encoding gene, or a CCL4-encoding gene), a dendritic cell growth factor or activator-encoding gene (e.g., a FLT3L-encoding gene (e.g., a sFLT3L-encoding gene), a GM-CSF-encoding gene, or a CD40L-encoding gene), and a CCL4-encoding gene. In other particular embodiments, an immunomodulatory sequence (e.g., an immunomodulatory sequence that has three transcription units, or a tricistronic immunomodulatory sequence) may include a dendritic cell chemoattractant-encoding gene (e.g., an XCL1-encoding gene, an XCL2-encoding gene, a CCL5-encoding gene, or a CCL4-encoding gene), a dendritic cell growth factor or activator-encoding gene (e.g., a FLT3L-encoding gene (e.g., a sFLT3L-encoding gene), a GM-CSF-encoding gene, or a CD40L-encoding gene), and a CCL5-encoding gene.

In some instances, an immunomodulatory sequence (e.g., an immunomodulatory sequence that has three transcription units, or a tricistronic immunomodulatory sequence) includes a dendritic cell chemoattractant-encoding gene (e.g., an XCL1-encoding gene, an XCL2-encoding gene, a CCL5-encoding gene, or a CCL4-encoding gene), a FLT3L-encoding gene (e.g., a sFLT3L-encoding gene), and a lymphocyte signaling protein-encoding gene (e.g., a lymphocyte-activating cytokine-encoding gene (e.g., an IL-12-encoding gene, an IL-15-encoding gene) or a chemokine-encoding gene (e.g., a CXCL9-encoding gene or a CXCL10-encoding gene)).

In particular instances, an immunomodulatory sequence (e.g., an immunomodulatory sequence that has three transcription units, or a tricistronic immunomodulatory sequence) includes a dendritic cell chemoattractant-encoding gene (e.g., an XCL1-encoding gene, an XCL2-encoding gene, a CCL5-encoding gene, or a CCL4-encoding gene), a FLT3L-encoding gene (e.g., a sFLT3L-encoding gene), and a lymphocyte-activating cytokine-encoding gene (e.g., an IL-12-encoding gene, an IL-15-encoding gene). Alternatively, the immunomodulatory sequence (e.g., the immunomodulatory sequence that has three transcription units, or the tricistronic immunomodulatory sequence) includes a dendritic cell chemoattractant-encoding gene (e.g., an XCL1-encoding gene, an XCL2-encoding gene, a CCL5-encoding gene, or a CCL4-encoding gene), a FLT3L-encoding gene (e.g., a sFLT3L-encoding gene), and a chemokine-encoding gene (e.g., a CXCL9-encoding gene or a CXCL10-encoding gene). In some instances, the immunomodulatory sequence (e.g., the immunomodulatory sequence that has three transcription units, or the tricistronic immunomodulatory sequence) includes a dendritic cell chemoattractant-encoding gene (e.g., an XCL1-encoding gene, an XCL2-encoding gene, a CCL5-encoding gene, or a CCL4-encoding gene), a FLT3L-encoding gene (e.g., a sFLT3L-encoding gene), and an IL-12-encoding gene. In other instances, the immunomodulatory sequence (e.g., the immunomodulatory sequence that has three transcription units, or the tricistronic immunomodulatory sequence) includes a dendritic cell chemoattractant-encoding gene (e.g., an XCL1-encoding gene, an XCL2-encoding gene, a CCL5-encoding gene, or a CCL4-encoding gene), a FLT3L-encoding gene (e.g., a sFLT3L-encoding gene), and an IL-15-encoding gene. In sill other instances, the immunomodulatory sequence (e.g., the immunomodulatory sequence that has three transcription units, or the tricistronic immunomodulatory sequence) includes a dendritic cell chemoattractant-encoding gene (e.g., an XCL1-encoding gene, an XCL2-encoding gene, a CCL5-encoding gene, or a CCL4-encoding gene), a FLT3L-encoding gene (e.g., a sFLT3L-encoding gene), and an CXCL9-encoding gene. In alternative instances, the immunomodulatory sequence (e.g., the immunomodulatory sequence that has three transcription units, or the tricistronic immunomodulatory sequence) includes a dendritic cell chemoattractant-encoding gene (e.g., an XCL1-encoding gene, an XCL2-encoding gene, a CCL5-encoding gene, or a CCL4-encoding gene), a FLT3L-encoding gene (e.g., a sFLT3L-encoding gene), and an CXCL10-encoding gene.

In particular instances, the immunomodulatory sequence (e.g., the immunomodulatory sequence that has three transcription units, or the tricistronic immunomodulatory sequence) includes an XCL1-encoding gene, a FLT3L-encoding gene (e.g., a sFLT3L-encoding gene), and a lymphocyte signaling protein-encoding gene (e.g., a lymphocyte-activating cytokine-encoding gene (e.g., an IL-12-encoding gene, an IL-15-encoding gene) or a chemokine-encoding gene (e.g., a CXCL9-encoding gene or a CXCL10-encoding gene)). In some instances, the immunomodulatory sequence (e.g., the immunomodulatory sequence that has three transcription units, or the tricistronic immunomodulatory sequence) includes an XCL1-encoding gene, a FLT3L-encoding gene (e.g., a sFLT3L-encoding gene), and an IL-12-encoding gene. In other instances, the immunomodulatory sequence (e.g., the immunomodulatory sequence that has three transcription units, or the tricistronic immunomodulatory sequence) includes an XCL1-encoding gene, a FLT3L-encoding gene (e.g., a sFLT3L-encoding gene), and an IL-15-encoding gene. In still other instances, the immunomodulatory sequence (e.g., the immunomodulatory sequence that has three transcription units, or the tricistronic immunomodulatory sequence) includes an XCL1-encoding gene, a FLT3L-encoding gene (e.g., a sFLT3L-encoding gene), and an CXCL9-encoding gene. In alternative instances, the immunomodulatory sequence (e.g., the immunomodulatory sequence that has three transcription units, or the tricistronic immunomodulatory sequence) includes an XCL1-encoding gene, a FLT3L-encoding gene (e.g., a sFLT3L-encoding gene), and an CXCL10-encoding gene.

In particular instances, the immunomodulatory sequence (e.g., the immunomodulatory sequence that has three transcription units, or the tricistronic immunomodulatory sequence) includes an XCL2-encoding gene, a FLT3L-encoding gene (e.g., a sFLT3L-encoding gene), and a lymphocyte signaling protein-encoding gene (e.g., a lymphocyte-activating cytokine-encoding gene (e.g., an IL-12-encoding gene, an IL-15-encoding gene) or a chemokine-encoding gene (e.g., a CXCL9-encoding gene or a CXCL10-encoding gene)). In some instances, the immunomodulatory sequence (e.g., the immunomodulatory sequence that has three transcription units, or the tricistronic immunomodulatory sequence) includes an XCL2-encoding gene, a FLT3L-encoding gene (e.g., a sFLT3L-encoding gene), and an IL-12-encoding gene. In other instances, the immunomodulatory sequence (e.g., the immunomodulatory sequence that has three transcription units, or the tricistronic immunomodulatory sequence) includes an XCL2-encoding gene, a FLT3L-encoding gene (e.g., a sFLT3L-encoding gene), and an IL-15-encoding gene. In still other instances, the immunomodulatory sequence (e.g., the immunomodulatory sequence that has three transcription units, or the tricistronic immunomodulatory sequence) includes an XCL2-encoding gene, a FLT3L-encoding gene (e.g., a sFLT3L-encoding gene), and an CXCL9-encoding gene. In alternative instances, the immunomodulatory sequence (e.g., the immunomodulatory sequence that has three transcription units, or the tricistronic immunomodulatory sequence) includes an XCL2-encoding gene, a FLT3L-encoding gene (e.g., a sFLT3L-encoding gene), and an CXCL10-encoding gene.

In particular instances, the immunomodulatory sequence (e.g., the immunomodulatory sequence that has three transcription units, or the tricistronic immunomodulatory sequence) includes a CCL4-encoding gene, a FLT3L-encoding gene (e.g., a sFLT3L-encoding gene), and a lymphocyte signaling protein-encoding gene (e.g., a lymphocyte-activating cytokine-encoding gene (e.g., an IL-12-encoding gene, an IL-15-encoding gene) or a chemokine-encoding gene (e.g., a CXCL9-encoding gene or a CXCL10-encoding gene)). In some instances, the immunomodulatory sequence includes a CCL4-encoding gene, a FLT3L-encoding gene (e.g., a sFLT3L-encoding gene), and an IL-12-encoding gene. In other instances, the immunomodulatory sequence includes a CCL4-encoding gene, a FLT3L-encoding gene (e.g., a sFLT3L-encoding gene), and an IL-15-encoding gene. In still other instances, the immunomodulatory sequence includes a CCL4-encoding gene, a FLT3L-encoding gene (e.g., a sFLT3L-encoding gene), and an CXCL9-encoding gene. In alternative instances, the immunomodulatory sequence includes a CCL4-encoding gene, a FLT3L-encoding gene (e.g., a sFLT3L-encoding gene), and an CXCL10-encoding gene.

In particular instances, the immunomodulatory sequence (e.g., the immunomodulatory sequence that has three transcription units, or the tricistronic immunomodulatory sequence) includes a CCL5-encoding gene, a FLT3L-encoding gene (e.g., a sFLT3L-encoding gene), and a lymphocyte signaling protein-encoding gene (e.g., a lymphocyte-activating cytokine-encoding gene (e.g., an IL-12-encoding gene, an IL-15-encoding gene) or a chemokine-encoding gene (e.g., a CXCL9-encoding gene or a CXCL10-encoding gene)). In some instances, the immunomodulatory sequence includes a CCL5-encoding gene, a FLT3L-encoding gene (e.g., a sFLT3L-encoding gene), and an IL-12-encoding gene. In other instances, the immunomodulatory sequence includes a CCL5-encoding gene, a FLT3L-encoding gene (e.g., a sFLT3L-encoding gene), and an IL-15-encoding gene. In still other instances, the immunomodulatory sequence includes a CCL5-encoding gene, a FLT3L-encoding gene (e.g., a sFLT3L-encoding gene), and an CXCL9-encoding gene. In alternative instances, the immunomodulatory sequence includes a CCL5-encoding gene, a FLT3L-encoding gene (e.g., a sFLT3L-encoding gene), and an CXCL10-encoding gene.

In some instances, the immunomodulatory sequence (e.g., the immunomodulatory sequence that has three transcription units, or the tricistronic immunomodulatory sequence) includes a dendritic cell chemoattractant-encoding gene (e.g., an XCL1-encoding gene, an XCL2-encoding gene, a CCL5-encoding gene, or a CCL4-encoding gene), a GM-CSF-encoding gene, and a lymphocyte-activating cytokine-encoding gene (e.g., an IL-12-encoding gene, an IL-15-encoding gene). Alternatively, the immunomodulatory sequence includes a dendritic cell chemoattractant-encoding gene (e.g., an XCL1-encoding gene, an XCL2-encoding gene, a CCL5-encoding gene, or a CCL4-encoding gene), a GM-CSF-encoding gene, and a chemokine-encoding gene (e.g., a CXCL9-encoding gene or a CXCL10-encoding gene). In some instances, the immunomodulatory sequence includes a dendritic cell chemoattractant-encoding gene (e.g., an XCL1-encoding gene, an XCL2-encoding gene, a CCL5-encoding gene, or a CCL4-encoding gene), a GM-CSF-encoding gene, and an IL-12-encoding gene. In other instances, the immunomodulatory sequence includes a dendritic cell chemoattractant-encoding gene (e.g., an XCL1-encoding gene, an XCL2-encoding gene, a CCL5-encoding gene, or a CCL4-encoding gene), a GM-CSF-encoding gene, and an IL-15-encoding gene. In still other instances, the immunomodulatory sequence includes a dendritic cell chemoattractant-encoding gene (e.g., an XCL1-encoding gene, an XCL2-encoding gene, a CCL5-encoding gene, or a CCL4-encoding gene), a GM-CSF-encoding gene, and an CXCL9-encoding gene. In alternative instances, the immunomodulatory sequence includes a dendritic cell chemoattractant-encoding gene (e.g., an XCL1-encoding gene, an XCL2-encoding gene, a CCL5-encoding gene, or a CCL4-encoding gene), a GM-CSF-encoding gene, and an CXCL10-encoding gene.

In particular instances, the immunomodulatory sequence (e.g., the immunomodulatory sequence that has three transcription units, or the tricistronic immunomodulatory sequence) includes an XCL1-encoding gene, a GM-CSF-encoding gene, and a lymphocyte signaling protein-encoding gene (e.g., a lymphocyte-activating cytokine-encoding gene (e.g., an IL-12-encoding gene, an IL-15-encoding gene) or a chemokine-encoding gene (e.g., a CXCL9-encoding gene or a CXCL10-encoding gene)). In some instances, the immunomodulatory sequence includes an XCL1-encoding gene, a GM-CSF-encoding gene, and an IL-12-encoding gene. In other instances, the immunomodulatory sequence includes an XCL1-encoding gene, a GM-CSF-encoding gene, and an IL-15-encoding gene. In still other instances, the immunomodulatory sequence includes an XCL1-encoding gene, a GM-CSF-encoding gene, and an CXCL9-encoding gene. In alternative instances, the immunomodulatory sequence includes an XCL1-encoding gene, a GM-CSF-encoding gene, and an CXCL10-encoding gene.

In particular instances, the immunomodulatory sequence (e.g., the immunomodulatory sequence that has three transcription units, or the tricistronic immunomodulatory sequence) includes an XCL2-encoding gene, a GM-CSF-encoding gene, and a lymphocyte signaling protein-encoding gene (e.g., a lymphocyte-activating cytokine-encoding gene (e.g., an IL-12-encoding gene, an IL-15-encoding gene) or a chemokine-encoding gene (e.g., a CXCL9-encoding gene or a CXCL10-encoding gene)). In some instances, the immunomodulatory sequence includes an XCL2-encoding gene, a GM-CSF-encoding gene, and an IL-12-encoding gene. In other instances, the immunomodulatory sequence includes an XCL2-encoding gene, a GM-CSF-encoding gene, and an IL-15-encoding gene. In still other instances, the immunomodulatory sequence includes an XCL2-encoding gene, a GM-CSF-encoding gene, and an CXCL9-encoding gene. In alternative instances, the immunomodulatory sequence includes an XCL2-encoding gene, a GM-CSF-encoding gene, and an CXCL10-encoding gene.

In particular instances, the immunomodulatory sequence (e.g., the immunomodulatory sequence that has three transcription units, or the tricistronic immunomodulatory sequence) includes a CCL4-encoding gene, a GM-CSF-encoding gene, and a lymphocyte signaling protein-encoding gene (e.g., a lymphocyte-activating cytokine-encoding gene (e.g., an IL-12-encoding gene, an IL-15-encoding gene) or a chemokine-encoding gene (e.g., a CXCL9-encoding gene or a CXCL10-encoding gene)). In some instances, the immunomodulatory sequence includes a CCL4-encoding gene, a GM-CSF-encoding gene, and an IL-12-encoding gene. In other instances, the immunomodulatory sequence includes a CCL4-encoding gene, a GM-CSF-encoding gene, and an IL-15-encoding gene. In still other instances, the immunomodulatory sequence includes a CCL4-encoding gene, a GM-CSF-encoding gene, and an CXCL9-encoding gene. In alternative instances, the immunomodulatory sequence includes a CCL4-encoding gene, a GM-CSF-encoding gene, and an CXCL10-encoding gene.

In particular instances, the immunomodulatory sequence (e.g., the immunomodulatory sequence that has three transcription units, or the tricistronic immunomodulatory sequence) includes a CCL5-encoding gene, a GM-CSF-encoding gene, and a lymphocyte signaling protein-encoding gene (e.g., a lymphocyte-activating cytokine-encoding gene (e.g., an IL-12-encoding gene, an IL-15-encoding gene) or a chemokine-encoding gene (e.g., a CXCL9-encoding gene or a CXCL10-encoding gene)). In some instances, the immunomodulatory sequence includes a CCL5-encoding gene, a GM-CSF-encoding gene, and an IL-12-encoding gene. In other instances, the immunomodulatory sequence includes a CCL5-encoding gene, a GM-CSF-encoding gene, and an IL-15-encoding gene. In still other instances, the immunomodulatory sequence includes a CCL5-encoding gene, a GM-CSF-encoding gene, and an CXCL9-encoding gene. In alternative instances, the immunomodulatory sequence includes a CCL5-encoding gene, a GM-CSF-encoding gene, and an CXCL10-encoding gene.

In some instances, the immunomodulatory sequence (e.g., the immunomodulatory sequence that has three transcription units, or the tricistronic immunomodulatory sequence) includes a dendritic cell chemoattractant-encoding gene (e.g., an XCL1-encoding gene, an XCL2-encoding gene, a CCL5-encoding gene, or a CCL4-encoding gene), a CD40L-encoding gene, and a lymphocyte-activating cytokine-encoding gene (e.g., an IL-12-encoding gene, an IL-15-encoding gene). Alternatively, the immunomodulatory sequence includes a dendritic cell chemoattractant-encoding gene (e.g., an XCL1-encoding gene, an XCL2-encoding gene, a CCL5-encoding gene, or a CCL4-encoding gene), a CD40L-encoding gene, and a chemokine-encoding gene (e.g., a CXCL9-encoding gene or a CXCL10-encoding gene). In some instances, the immunomodulatory sequence includes a dendritic cell chemoattractant-encoding gene (e.g., an XCL1-encoding gene, an XCL2-encoding gene, a CCL5-encoding gene, or a CCL4-encoding gene), a CD40L-encoding gene, and an IL-12-encoding gene. In other instances, the immunomodulatory sequence includes a dendritic cell chemoattractant-encoding gene (e.g., an XCL1-encoding gene, an XCL2-encoding gene, a CCL5-encoding gene, or a CCL4-encoding gene), a CD40L-encoding gene, and an IL-15-encoding gene. In still other instances, the immunomodulatory sequence includes a dendritic cell chemoattractant-encoding gene (e.g., an XCL1-encoding gene, an XCL2-encoding gene, a CCL5-encoding gene, or a CCL4-encoding gene), a CD40L-encoding gene, and an CXCL9-encoding gene. In alternative instances, the immunomodulatory sequence includes a dendritic cell chemoattractant-encoding gene (e.g., an XCL1-encoding gene, an XCL2-encoding gene, a CCL5-encoding gene, or a CCL4-encoding gene), a CD40L-encoding gene, and an CXCL10-encoding gene.

In particular instances, the immunomodulatory sequence (e.g., the immunomodulatory sequence that has three transcription units, or the tricistronic immunomodulatory sequence) includes an XCL1-encoding gene, a CD40L-encoding gene, and a lymphocyte signaling protein-encoding gene (e.g., a lymphocyte-activating cytokine-encoding gene (e.g., an IL-12-encoding gene, an IL-15-encoding gene) or a chemokine-encoding gene (e.g., a CXCL9-encoding gene or a CXCL10-encoding gene)). In some instances, the immunomodulatory sequence includes an XCL1-encoding gene, a CD40L-encoding gene, and an IL-12-encoding gene. In other instances, the immunomodulatory sequence includes an XCL1-encoding gene, a CD40L-encoding gene, and an IL-15-encoding gene. In still other instances, the immunomodulatory sequence includes an XCL1-encoding gene, a CD40L-encoding gene, and an CXCL9-encoding gene. In alternative instances, the immunomodulatory sequence includes an XCL1-encoding gene, a CD40L-encoding gene, and an CXCL10-encoding gene.

In particular instances, the immunomodulatory sequence (e.g., the immunomodulatory sequence that has three transcription units, or the tricistronic immunomodulatory sequence) includes an XCL2-encoding gene, a CD40L-encoding gene, and a lymphocyte signaling protein-encoding gene (e.g., a lymphocyte-activating cytokine-encoding gene (e.g., an IL-12-encoding gene, an IL-15-encoding gene) or a chemokine-encoding gene (e.g., a CXCL9-encoding gene or a CXCL10-encoding gene)). In some instances, the immunomodulatory sequence includes an XCL2-encoding gene, a CD40L-encoding gene, and an IL-12-encoding gene. In other instances, the immunomodulatory sequence includes an XCL2-encoding gene, a CD40L-encoding gene, and an IL-15-encoding gene. In still other instances, the immunomodulatory sequence includes an XCL2-encoding gene, a CD40L-encoding gene, and an CXCL9-encoding gene. In alternative instances, the immunomodulatory sequence includes an XCL2-encoding gene, a CD40L-encoding gene, and an CXCL10-encoding gene.

In particular instances, the immunomodulatory sequence (e.g., the immunomodulatory sequence that has three transcription units, or the tricistronic immunomodulatory sequence) includes a CCL4-encoding gene, a CD40L-encoding gene, and a lymphocyte signaling protein-encoding gene (e.g., a lymphocyte-activating cytokine-encoding gene (e.g., an IL-12-encoding gene, an IL-15-encoding gene) or a chemokine-encoding gene (e.g., a CXCL9-encoding gene or a CXCL10-encoding gene)). In some instances, the immunomodulatory sequence includes a CCL4-encoding gene, a CD40L-encoding gene, and an IL-12-encoding gene. In other instances, the immunomodulatory sequence includes a CCL4-encoding gene, a CD40L-encoding gene, and an IL-15-encoding gene. In still other instances, the immunomodulatory sequence includes a CCL4-encoding gene, a CD40L-encoding gene, and an CXCL9-encoding gene. In alternative instances, the immunomodulatory sequence includes a CCL4-encoding gene, a CD40L-encoding gene, and an CXCL10-encoding gene.

In particular instances, the immunomodulatory sequence (e.g., the immunomodulatory sequence that has three transcription units, or the tricistronic immunomodulatory sequence) includes a CCL5-encoding gene, a CD40L-encoding gene, and a lymphocyte signaling protein-encoding gene (e.g., a lymphocyte-activating cytokine-encoding gene (e.g., an IL-12-encoding gene, an IL-15-encoding gene) or a chemokine-encoding gene (e.g., a CXCL9-encoding gene or a CXCL10-encoding gene)). In some instances, the immunomodulatory sequence includes a CCL5-encoding gene, a CD40L-encoding gene, and an IL-12-encoding gene. In other instances, the immunomodulatory sequence includes a CCL5-encoding gene, a CD40L-encoding gene, and an IL-15-encoding gene. In still other instances, the immunomodulatory sequence includes a CCL5-encoding gene, a CD40L-encoding gene, and an CXCL9-encoding gene. In alternative instances, the immunomodulatory sequence includes a CCL5-encoding gene, a CD40L-encoding gene, and an CXCL10-encoding gene.

In particular embodiments, the immunomodulatory sequence has three transcription units, wherein a first promoter is 5′ to an XCL1-encoding gene, a second promoter is 5′ to an sFLT3L-encoding gene, and a third promoter is 5′ to an IL-12-encoding gene. In an alternative embodiment, the immunomodulatory sequence is tricistronic, wherein a single promoter is 5′ to an XCL1-encoding gene, an sFLT3L-encoding gene, and an IL-12-encoding gene.

In particular embodiments, the immunomodulatory sequence has three transcription units, wherein a first promoter is 5′ to an XCL1-encoding gene, a second promoter is 5′ to an sFLT3L-encoding gene, and a third promoter is 5′ to an IL-15-encoding gene. In an alternative embodiment, the immunomodulatory sequence is tricistronic, wherein a single promoter is 5′ to an XCL1-encoding gene, an sFLT3L-encoding gene, and an IL-15-encoding gene.

Alternatively, in some instances, the immunomodulatory sequence has three transcription units, wherein a first promoter is 5′ to an XCL1-encoding gene, a second promoter is 5′ to an GM-CSF-encoding gene, and a third promoter is 5′ to an IL-12-encoding gene. In an alternative embodiment, the immunomodulatory sequence is tricistronic, wherein a single promoter is 5′ to an XCL1-encoding gene, an GM-CSF-encoding gene, and an IL-12-encoding gene.

In particular embodiments, the immunomodulatory sequence has three transcription units, wherein a first promoter is 5′ to an XCL1-encoding gene, a second promoter is 5′ to an GM-CSF-encoding gene, and a third promoter is 5′ to an IL-15-encoding gene. In an alternative embodiment, the immunomodulatory sequence is tricistronic, wherein a single promoter is 5′ to an XCL1-encoding gene, an GM-CSF-encoding gene, and an IL-15-encoding gene.

In some embodiments of any of the preceding nucleic acid vectors (e.g., circular DNA vectors), the immunomodulatory sequence comprises, operatively linked in a 5′ to 3′ direction, the dendritic cell chemoattractant-encoding gene, the dendritic cell growth factor or activator-encoding gene, and the lymphocyte signaling protein-encoding gene (e.g., the replicase-encoding sequence, the dendritic call chemoattractant-encoding gene, the dendritic cell growth factor or activator-encoding gene, and the lymphocyte signaling protein-encoding gene). In other embodiments, the immunomodulatory sequence comprises, operatively linked in a 5′ to 3′ direction, the dendritic cell growth factor or activator-encoding gene, the dendritic cell chemoattractant-encoding gene, and the lymphocyte signaling protein-encoding gene (e.g., the replicase-encoding sequence, the dendritic cell growth factor or activator-encoding gene, the dendritic cell chemoattractant-encoding gene, and the lymphocyte signaling protein-encoding gene). In other embodiments, the immunomodulatory sequence comprises, operatively linked in a 5′ to 3′ direction, the dendritic cell chemoattractant-encoding gene, the lymphocyte signaling protein-encoding gene, and the dendritic cell growth factor or activator-encoding gene (e.g., the replicase-encoding sequence, the dendritic cell chemoattractant-encoding gene, the lymphocyte signaling protein-encoding gene, and the dendritic cell growth factor or activator-encoding gene). In other embodiments, the immunomodulatory sequence comprises, operatively linked in a 5′ to 3′ direction, the dendritic cell growth factor or activator-encoding gene, the lymphocyte signaling protein-encoding gene, and the dendritic cell chemoattractant-encoding gene (e.g., the replicase-encoding sequence, the dendritic cell growth factor or activator-encoding gene, the lymphocyte signaling protein-encoding gene, and the dendritic cell chemoattractant-encoding gene). In other embodiments, the immunomodulatory sequence comprises, operatively linked in a 5′ to 3′ direction, the lymphocyte signaling protein-encoding gene, the dendritic cell chemoattractant-encoding gene, and the dendritic cell growth factor or activator-encoding gene (e.g., the replicase-encoding sequence, the lymphocyte signaling protein-encoding gene, the dendritic cell chemoattractant-encoding gene, and the dendritic cell growth factor or activator-encoding gene). In other embodiments, the immunomodulatory sequence comprises, operatively linked in a 5′ to 3′ direction, the lymphocyte signaling protein-encoding gene, the dendritic cell growth factor or activator-encoding gene, and the dendritic cell chemoattractant-encoding gene (e.g., the replicase-encoding sequence, the lymphocyte signaling protein-encoding gene, the dendritic cell growth factor or activator-encoding gene, and the dendritic cell chemoattractant-encoding gene).

In particular embodiments, an immunomodulatory sequence encodes an amino acid sequence having at least 95% sequence identity to, at least 96% sequence identity to, at least 97% sequence identity to, at least 98% sequence identity to, at least 99% identity to, or 100% identity to SEQ ID NO: 34. Additionally, or alternatively, in some embodiments, the immunomodulatory sequence comprises a nucleic acid sequence having at least 95% sequence identity to, at least 96% sequence identity to, at least 97% sequence identity to, at least 98% sequence identity to, at least 99% Identity to, or 100% Identity to SEQ ID NO: 33 or 33A.

In certain instances of any of the aforementioned embodiments of the invention, tricistronic immunomodulatory sequences include no more than three genes.

Heterologous genes of any of the self-replicating RNA molecules described herein may encode a functionally equivalent fragment of any of the proteins described herein, or variants thereof (e.g., dendritic cell chemoattractants, dendritic cell growth factors or activators, and/or lymphocyte signaling proteins). A fragment of a protein or a variant thereof encoded by the self-replicating RNA molecule according to the invention may include an amino acid sequence having a sequence identity of at least 5%, 10%, 20%, 30%, 40%, 50%, 60%, 70%, 80%, 85%, 88%, 87%, 88%, 89%, 90%, 91%, 92%, 93%, 94%, 95%, 96%, 97%, 98%, or 99% (e.g., at least 70%, at least 80%, at least 85%, at least 90%, at least 95%, or at least 97% sequence identity) with a reference amino acid sequence (e.g., the amino acid sequence of the respective naturally occurring full-length protein or a variant thereof).

Other immunomodulatory protein-encoding genes useful within the immunomodulatory sequence include genes that encode other cytokines, chemokines, and growth factors. Other cytokines useful as part of the present invention include TNFα, IFN-γ, IFN-α, TGF-β, IL-1, IL-2, IL-4, IL-10, IL-13, IL-17, and IL-18. Non-limiting examples of chemokines useful as part of the present invention include CCL14, CCL19, CCL20, CCL21, CCL25, CCL27, CXCL12, CXCL13, CXCL-8, CCL2. CCL3, CCL4, CCL5, CCL11, and CXCL10. Non-limiting examples of growth factors include adrenomedullin (AM), angiopoietin (Ang), autocrine motility factor, bone morphogenetic proteins (BMPs), ciliary neurotrophic factor (CNTF), leukemia inhibitory factor (LIF), macrophage colony-stimulating factor (m-CSF), granulocyte colony-stimulating factor (G-CSF), epidermal growth factor (EGF), ephrin A1, ephrin A2, ephrin A3, ephrin A4, ephrin AS, ephrin B1, ephrin B2, ephrin B3, erythropoietin (EPO), fibroblast growth factor 1 (FGF1), FGF2, FGF3, FGF4, FGF5, FGF6, FGF7, FGF8, FGF9, FGF10, FGF11, FGF12, FGF13, FGF14, FGF15, FGF16. FGF17, FGF18, FGF19, FGF20. FGF21. FGF22, FGF23, fetal bovine somatotrophin (FBS), glial cell line-derived neurotrophic factor (GDNF), neurturin, persephin, artemin, growth differentiation factor-9 (GDF9), hepatocyte growth factor (HGF), hepatoma-derived growth factor (HDGF), insulin, insulin-like growth factor-1 (IGF-1), insulin-like growth factor-2 (IGF-2), interleukin-1 (IL-1), IL-2, IL-3, IL-4, IL-5, IL-6, IL-7, keratinocyte growth factor (KGF), migration-stimulating factor (MSF), macrophage-stimulating protein (MSP), myostatin (GDF-8), neuregulin 1 (NRG1), NRG2, NRG3, NRG4, brain-derived neurotrophic factor (BDNF), nerve growth factor (NGF), neurotrophin-3 (NT-3), NT-4, placental growth factor (PGF), platelet-derived growth factor (PDGF), renalase (RNLS), T-cell growth factor (TCGF), thrombopoietin (TPO), transforming growth factor alpha (TGF-α), transforming growth factor beta (TGF-β), tumor necrosis factor-alpha (TNF-α), and vascular endothelial growth factor (VEGF).

In any of the polycistronic nucleic acid vectors (e.g., DNA vectors, e.g., circular DNA vectors) described herein, cleavage sites can be designed between protein-coding regions. For example, furin-P2A sites can separate any of the protein-coding genes described herein. Ribozymes can also be incorporated into a self-replicating RNA molecule to cleave sites downstream of a protein-coding gene. In some embodiments, T2A, E2A, F2A, or any other suitable self-cleavage site (e.g., virus-derived cleavage site) can separate any of the protein-coding genes described herein.

Circular DNA Vectors

Some embodiments of the present invention involve circular DNA vectors (e.g., circular DNA vectors having multiple transcription units, each encoding an immunodulatory gene; circular DNA vectors having a single transcription unit encoding multiple immunomodulatory genes; and circular DNA vectors encoding a self-replicating RNA molecule encoding a replicase and one or more immunomodulatory genes). In some instances, circular DNA vectors useful to encode the immunomodulatory sequences described herein can be plasmid DNA vectors. In particular instances of the present invention, circular DNA vectors differ from conventional plasmid DNA vectors in that they lack plasmid backbone elements (e.g., bacterial elements such as (i) a bacterial origin of replication and/or (ii) a drug resistance gene). In some embodiments, circular DNA vectors described herein (e.g., DNA vectors encoding any of the self-replicating RNA molecules described herein) lack a recombination site (e.g., synthetic circular DNA vectors produced by a cell-free process). In alternative embodiments, circular DNA vectors described herein (e.g., encoding any of the self-replicating RNA molecules described herein) include a recombination site (e.g., minicircle DNA vectors).

A circular DNA vector of the invention may include a promoter operably linked 5 to a self-replicating RNA molecule-encoding sequence. A promoter is operably linked to a self-replicating RNA molecule-encoding sequence if the promoter is capable of effecting transcription of that self-replicating RNA molecule-encoding sequence. Promoters that can be used as part of circular DNA vectors include constitutive promoters, inducible promoters, native-promoters, and tissue-specific promoters. Examples of constitutive promoters include, a cytomegalovirus (CMV) promoter (optionally with the CMV enhancer), a retroviral Rous sarcoma virus (RSV) LTR promoter (optionally with the RSV enhancer), an SV40 promoter, a dihydrofolate reductase promoter, a β-actin promoter, a phosphoglycerol kinase (PGK) promoter, and an EF1 a promoter. In particular embodiments of the invention, the circular DNA vector includes a CMV promoter. In some embodiments, the circular DNA vector includes a CAG promoter.

Alternatively, circular DNA vectors of the invention include inducible promoters. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources. Many other systems have been described and can be readily selected by one of skill in the art. Examples of inducible promoters regulated by exogenously supplied promoters include zinc-inducible sheep metallothionine (MT) promoters, dexamethasone-inducible mouse mammary tumor virus promoters, T7 polymerase promoter systems, ecdysone insect promoters, tetracycline-repressible systems, tetracycline-inducible systems, RU486-inducible systems, and rapamycin-inducible systems. Still other types of inducible promoters which may be useful in this context are those which are regulated by a specific physiological state, e.g., temperature, acute phase, a particular differentiation state of the call, or in replicating cells only.

A circular DNA vector of the invention may also include a polyadenylation sequence 3 to the self-replicating RNA molecule-encoding sequence. Useful polyadenylation sequences include elongated polyadenylation sequences of greater than 20 nt (e.g., greater than 25 nt, greater that 30 nt, greater than 35 nt, greater then 40 nt, greater than 50 nt, greater than 60 nt, greater than 70 nt, or greater than 80 nt, e.g., from 20 to 100 nt, from 30 to 100 nt, from 40 to 100 nt, from 50 to 100 nt, from 60 to 100 nt, from 70 to 100 nt, from 80 to 100 nt, from 100 to 200 nt, from 200 to 300 nt, or from 300 to 400 nt, or greater).

Circular DNA vectors that lack bacterial elements such as a DNA origin of replication and/or a drug resistance gene can persist in an individual longer than conventional DNA vectors (e.g., plasmids) and longer than naked RNA. In certain embodiments involving self-replicating RNA, circular DNA vectors confer enhanced stability to the self-replicating RNA molecule encoded therein.

Circular DNA vectors can have various sizes and shapes. A circular DNA vector encoding a self-replicating RNA molecule of the invention can be from 5 kb to 20 kb in length (e.g., from 6 kb to 18 kb, from 7 kb to 16 kb, from 8 kb to 14 kb, or from 9 kb to 12 kb in length, e.g., from 5 kb to 6 kb, from 6 kb to 7 kb, from 7 kb to 8 kb, from 8 kb to 9 kb, from 9 kb to 10 kb, from 10 kb to 11 kb, from 11 kb to 12 kb, from 12 kb to 13 kb, from 13 kb to 14 kb, from 14 kb to 15 kb, from 15 kb to 16 kb, from 16 kb to 18 kb, or from 18 kb to 20 kb in length. e.g., about 5 kb, about 6 kb, about 7 kb, about 8 kb, about 9 kb, about 10 kb, about 10.5 kb, about 11 kb, about 11.5 kb, about 12 kb, about 12.5 kb, about 13 kb, about 14 kb, about 15 kb, about 16 kb, about 17 kb, about 18 kb, about 19 kb, or about 20 kb in length).

Circular DNA vectors useful as part of the present invention can be readily synthesized through various means known in the art and described herein. For example, circular DNA vectors that lack plasmid backbone elements (e.g., bacterial elements such as (i) a bacterial origin of replication and/or (ii) a drug resistance gene) can be made using in-vitro (cell-free) methods, which can provide purer compositions relative to bacterial-based methods. Such in-vitro synthesis methods may involve use of phage polymerase, such as Phi29 polymerase, as a replication tool using, e.g., rolling circle amplification. Particular methods of in-vitro synthesis of circular DNA vectors are further described in international Patent Publication WO 2019/178500, which is incorporated herein by reference.

Self-Replicating RNA Molecules and DNA Vectors Encoding them

Provided herein are self-replicating RNA molecules useful as modulatory therapies, e.g., immunomodulatory therapies. Upon delivery to a vertebrate cell, a self-replicating RNA molecule can lead to the production of multiple daughter RNAs by transcription from itself (via an antisense copy generated from itself). In some instances, a self-replicating RNA molecule is a positive-strand molecule which can be directly translated after delivery to a cell, and this translation produces a replicase, which then produces antisense and sense transcripts from the delivered RNA. Thus, the delivered RNA leads to the production of multiple daughter RNAs. These daughter RNAs, as well as subgenomic transcripts, can be translated to provide in-situ expression of an encoded protein (e.g., a modulatory protein, e.g., an immunomodulatory protein) or may be transcribed to provide further transcripts with the same sense as the delivered RNA which are translated to provide in-situ expression of the modulatory protein. The overall results of this sequence of transcriptions is an amplification in the number of the self-replicating RNA molecules, and the encoded modulatory proteins can become a major polypeptide product of the target cells (e.g., tumor cells and/or tumor-resident cells).

In some instances, any of the aforementioned DNA vectors (e.g., circular DNA vectors) encodes a self-replicating RNA molecule containing the modulatory sequence. Such self-replicating RNA molecules include replicase sequences derived from alphavirus, which are characterized as having positive-stranded replicons that are translated after delivery to a target cell into a replicase (or replicase-transcriptase). The replicase is translated as a polyprotein which auto-cleaves to provide a replication complex which creates genomic negative-strand copies of the positive-strand delivered RNA. These negative-strand transcripts can themselves be transcribed to give further copies of the positive-stranded parent RNA and also to give a subgenomic transcript (e.g., a modulatory sequence). Translation of the subgenomic transcript thus leads to in situ expression of the modulatory protein by the infected cell.

Non-limiting examples of alphaviruses from which replicase-encoding sequences of the present invention can be derived include Venezuelan equine encephalitis virus (VEE), Semliki Forest virus (SF), Sindbis virus (SIN), Eastern Equine Encephalitis virus (EEE), Western equine encephalitis virus (WEE), Everglades virus (EVE), Mucambo virus (MUC), Pixuna virus (PIX), Semliki Forest virus (SF), Middelburg virus (MID), Chikungunya virus (CHIK), O'Nyong-Nyong virus (ONN), Ross River virus (RR), Barmah Forest virus (BF), Getah virus (GET), Saglyama virus (SAG), Bebaru virus (BEB), Mayaro virus (MAY), Una virus (UNA), Aura virus (AURA), Babanki virus (BAB), Highlands J virus (HJ), and Fort Morgan virus (FM). In particular instances of the invention, the self-replicating RNA molecule comprises a VEE replicase or a variant thereof. In some embodiments, the self-replicating RNA molecule comprises a replicase-encoding sequence of SEQ ID NO: 1 or 1A. In some embodiments, the self-replicating RNA molecule comprises a replicase-encoding sequence comprising a nucleic acid sequence that is at least 95% Identical to SEQ ID NO: 1 or 1A (e.g., at least 98%, at least 97%, at least 98%, or at least 99% (e.g., at least 99.1%, at least 99.2%, at least 99.3%, at least 99.4%, at least 99.5%, at least 99.6%, at least 99.7%, at least 99.8%, or at least 99.9%) identical to SEQ ID NO: 1 or 1A). In some embodiments, the self-replicating RNA molecule comprises a replicase-encoding sequence comprising a nucleic acid sequence that is at least 95% Identical to SEQ ID NO: 3 or 3A (e.g., at least 96%, at least 97%, at least 98%, or at least 99% (e.g., at least 99.1%, at least 99.2%, at least 99.3%, at least 99.4%, at least 99.5%, at least 99.6%, at least 99.7%, at least 99.8%, or at least 99.9%) identical to SEQ ID NO: 3 or 3A). In some embodiments, the self-replicating RNA molecule comprises a replicase-encoding sequence comprising a nucleic acid sequence that is at least 95% identical to SEQ ID NO: 5 or 5A (e.g., at least 96%, at least 97%, at least 98%, or at least 99% (e.g., at least 99.1%, at least 99.2%, at least 99.3%, at least 99.4%, at least 99.5%, at least 99.6%, at least 99.7%, at least 99.8%, or at least 99.9%) identical to SEQ ID NO: 5 or 5A). In some embodiments, the self-replicating RNA molecule comprises a replicase-encoding sequence comprising a nucleic acid sequence that is at least 95% Identical to SEQ ID NO: 7 or 7A (e.g., at least 96%, at least 97%, at least 98%, or at least 99% (e.g., at least 99.1%, at least 99.2%, at least 99.3%, at least 99.4%, at least 99.5%, at least 99.6%, at least 99.7%, at least 99.8%, or at least 99.9%) identical to SEQ ID NO: 7 or 7A). In some embodiments, the self-replicating RNA molecule comprises a replicase-encoding sequence comprising a first nucleic acid sequence that is at least 95% identical to SEQ ID NO: 1 or 1A (at least 96%, at least 97%, at least 98%, at least 99%, or 100% Identical to SEQ ID NO: 1 or 1A), a second nucleic acid sequence that is at least 95% Identical to SEQ ID NO: 3 or 3A (at least 96%, at least 97%, at least 98%, at least 99%, or 100% identical to SEQ ID NO: 3 or 3A), a third nucleic acid sequence that is at least 95% identical to SEQ ID NO: 5 or 5A (at least 96%, at least 97%, at least 98%, at least 99%, or 100% identical to SEQ ID NO: 5 or 5A), and a fourth nucleic acid sequence that is at least 95% Identical to SEQ ID NO: 7 or 7A (at least 96%, at least 97%, at least 98%, at least 99%, or 100% identical to SEQ ID NO: 7 or 7A). In some embodiments, the self-replicating RNA molecule comprises a nucleic acid sequence comprising SEQ ID NOs: 1, 1A, 3, 3A, 5, 5A, 7, and 7A. In some embodiments, the replicase-encoding sequence encodes an amino acid sequence that is at least 95% identical to SEQ ID NO: 2 (e.g., at least 96%, at least 97%, at least 98%, or at least 99% (e.g., at least 99.1%, at least 99.2%, at least 99.3%, at least 99.4%, at least 99.5%, at least 99.6%, at least 99.7%, at least 99.8%, or at least 99.9%) identical to SEQ ID NO: 2). In some embodiments, the replicase-encoding sequence encodes an amino acid sequence that is at least 95% Identical to SEQ ID NO: 4 (e.g., at least 96%, at least 97%, at least 98%, or at least 99% (e.g., at least 99.1%, at least 99.2%, at least 99.3%, at least 99.4%, at least 99.5%, at least 99.6%, at least 99.7%, at least 99.8%, or at least 99.9%) identical to SEQ ID NO: 4).

In some embodiments, the replicase-encoding sequence encodes an amino acid sequence that is at least 95% identical to SEQ ID NO: 6 (e.g., at least 96%, at least 97%, at least 98%, or at least 99% (e.g., at least 99.1%, at least 99.2%, at least 99.3%, at least 99.4%, at least 99.5%, at least 99.6%, at least 99.7%, at least 99.8%, or at least 99.9%) identical to SEQ ID NO: 6). In some embodiments, the replicase-encoding sequence encodes an amino acid sequence that is at least 95% identical to SEQ ID NO: 8 (e.g., at least 96%, at least 97%, at least 98%, or at least 99% (e.g., at least 99.1%, at least 99.2%, at least 99.3%, at least 99.4%, at least 99.5%, at least 99.6%, at least 99.7%, at least 99.8%, or at least 99.9%) identical to SEQ ID NO: 8). In some embodiments, the replicase-encoding sequence encodes a first amino acid sequence that is at least 95% Identical to SEQ ID NO: 2 (at least 96%, at least 97%, at least 98%, at least 99%, or 100% identical to SEQ ID NO: 2), a second amino acid sequence that is at least 95% identical to SEQ ID NO: 4 (at least 96%, at least 97%, at least 98%, at least 99%, or 100% Identical to SEQ ID NO: 4), a third amino acid sequence that is at least 95% identical to SEQ ID NO: 6 (at least 96%, at least 97%, at least 98%, at least 99%, or 100% identical to SEQ ID NO: 6), and a fourth amino acid sequence that is at least 95% identical to SEQ ID NO: 8 (at least 96%, at least 97%, at least 98%, at least 99%, or 100% identical to SEQ ID NO: 8). In some embodiments, the replicase-encoding sequence encodes an amino acid sequence comprising SEQ ID NOs: 2, 4, 6, and 8.

Mutant or wild-type virus sequences can be used. For example, in some instances, the self-replicating RNA includes an attenuated TC83 mutant of VEE replicase. Other mutations in the replicase are contemplated herein, including replicase mutated replicases (e.g., mutated VEE replicases) obtained by in-vitro evolution methods, e.g., as taught by Yingzhong et al., Sci Rep. 2019, 9: 6932, the methodology of which is incorporated herein by reference.

In some instances, a self-replicating RNA molecule includes (i) a replicase-encoding sequence (e.g., an RNA sequence that encodes an RNA-dependent RNA polymerase which can transcribe RNA from the self-replicating RNA molecule) and (ii) a heterologous modulatory gene. The polymerase can be an alphavirus replicase, e.g., an alphavirus replicase comprising one, two, three, or all four alphavirus nonstructural proteins nsP1, nsP2, nsP3, and nsP4. In some instances, the polymerase is a VEE replicase, e.g., a VEE replicase comprising one, two, three, or all four alphavirus nonstructural proteins nsP1, nsP2, nsP3, and nsP4.

In some instances of the present invention, a self-replicating RNA molecule does not encode alphavirus structural proteins (e.g., capsid proteins). Such self-replicating RNA can lead to the production of genomic RNA copies of itself in a cell, but not to the production of RNA-containing virions. The inability to produce these virions means that, unlike a wild-type alphavirus, the self-replicating RNA molecule cannot perpetuate itself in infectious form. The alphavirus structural proteins can be replaced by gene(s) encoding the heterologous modulatory protein(s) of interest, such that the subgenomic transcript encodes the heterologous modulatory protein(s) rather than the structural alphavirus virion proteins.

Accordingly, in some instances, a self-replicating RNA molecule of the invention can have two open reading frames. The first (5′) open reading frame encodes a replicase; the second (3′) open reading frame encodes one or more (e.g., two or three) heterologous modulatory proteins (e.g., any of the immunomodulatory proteins, or combinations thereof, described herein). In some embodiments, the RNA may have additional (e.g., downstream) open reading frames, e.g., to encode further heterologous genes or to encode accessory polypeptides.

Suitable self-replicating RNA molecules can have various lengths. In some embodiments of the invention, the length of the self-replicating RNA molecule is from 5,000 to 50,000 nucleotides (i.e., 5 kb to 50 kb). In some instances, the self-replicating RNA molecule is 5 kb to 20 kb in length (e.g., from 6 kb to 18 kb, from 7 kb to 16 kb, from 8 kb to 14 kb, or from 9 kb to 12 kb in length, e.g., from 5 kb to 6 kb, from 6 kb to 7 kb, from 7 kb to 8 kb, from 8 kb to 9 kb, from 9 kb to 10 kb, from 10 kb to 11 kb, from 11 kb to 12 kb, from 12 kb to 13 kb, from 13 kb to 14 kb, from 14 kb to 15 kb, from 15 kb to 16 kb, from 16 kb to 18 kb, or from 18 kb to 20 kb in length, e.g., about 5 kb, about 6 kb, about 7 kb, about 8 kb, about 9 kb, about 10 kb, about 10.5 kb, about 11 kb, about 11.5 kb, about 12 kb, about 12.5 kb, about 13 kb, about 14 kb, about 15 kb, about 16 kb, about 17 kb, about 18 kb, about 19 kb, or about 20 kb in length).

A self-replicating RNA molecule may have a 3′ poly-A tail. Additionally, the self-replicating RNA molecule may include a poly-A polymerase recognition sequence (e.g., AAUAAA).

Self-replicating RNA molecules can be prepared through any method known in the art or described herein, e.g., by in-vitro transcription (IVT). IVT can use a (cDNA) template created and propagated in plasmid form in bacteria or created synthetically (for example by gene synthesis and/or polymerase chain-reaction (PCR) engineering methods). For instance, a DNA-dependent RNA polymerase (such as the bacteriophage T7, T3 or SP6 RNA polymerases) can be used to transcribe the RNA from a DNA template. Appropriate capping and poly-A addition reactions can be used as required (although the replicon's poly-A is usually encoded within the DNA template). These RNA polymerases can have stringent requirements for the transcribed 5′ nucleotide(s) and in some embodiments these requirements must be matched with the requirements of the encoded replicase, to ensure that the IVT-transcribed RNA can function efficiently as a substrate for its self-encoded replicase.

In some instances, self-replicating RNA can include (in addition to any 5′ cap structure) one or more nucleotides having a modified nucleobase. For example, the self-replicating RNA molecule can include m5C (5-methylcytidine), m5U (5-methyluridine), m6A (N-6-methyladenosine), s2U (2-thiouridine), Um (2′O-methyluridine), m1A (1-methyladenosine); m2A (2-methyladenosine); Am (2′-β-methyladenosine); ms2m6A (2-methylthio-N-6-methyladenosine); i6A (N-6-isopentenyladenosine); ms2i6A (2-methylthio-N6 isopentenyladenosine); io6A (N-6-(ci-hydroxyisopentenyl)adenosine); ms2io6A (2-methylthio-N-6-(cis-hydroxyisopentenyl) adenosine); g6A (N-6-glycinylcarbamoyladenosine); t6A (N-6-threonyl carbamoyladenosine); ms2t6A (2-methylthio-N-6-threonyl carbamoyladenosine); m6t6A (N-6-methyl-N-6-threonylcarbamoyladenosine); hn6A(N6-hydroxynorvalylcarbamoyl adenosine); ms2hn6A (2-methylthio-N-6-hydroxynorvalyl carbamoyladenosine); Ar(p) (2-O-ribosyladenosine (phosphate)); I (inosine); m11 (1-methylinosine); m′1m (1,2-O-dimethylinoline); m3C (3-methylcytidine); Cm (2T-O-methylcytidine); s2C (2-thiocytidine); ac4C(N-4-acetylcytidine); f5C (5-formylcytidine); m5Cm (5,2-O-dimethylcytidine); ao4Cm (N4acetyl2TOmethylcytidine); k2C (lysidine); m1G (1-methylguanosine); m2G (N2-methylguanosine); m7G (7-methylguanosine); Gm (2′-O-methylguanosine); m22G (N2,N2-dimethylguanosine); m2Gm (N2,2′-O-dimethylguanosine); m22Gm (N2,N2,2′-β-trimethylguanosine); Gr(p) (2′-O-ribosylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (gaitactosyl-queuosine); manQ (mannosyl-queuosine); preQo (7-cyano-7-deazaguanosine); preQi (7-aminomethyl-7-deazaguanosine); G (archaeosine); D (dihydrouridine); m5Um (52′-O-dimethylidine); s4U (4-thiouridine); m5s2U (5-methyl-2-thiouridine); s2Um (2-thio-2′-O-methyluridine); acp3U (3-(3-amino-3-carboxypropyl)uridine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl)uridine)); mchm5U (5-(cerboxyhydroxymethyl)uridine methyl ester); mcm5U (5-methoxycarbonyl methyluridine); mcm5Um (S-methoxycarbonylmethyl-2-O-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-O-methyluridine); cmnm5U (5-carboxymethylaminomethyluridine); cnmm5Um (5-carboxymethylaminomethyl-2-L-Omethyluridine); cmnm5s2U (5-carboxymethylaminomethyl-2-thiouridine); m62A (N6,N6-dimethyladenosine); Tm (2′-O-methylinosine); m4C(N-4-methylcysteine); m4Cm (N4,2-O-dimethylcytidine); hm5C (5-hydroxymethylcytidine); m3U (3-methyluridine); cm5U (5-carboxymethyluridine); m6 Am (N6,T-O-dimethyladenosine); m62 Am (N6,N6,O-2-trimethyladenosine); m27G (N2,7-dimethylguanosine); m227G (N2,N2,7-trimethylguanosine); m3Um (3,2T-O-dimethyluridine); m5D (5-methyldihydrouridine); f5Cm (5-formyl-2′-O-methylcytidine); m1Gm (1,2′-O-dimethylguanosine); m′Am (1,2-O-dimethyl adenosine) iriomethyluridine); tm5s2U (S-taurinomethyl-2-thiouridine)); imG-14 (4-demethyl guanosine); imG2 (isoguanosine); or ac6A (N-6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(C1-C6)-alkyluracil, 5-methyluracil, 5-(C2-C6)-alkenyluracil, 5-(C2-C6)-alkynyluracil, 5-(hydroxymethyl)uracil, 5-chlorouracil, 5-fluorouracil, 5-bromouracil, 5-hydroxycytosine, 5-(C1-C6)-alkylcytosine, 5-methylcytosine, 5-(C2-C6)-alkenylcytosine, 5-(C2-C6)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, or an abasic nucleotide. For instance, a self-replicating RNA molecule may include one or more modified pyrimidine nucleobases, such as pseudouridine and/or 5-methylcytosine residues.

In other embodiments of the present invention, the self-replicating RNA molecule is substantially free of nucleotides having a modified nuclease. For example, in some instances, the self-replicating RNA molecule includes no modified nucleobases, and may include no modified nucleotides, i.e., all of the nucleotides in the RNA are standard A, C, G and U ribonucleotides (except for any 5′ cap structure, which may include a T-methylguanosine).

In some instances, a self-replicating RNA includes only phosphodiester linkages between nucleosides. In some instances, a self-replicating RNA includes phosphoramidate, phosphorothioate, and/or methylphosphonate linkages. In any of the polycistronic self-replicating RNA molecules described herein, cleavage sites can be designed between protein-coding regions.

In a particular embodiment, the RNA according to the invention does not encode a reporter molecule, such as luciferase or a fluorescent protein, such as green fluorescent protein (GFP).

In some embodiments, the heterologous protein encoded by the self-replicating RNA is a variant of any of the heterologous proteins described herein. Additionally, or alternatively, the replicase encoded by the self-replicating RNA can be a variant of any of the replicases described herein. In some embodiments, the variant is a functional fragment (e.g., a fragment of the protein that is functionally similar or functionally equivalent to the protein).

Pharmaceutical Compostions

Provided herein are pharmaceutical compositions having any of the nucleic acid vectors (e.g., circular DNA vector or self-replicating RNA molecules) described herein in a pharmaceutically acceptable carrier. Thus, in some instances, pharmaceutical compositions of the invention include a nucleic acid vector (e.g., a circular DNA vector or self-replicating RNA molecules) having an immunomodulatory sequence (e.g., an immunomodulatory sequence having multiple transcription units, or a polycistronic immunomodulatory sequence) comprising a dendritic cell chemoattractant-encoding gene (e.g., an XCL1-encoding gene, an XCL2-encoding gene, a CCL5-encoding gene, or a CCL4-encoding gene) and one or more immunomodulatory protein-encoding genes (e.g., a dendritic cell growth factor or activator-encoding gene (e.g., a FLT3L-encoding gene, a GMCSF-encoding gene, or a CD40L-encoding gene) or a lymphocyte signaling protein-encoding gene (e.g., a cytokine-encoding gene (e.g., an IL-12-encoding gene or an IL-15-encoding gene) or a cytokine-encoding gene (e.g., a CXCL9-encoding gene or a CXCL10-encoding gene)) in a pharmaceutically acceptable carrier.

In other instances, the pharmaceutical compositions of the invention include one or more nucleic acid vectors (e.g., one or more circular DNA vectors, e.g., a heterogeneous composition nucleic acid vectors (e.g., circular DNA vectors), each encoding a different immunomodulatory genes). For example, in such instances, pharmaceutical compositions of the invention include two or more (e.g., two or three) nucleic acid vectors (e.g., circler DNA vectors), wherein each vector includes an immunomodulatory sequence (e.g., a monocistronic immunomodulatory sequence) comprising a dendritic cell chemoattractant-encoding gene (e.g., an XCL1-encoding gene, an XCL2-encoding gene, a CCL5-encoding gene, or a CCL4-encoding gene), a dendritic cell growth factor or activator-encoding gene (e.g., a FLT3L-encoding gene, a GMCSF-encoding gene, or a CD40L-encoding gene), or a lymphocyte signaling protein-encoding gene (e.g., a cytokine-encoding gene (e.g., an IL-12-encoding gene or an IL-15-encoding gene) or a chemokine-encoding gene (e.g., a CXCL9-encoding gene or a CXCL10-encoding gene)) in a pharmaceutically acceptable carrier.

In some embodiments, the pharmaceutical composition includes two nucleic acid vectors (e.g., circular DNA vectors), wherein: (i) the first nucleic acid vector (e.g., circular DNA vector) includes a immunomodulatory sequence (e.g., a monocistronic immunomodulatory sequence) comprising a dendritic cell chemoattractant-encoding gene (e.g., an XCL1-encoding gene, an XCL2-encoding gene, a CCL5-encoding gene, or a CCL4-encoding gene) or a dendritic cell growth factor or activator-encoding gene (e.g., a FLT3L-encoding gene, a GMCSF-encoding gene, or a CD40L-encoding gene); and (ii) the second nucleic acid vector (e.g., circular DNA vector) includes a lymphocyte signaling protein-encoding gene (e.g., a cytokine-encoding gene (e.g., an IL-12-encoding gene or an IL-15-encoding gene) or a chemokine-encoding gene (e.g., a CXCL9-encoding gene or a CXCL10-encoding gene)) in a pharmaceutically acceptable carrier.

In some instances, the pharmaceutical composition includes two nucleic acid vectors (e.g., circular DNA vectors), wherein: (i) the first nucleic acid vector (e.g., circular DNA vector) includes a immunomodulatory sequence (e.g., a monocistronic immunomodulatory sequence) comprising a dendritic cell chemoattractant-encoding gene (e.g., an XCL1-encoding gene, an XCL2-encoding gene, a CCL5-encoding gene, or a CCL4-encoding gene); and (ii) the second nucleic acid vector (e.g., circular DNA vector) includes a lymphocyte signaling protein-encoding gene (e.g., a cytokine-encoding gene (e.g., an IL-12-encoding gene or an IL-15-encoding gene) or a cytokine-encoding gene (e.g., a CXCL9-encoding gene or a CXCL10-encoding gene)) in a pharmaceutically acceptable carrier. In some instances, the pharmaceutical composition includes two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL1-encoding gene; and (1) the second nucleic acid vector (e.g., circular DNA vector) includes a cytokine-encoding gene (e.g., an IL-12-encoding gene or an IL-15-encoding gene) or a cytokine-encoding gene (e.g., a CXCL9-encoding gene or a CXCL10-encoding gene)) In a pharmaceutically acceptable carrier. In some instances, the pharmaceutical composition includes two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL1-encoding gene; and (ii) the second nucleic acid vector (e.g., circular DNA vector) includes an IL-12-encoding gene in a pharmaceutically acceptable carrier. In some instances, the pharmaceutical composition includes two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL1-encoding gene; and (ii) the second nucleic acid vector (e.g., circular DNA vector) includes an IL-15-encoding gene in a pharmaceutically acceptable carrier. In some instances, the pharmaceutical composition includes two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL1-encoding gene; and (ii) the second nucleic acid vector (e.g., circular DNA vector) includes a CXCL9-encoding gene in a pharmaceutically acceptable carrier. In some instances, the pharmaceutical composition includes two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL1-encoding gene; and (ii) the second nucleic acid vector (e.g., circular DNA vector) includes a CXCL10-encoding gene in a pharmaceutically acceptable carrier.

In some instances, the pharmaceutical composition includes two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL2-encoding gene; and (ii) the second nucleic acid vector (e.g., circular DNA vector) includes a cytokine-encoding gene (e.g., an IL-12-encoding gene or an IL-15-encoding gene) or a chemokine-encoding gene (e.g., a CXCL9-encoding gene or a CXCL10-encoding gene)) in a pharmaceutically acceptable carrier. In some instances, the pharmaceutical composition includes two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL2-encoding gene; and (1) the second nucleic acid vector (e.g., circular DNA vector includes an IL-12-encoding gene in a pharmaceutically acceptable carrier. In some instances, the pharmaceutical composition includes two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL2-encoding gene; and (ii) the second nucleic acid vector (e.g., circular DNA vector) includes an IL-15-encoding gene in a pharmaceutically acceptable carrier. In some instances, the pharmaceutical composition includes two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL2-encoding gene; and 01) the second nucleic acid vector (e.g., circular DNA vector) includes a CXCL9-encoding gene in a pharmaceutically acceptable carrier. In some instances, the pharmaceutical composition includes two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL2-encoding gene; and (i) the second nucleic acid vector (e.g., circular DNA vector) includes a CXCL10-encoding gene in a pharmaceutically acceptable carrier.

In some instances, the pharmaceutical composition includes two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an CCL4-encoding gene; and (ii) the second nucleic acid vector (e.g., circular DNA vector) includes a cytokine-encoding gene (e.g., an IL-12-encoding gene or an IL-15-encoding gene) or a chemokine-encoding gene (e.g., a CXCL9-encoding gene or a CXCL10-encoding gene)) In a pharmaceutically acceptable carrier. In some instances, the pharmaceutical composition includes two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an CCL4-encoding gene; and (ii) the second nucleic acid vector (e.g., circular DNA vector) includes an IL-12-encoding gene in a pharmaceutically acceptable carrier. In some instances, the pharmaceutical composition includes two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an CCL4-encoding gene; and (11) the second nucleic acid vector (e.g., circular DNA vector) includes an IL-15-encoding gene in a pharmaceutically acceptable carrier. In some instances, the pharmaceutical composition includes two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an CCL4-encoding gene; and (ii) the second nucleic acid vector (e.g., circular DNA vector) includes a CXCL9-encoding gene in a pharmaceutically acceptable carrier. In some instances, the pharmaceutical composition includes two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an CCL4-encoding gene; and (ii) the second nucleic acid vector (e.g., circular DNA vector) includes a CXCL10-encoding gene in a pharmaceutically acceptable carrier.

In some instances, the pharmaceutical composition includes two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an CCL5-encoding gene; and (i) the second nucleic acid vector (e.g., circular DNA vector) includes a cytokine-encoding gene (e.g., an IL-12-encoding gene or an IL-15-encoding gene) or a chemokine-encoding gene (e.g., a CXCL9-encoding gene or a CXCL10-encoding gene)) in a pharmaceutically acceptable carrier. In some instances, the pharmaceutical composition includes nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an CCL5-encoding gene; and (ii) the second nucleic acid vector (e.g., circular DNA vector) includes an IL-12-encoding gene in a pharmaceutically acceptable carrier. In some instances, the pharmaceutical composition includes two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an CCL5-encoding gene; and (ii) the second nucleic acid vectors (e.g., circular DNA vector) includes an IL-15-encoding gene in a pharmaceutically acceptable carrier. In some instances, the pharmaceutical composition includes two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an CCL5-encoding gene; and (ii) the second nucleic acid vector (e.g., circular DNA vector) includes a CXCL9-encoding gene in a pharmaceutically acceptable carrier. In some instances, the pharmaceutical composition includes two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an CCL5-encoding gene; and (ii) the second nucleic acid vector (e.g., circular DNA vector) includes a CXCL10-encoding gene in a pharmaceutically acceptable carrier.

In some embodiments, the pharmaceutical composition includes two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising a dendritic cell growth factor or activator-encoding gene (e.g., a FLT3L-encoding gene, a GMCSF-encoding gene, or a CD40L-encoding gene); and (ii) the second nucleic acid vector (e.g., circular DNA vector) includes a lymphocyte signaling protein-encoding gene (e.g., a cytokine-encoding gene (e.g., an IL-12-encoding gene or an IL-15-encoding gene) or a chemokine-encoding gene (e.g., a CXCL9-encoding gene or a CXCL10-encoding gene)) in a pharmaceutically acceptable carrier.

In some embodiments, the pharmaceutical composition includes three nucleic acid vectors (e.g., circular DNA vectors), wherein: (i) the first nucleic acid vector (e.g., circular DNA vector) includes (a) an immunomodulatory sequence (e.g., a monocistronic immunomodulatory sequence) comprising a dendritic cell chemoattractant-encoding gene (e.g., an XCL1-encoding gene, an XCL2-encoding gene, a CCL5-encoding gene, or a CCL4-encoding gene); (ii) the second nucleic acid vector (e.g., circular DNA vector) includes a dendritic cell growth factor or activator-encoding gene (e.g., a FLT3L-encoding gene, a GMCSF-encoding gene, or a CD40L-encoding gene); and (iii) the third nucleic acid vector (e.g., circular DNA vector) includes a lymphocyte signaling protein-encoding gene (e.g., a cytokine-encoding gene (e.g., an IL-12-encoding gene or an IL-15-encoding gene) or a chemokine-encoding gene (e.g., a CXCL9-encoding gene or a CXCL10-encoding gene)) in a pharmaceutically acceptable carrier.

In some embodiments, the pharmaceutical composition includes three nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL1-encoding gene; (ii) the second nucleic acid vector (e.g., circular DNA vector) includes a FLT3L-encoding gene; and (iii) the third nucleic acid vector (e.g., circular DNA vector) includes a lymphocyte signaling protein-encoding gene (e.g., a cytokine-encoding gene (e.g., an IL-12-encoding gene or an IL-15-encoding gene) or a chemokine-encoding gene (e.g., a CXCL9-encoding gene or a CXCL10-encoding gene)) in a pharmaceutically acceptable carrier. In some embodiments, the pharmaceutical composition includes three nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL1-encoding gene; (ii) the second nucleic acid vector (e.g., circular DNA vector) includes a GM-CSF-encoding gene; and (iii) the third nucleic acid vector (e.g., circular DNA vector) includes a lymphocyte signaling protein-encoding gene (e.g., a cytokine-encoding gene (e.g., an IL-12-encoding gene or an IL-15-encoding gene) or a chemokine-encoding gene (e.g., a CXCL9-encoding gene or a CXCL10-encoding gene)) in a pharmaceutically acceptable carrier.

In some embodiments, the pharmaceutical composition includes three nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL1-encoding gene; (ii) the second nucleic acid vector (e.g., circular DNA vector) includes a FLT3-encoding gene; and (iii) the third nucleic acid vector (e.g., circular DNA vector) includes an IL-12-encoding gene in a pharmaceutically acceptable carrier. In some embodiments, the pharmaceutical composition includes three nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL1-encoding gene; (ii) the second nucleic acid vector (e.g., circular DNA vector) includes a FLT3-encoding gene; and (11) the third nucleic acid vector (e.g., circular DNA vector) includes an IL-15-encoding gene in a pharmaceutically acceptable carrier. In some embodiments, the pharmaceutical composition includes three nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL1-encoding gene; (1) the second nucleic acid vector (e.g., circular DNA vector) includes a FLT3-encoding gene; and (iii) the third nucleic acid vector (e.g., circular DNA vector) includes a CXCL9-encoding gene in a pharmaceutically acceptable carrier. In some embodiments, the pharmaceutical composition includes three nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL1-encoding gene; (ii) the second nucleic acid vector (e.g., circular DNA vector) includes a FLT3-encoding gene; and (11) the third nucleic acid vector (e.g., circular DNA vector) includes a CXCL10-encoding gene in a pharmaceutically acceptable carrier.

In some embodiments, the pharmaceutical composition includes three nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL1-encoding gene; (ii) the second nucleic acid vector (e.g., circular DNA vector) includes a GM-CSF-encoding gene; and (iii) the third nucleic acid vector (e.g., circular DNA vector) includes an IL-12-encoding gene in a pharmaceutically acceptable carrier. In some embodiments, the pharmaceutical composition includes three nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL1-encoding gene; (ii) the second nucleic acid vector (e.g., circular DNA vector) includes a GM-CSF-encoding gene; and (iii) the third nucleic acid vector (e.g., circular DNA vector) includes an IL-15-encoding gene in a pharmaceutically acceptable carrier. In some embodiments, the pharmaceutical composition includes three nucleic acid vector (e.g., circular DNA vector), wherein: (i) the first nucleic acid vector (e.g., circular DNA vector) includes an immunomodulatory sequence (e.g., a monocistronic immunomodulatory sequence) comprising an XCL1-encoding gene; (1) the second nucleic acid vector (e.g., circular DNA vector) includes a GM-CSF-encoding gene; and (iii) the third nucleic acid vector (e.g., circular DNA vector) includes an CXCL9-encoding gene in a pharmaceutically acceptable carrier. In some embodiments, the pharmaceutical composition includes three nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL1-encoding gene; (ii) the second nucleic acid vector (e.g., circular DNA vector) includes a GM-CSF-encoding gene; and (iii) the third nucleic acid vector (e.g., circular DNA vector) includes a CXCL10-encoding gene in a pharmaceutically acceptable carrier.

In any of the aforementioned pharmaceutical compositions, the immunomodulatory sequence can be within a self-replicating RNA-encoding sequence containing a replicase-encoding sequence that transcribes RNA from the self-replicating RNA molecule.

In some instances, the pharmaceutical composition includes two self-replicating RNA molecules, wherein: (i) the first self-replicating RNA molecule includes (a) a replicase-encoding sequence that transcribes RNA from the self-replicating RNA molecule and (b) an immunomodulatory sequence (e.g., a monocistronic immunomodulatory sequence) comprising a dendritic cell chemoattractant-encoding gene (e.g., an XCL1-encoding gene, an XCL2-encoding gene, a CCL5-encoding gene, or a CCL4-encoding gene); and (ii) the second self-replicating RNA molecule includes (a) a replicase-encoding sequence that transcribes RNA from the self-replicating RNA molecule and (b) a lymphocyte signaling protein-encoding gene (e.g., a cytokine-encoding gene (e.g., an IL-12-encoding gene or an IL-15-encoding gene) or a chemokine-encoding gene (e.g., a CXCL9-encoding gene or a CXCL10-encoding gene)) in a pharmaceutically acceptable carrier.

In some embodiments, the pharmaceutical composition includes three self-replicating RNA molecules, wherein: (i) the first self-replicating RNA molecule includes (a) a replicase-encoding sequence that transcribes RNA from the self-replicating RNA molecule and (b) an immunomodulatory sequence (e.g., a monocistronic immunomodulatory sequence) comprising a dendritic cell chemoattractant-encoding gene (e.g., an XCL1-encoding gene, an XCL2-encoding gene, a CCL5-encoding gene, or a CCL4-encoding gene); (ii) the second self-replicating RNA molecule includes (a) a replicase-encoding sequence that transcribes RNA from the self-replicating RNA molecule and (b) or a dendritic cell growth factor or activator-encoding gene (e.g., a FLT3L-encoding gene, a GMCSF-encoding gene, or a CD40L-encoding gene); and (iii) the third self-replicating RNA molecule includes (a) a replicase-encoding sequence that transcribes RNA from the self-replicating RNA molecule and (b) a lymphocyte signaling protein-encoding gene (e.g., a cytokine-encoding gene (e.g., an IL-12-encoding gene or an IL-15-encoding gene) or a cytokine-encoding gene (e.g., a CXCL9-encoding gene or a CXCL10-encoding gene)) in a pharmaceutically acceptable carrier. Pharmaceutically acceptable carriers may include excipients and/or stabilizers that are nontoxic to the individual at the dosages and concentrations employed. In some embodiments, the pharmaceutically acceptable carrier is an aqueous pH buffered solution. Examples of pharmaceutically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrroldine; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as tween, polyethylene glycol (PEG), and pluronics.

A pharmaceutical composition having a self-replicating RNA molecule of the invention may contain a pharmaceutically acceptable carrier. If the composition is provided in liquid form, the carrier may be water (e.g., pyrogen-free water), isotonic saline, or a buffered aqueous solution, e.g., a phosphate buffered solution or a citrate buffered solution. Injection of the pharmaceutical composition may be carried out in water or a buffer, such as an aqueous buffer, e.g., containing a sodium salt (e.g., at least 50 mM of a sodium salt), a calcium salt (e.g., at least 0.01 mM of a calcium salt), or a potassium salt (e.g., at least 3 mM of a potassium salt). According to a particular embodiment, the sodium, calcium, or potassium salt may occur in the form of their halogenides, e.g., chlorides, iodides, or bromides, in the form of their hydroxides, carbonates, hydrogen carbonates, or sulfates, etc. Without being limited thereto, examples of sodium salts include NaCl, NaI, NaBr, Na₂CO₂, NaHCO₂, and Na₂SO₄. Examples of potassium salts include, e.g., KCl, KI, KBr, K₂CO₂, KHCO₂, and K₂SO₄. Examples of calcium salts include, e.g., CaCl₂, CaI₂, CaBr₂, CaCO₂, CaSO₄, and Ca(OH)₂. Additionally, organic anions of the aforementioned cations may be contained in the buffer. According to a particular embodiment, the buffer suitable for injection purposes as defined above, may contain salts selected from sodium chloride (NaCl), calcium chloride (CaCl₂) or potassium chloride (KCl), wherein further anions may be present. CaCl₂) can also be replaced by another salt, such as KCl. In some embodiments, salts in the injection buffer are present in a concentration of at least 50 mM sodium chloride (NaCl), at least 3 mM potassium chloride (KCl), and at least 0.01 mM calcium chloride (CaCl₂). The injection buffer may be hypertonic, isotonic, or hypotonic with reference to the specific reference medium, i.e., the buffer may have a higher, identical or lower salt content with reference to the specific reference medium, wherein preferably such concentrations of the afore mentioned salts may be used, which do not lead to damage of calls due to osmosis or other concentration effects. Reference media are can be liquids such as blood, lymph, cytosolic liquids, other body liquids, or common buffers. Such common buffers or liquids are known to a skilled person. Ringer-Lactate solution is particularly preferred as a liquid basis.

One or more compatible solid or liquid filers, diluents, or encapsulating compounds may be suitable for administration to a person. The constituents of the pharmaceutical composition according to the invention are capable of being mixed with the nucleic acid vector according to the invention as defined herein. In such a manner that no interaction occurs, which would substantially reduce the pharmaceutical effectiveness of the (pharmaceutical) composition according to the invention under typical use conditions. Pharmaceutically acceptable carriers, fillers and diluents can have sufficiently high purity and sufficiently low toxicity to make them suitable for administration to an individual being treated. Some examples of compounds which can be used as pharmaceutically acceptable carriers, filers, or constituents thereof are sugars, such as lactose, glucose, trehalose, and sucrose; starches, such as corn starch or potato starch; dextrose; cellulose and its derivatives, such as sodium carboxymethylcellulose, ethylcellulose, cellulose acetate; powdered tragacanth; malt; gelatin; tallow; solid glidants, such as stearic acid, magnesium stearate; calcium sulfate; vegetable oils, such as groundnut oil, cottonseed oil, sesame oil, olive oil, corn oil and oil from Theobroma; polyols, such as polypropylene glycol, glycerol, sorbitol, mannitol, and polyethylene glycol; or alginic acid.

The choice of a pharmaceutically acceptable carrier can be determined, according to the manner in which the pharmaceutical composition is administered.

Suitable unit dose forms for injection include sterile solutions of water, physiological saline, and mixtures thereof. The pH of such solutions may be adjusted to about 7.4. Suitable carriers for injection include hydrogels, devices for controlled or delayed release, polylactic acid, and collagen matrices. Suitable pharmaceutically acceptable carriers for topical application include those which are suitable for use in lotions, creams, gels and the like. If the pharmaceutical composition is to be administered perorally, tablets, capsules and the like are the preferred unit dose form.

Further additives which may be included in the pharmaceutical composition are emulsifiers, such as tween; wetting agents, such as sodium lauryl sulfate; coloring agents; pharmaceutical carriers; stabilizers; antioxidants; and preservatives.

The pharmaceutical composition according to the present invention may be provided in liquid or in dry (e.g., lyophilized) form. In a particular embodiment, the nucleic acid vector of the pharmaceutical composition is provided in lyophilized form. Lyophilized compositions including nucleic acid vector of the invention may be reconstituted in a suitable buffer, advantageously based on an aqueous carrier, prior to administration, e.g., Ringer-Lactate solution, Ringer solution, or a phosphate buffer solution.

In certain embodiments of the invention, any of the nucleic acid vectors of the invention can be complexed with one or more cationic or polycationic compounds, e.g., cationic or polycationic polymers, cationic or polycationic peptides or proteins, e.g., protamine, cationic or polycationic polysaccharides, and/or cationic or polycationic lipids.

According to a particular embodiment, the nucleic acid vector of the invention may be complexed with lipids to form one or more liposomes, lipoplexes, or lipid nanoparticles. Therefore. In one embodiment, the inventive composition comprises liposomes, lipoplexes, and/or lipid nanoparticles comprising a nucleic acid vector.

Lipid-based formulations can be effective delivery systems for nucleic acid vectors due to their biocompatibility and their ease of large-scale production. Cationic lipids have been widely studied as synthetic materials for delivery of nucleic acids. After mixing together, nucleic acids are condensed by cationic lipids to form lipid/nucleic acid complexes known as lipoplexes. These lipid complexes are able to protect genetic material from the action of nucleases and deliver it into cells by interacting with the negatively charged cell membrane. Lipoplexes can be prepared by directly mixing positively charged lipids at physiological pH with negatively charged nucleic acids.

Conventional liposomes include of a lipid bilayer that can be composed of cationic, anionic, or neutral phospholipids and cholesterol, which encloses an aqueous core. Both the lipid bilayer and the aqueous space can incorporate hydrophobic or hydrophilic compounds, respectively. Liposome characteristics and behavior in-vivo can be modified by addition of a hydrophilic polymer coating, e.g., polyethylene glycol (PEG), to the liposome surface to confer steric stabilization. Furthermore, liposomes can be used for specific targeting by attaching ligands (e.g., antibodies, peptides, and carbohydrates) to its surface or to the terminal end of the attached PEG chains.

Liposomes are colloidal lipid-based and surfactant-based delivery systems composed of a phospholipid bilayer surrounding an aqueous compartment. They may present as spherical vesicles and can range in size from 20 nm to a few microns. Cationic lipid-based liposomes are able to complex with negatively charged nucleic acids via electrostatic interactions, resulting in complexes that offer biocompatibility, low toxicity, and the possibility of the large-scale production required for in vivo clinical applications. Liposomes can fuse with the plasma membrane for uptake; once inside the cell, the liposomes are processed via the endocytic pathway and the genetic material is then released from the endosome/carrier into the cytoplasm.

Cationic liposomes can serve as delivery systems RNAs. Cationic lipids, such as MAP, (1,2-dioleoyl-3-trimethylammonium-propene) and DOTMA (N-[1-(2,3-dioleoyloxy)propyl]-N,N,N-trimethyl-ammonium methyl sulfate) can form complexes or lipoplexes with negatively charged nucleic acids to form nanoparticles by electrostatic interaction, providing high in vitro transfection efficiency. Furthermore, neutral lipid-based nanoliposomes for nucleic acid vector delivery as e.g., neutral 1,2-dioleoyl-sn-glycero-3-phosphatidylcholine (DOPC)-based nanoliposomes are available.

Thus, in one embodiment of the invention, the nucleic acid vector of the invention is complexed with cationic lipids and/or neutral lipids and thereby forms liposomes, lipid nanoparticles, lipoplexes or neutral lipid-based nanoliposomes.

In a particular embodiment, a pharmaceutical composition according to the invention comprises the nucleic acid vector of the invention that is formulated together with a cationic or polycationic compound and/or with a polymeric carder. Accordingly, in a further embodiment of the invention, the nucleic acid vector as defined herein is associated with or complexed with a cationic or polycationic compound or a polymeric carrier, optionally in a weight ratio selected from a range of about 5:1 (w/w) to about 0.25:1 (w/w), e.g., from about 5:1 (w/w) to about 0.5:1 (w/w), e.g., from about 4:1 (w/w) to about 1:1 (w/w) or of about 3:1 (w/w) to about 1:1 (w/w). e.g., from about 3:1 (w/w) to about 2:1 (w/w) of nucleic acid vector to cationic or polycationic compound and/or with a polymeric carrier; or optionally in a nitrogen/phosphate (N/P) ratio of nucleic acid vector to cationic or polycationic compound and/or polymeric carrier in the range of about 0.1-10, e.g., in a range of about 0.3-4 or 0.3-1, e.g., in a range of about 0.5-1 or 0.7-1, e.g., in a range of about 0.3-0.9 or 0.5-0.9. For example, the N/P ratio of the nucleic acid vector to the one or more polycations is in the range of about 0.1 to 10, including a range of about 0.3 to 4, of about 0.5 to 2, of about 0.7 to 2 and of about 0.7 to 1.5.

The nucleic acid vectors described herein can also be associated with a vehicle, transfection or complexation agent for increasing the transfection efficiency and/or the expression of the modulatory gene according to the invention.

In some instances, the nucleic acid vector according to the invention is complexed with one or more polycations, preferably with protamine or oligofectamine. Further cationic or polycationic compounds, which can be used as transfection or complexation agent may include cationic polysaccharides, for example chitosan, polybrene, cationic polymers, e.g. polyethyleneimine (PEI), cationic lipids, e.g. DOTMA: [1-(2,3-sioleyloxy)propyl)]-N,N,N-trimethylammonium chloride, DMRIE, di-C14-amidine, DOTIM, SAINT, DC-Chol, BGTC, CTAP, DOPE, LEAP, DOPE: Dioleyl phosphatidylethanol-amine, DOSPA, DODAB, DOIC, DMEPC, DOGS: Dioctadecylamidoglicylspermin, DIMRI: Dimyristo-oxypropyl dimethyl hydroxyethyl ammonium bromide, MAP dioleoyloxy-3-(trimethylammonio)propane, DC-6-14: O,O-ditetradecanoyl-N-(α-trimethylammonioacetyl)diethanolamine chloride, CLIP1: rac-[(2,3-dioctadecyloxypropyl)(2-hydroxyethyl)]-dimethylammonium chloride, CLIP6: rac-[2(2,3-dihexadecyloxypropyl-oxymethyloxy)ethyl]trimethylammonium, CLIP9: rac-[2(2,3-dihexadecyloxypropyl-oxysuccinyloxy)ethyl]-trimethylammonium, oligofectamine, or cationic or polycationic polymers, e.g. modified polyaminoacids, such as β-aminoacid-polymers or reversed polyamides, etc., modified polyethylenes, such as PVP (poly(N-ethyl-4-vinylpydidinium bromide)), etc., modified acrylates, such as pDMAEMA (poly(dimethylaminoethyl methylacrylate)), etc., modified amidoamines such as pAMAM (poly(amidoamine)), etc., modified polybetaaminoester (PBAE), such as damine end modified 1,4 butanediol diacrylate-co-5-amino-1-pentanol polymers, etc., dendrimers, such as polypropylamine dendimers or pAMAM based dendrimers, etc., polyimine(s), such as PEI: poly(ethyleneimine), poly(propyleneimine), etc., polyallylamine, sugar backbone based polymers, such as cyclodextrin based polymers, dextran based polymers, chitosan, etc., slian backbone based polymers, such as PMOXA-PDMS copolymers, etc., block polymers consisting of a combination of one or more cationic blocks (e.g. selected from a cationic polymer as mentioned above) and of one or more hydrophilic or hydrophobic blocks (e.g. polyethyleneglycole); etc.

According to a particular embodiment, the pharmaceutical composition of the invention includes the nucleic acid vector (e.g., circular DNA vector) encapsulated within or attached to a polymeric carrier. A polymeric carrier used according to the invention might be a polymeric carrier formed by disulfide-crosslinked cationic components. The disulfide-crosslinked cationic components may be the same or different from each other. The polymeric carrier can also contain further components. It is also particularly preferred that the polymeric carder used according to the present invention comprises mixtures of cationic peptides, proteins or polymers and optionally further components as defined herein, which are crosslinked by disulfide bonds as described herein. In this context, the disclosure of WO 2012/013326 is incorporated herewith by reference. In this context, the cationic components that form basis for the polymeric carrier by disulfide-crosslinkage are typically selected from any suitable cationic or polycationic peptide, protein or polymer suitable for this purpose, particular any cationic or polycationic peptide, protein or polymer capable of complexing the nucleic acid vector as defined herein or a further nucleic acid comprised in the composition, and thereby preferably condensing the nucleic acid vector. The cationic or polycationic peptide, protein or polymer, may be a linear molecule; however, branched cationic or polycationic peptides, proteins or polymers may also be used.

Every disulfide-crosslinking cationic or polycationic protein, peptide or polymer of the polymeric carrier, which may be used to complex the nucleic acid vector according to the invention included as part of the pharmaceutical composition of the invention may contain at least one SH moiety (e.g., at least one cysteine residue or any further chemical group exhibiting an SH moiety) capable of forming a disulfide linkage upon condensation with at least one further cationic or polycationic protein, peptide or polymer as cationic component of the polymeric carrier as mentioned herein.

Such polymeric carriers used to complex the nucleic acid vectors of the present invention may be formed by disulfide-crosslinked cationic (or polycationic) components. In particular, such cationic or polycationic peptides or proteins or polymers of the polymeric carder, which comprise or are additionally modified to comprise at least one SH moiety, can be selected from proteins, peptides, and polymers as a complexation agent.

In other embodiments, the pharmaceutical composition according to the invention may be administered naked without being associated with any further vehicle, transfection, or complexation agent.

Pharmaceutical compositions provided herein may also contain more than one active ingredient as necessary for the particular cancer being treated. For example, in some instances, any of the pharmaceutical compositions described herein may include an additional therapeutic agent, such as an anti-cancer agent (e.g., a chemotherapeutic agent, a checkpoint inhibitor, a cytotoxic agent, a growth inhibitory agent, an anti-angiogenic agent, a cytokine, a cytokine antagonist, an antibody-drug conjugate, a cancer vaccine, or a combination thereof). In particular embodiments, the additional therapeutic agent is a checkpoint inhibitor, e.g., a PD-1 axis binding antagonist (e.g., an anti-PD-1 antagonist or an anti-PD-L1 antibody), such as pembrolizumab (MK-3475), nivolumab (ONO-4538/BMS-936558, MDX1106), pidilizumab (CT-011), atezolizumab (MPDL3280A), or AMP-224). In some embodiments, the anti-cancer agent is a BCL-2 inhibitor (such as GDC-0199/ABT-199), lenalidomide (REVLIMID®), a PI3K-delta inhibitor (such as idelalisib (ZYDELIG®)), an agonist (e.g., agonist antibody, directed against an activating co-stimulatory molecule, e.g., CD40, CD226, CD28, OX40 (e.g., AgonOX), GITR, CD137 (also known as TNFRSF9, 4-1BB, or ILA), CD27 (e.g., CDX-1127), HVEM, or CD127), an antagonist, e.g., antagonist antibody, directed against an inhibitory co-stimulatory molecule. e.g., CTLA-4 (also known as CD152), PD-1, TIM-3, BTLA, VISTA, LAG-3, B7-H3, B7-H4, IDO (e.g., 1-methyl-D-tryptophan (also known as 1-D-MT)), TIGIT, MICA/B, GITR (e.g., TRX518) or arginase, ipillmumab (also known as MDX-410, MDX-101, or YERVOY®), tremelimumab (also known as ticilimumab or CP-675,206, urelumab (also known as BMS-663513), MGA271, an antagonist directed against a TGF beta, e.g., metelimumab (also known as CAT-192), fresolimumab (also known as GC1008), LY2157299k, and an adoptive transfer of a T cell (e.g., a cytotoxic T cell or CTL) expressing a chimeric antigen receptor (CAR), e.g., adoptive transfer of a T cell comprising a dominant-negative TGF beta receptor, e.g., a dominant-negative TGF beta type II receptor.

### III. Therapeutic Methods

Provided herein are methods of treating cancer in an individual in need thereof by administering any of the nucleic acid vectors (e.g., circular DNA vectors or self-replicating RNA molecules), or compositions thereof, to the individual in a therapeutically effective amount. In particular instances, the therapeutic effect of the nucleic acid vector (e.g., immunomodulation of the tumor microenvironment) occurs after transmission of an electric field to the tumor microenvironment.

### Administration of Nucleic Acid Vectors and Compostions Thereof

The present invention involves administration of the nucleic acid vectors (e.g., circular DNA vectors and self-replicating RNA molecules) described herein, or pharmaceutical compositions thereof, to an individual having cancer (e.g., a human cancer patient). In some embodiments, the nucleic acid vector (e.g., circular DNA vector) administered (e.g., as part of a pharmaceutical composition or any of the therapeutic methods described herein) is any of the polycistronic nucleic acid vectors described above. For instance, methods of the invention include administering to an individual (e.g., a human cancer patient) an immunomodulatory nucleic acid vector comprising a polycistronic (e.g., bicistronic or tricistronic) immunomodulatory sequence comprising a dendritic cell chemoattractant-encoding gene (e.g., an XCL1-encoding gene, an XCL2-encoding gene, a CCL5-encoding gene, or a CCL4-encoding gene) and one or more immunomodulatory protein-encoding genes (e.g., a dendritic cell growth factor or activator-encoding gene (e.g., a FLT3L-encoding gene, a GMCSF-encoding gene, or a CD40L-encoding gene) or a lymphocyte signaling protein-encoding gene (e.g., a cytokine-encoding gene (e.g., an IL-12-encoding gene or an IL-15-encoding gene) or a chemokine-encoding gene (e.g., a CXCL9-encoding gene or a CXCL10-encoding gene)). Thus, in particular instances, methods of the invention include administering to an individual (e.g., a human cancer patient) an immunomodulatory nucleic acid vector (e.g., circular DNA vector) including a tricistronic immunomodulatory sequence comprising an XCL1-encoding gene, a FLT3L-encoding gene, and an IL-12-encoding gene; an immunomodulatory nucleic acid vector (e.g., circular DNA vector) including a tricistronic immunomodulatory sequence comprising an XCL1-encoding gene, a FLT3L-encoding gene, and an IL-15-encoding gene; or an immunomodulatory nucleic acid vector (e.g., circular DNA vector) comprising a tricistronic immunomodulatory sequence comprising an XCL1-encoding gene, a GM-CSF-encoding gene, and an IL-15-encoding gene.

Alternative methods of the invention include administering to an individual (e.g., a human cancer patient) an immunomodulatory nucleic acid vector comprising an immunomodulatory sequence comprising multiple transcription units, wherein one transcription unit includes a dendritic cell chemoattractant-encoding gene (e.g., an XCL1-encoding gene, an XCL2-encoding gene, a CCL5-encoding gene, or a CCL4-encoding gene) and another transcription unit includes one or more immunomodulatory protein-encoding genes (e.g., a dendritic cell growth factor or activator-encoding gene (e.g., a FLT3L-encoding gene, a GMCSF-encoding gene, or a CD40L-encoding gene) or a lymphocyte signaling protein-encoding gene (e.g., a cytokine-encoding gene (e.g., an IL-12-encoding gene or an IL-15-encoding gene) or a chemokine-encoding gene (e.g., a CXCL9-encoding gene or a CXCL10-encoding gene)). Thus, in particular instances, methods of the invention include administering to an individual (e.g., a human cancer patient) an immunomodulatory nucleic acid vector (e.g., circular DNA vector) including a immunomodulatory sequence comprising three transcription units, wherein the first transcription unit includes an XCL1-encoding gene, the second transcription unit includes a FLT3L-encoding gene, and the third transcription unit includes an IL-12-encoding gene; an immunomodulatory nucleic acid vector (e.g., circular DNA vector) including an immunomodulatory sequence comprising three transcription units, wherein the first transcription unit includes an XCL1-encoding gene, the second transcription unit includes a FLT3L-encoding gene, and the third transcription unit includes an IL-15-encoding gene; or an immunomodulatory nucleic acid vector (e.g., circular DNA vector) Including an immunomodulatory sequence comprising three transcription units, wherein the first transcription unit includes an XCL1-encoding gene, the second transcription unit includes a GM-CSF-encoding gene, and the third transcription unit includes an IL-15-encoding gene.

In other instances, the therapeutic methods of the invention include administration of one or more monocistronic nucleic acid vectors (e.g., circular DNA vectors). For example, in such instances, methods of the invention include administering to an individual (e.g., a human cancer patient) one or more immunomodulatory nucleic acid vectors (e.g., circular DNA vectors) having an immunomodulatory sequence (e.g., a monocistronic immunomodulatory sequence) comprising a dendritic cell chemoattractant-encoding gene (e.g., an XCL1-encoding gene, an XCL2-encoding gene, a CCL5-encoding gene, or a CCL4-encoding gene), a dendritic cell growth factor or activator-encoding gene (e.g., a FLT3L-encoding gene, a GMCSF-encoding gene, or a CD40L-encoding gene), or a lymphocyte signaling protein-encoding gene (e.g., a cytokine-encoding gene (e.g., an IL-12-encoding gene or an IL-15-encoding gene) or a chemokine-encoding gene (e.g., a CXCL9-encoding gene or a CXCL10-encoding gene)).

In some embodiments, the method includes administering two nucleic acid vectors (e.g., circular DNA vectors), wherein: (i) the first nucleic acid vector (e.g., circular DNA vector) includes a immunomodulatory sequence (e.g., a monocistronic immunomodulatory sequence) comprising a dendritic cell chemoattractant-encoding gene (e.g., an XCL1-encoding gene, an XCL2-encoding gene, a CCL5-encoding gene, or a CCL4-encoding gene) or a dendritic cell growth factor or activator-encoding gene (e.g., a FLT3L-encoding gene, a GMCSF-encoding gene, or a CD40L-encoding gene); and (ii) the second nucleic acid vector (e.g., circular DNA vector) includes a lymphocyte signaling protein-encoding gene (e.g., a cytokine-encoding gene (e.g., an IL-12-encoding gene or an IL-15-encoding gene) or a chemokine-encoding gene (e.g., a CXCL9-encoding gene or a CXCL10-encoding gene)).

In some instances, the method includes administering two nucleic acid vectors (e.g., circular DNA vectors), wherein: (i) the first nucleic acid vector (e.g., circular DNA vector) includes a immunomodulatory sequence (e.g., a monocistronic immunomodulatory sequence) comprising a dendritic cell chemoattractant-encoding gene (e.g., an XCL1-encoding gene, an XCL2-encoding gene, a CCL5-encoding gene, or a CCL4-encoding gene); and (ii) the second nucleic acid vector (e.g., circular DNA vector) includes a lymphocyte signaling protein-encoding gene (e.g., a cytokine-encoding gene (e.g., an IL-12-encoding gene or an IL-15-encoding gene) or a chemokine-encoding gene (e.g., a CXCL9-encoding gene or a CXCL10-encoding gene)). In some instances, the method includes administering two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL1-encoding gene; and (ii) the second nucleic acid vector (e.g., circular DNA vector) includes a cytokine-encoding gene (e.g., an IL-12-encoding gene or an IL-15-encoding gene) or a chemokine-encoding gene (e.g., a CXCL9-encoding gene or a CXCL10-encoding gene)). In some instances, the method includes administering two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL1-encoding gene; and (ii) the second nucleic acid vector (e.g., circular DNA vector) includes an IL-12-encoding gene. In some instances, the method includes administering two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL1-encoding gene; and (i) the second nucleic acid vector (e.g., circular DNA vector) includes an IL-15-encoding gene. In some instances, the method includes administering two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL1-encoding gene; and (ii) the second nucleic acid vector (e.g., circular DNA vector) includes a CXCL9-encoding gene. In some instances, the method includes administering two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL1-encoding gene; and (ii) the second nucleic acid vector (e.g., circular DNA vector) includes a CXCL10-encoding gene.

In some instances, the method includes administering two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL2-encoding gene; and (i) the second nucleic acid vector (e.g., circular DNA vector) includes a cytokine-encoding gene (e.g., an IL-12-encoding gene or an IL-15-encoding gene) or a chemokine-encoding gene (e.g., a CXCL9-encoding gene or a CXCL10-encoding gene)). In some instances, the method includes administering two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL2-encoding gene; and (ii) the second nucleic acid vector (e.g., circular DNA vector includes an IL-12-encoding gene. In some instances, the method includes administering two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL2-encoding gene; and (ii) the second nucleic acid vector (e.g., circular DNA vector) includes an IL-15-encoding gene. In some instances, the method includes administering two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL2-encoding gene; and (ii) the second nucleic acid vector (e.g., circular DNA vector) includes a CXCL9-encoding gene. In some instances, the method includes administering two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL2-encoding gene; and (i) the second nucleic acid vector (e.g., circular DNA vector) includes a CXCL10-encoding gene.

In some instances, the method includes administering two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an CCL4-encoding gene; and (ii) the second nucleic acid vector (e.g., circular DNA vector) includes a cytokine-encoding gene (e.g., an IL-12-encoding gene or an IL-15-encoding gene) or a chemokine-encoding gene (e.g., a CXCL9-encoding gene or a CXCL10-encoding gene). In some instances, the method includes administering two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an CCL4-encoding gene; and (ii) the second nucleic acid vector (e.g., circular DNA vector) includes an IL-12-encoding gene. In some instances, the method includes administering two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an CCL4-encoding gene; and (ii) the second nucleic acid vector (e.g., circular DNA vector) includes an IL-15-encoding gene. In some instances, the method includes administering two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an CCL4-encoding gene; and (ii) the second nucleic acid vector (e.g., circular DNA vector) includes a CXCL9-encoding gene. In some instances, the method includes administering two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an CCL4-encoding gene; and (i) the second nucleic acid vector (e.g., circular DNA vector) includes a CXCL10-encoding gene.

In some instances, the method includes administering two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an CCL5-encoding gene; and (ii) the second nucleic acid vector (e.g., circular DNA vector) includes a cytokine-encoding gene (e.g., an IL-12-encoding gene or an IL-15-encoding gene) or a cytokine-encoding gene (e.g., a CXCL9-encoding gene or a CXCL10-encoding gene). In some instances, the method includes administering two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an CCL5-encoding gene; and (ii) the second nucleic acid vector (e.g., circular DNA vector) includes an IL-12-encoding gene. In some instances, the method includes administering two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an CCL5-encoding gene; and (ii) the second nucleic acid vector (e.g., circular DNA vector) includes an IL-15-encoding gene. In some instances, the method includes administering two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an CCL5-encoding gene; and (i) the second nucleic acid vector (e.g., circular DNA vector) includes a CXCL9-encoding gene. In some instances, the method includes administering two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an CCL5-encoding gene; and (ii) the second nucleic acid vector (e.g., circular DNA vector) includes a CXCL10-encoding gene.

In some embodiments, the method includes administering two nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising a dendritic cell growth factor or activator-encoding gene (e.g., a FLT3L-encoding gene, a GMCSF-encoding gene, or a CD40L-encoding gene); and (ii) the second nucleic acid vector (e.g., circular DNA vector) includes a lymphocyte signaling protein-encoding gene (e.g., a cytokine-encoding gene (e.g., an IL-12-encoding gene or an IL-15-encoding gene) or a chemokine-encoding gene (e.g., a CXCL9-encoding gene or a CXCL10-encoding gene)).

In some embodiments, the method includes administering three nucleic acid vectors (e.g., circular DNA vectors), wherein: (i) the first nucleic acid vector (e.g., circular DNA vector) includes (a) an immunomodulatory sequence (e.g., a monocistronic immunomodulatory sequence) comprising a dendritic cell chemoattractant-encoding gene (e.g., an XCL1-encoding gene, an XCL2-encoding gene, a CCL5-encoding gene, or a CCL4-encoding gene); (ii) the second nucleic acid vector (e.g., circular DNA vector) includes a dendritic cell growth factor or activator-encoding gene (e.g., a FLT3L-encoding gene, a GMCSF-encoding gene, or a CD40L-encoding gene); and (iii) the third nucleic acid vector (e.g., circular DNA vector) includes a lymphocyte signaling protein-encoding gene (e.g., a cytokine-encoding gene (e.g., an IL-12-encoding gene or an IL-15-encoding gene) or a chemokine-encoding gene (e.g., a CXCL9-encoding gene or a CXCL10-encoding gene)).

In some embodiments, the method includes administering three nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL1-encoding gene; (ii) the second nucleic acid vector (e.g., circular DNA vector) includes a FLT3L-encoding gene; and (iii) the third nucleic acid vector (e.g., circular DNA vector) includes a lymphocyte signaling protein-encoding gene (e.g., a cytokine-encoding gene (e.g., an IL-12-encoding gene or an IL-15-encoding gene) or a chemokine-encoding gene (e.g., a CXCL9-encoding gene or a CXCL10-encoding gene)). In some embodiments, the method includes administering three nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL1-encoding gene; (ii) the second nucleic acid vector (e.g., circular DNA vector) includes a GM-CSF-encoding gene; and (iii) the third nucleic acid vector (e.g., circular DNA vector) includes a lymphocyte signaling protein-encoding gene (e.g., a cytokine-encoding gene (e.g., an IL-12-encoding gene or an IL-15-encoding gene) or a chemokine-encoding gene (e.g., a CXCL9-encoding gene or a CXCL10-encoding gene)).

In some embodiments, the method includes administering three nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL1-encoding gene; (ii) the second nucleic acid vector (e.g., circular DNA vector) includes a FLT3-encoding gene; and (iii) the third nucleic acid vector (e.g., circular DNA vector) includes an IL-12-encoding gene. In some embodiments, the method includes administering three nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL1-encoding gene; (ii) the second nucleic acid vector (e.g., circular DNA vector) includes a FLT3-encoding gene; and (iii) the third nucleic acid vector (e.g., circular DNA vector) includes an IL-15-encoding gene. In some embodiments, the method includes administering three nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL1-encoding gene; (ii) the second nucleic acid vector (e.g., circular DNA vector) includes a FLT3-encoding gene; and (iii) the third nucleic acid vector (e.g., circular DNA vector) includes a CXCL9-encoding gene. In some embodiments, the method includes administering three nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL1-encoding gene; (i) the second nucleic acid vector (e.g., circular DNA vector) includes a FLT3-encoding gene; and (iii) the third nucleic acid vector (e.g., circular DNA vector) includes a CXCL10-encoding gene in a pharmaceutically acceptable carrier.

In some embodiments, the method includes administering three nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL1-encoding gene; (ii) the second nucleic acid vector (e.g., circular DNA vector) includes a GM-CSF-encoding gene; and (iii) the third nucleic acid vector (e.g., circular DNA vector) includes an IL-12-encoding gene. In some embodiments, the method includes administering three nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL1-encoding gene; (ii) the second nucleic acid vector (e.g., circular DNA vector) includes a GM-CSF-encoding gene; and (iii) the third nucleic acid vector (e.g., circular DNA vector) includes an IL-15-encoding gene. In some embodiments, the method includes administering three nucleic acid vector (e.g., circular DNA vector), wherein: (i) the first nucleic acid vector (e.g., circular DNA vector) includes an immunomodulatory sequence (e.g., a monocistronic immunomodulatory sequence) comprising an XCL1-encoding gene; (ii) the second nucleic acid vector (e.g., circular DNA vector) includes a GM-CSF-encoding gene; and (iii) the third nucleic acid vector (e.g., circular DNA vector) includes an CXCL9-encoding gene. In some embodiments, the method includes administering three nucleic acid vectors (e.g., circular DNA vectors), wherein: (i) the first nucleic acid vector (e.g., circular DNA vector) includes an immunomodulatory sequence (e.g., a monocistronic immunomodulatory sequence) comprising an XCL1-encoding gene; (ii) the second nucleic acid vector (e.g., circular DNA vector) includes a GM-CSF-encoding gene; and (iii) the third nucleic acid vector (e.g., circular DNA vector) includes a CXCL10-encoding gene.

In any of the aforementioned methods, the immunomodulatory sequence can be within a self-replicating RNA-encoding sequence containing a replicase-encoding sequence that transcribes RNA from the self-replicating RNA molecule.

Various routes of administration are provided by the present invention. In particular embodiments, the nucleic acid vector (e.g., circular DNA vector) or pharmaceutical composition thereof is administered intratumorally. In other embodiments, the nucleic acid vector (e.g., circular DNA vector) or pharmaceutical composition thereof is administered peritumorally. In other embodiments, the nucleic acid vector (e.g., circular DNA vector) or pharmaceutical composition thereof is administered subdermally (e.g., subdermally near the tumor). In other embodiments, the nucleic acid vector (e.g., circular DNA vector) or composition thereof is administered systemically (e.g., intravenously).

In some instances, a single injection of the nucleic acid vector (e.g., circular DNA vector) or pharmaceutical composition thereof is administered to the individual over the course of the treatment in some embodiments, the single infection of the nucleic acid vector (e.g., circular DNA vector) or pharmaceutical composition thereof is administered intratumorally to the individual.

The nucleic acid vector (e.g., circular DNA vector) or pharmaceutical composition thereof can be administered as a single dose. Alternatively, the nucleic acid vector (e.g., circular DNA vector) or pharmaceutical composition thereof can be administered in multiple doses (e.g., two or more doses, three or more doses, four or more doses, five or more doses, six or more doses. e.g., two doses, three doses, four doses, five doses, or six doses) over the course of a treatment.

In some embodiments, dosing frequency is once every week, once every two weeks, once every three weeks, once every four weeks, once every five weeks, once every six weeks, once every seven weeks, once every eight weeks, once every nine weeks, or once every ten weeks; or once every month, once every two months, or once every three months, or less frequently. The progress of therapy (e.g., modulation of the tumor microenvironment) can be readily monitored (e.g., detected or quantified), according to methods known in the art and described herein. The dosing regimen of the nucleic acid vector (e.g., circular DNA vector) or pharmaceutical composition thereof can vary over time.

Dosages for a nucleic acid vector (e.g., circular DNA vector) or pharmaceutical composition thereof as described herein may be determined empirically in individuals who have been given one or more administrations of the nucleic acid vector (e.g., circular DNA vector) or pharmaceutical composition thereof. Individuals are given incremental dosages of the nucleic acid vector (e.g., circular DNA vector) or pharmaceutical composition thereof. To assess efficacy of the nucleic acid vector (e.g., circular DNA vector) or pharmaceutical composition thereof, an indicator of the disease/disorder can be monitored. For repeated administrations over several days or longer, depending on the condition, the treatment is sustained until a desired level of modulation of the tumor microenvironment is achieved.

In some instances, the appropriate dosage of the nucleic acid vector (e.g., circular DNA vector) described herein will depend on the specific nucleic acid vector (e.g., circular DNA vector), the type and severity of the cancer, previous therapy, the individual's clinical history and response to the nucleic acid vector (e.g., circular DNA vector), and the discretion of the attending physician. A clinician may administer a nucleic acid vector (e.g., circular DNA vector), or pharmaceutical composition thereof, until a dosage is reached that achieves the desired result (e.g., tumor microenvironment modulation and defined herein). In some embodiments, the desired result is a decrease in expression levels of a biomarker associated with tumor progression or an increase in expression levels of a biomarker associated with tumor clearance (e.g., an immunomodulatory protein). In other embodiments, the desired result is a decrease in tumor burden, a decrease in tumor cell number or metabolic activity, or an increase in immune cell activity. Further methods of determining whether a dosage resulted in the desired result are evident to one of skill in the art. Administration of one or more nucleic acid vectors (e.g., circular DNA vectors) can be continuous or intermittent, depending, for example, upon the individual's physiological condition and other factors known to skilled practitioners. The administration of nucleic acid vectors (e.g., circular DNA vectors) may be essentially continuous over a preselected period of time or may be in a series of spaced dose, e.g., either before, during, or after developing a target disease or disorder.

As a general proposition, the therapeutically effective amount of the nucleic acid vector (e.g., circular DNA vector) administered to the human may be in the range from 1.0 pg to 1 mg of nucleic acid (e.g., from 0.01 ng to 100 μg, from 0.1 ng to 50 μg, from 1 ng to 10 μg, or from 10 ng to 1 μg, e.g., from 0.01 ng to 0.05 ng, from 0.05 ng to 0.1 ng, from 0.1 ng to 0.5 ng, from 0.5 ng to 1 ng, from 1 ng to 5 ng, from 5 ng to 10 ng, from 10 ng to 50 ng, from 50 ng to 100 ng, from 100 ng to 500 ng, from 500 ng to 1 μg, from 1 μg to 5 μg, or from 5 μg to 10 μg, e.g., about 1 μg, about 5 μg, about 10 μg, about 20 μg, about 25 μg, about 50 μg, about 75 μg, about 100 μg, about 1 ng, about 5 ng, about 10 ng, about 20 ng, about 25 ng, about 50 ng, about 75 ng, about 100 ng, about 1 μg, about 2 μg, about 3 μg, about 4 μg, about 5 μg, about 6 μg, about 7 μg, about 8 μg, about 9 μg, about 10 μg, about 15 μg, about 20 μg, about 25 μg, about 50 μg, about 75 μg, about 100 μg, or about 500 μg).

Treatments of the invention may also include administration of one or more additional therapeutic agents (e.g., anti-cancer agents) suitable for the particular cancer being treated. For example, it may be desirable to further administer an additional therapeutic agent (e.g., an anti-cancer agent) selected from a chemotherapeutic agent, a cytotoxic agent, a growth inhibitory agent, and/or an anti-hormonal agent.

### Transmission of Electric Fields

In some aspects of the present invention, an electric field is transmitted into a tumor microenvironment. An electric field transmitted into a tumor microenvironment can promote transfer of a nucleic acid vector (e.g., circular DNA vector) into a target cell in the tumor microenvironment (e.g., a tumor cell or a tumor-infiltrating immune cell (e.g., a tumor infiltrating lymphocyte or a tumor-resident antigen-presenting cell (e.g., a tumor-resident dendritic cell))). Such electric field-mediated nucleic acid transfer (electrotransfer) can occur through any one of several mechanisms (and combinations thereof), including electrophoresis, electrokinetically driven drug uptake, and/or electroporation. Without wishing to be bound by theory, in certain instances, an electric field transmitted into a tumor microenvironment can facilitate anti-tumor adaptive immunity propagated by the nucleic acid vector (e.g., circular DNA vector). Suitable means of generating electric fields for electrotransfer of nucleic acids in mammalian tissue are known in the art, and any suitable means known in the art or described herein can be adapted for use as part of the present invention.

An electric field suitable for electrotransfer can be transmitted to a tumor microenvironment at or near the time of administration of a nucleic acid vector (e.g., circular DNA vector) or pharmaceutical composition thereof (e.g., as part of the same procedure). For example, the present invention includes methods in which an electric field is transmitted within one hour of administration of the nucleic acid vector (e.g., circular DNA vector) or pharmaceutical composition thereof (e.g., within 55 minutes, within 50 minutes, within 45 minutes, within 40 minutes, within 35 minutes, within 30 minutes, within 25 minutes, within 20 minutes, within 15 minutes, within 10 minutes, within 5 minutes, within 4 minutes, within 3 minutes, within 2 minutes, within 90 seconds, within 60 seconds, within 45 seconds, with 30 seconds, within 20 seconds, within 15 seconds, within 10 seconds, within 9 seconds, within 8 seconds, within 7 seconds, within 6 seconds, within 5 seconds, within 4 seconds, within 3 seconds, within 2 seconds, or within 1 second) of administration of the nucleic acid vector (e.g., circular DNA vector) or pharmaceutical composition thereof (e.g., simultaneously with administration of the nucleic acid vector (e.g., circular DNA vector) or pharmaceutical composition thereof or after administration but within any of the aforementioned durations). In some embodiments, an electric field is transmitted within 24 hours of administration of the nucleic acid vector (e.g., circular DNA vector) or pharmaceutical composition thereof (e.g., within 22 hours, within 20 hours, within 18 hours, within 16 hours, within 14 hours, within 12 hours, within 10 hours, within 8 hours, within 6 hours, within 5 hours, within 4 hours, within 3 hours, within 2 hours, within 90 minutes, within 60 minutes, within 45 minutes, within 30 minutes, within 20 minutes, within 15 minutes, within 10 minutes, within 8 minutes, within 6 minutes, within 5 minutes, within 4 minutes, within 3 minutes, or within 2 minutes) of administration of the nucleic acid vector (e.g., circular DNA vector) or pharmaceutical composition thereof. In some embodiments, an electric field is transmitted within 7 days of administration of the nucleic acid vector (e.g., circular DNA vector) or pharmaceutical composition thereof (e.g., within 6 days, within 5 days, within 4 days, within 3 days, or within 2 days) of administration of the nucleic acid vector (e.g., circular DNA vector) or pharmaceutical composition thereof.

Various means of generating and transmitting an electric field into a tumor microenvironment are contemplated herein as part of the present methods. Devices and systems having electrodes suitable for transmitting electric fields in mammalian tissues (e.g., at conditions suitable for electrotransfer) are commercially available and can be useful in the methods disclosed herein. In some instances, the electric field is transmitted through an electrode comprising one or more needles (e.g., one or more needles positioned within the tumor (intratumorally) or near the tumor (peritumorally), e.g., a single needle electrode, a multi-needle electrode, or a needle array electrode). Suitable needle electrodes include CLINIPORATOR® electrodes marketed by IGEA® and needle electrodes marketed by AMBU®. Such needle electrodes may be configured for transmission of an electric field into a superficial tumor or tumor that is accessible from beyond an epithelial surface (e.g., outside the body) (e.g., a melanoma, carcinoma, or a sarcoma (e.g., a head and neck tumor)), e.g., a tumor that is situated (in whole or in part) within 4 cm from an epithelial surface (e.g., within 3 cm from an epithelial surface, within 2 cm from an epithelial surface, or within 1 cm from an epithelial surface, e.g., 1 cm to 4 cm from an epithelial surface, 1 cm to 3 cm from an epithelial surface, 1 cm to 2 cm from an epithelial surface, 2 cm to 4 cm from an epithelial surface, 2 cm to 3 cm from an epithelial surface, or 3 cm to 4 cm from an epithelial surface).

In other instances, the electric field is transmitted through a non-needle electrode (e.g., a tweezer electrode (e.g., BTX® tweezertrodes), a plate electrode, or a caliper electrode). Such non-needle electrode may be configured for minimally invasive or non-invasive transmission of an electric field into a tumor (e.g., a superficial tumor or tumor that is accessible from beyond an epithelial surface (e.g., outside the body) (e.g., a melanoma, carcinoma, or a sarcoma (e.g., a head and neck tumor)), e.g., a tumor that is situated (in whole or in part) within 4 cm from an epithelial surface (e.g., within 3 cm from an epithelial surface, within 2 cm from an epithelial surface, or within 1 cm from an epithelial surface, e.g., 1 cm to 4 cm from an epithelial surface, 1 cm to 3 cm from an epithelial surface, 1 cm to 2 cm from an epithelial surface, 2 cm to 4 cm from an epithelial surface, 2 cm to 3 cm from an epithelial surface, or 3 cm to 4 cm from an epithelial surface)). Suitable electrodes include monopolar electrodes (e.g., monopolar needle electrodes) and bipolar electrodes (e.g., bipolar tweezer electrodes, bipolar plate electrodes, bipolar caliper electrodes, or bipolar multi-needle electrodes).

The electric field suitable for electrotransfer can have any suitable voltages, waveforms, frequencies, etc. In some embodiments of any of the methods of transmitting an electric field described herein, a pulsed electric field is transmitted. Pulsed electric fields suitable for electrotransfer can be transmitted at a range of voltages, waveforms, and frequencies. In some instances, the pulsed electric field is transmitted in a series of pulses comprising a pulse duration of 0.001 ms to 100 ms (e.g., from 0.005 ms to 50 ms, from 0.05 ms to 25 ms, or from 0.1 ms to 5 ms, e.g., from 0.001 ms to 0.01 ms, from 0.01 ms to 0.1 ms, from 0.1 ms to 1.0 ms, from 1 ms to 2 ms, from 2 ms to 4 ms, from 4 ms to 6 ms, from 6 ms to 10 ms, from 10 ms to 20 ms, from 20 ms to 50 ms, or from 50 ms to 100 ms, e.g., about 0.005 ms, about 0.01 ms, about 0.05 ms, about 0.1 me, about 0.5 ms, about 0.8 ms, about 0.7 ms, about 0.8 ms, about 0.9 ms, about 1 ms, about 1.5 ms, about 2 me, about 2.5 me, about 3 ms, about 4 ms, about 5 me, about 6 ms, about 7 ms, about 8 me, about 9 ms, about 10 me, about 12 ms, about 15 ms, about 20 me, about 25 ms, about 30 ms, about 35 ms, about 40 me, about 50 ms, about 60 ms, about 70 ms, about 80 ms, about 90 ms, or about 100 ms).

In some embodiments, a pulsed electric field comprises 2 to 100 pulses (e.g., 4 to 50 pulses, 5 to 40 pulses, 6 to 30 pulses, 7 to 25 pulses, or 8 to 20 pulses, e.g., 1 to 5 pulses, 5 to 10 pulses, 8 to 12 pulses, 10-15 pulses, 15-20 pulses, 20 to 50 pulses, or 50 to 100 pulses). Pulses may have any suitable waveform, such as square, sinusoidal, or sawtooth. In some embodiments, a pulsed electric field is transmitted with a frequency

In some of any of the aforementioned embodiments of methods involving transmission of pulsed electric fields, the energy of one or more of the pulses (e.g., a voltage at the target tissue) is from 50 V to 10,000 V (e.g., 100 V to 5,000 V, 200 V to 4,000 V, 300 V to 3,000 V, 400 V to 2,000 V, 500 V to 1,500 V, or 600 V to 100 V, e.g., 100 V to 200 V, 200 V to 300 V, 300 V to 400 V, 400 V to 500 V, 500 V to 600 V, 600 V to 700 V, 700 V to 800 V, 800 V to 900 V, 900 V to 1,000 V, 1,000 V to 1,500 V, 1,500 V to 2,000 V, 2,000 V to 3,000 V, 3,000 V to 5,000 V, or 5,000 V to 10,000 V, e.g., about 100 V, about 150 V, about 200 V, about 250 V, about 300 V, 400 V, 500 V, 600 V, 700 V, 800 V, 900 V, 1,000 V, 1,500 V, about 2,000 V, about 3.000 V, about 4,000 V, about 5,000 V, about 6,000 V, about 7,000 V, about 8,000 V, about 9.000 V. or about 10,000 V). In some instances, the amplitude of one or more (e.g., all) of the pulses is from 50 V to 10,000 V (e.g., 100 V to 5,000 V, 200 V to 4,000 V, 300 V to 3,000 V, 400 V to 2,000 V, 500 V to 1,500 V, or 600 V to 100 V, e.g., 100 V to 200 V, 200 V to 300 V, 300 V to 400 V, 400 V to 500 V, 500 V to 600 V, 600 V to 700 V, 700 V to 800 V, 800 V to 900 V, 900 V to 1,000 V, 1,000 V to 1,500 V, 1,500 V to 2,000 V, 2,000 V to 3,000 V, 3,000 V to 5,000 V, or 5,000 V to 10,000 V, e.g., about 100 V, about 150 V, about 200 V, about 250 V, about 300 V, 400 V, 500 V, 600 V, 700 V, 800 V, 900 V, 1,000 V, 1,500 V, about 2,000 V, about 3,000 V, about 4.000 V, about 5,000 V, about 6,000 V, about 7,000 V, about 8.000 V, about 9,000 V, or about 10,000 V). Any of the aforementioned voltages can be the tops of square-waveforms, peaks in sinusoidal waveforms, peaks in sawtooth waveforms, root mean square (RMS) voltages of sinusoidal waveforms, or RMS voltages of sawtooth waveforms.

An electric field suitable for electrotransfer can be transmitted at or near the site of administration of the nucleic acid vector (e.g., circular DNA vector) or pharmaceutical composition thereof. For instance, in some embodiments, the electric field is transmitted into the same tumor microenvironment as that in which the nucleic acid vector (e.g., circular DNA vector) or pharmaceutical composition thereof is administered. In some instances, the nucleic acid vector (e.g., circular DNA vector) or pharmaceutical composition thereof is delivered at a location that is exposed to the electric field (or will be exposed to the electric field, in the event of subsequent electric field transmission). In some embodiments, the nucleic acid vector (e.g., circular DNA vector) or pharmaceutical composition thereof is delivered at a location that is exposed to (or will be exposed to) a voltage that is 1% or more of the maximum tissue voltage (e.g., at least 5% of the maximum tissue voltage, at least 10% of the maximum tissue voltage, at least 20% of the maximum tissue voltage, at least 30% of the maximum tissue voltage, at least 40% of the maximum tissue voltage, at least 50% of the maximum tissue voltage, at least 60% of the maximum tissue voltage, at least 70% of the maximum tissue voltage, at least 80% of the maximum tissue voltage, or at least 90% of the maximum tissue voltage, e.g., from 1% to 10% of the maximum tissue voltage, from 10% to 20% of the maximum tissue voltage, from 20% to 30% of the maximum tissue voltage, from 30% to 40% of the maximum tissue voltage, from 40% to 50% of the maximum tissue voltage, from 50% to 60% of the maximum tissue voltage, from 60% to 70% of the maximum tissue voltage, from 70% to 80% of the maximum tissue voltage, from 80% to 90% of the maximum tissue voltage, from 90% to 95% of the maximum tissue voltage, or from 95% to 100% of the maximum tissue voltage). In some instances, the nucleic acid vector (e.g., circular DNA vector) or pharmaceutical composition thereof is administered intratumorally (e.g., intratumorally in the same tumor (e.g., the same tumor microenvironment) as that into which the electric field is delivered). In some instances, the nucleic acid vector (e.g., circular DNA vector) or pharmaceutical composition thereof is administered peritumorally.

Alternatively, the site of administration can be in a region of tissue away from the electric field. For example, administration of the nucleic acid vector (e.g., circular DNA vector) or pharmaceutical composition thereof may be systemic (e.g., intravenous), while the electric field is transmitted at the tumor. In some embodiments, the nucleic acid vector (e.g., circular DNA vector) or pharmaceutical composition thereof is administered to a non-tumor tissue (e.g., intramuscularly, subcutaneously, or subdermally).

### Additional Therapies

In certain embodiments of the therapeutic methods provided herein, one or more additional therapies are administered in conjunction with administration of the nucleic acid vector (e.g., circular DNA vector) or composition thereof, e.g., in lieu of, or in combination with, electric field transmission. Exemplary additional therapies include anti-cancer therapies, such as radiation therapy (e.g., a cytotoxic radiotherapy), photodynamic therapy, hyperthermic therapy, oncolytic therapy (e.g., administration of an oncolytic virus), or an anti-cancer agent (e.g., a chemotherapeutic agent, a checkpoint inhibitor, a cytotoxic agent, a growth inhibitory agent, an anti-angiogenic agent, a cytokine, a cytokine antagonist, an antibody-drug conjugate, a cancer vaccine, or a combination thereof). Thus, in some instances, the present invention includes any method of administering any of the nucleic acid vectors (e.g., circular DNA vectors described herein (e.g., monocistronic or polycistronic (e.g., bicistronic or tricistronic) nucleic acid vector (e.g., circular DNA vector), or nucleic acid vectors (e.g., circular DNA vectors) having multiple transcription units) in combination with a radiation therapy (e.g., a cytotoxic radiotherapy), a photodynamic therapy, a hyperthermic therapy, or an oncolytic therapy (e.g., an oncolytic virus), wherein the radiation therapy, photodynamic therapy, hyperthermic therapy, or oncolytic therapy is administered according to any known or readily obtainable protocols (e.g., in lieu of transmission of an electric field).

In some embodiments, the additional therapy is an additional therapeutic agent, such as an anti-cancer agent (e.g., a chemotherapeutic agent, a checkpoint inhibitor, a cytotoxic agent, a growth inhibitory agent, an anti-angiogenic agent, a cytokine, a cytokine antagonist, an antibody-drug conjugate, a cancer vaccine, or a combination thereof). In particular embodiments, the additional therapy is a chemotherapeutic agent. In other particular embodiments, the additional therapy is a checkpoint inhibitor, e.g., a PD-1 axis binding antagonist (e.g., an anti-PD-1 antagonist or an anti-PD-L1 antibody), such as pembrolizumab (MK-3475), nivolumab (ONO-4538/BMS-936558, MDX1106), pidilizumab (CT-011), atezolizumab (MPDL3280A), or AMP-224). In some embodiments, the additional therapy is an additional therapeutic agent selected from a BCL-2 inhibitor (such as GDC-0199/ABT-199), lenalidomide (REVLIMID®), a PI₃K-delta inhibitor (such as idelalisib (ZYDELIG®)), an agonist (e.g., agonist antibody, directed against an activating co-stimulatory molecule, e.g., CD40, CD226, CD28, OX40 (e.g., AgonOX), GITR, CD137 (also known as TNFRSF9, 4-1BB, or ILA), CD27 (e.g., CDX-1127), HVEM, or CD127), an antagonist, e.g., antagonist antibody, directed against an inhibitory co-stimulatory molecule, e.g., CTLA-4 (also known as CD152), PD-1, TIM-3, BTLA, VISTA, LAG-3, B7-H3, B7-H4, IDO (e.g., 1-methyl-D-tryptophan (also known as 1-D-MT)), TIGIT, MICA/B, GITR (e.g., TRX518) or arginase, ipilimumab (also known as MDX-010, MDX-101, or YERVOY®), tremelimumab (also known as ticilimumab or CP-675,206, urelumab (also known as BMS-663513), MGA271, an antagonist directed against a TGF beta, e.g., metelimumab (also known as CAT-192), fresolimumab (also known as GC1008), LY2157299k, and an adoptive transfer of a T cell (e.g., a cytotoxic T cell or CTL) expressing a chimeric antigen receptor (CAR), e.g., adoptive transfer of a T cell comprising a dominant-negative TGF beta receptor, e.g., a dominant-negative TGF beta type II receptor.

In further instances, the additional therapy includes an anti-CD20 antibody, such as rituximab or a humanized B-Ly1 antibody (e.g., obinutuzumab). In some embodiments, the anti-CD20 antibody is ofatumumab, ublituximab, and/or ibritumomab tiuxetan.

In some instances, the additional therapy includes an alkylating agent, such as 4-[5-[Bis(2-chloroethyl)amino]-1-methylbenzimidazol-2-yl]butanoic acid, or a salt thereof. In some embodiments, the alkylating agent is bendamustine.

In a further aspect of the invention, the additional therapy comprises a BCL-2 inhibitor. In some embodiments, the BCL-2 inhibitor is 4-(4-([2-(4-chlorophenyl)-4,4-dimethylcyclohex-1-en-1-yl]methyl}piperazin-1-yl)-N-({3-nitro-4-[(tetrahydro-2H-pyran-4-ylmethyl)amino]phenyl}sulfony-l)-2-(1H-pyrrolo[2,3-b]pyridin-5-yloxy)benzamide and salts thereof. In one embodiment, the BCL-2 inhibitor is venetoclax (CAS #: 1257044-40-8).

In a further aspect of the invention, the additional therapy comprises a phosphoinositide 3-kinase (PI3K) Inhibitor. In one embodiment, the PI3K inhibitor inhibits delta isoform PI3K (i.e., P110.delta.). In some embodiments, the PI3K inhibitor is 5-Fluoro-3-phenyl-2-[(1S)-1-(7H-purin-6-ylamino)propyl]4(3H)-quinazolino-ne and salts thereof. In some embodiments, the PI3K inhibitor is idelalisib (CAS #: 870281-82-6). In one embodiment, the PI3K inhibitor inhibits alpha and delta isoforms of PI3K. In some embodiments, the PI3K inhibitor is 2-(3-(2-(1-Isopropyl-3-methyl-1H-1,2-4-triazol-5-yl)-5,6-dihydrobenzo[f]i-midazo[1,2-d][1,4]oxazepin-9-yl-1H-pyrazol-1-yl-2-methylpropanamide and salt thereof.

In a further aspect of the invention, the additional therapy comprises a Bruton's tyrosine kinase (BTK) inhibitor. In one embodiment, the BTK inhibitor is 1-[(3R)-3-[4-Amino-3-(4-phenoxyphenyl)-1H-pyrazolo[3,4-d]pyrimidin-1-yl]p-iperidin-1-yl]prop-2-en-1-one and salts thereof. In one embodiment, the BTK inhibitor is ibrutinib (CAS #: 936563-96-1).

In a further aspect of the invention, the additional therapy comprises thalidomide or a derivative thereof. In one embodiment, the thalidomide or a derivative thereof is (RS)-3-(4-Amino-1-oxo 1,3-dihydro-2H-isoindol-2-yl)piperidine-2,6-dione and salts thereof. In one embodiment, the thalidomide or a derivative thereof is lenalidomide (CAS #: 191732-72-6).

In a further aspect of the invention, the additional therapy comprises one or more of cyclophosphamide, doxorubicin, vincristine, or prednisolone (CHOP). In one embodiment, the additional therapy further comprises an anti-CD20 antibody as described above (e.g., GA-101 and/or RITUXAN®). Any of the above methods and therapies may be used, without limitation, for any cancer, including, for example, solid tumors.

In certain embodiments, an additional therapeutic agent is a chemotherapeutic agent, growth inhibitory agent, cytotoxic agent, agent used in radiation therapy, anti-angiogenesis agent, apoptotic agent, anti-tubulin agent, or other agent, such as an epidermal growth factor receptor (EGFR) antagonist (e.g., a tyrosine kinase inhibitor), HER1/EGFR inhibitor (e.g., erlotinib (Tarceva), platelet derived growth factor inhibitor (e.g., Gleevec (imatinib Mesylate)), a COX-2 Inhibitor (e.g., celecoxib), interferon, cytokine, antibody other than the anti-CD3 antibody of the invention, such as an antibody that bind to one or more of the following targets ErbB2, ErbB3, ErbB4, PDGFR-beta, BlyS, APRIL, BCMA VEGF, or VEGF receptor(s), TRAIL/Apo2, PD-1, PD-1, PD-L2, or another bioactive or organic chemical agent.

In some embodiments, the invention provides a method wherein the additional therapeutic agent is a glucocorticoid. In one embodiment, the glucocorticoid is dexamethasone.

### Tumors and Individuals

The compostions and methods described herein are suitable for treatment of various cancer types. In particular instances, the cancer being treated is a solid tumor (e.g., a sarcoma, a carcinoma, or a lymphoma). Sold tumors treatable by methods of the present invention include solid tumors of epithelial cell origin and solid tumors of non-epithelial cell origin. Examples of solid tumors of epithelial cell origin include the tumors of the head and neck, gastrointestinal tract, colon, breast, prostate, lungs, kidneys, liver, pancreas, ovary, oral cavity, stomach, duodenum, small intestine, large intestine, anus, gallbladder, labia, nasopharynx, skin, uterus, male reproductive organs, urine organs, bladder, and skin tumors. Solid tumors of non-epithelial cell origin include sarcomas and brain tumors.

In some instances, the present compositions and methods are suitable for treating a melanoma. Melanomas that can be treated by methods of the compositions of the present invention include superficial spreading melanoma, nodular melanoma, lentigo melanoma, acral lentiginous melanoma, amelanotic melanoma, nevoid melanoma, spitzoid melanoma, and desmoplastic melanoma.

In some instances, the present compositions and methods are suitable for treating head and neck cancers. Head and neck cancers that can be treated by methods and compositions of the present invention include squamous cell carcinomas (e.g., oral cavity squamous cell carcinoma). In some instances, the head and neck cancer treatable by the present invention is a cancer (e.g., carcinoma, e.g., squamous cell carcinoma) of the nasopharynx, oropharynx, hypopharynx, larynx, or trachea. In some embodiments, the heed and neck cancer is characterized by a tumor in the lip, oral cavity, oropharynx, hypopharynx, nasopharynx, glottic larynx, or supraglottic larynx. In some embodiments, the head and neck cancer is characterized by an ethmoid sinus tumor, a maxillary sinus tumor, a salivary gland tumor, an occult primary tumor, or a mucosal melanoma.

In other embodiments, the cancer is selected from the group consisting of a desmoid tumor, non-small cell lung cancer, a small cell lung cancer, a renal cell cancer, a colorectal cancer, an ovarian cancer, a breast cancer, a pancreatic cancer, a gastric carcinoma, a kidney cancer, a bladder cancer, an esophageal cancer, a mesothelioma, a thyroid cancer, a sarcoma, a prostate cancer, a glioblastoma, a cervical cancer, a thymic carcinoma, or a lymphoma. In some embodiments, the tumor is a Klatskin tumor, a hilar tumor, a germ cell tumor, an Ewing's tumor, an Askin's tumor, a primitive neuroectodermal tumor, a Leydig cell tumor, a Wilms' tumor, or a Sertoli cell tumor. In some embodiments, the tumor is a carcinoma selected from the group consisting of a squamous cell carcinoma, a cloacogenic carcinoma, an adenocarcinoma, an adenosquamous carcinoma, a cholangiocarcinoma, a hepatocellular carcinoma, an invasive papillary urothelial carcinoma, and a flat urothelial carcinoma.

In some embodiments, the tumor is a resectable tumor. In other embodiments, the tumor is a non-resectable tumor. In some embodiments, the tumor is an advanced-stage tumor. In other embodiments, the tumor is an early-stage tumor. In some embodiments, the cancer is a relapsed and/or refractory cancer (e.g., the method is a second-line or third-line therapy).

Nucleic acid vectors (e.g., circular DNA vectors) and pharmaceutical compositions prepared according to the invention may be used to treat children or adults. Thus, an individual (e.g., a human patient) may be less than 1 year old, less than 5 years old, 1-5 years old, 5-15 years old, 15-55 years old, or at least 55 years old.

### Detecting Tumor Microenvironment Modulation

Modulation of the tumor microenvironment by the therapeutic methods provided herein can be detected using any method known in the art (e.g., by detecting a genetic or protein biomarker for tumor microenvironment modulation). Genetic and protein biomarkers useful to determine whether a treatment is modulating a particular tumor microenvironment are readily available to a skilled artisan, e.g., through information made available by the U.S. National Comprehensive Cancer Network (NCCN). Additionally, or alternatively, modulation of tumor microenvironment can be detected or quantified in terms of clinical response criteria, such as readouts defined in the Response Evaluation Criteria in Solid Tumors (RECIST) Guidelines.

In some instances, a tumor microenvironment is modulated by a treatment (e.g., a treatment of the present invention. e.g., a treatment involving administration of a nucleic acid vector (e.g., circular DNA vector) and/or transmission of an electric field) if the treatment increases the relative expression of biomarker associated with tumor clearance (e.g., an immunogenic protein (e.g., a pro-Inflammatory cytokine or a dendritic cell activating protein)) in the tumor microenvironment). Additionally, or alternatively, a tumor microenvironment is said to be modulated by a treatment if the treatment decreases the relative expression of a biomarker associated with tumor progression (e.g., a biomarker of T cell exhaustion, e.g., PD-1 expression).

In some embodiments, a measurable change in expression level of the biomarker indicative of tumor microenvironment modulation is at least 10%, at least 20%, at least 30%, at least 50%, at least 75%, or at least 90% change in expression level of the biomarker. For example, a measurable decrease in expression level of a biomarker associated with tumor progression indicates desirable tumor microenvironment modulation when the decrease in expression of the biomarker is at least 10%, at least 20%, at least 30%, at least 50%, at least 75%, or at least 90% relative to a reference level. Additionally, or alternatively, a measurable increase in expression level of a biomarker associated with tumor clearance (e.g., an immunogenic protein) indicates desirable tumor microenvironment modulation when the increase in expression of the biomarker is at least at least 1%, at least 2%, at least 3%, at least 4%, at least 5%, at least 6%, at least 7%, at least 8%, at least 9%, at least 10%, at least 20%, at least 30%, at least 50%, at least 75%, at least 90%, at least 2-fold, at least 3-fold, at least 4-fold, at least 5-fold, at least 10-fold, at least 20-fold, at least 50-fold, or at least 100-fold increased relative to a reference level (e.g., from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 2-fold to 3-fold, from 3-fold to 5-fold, from 5-fold to 10-fold, from 10-fold to 20-fold, from 20-fold to 50-fold, from 50-fold to 100-fold, or more, relative to a reference level; e.g., from 1% to 100%, from 2-fold to 10-fold, from 10-fold to 50-fold, from 50-fold to 100-fold, or more, relative to a reference level).

In some embodiments, a biomarker associated with tumor clearance is a circulating biomarker, such as a circulating tumor DNA (ctDNA), e.g., a cell free ctDNA, a cell free RNA, an mRNA, or a non-coding RNA (e.g., microRNA, short interfering, piwi-interacting RNA, small nuclear RNA, small nucleolar RNA, long non-coding RNA, microRNA).

In particular instances, modulation of the tumor microenvironment is characterized by the detection of an anti-tumor adaptive immune response (e.g., the generation, propagation, or enhancement of an anti-tumor adaptive immune response relative to a reference level), e.g., an adaptive immune response mounted against one or more tumor antigens expressed by the tumor in the individual being treated (e.g., wherein the tumor antigen generated and recognized in-situ and is not provided by treatment). Thus, in some instances, any of the methods of the invention further include a step of detecting an anti-tumor adaptive immune response in the individual (e.g., after treatment according to the invention, before treatment according to the invention, or both).

Tissue or cell samples can be assayed, e.g., for mRNA or DNA from a genetic biomarker using Northern, dot-blot, polymerase chain reaction (PCR) analysis, array hybridization, RNase protection assay, or DNA SNP chip microarrays, which are commercially available, including DNA microarray snapshots. For example, real-time PCR (RT-PCR) assays such as quantitative PCR assays are well known in the art. In some instances of the invention, a method for detecting mRNA from a genetic biomarker of interest in a biological sample, such as a tumor sample (e.g., a solid tumor sample), comprises producing cDNA from the sample by reverse transcription using at least one primer; amplifying the cDNA so produced; and detecting the presence of the amplified cDNA. In addition, such methods can include one or more steps that allow one to determine the levels of mRNA in a biological sample (e.g., by simultaneously examining the levels a comparative control mRNA sequence of a “housekeeping” gene such as an actin family member). Optionally, the sequence of the amplified cDNA can be determined.

In one particular embodiment, expression of the genetic biomarker as described herein can be performed by RT-PCR technology. Probes used for PCR may be labeled with a detectable marker, such as, for example, a radioisotope, fluorescent compound, bioluminescent compound, a chemiluminescent compound, metal chelator, or enzyme. Such probes and primers can be used to detect the presence of expressed genes (e.g., Immunomodulatory genes) in a sample. As will be understood by the skilled artisan, numerous different primers and probes may be prepared and used effectively to amplify, clone and/or determine the presence and/or levels expressed of one or more modulatory (e.g., immunomodulatory) genes. Additionally or alternatively, tumor sample-derived genetic biomarkers can be detected and/or quantified using known methods, such as Sanger sequencing, pyrosequencing, aisle-specific arrayed primer extension, next-generation sequencing (NGS), mutant-enriched liquid chip, amplification refractory mutation system, co-amplification at lower denaturation temperature-PCR, and bead/emulsion/amplification/magnetic PCR.

Other methods include protocols that examine or detect mRNAs from at least one genetic biomarker in a tissue (e.g., a tumor tissue, e.g., a solid tumor tissue) or cell sample by microarray technologies. Using nucleic acid microarrays, test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of genes that have potential to be expressed in certain disease states may be arrayed on a solid support. Hybridization of a labeled probe with a particular array member indicates that the sample from which the probe was derived expresses that gene. Differential gene expression analysis of disease tissue can provide valuable information. Microarray technology utilizes nucleic acid hybridization techniques and computing technology to evaluate the mRNA expression profile of thousands of genes within a single experiment (see, e.g., WO 2001/75166). See, for example, U.S. Pat. Nos. 5,700,637, 5,445,934, and 5,807,522, Lockart, Nat. Biotechnol. 14:1675-1680 (1996); and Cheung et al., Nat Genet 21(Suppl):15-19 (1999) for a discussion of array fabrication.

In addition, the DNA profiling and detection method utilizing microarrays may be employed, e.g., as described in EP 1753878. Such methods rapidly identify and distinguish between different DNA sequences utilizing short tandem repeat (STR) analysis and DNA microarrays. In an embodiment, a labeled STR target sequence is hybridized to a DNA microarray carrying complementary probes. These probes vary in length to cover the range of possible STRs. The labeled single-stranded regions of the DNA hybrids are selectively removed from the microarray surface utilizing a post-hybridization enzymatic digestion. The number of repeats in the unknown target is deduced based on the pattern of target DNA that remains hybridized to the microarray. One example of a microarray processor is the Affymetrix GENECHIP® system, which is commercially available and comprises arrays fabricated by direct synthesis of oligonucleotides on a glass surface. Other systems may be used as known to one skilled in the art.

Other methods for determining the level of the biomarker besides RT-PCR or another PCR-based method include proteomics techniques, as well as individualized genetic profiles that can be useful to treat cancer based on patient response at a molecular level. The specialized microarrays herein, e.g., oligonucleotide microarrays or cDNA microarrays, may comprise one or more biomarkers having expression profiles that correlate with tumor progression or tumor clearance (e.g., through anti-tumor immunity). Other methods that can be used to detect nucleic acids, for use in the invention, involve high throughput RNA sequence expression analysis, including RNA-based genomic analysis, such as, for example, RNASeq.

In particular instances, modulation of a tumor microenvironment can be detected, quantified, and/or monitored using available NGS techniques, e.g., tumor specific NGS kits, such as AVENIO® tumor tissue and ctDNA analysis kits.

Various assays are available for detection and quantification of protein biomarkers including, for example, antibody-based methods as well as mass spectroscopy and other similar means known in the art, including, but not limited to, immunocytochemistry (IHC), Western blot analysis, immunoprecipitation, molecular binding assays, enzyme-linked immunosorbent assay (ELISA), enzyme-linked immunofiltration assay (ELIFA), fluorescence activated cell sorting (FACS), MassARRAY, proteomics, quantitative blood based assays (e.g., serum ELISA), biochemical enzymatic activity assays, in situ hybridization, fluorescence in situ hybridization (FISH), Southern analysis, or Northern analysis. In the case of antibody-based methods, for example, the sample may be contacted with an antibody specific for said biomarker under conditions sufficient for an antibody-biomarker complex to form, and then detecting said complex. Detection of the presence of the protein biomarker may be accomplished in a number of ways, such as immunohistochemistry (IHC), Western blotting (with or without immunoprecipitation), 2-dimensional SDS-PAGE, immunoprecipitation, fluorescence activated cell sorting (FACS), flow cytometry, and ELISA procedures for assaying a wide variety of tissues and samples, including plasma or serum. A wide range of immunoassay techniques using such an assay format are available, see, e.g., U.S. Pat. Nos. 4,016,043, 4,424,279, and 4,018,653. These include both single-site and two-site or “sandwich” assays of the non-competitive types, as well as in the traditional competitive binding assays. These assays also include direct binding of a labeled antibody to a target biomarker.

Sandwich assays are among the most commonly used assays. A number of variations of the sandwich assay technique exist, and all are intended to be encompassed by the present invention. Briefly, in a typical forward assay, an unlabeled antibody is Immobilized on a solid substrate, and the sample to be tested brought into contact with the bound molecule. After a suitable period of incubation, for a period of time sufficient to allow formation of an antibody-antigen complex, a second antibody specific to the antigen, labeled with a reporter molecule capable of producing a detectable signal is then added and incubated, allowing time sufficient for the formation of another complex of antibody-antigen-labeled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of biomarker.

Variations on the forward assay include a simultaneous assay, in which both sample and labeled antibody are added simultaneously to the bound antibody. These techniques are well known to those skilled in the art, including any minor variations as will be readily apparent. In a typical forward sandwich assay, a first antibody having specificity for the biomarker is either covalently or passively bound to a solid surface. The solid surface is typically glass or a polymer, the most commonly used polymers being cellulose, polyacrylamide, nylon, polystyrene, polyvinyl chloride, or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g., 2-40 minutes or overnight if more convenient) and under suitable conditions (e.g., from room temperature to 40° C. such as between 25° C. and 32° C., Inclusive) to allow binding of any subunit present in the antibody. Following the incubation period, the antibody subunit solid phase is washed and dried and incubated with a second antibody specific for a portion of the biomarker. The second antibody is linked to a reporter molecule which is used to indicate the binding of the second antibody to the molecular marker.

An alternative method involves immobilizing the target biomarkers in the sample and then exposing the immobilized target to specific antibody which may or may not be labeled with a reporter molecule. Depending on the amount of target and the strength of the reporter molecule signal, a bound target may be detectable by direct labeling with the antibody. Alternatively, a second labeled antibody, specific to the first antibody is exposed to the target-first antibody complex to form a target-first antibody-second antibody tertiary complex. The complex is detected by the signal emitted by the reporter molecule. A reporter molecule is a molecule which, by its chemical nature, provides an analytically identifiable signal which allows the detection of antigen-bound antibody. The most commonly used reporter molecules in this type of assay are either enzymes, fluorophores or radionuclide containing molecules (i.e., radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase, and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather then the chromogenic substrates noted above. In all cases, the enzyme-labeled antibody is added to the first antibody-molecular marker complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of biomarker which was present in the sample. Alternately, fluorescent compounds, such as fluorescein and rhodamine, may be chemically coupled to antibodies without altering their binding capacity. When activated by illumination with light of a particular wavelength, the fluorochrome-labeled antibody adsorbs the light energy, inducing a state to excitability in the molecule, followed by emission of the light at a characteristic color visually detectable with a light microscope. As in the EIA, the fluorescent labeled antibody is allowed to bind to the first antibody-molecular marker complex. After washing off the unbound reagent, the remaining tertiary complex is then exposed to the light of the appropriate wavelength, the fluorescence observed indicates the presence of the molecular marker of interest immunofluorescence and EIA techniques are both very well established in the art. Other reporter molecules, such as radioisotope, chemiluminescent or bioluminescent molecules, may also be employed.

Clinical response criteria can also be employed to determine whether a tumor microenvironment is modulated. Any established clinical response criteria may be utilized, including the RECIST Guidelines. For example, in some embodiments, a tumor microenvironment is determined to have been modulated by a treatment that extends progression-free survival, results in an objective response, including a partial response or a complete response, increases overall survival time, and/or improves one or more symptoms of the cancer.

### IV. Kits and Articles of Manufacture

In another aspect of the invention, an article of manufacture or a kit containing materials useful for the treatments described above is provided. The article of manufacture includes a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a nucleic acid vector (e.g., circular DNA vector or self-replicating RNA molecule) of the invention or a pharmaceutical composition comprising the nucleic acid vector of the invention. The label or package insert indicates that the composition is used for treating the cancer of choice. Moreover, the article of manufacture may comprise (a) a first container with a composition contained therein, wherein the composition comprises a nucleic acid vector (e.g., circular DNA vector) and/or composition of the invention; and (b) a second container with a composition contained therein, wherein the composition comprises an additional therapeutic agent (e.g., an additional anti-cancer agent). The article of manufacture may further comprise a package insert indicating that the compositions can be used to treat a particular condition. Alternatively, or additionally, the article of manufacture may further comprise a second (or third) container comprising a pharmaceutically acceptable carrier, such as bacteriostatic water for injection (BWFI), phosphate-buffered saline, Ringer's solution, dextrose solution, or any of the pharmaceutically acceptable carriers disclosed above. It may further include other materials desirable from a commercial and user standpoint, including other buffers, diluents, filters, needles, and syringes.

In particular instances of the invention, provided is a kit that includes (i) any one or more of the materials described above (e.g., a nucleic acid vector (e.g., circular DNA vector or self-replating RNA molecule) of the invention, or a composition comprising the nucleic acid vector (e.g., circular DNA vector), an additional therapeutic agent (e.g., an additional anti-cancer agent), and/or one or more pharmaceutically acceptable carriers) and (ii) one or more elements of an energy delivery device (e.g., a device including an electrode for transmitting an electric field to a tumor microenvironment, such as any suitable devices or systems described above). In some embodiments, provided herein is a kit that includes a nucleic acid vector (e.g., circular DNA vector) of the invention and an electrode. In some embodiments, provided herein is a kit that includes a pharmaceutical composition comprising a nucleic acid vector (e.g., circular DNA vector) of the invention and an electrode.

TABLE 1
Sequences

| SEQ | | | |
|---|---|---|---|
| ID NO | Description | Type | Sequence |
| 1 | VEE non- | Viral RNA | AUGGAGAAAGUUCACGUUGACAUCGAGGAAGACAGCCCAUUCCU |
| | structural | | CAGAGCUUUGCAGCGGAGCUUCCCGCAGUUUGAGGUAGAAGCC |
| | protein 1 | | AAGCAGGUCACUGAUAAUGACCAUGCUAAUGCCAGAGCGUUUUC |
| | RNA | | GCAUCUGGCUUCAAAACUGAUCGAAACGGAGGUGGACCCAUCCG |
| | | | ACACGAUCCUUGACAUUGGAAGUGCGCCCGCCCGCAGAAUGUAU |
| | | | UCUAAGCACAAGUAUCAUUGUAUCUGUCCGAUGAGAUGUGCGGA |
| | | | AGAUCCGGACAGAUUGUAUAAGUAUGCAACUAAGCUGAAGAAAA |
| | | | ACUGUAAGGAAAUAACUGAUAAGGAAUUGGACAAGAAAAUGAAG |
| | | | GAGCUCGCCGCCGUCAUGAGCGACCCUGACCUGGAAACUGAGA |
| | | | CUAUGUGCCUCCACGACGACGAGUCGUGUCGCUACGAAGGGCA |
| | | | AGUCGCUGUUUACCAGGAUGUAUACGCGGUUGACGGACCGACAA |
| | | | GUCUCUAUCACCAAGCCAAUAAGGGAGUUAGAGUCGCCUACUGG |
| | | | AUAGGCUUUGACACCACCCCUUUUAUGUUUAAGAACUUGGCUGG |
| | | | AGCAUAUCCAUCAUACUCUACCAACUGGGCCGACGAAACCGUGU |
| | | | UAACGGCUCGUAACAUAGGCCUAUGCAGCUCUGACGUUAUGGAG |
| | | | CGGUCACGUAGAGGGAUGUCCAUUCUUAGAAAGAAGUAUUUGAA |
| | | | ACCAUCCAACAAUGUUCUAUUCUCUGUUGGCUCGACCAUCUACC |
| | | | ACGAGAAGAGGGACUUACUGAGGAGCUGGCACCUGCCGUCUGU |
| | | | AUUUCACUUACGUGGCAAGCAAAAUUACACAUGUCGGUGUGAGA |
| | | | CUAUAGUUAGUUGCGACGGGUACGUCGUUAAAAGAAUAGCUAUC |
| | | | AGUCCAGGCCUGUAUGGGAAGCCUUCAGGCUAUGCUGCUACGA |
| | | | UGGACCGGGAGGGAUUCUUGUGCUGCAAAGUGACAGACACAUUG |
| | | | AACGGGGAGAGGGUCUCUUUUCCCGUGUGCACGUAUGUGCCAG |
| | | | CUACAUUGUGUGACCAAAUGACUGGCAUACUGGCAACAGAUGUC |
| | | | AGUGCGGACGACGCGCAAAAACUGCUGGUUGGGCUCAACCAGC |
| | | | GUAUAGUCGUCAACGGUGGCACCCAGAGAAACACCAAUACCAUG |
| | | | AAAAAUUACCUUUUGCCCGUAGUGGCCCAGGCAUUUGCUAGGUG |
| | | | GGCAAAGGAAUAUAAGGAAGAUCAAGAAGAUGAAAGGGCACUAG |
| | | | GACUACGAGAUAGACAGUUAGUCAUGGGGUGUUGUUGGGCUUU |
| | | | UAGAAGGCACAAGAUAACAUCUAUUUAUAAGCGCCCGGAUACCC |
| | | | AAACCAUCAUCAAAGUGAACAGCGAUUUCCACUCAUUCGUGCUG |
| | | | CCCAGGAUAGGCAGUAACACAUUGGAGAUGGGGCUGAGAACAAG |
| | | | AAUCAGGAAAAUGUUAGAGGAGCACAAGGAGCCGUCACCUCUCA |
| | | | UUACCGCCGAGGACGUACAAGAAGCUAAGUGCGCAGCCGAUGAG |
| | | | GCUAAGGAGGUGCGUGAAGCCGAGGAGUUGCGCGCAGCUCUAC |
| | | | CACCUUUGGCAGCUGAUGUUGAGGAGCCCACUCUGGAAGCCGA |
| | | | UGUCGACUUGAUGUUACAAGAGGCUGGGGCC |
| 1A | DNA sequence | | ATGGAGAAAGTTCACGTTGACATCGAGGAAGACAGCCCATTCCTC |
| | corresponding to | | AGAGCTTTGCAGCGGAGCTTCCCGCAGTTTGAGGTAGAAGCCAAG |
| | SEQ ID NO: 1 | | CAGGTCACTGATAATGACCATGCTAATGCCAGAGCGTTTTCGCATC |
| | | | TGGCTTCAAAACTGATCGAAACGGAGGTGGACCCATCCGACACGA |
| | | | TCCTTGACATTGGAAGTGCGCCCGCCCGCAGAATGTATTCTAAGC |
| | | | AGAAGTATCATTGTATGTGTCCGATGAGATGTGCGGAAGATCCGGA |
| | | | CAGATTGTATAAGTATGCAACTAAGCTGAAGAAAAACTGTAAGGAA |
| | | | ATAACTGATAAGGAATTGGACAAGAAAATGAAGGAGCTCGCCGCC |
| | | | GTCATGAGCGACCCTGACCTGGAAACTGAGACTATGTGCCTCCAC |
| | | | GACGACGAGTCGTGTCGCTACGAAGGGGAAGTCGCTGTTTACCAG |
| | | | GATGTATACGCGGTTGACGGACCGACAAGTCTCTATCACCAAGCC |
| | | | AATAAGGGAGTTAGAGTCGCCTACTGGATAGGCTTTGACACCACC |
| | | | CCTTTTATGTTTAAGAACTTGGCTGGAGCATATCCATCATACTCTAC |
| | | | CAACTGGGCCGACGAAACCGTGTTAAGGGCTCGTAACATAGGCCT |
| | | | ATGCAGCTCTGACGTTATGGAGCGGTCACGTAGAGGGATGTCCAT |
| | | | TCTTAGAAAGAAGTATTTGAAACCATCCAACAATGTTCTATTCTCTG |
| | | | TTGGCTCGACCATCTACCACGAGAAGAGGGACTTACTGAGGAGCT |
| | | | GGCACCTGCCGTGTGTATTTCACTTACGTGGCAAGCAAAATTACAC |
| | | | ATGTCGGTGTGAGACTATAGTTAGTTGCGAGGGGTACGTCGTTAAA |
| | | | AGAATAGCTATCAGTCCAGGCCTGTATGGGAAGCCTTCAGGCTAT |
| | | | GCTGCTACGATGCACCGCGAGGGATTCTTGTGCTGCAAAGTGACA |
| | | | GACACATTGAACGGGGAGAGGGTCTCTTTTCCCGTGTGCACGTAT |
| | | | GTGCCAGCTACATTGTGTGACCAAATGACTGGCATACTGGCAACA |
| | | | GATGTCAGTGCGGACGACGCGCAAAAACTGCTGGTTGGGCTCAAC |
| | | | CAGCGTATAGTCGTCAACGGTCGCACCCAGAGAAACACCAATACC |
| | | | ATGAAAAATTACCTTTTGCCCGTAGTGGCCCAGGCATTTGCTAGGT |
| | | | GGGCAAAGGAATATAAGGAAGATCAAGAAGATGAAAGGCCACTAG |
| | | | GACTACGAGATAGACAGTTAGTCATGGGGTGTTGTTGGGCTTTTAG |
| | | | AAGGCACAAGATAACATCTATTTATAAGCGCCCGGATACCCAAACC |
| | | | ATCATCAAAGTGAACAGGGATTTCCACTCATTCGTGCTGCCCAGGA |
| | | | TAGGCAGTAACACATTGGAGATCGGGCTGAGAACAAGAATCAGGA |
| | | | AAATGTTAGAGGAGCACAAGGAGCCGTCACCTCTCATTACCGCCG |
| | | | AGGACGTACAAGAAGCTAAGTGCGCAGCCGATGAGGCTAAGGAG |
| | | | GTGCGTGAAGCCGAGGAGTTGCGCGCAGCTCTACCACCTTTGGCA |
| | | | GCTGATGTTGAGGAGCCCACTCTGGAAGCCGATGTCGACTTGATG |
| | | | TTACAAGAGGCTGGGGCC |
| 2 | VEE non- | Viral protein | MEKVHVDIEEDSPFLRALQRSFPQFEVEAKQVTDNDHANARAFSHLA |
| | structural | | SKLIETEVDPSDTILDIGSAPARRMYSKHKYHCICPMRCAEDPDRLYKY |
| | protein 1 | | ATKLKKNCKEITDKELDKKMKELAAVMSDPDLETETMCLHDDESCRY |
| | amino acids | | EGQVAVYQDVYAVDGPTSLYHQANKGVRVAYWIGFDTTPFMFKNLA |
| | | | GAYPSYSTNWADETVLTARNIGLCSSDVMERSRRGMSILRKKYLKPS |
| | | | NNVLFSVGSTIYHEKRDLLRSWHLPSVFHLRGKQNYTCRCETIVSCD |
| | | | GYVVKRIAISPGLYGKPSGYAATMHREGFLCCKVTDTLNGERVSFPV |
| | | | CTYVPATLCDQMTGILATDVSADDAQKLLVGLNQRIVVNGRTQRNTN |
| | | | TMKNYLLPVVAQAFARWAKEYKEDQEDERPLGLRDRQLVMGCCWA |
| | | | FRRHKITSIYKRPDTQTIIKVNSDFHSFVLPRIGSNTLEIGLRTRIRKMLE |
| | | | EHKEPSPLITAEDVQEAKCAADEAKEVREAEELRAALPPLAADVEEPT |
| | | | LEADVDLMLQEAGA |
| 3 | VEE non- | Viral RNA | GGCUCAGUGGAGACACCUCGUGGCUUGAUAAAGGUUACCAGCUA |
| | structural protein 2 | | CGCUGGCGAGGACAAGAUCGGCUCUUACGCUGUGCUUUCUCCG |
| | RNA | | CAGGCUGUACUCAAGAGUGAAAAAUUAUCUUGCAUCCACCCUCU |
| | | | CGCUGAACAAGUCAUAGUGAUAACACACUCUGGCCGAAAAGGGC |
| | | | GUUAUGCCGUGGAACCAUACCAUGGUAAAGUAGUGGUGCCAGAG |
| | | | GGACAUGCAAUACCCGUCCAGGACUUUCAAGCUCUGAGUGAAAG |
| | | | UGCCACCAUUGUGUACAACGAACGUGAGUUCGUAAACAGGUACC |
| | | | UGCACCAUAUUGCCACACAUGGAGGAGCGCUGAACACUGAUGAA |
| | | | GAAUAUUACAAAACUGUCAAGCCCAGCGAGCACGACGGCGAAUA |
| | | | CCUGUACGACAUCGACAGGAAACAGUGCGUCAAGAAAGAACUAG |
| | | | UCACUGGGCUAGGGCUCACAGGCGAGCUGGUGGAUCCUCCCUU |
| | | | CCAUGAAUUCGCCUACGAGAGUCUGAGAACACGACCAGCCGCUC |
| | | | CUUACCAAGUACCAACCAUAGGGGUGUAUGGCGUGCCAGGAUCA |
| | | | GGCAAGUCUGGCAUCAUUAAAAGCGCAGUCACCAAAAAAGAUCU |
| | | | AGUGGUGAGCGCCAAGAAAGAAAACUGUGCAGAAAUUAUAAGGG |
| | | | ACGUCAAGAAAAUGAAAGGGCUGGACGUCAAUGCCAGAACUGUG |
| | | | GACUCAGUGCUCUUGAAUGGAUGCAAACACCCCGUAGAGACCCU |
| | | | GUAUAUUGACGAAGCUUUUGCUUGUCAUGCAGGUACUCUCAGAG |
| | | | CGCUCAUAGCCAUUAUAAGACCUAAAAAGGCAGUGCUCUGGGGG |
| | | | GAUCCCAAACAGUGCGGUUUUUUUAACAUGAUGUGCCUGAAAGU |
| | | | GCAUUUUAACCACGAGAUUUGCACACAAGUCUUCCACAAAAGCA |
| | | | UCUCUCGCCGUUGCACUAAAUCUGUGACUUCGGUCGUCUCAACC |
| | | | UUGUUUUACGACAAAAAAAUGAGAACGACGAAUCCGAAAGAGACU |
| | | | AAGAUUGUGAUUGACACUACCGGCAGUACCAAACCUAAGCAGGA |
| | | | CGAUCUCAUUCUCACUUGUUUCAGAGGGUGGGUGAAGCAGUUG |
| | | | CAAAUAGAUUACAAAGGCAACGAAAUAAUGACGGCAGCUGCCUC |
| | | | UCAAGGGCUGACCCGUAAAGGUGUGUAUGCCGUUCGGUACAAG |
| | | | GUGAAUGAAAAUCCUCUGUACGCACCCACCUCAGAACAUGUGAA |
| | | | CGUCCUACUGACCCGCACGGAGGACCGCAUCGUGUGGAAAACAC |
| | | | UAGCCGGCGACCAUGGAUAAAAACACUGACUGCCAAGUACCCU |
| | | | GGGAAUUUCACUGCCACGAUAGAGGAGUGGCAAGCAGAGCAUGA |
| | | | UGCCAUCAUGAGGCACAUCUUGGAGAGACCGGACCCUACCGACG |
| | | | UCUUCCAGAAUAAGGCAAACGUGUGUUGGGCCAAGGCUUUAGUG |
| | | | CCGGUGCUGAAGACCGCUGGCAUAGACAUGACCACUGAACAAUG |
| | | | GAACACUGUGGAUUAUUUUGAAACGGACAAAGCUCACUCAGCAG |
| | | | AGAUAGUAUUGAACCAACUAUGCGUGAGGUUCUUUGGACUCGAU |
| | | | CUGGACUCCGGUCUAUUUUCUGCACCCACUGUUCCGUUAUCCAU |
| | | | UAGGAAUAAUCACUGGGAUAACUCCCCGUCGCCUAACAUGUACG |
| | | | GGCUGAAUAAAGAAGUGGUCCGUCAGCUCUCUCGCAGGUACCCA |
| | | | CAACUGCCUCGGGCAGUUGCCACUGGAAGAGUCUAUGACAUGAA |
| | | | CACUGGUACACUGCGCAAUUAUGAUCCGCGCAUAAACCUAGUAC |
| | | | CUGUAAACAGAAGACUGCCUCAUGCUUUAGUCCUCCACCAUAAU |
| | | | GAACACCCACAGAGUGACUUUUCUUCAUUCGUCAGCAAAUUGAA |
| | | | GGGCAGAACUGUCCUGGUGGUCGGGGAAAAGUUGUCCGUCCCA |
| | | | GGCAAAAUGGUUGACUGGUUGUCAGACCGGCCUGAGGCUACCU |
| | | | UCAGAGCUCGGCUGGAUUUAGGCAUCCCAGGUGAUGUGCCCAA |
| | | | AUAUGACAUAAUAUUUGUUAAUGUGAGGACCCCAUAUAAAUACCA |
| | | | UCACUAUCAGCAGUGUGAAGACCAUGCCAUUAAGCUUAGCAUGU |
| | | | UGACCAAGAAAGCUUGUCUGCAUCUGAAUCCCGGCGGAACCUGU |
| | | | GUCAGCAUAGGUUAUGGUUACGCUGACAGGGCCAGCGAAAGCAU |
| | | | CAUUGGUGCUAUAGCGCGGCAGUUCAAGUUUUCCCGGGUAUGC |
| | | | AAACCGAAAUCCUCACUUGAAGAGACGGAAGUUCUGUUUGUAUU |
| | | | CAUUGGGUACGAUCGCAAGGCCCGUACGCACAAUCCUUACAAGC |
| | | | UUUCAUCAACCUUGACCAACAUUUAUACAGGUUCCAGACUCCAC |
| | | | GAAGCCGGAUGU |
| 3A | DNA sequence | | GGCTCAGTGGAGACACCTCGTGGCTTGATAAAGGTTACCAGCTAC |
| | corresponding to | | GCTGGCGAGGACAAGATCGGCTCTTACGCTGTGCTTTCTCCGCAG |
| | SEQ ID NO: 3 | | GCTGTACTCAAGAGTGAAAAATTATCTTGCATCCACCCTCTCGCTG |
| | | | AACAAGTCATAGTGATAACACACTCTGGCCGAAAAGGGCGTTATG |
| | | | CCGTGGAACCATACCATGGTAAAGTAGTGGTGCCAGAGGGACATG |
| | | | CAATACCCGTCCAGGACTTTCAAGCTCTGAGTGAAAGTGCCACCAT |
| | | | TGTGTACAACGAACGTGAGTTCGTAAACAGGTACCTGCACCATATT |
| | | | GCCACACATGGAGGAGCGCTGAACACTGATGAAGAATATTACAAA |
| | | | ACTGTCAAGCCCAGCGAGCACGACGGCGAATACCTGTACGACATC |
| | | | GACAGGAAACAGTGCGTCAAGAAAGAACTAGTCACTGGGCTAGGG |
| | | | CTCACAGGCGAGCTGGTGGATCCTCCCTTCCATGAATTCGCCTAC |
| | | | GAGAGTCTGAGAACACGACCAGCCGCTCCTTACCAAGTACCAACC |
| | | | ATAGGGGTGTATGGCGTGCCAGGATCAGGCAAGTCTGGCATCATT |
| | | | AAAAGCGCAGTCACCAAAAAAGATCTAGTGGTGAGCGCCAAGAAA |
| | | | GAAAACTGTGCAGAAATTATAAGGGACGTCAAGAAAATGAAAGGG |
| | | | CTGGACGTCAATGCCAGAACTGTGGACTCAGTGCTCTTGAATGGA |
| | | | TGCAAACACCCCGTAGAGACCCTGTATATTGACGAAGCTTTTGCTT |
| | | | GTCATGCAGGTACTCTCAGAGCGCTCATAGCCATTATAAGACCTAA |
| | | | AAAGGCAGTGCTCTGCGGGGATCCCAAACAGTGCGGTTTTTTTAA |
| | | | CATGATGTGCCTGAAAGTGCATTTTAACCACGAGATTTGCACACAA |
| | | | GTCTTCCACAAAAGCATCTCTCGCCGTTGCACTAAATCTGTGACTT |
| | | | CGGTCGTCTCAACCTTGTTTTACGACAAAAAAATGAGAACGACGAA |
| | | | TCCGAAAGAGACTAAGATTGTGATTGACACTACCGGCAGTACCAAA |
| | | | CCTAAGCAGGACGATCTCATTCTCACTTGTTTCAGAGGGTGGGTGA |
| | | | AGCAGTTGCAAATAGATTACAAAGGCAACGAAATAATGAGGGCAG |
| | | | CTGCCTCTCAAGGGCTGACCCGTAAAGGTGTGTATGCCGTTCGGT |
| | | | ACAAGGTGAATGAAAATCCTCTGTACGCACCCACCTCAGAACATGT |
| | | | GAACGTCCTACTGACCCGCACGGAGGACCGCATCGTGTGGAAAAC |
| | | | ACTAGCCGGCGACCCATGGATAAAAACACTGACTGCCAAGTACCC |
| | | | TGGGAATTTCACTGCCACGATAGAGGAGTGGCAAGCAGAGCATGA |
| | | | TGCCATCATGAGGCACATCTTGGAGAGACCGGACCCTACCGACGT |
| | | | CTTCCAGAATAAGGCAAACGTGTGTTGGGCGAAGGCTTTAGTGCC |
| | | | GGTGCTGAAGACCGCTGGCATAGACATGACCACTGAACAATGGAA |
| | | | CACTGTGGATTATTTTGAAACGGACAAAGCTCACTCAGCAGAGATA |
| | | | GTATTGAACCAACTATGGGTGAGGTTCTTTGGACTCGATCTGGACT |
| | | | CCGGTCTATTTTCTGCACCCACTGTTCCGTTATCCATTAGGAATAAT |
| | | | CACTGGGATAACTCCCCGTCGCCTAACATGTACGGGCTGAATAAA |
| | | | GAAGTGGTCCGTCAGCTCTCTCGCAGGTACCCACAACTGCCTCGG |
| | | | GCAGTTGCCACTGGAAGAGTCTATGACATGAACACTGGTACACTG |
| | | | CGCAATTATGATCCGCGCATAAACCTAGTACCTGTAAACAGAAGAC |
| | | | TGCCTCATGCTTTAGTCCTCCACCATAATGAACACCCACAGAGTGA |
| | | | CTTTTCTTCATTCGTCAGCAAATTGAAGGGCAGAACTGTCCTGGTG |
| | | | GTCGGGGAAAAGTTGTCCGTCCCAGGCAAAATGGTTGACTGGTTG |
| | | | TCAGACCGGCCTGAGGCTACCTTCAGAGCTCGGCTGGATTTAGGC |
| | | | ATCCCAGGTGATGTGCCCAAATATGACATAATATTTGTTAATGTGA |
| | | | GGACCCCATATAAATACCATCACTATCAGCAGTGTGAAGACCATGC |
| | | | CATTAAGCTTAGCATGTTGACCAAGAAAGCTTGTCTGCATCTGAAT |
| | | | CCCGGGGGAACCTGTGTCAGCATAGGTTATGGTTACGCTGACAGG |
| | | | GCCAGCGAAAGCATCATTGGTGCTATAGGGGGGCAGTTCAAGTTT |
| | | | TCCCGGGTATGCAAACCGAAATCCTCACTTGAAGAGACGGAAGTT |
| | | | CTGTTTGTATTCATTGGGTACGATCGCAAGGCCCGTACGCACAATC |
| | | | CTTACAAGCTTTCATCAACCTTGACCAACATTTATACAGGTTCCAGA |
| | | | CTCCACGAAGCCGGATGT |
| 4 | VEE non- | Viral protein | GSVETPRGLIKVTSYAGEDKIGSYAVLSPQAVLKSEKLSCIHPLAEQVI |
| | structural | | VITHSGRKGRYAVEPYHGKVVVPEGHAIPVQDFQALSESATIVYNERE |
| | protein 2 | | FVNRYLHHIATHGGALNTDEEYYKTVKPSEHDGEYLYDIDRKQCVKKE |
| | amino acids | | LVTGLGLTGELVDPPFHEFAYESLRTRPAAPYQVPTIGVYGVPGSGKS |
| | | | GIIKSAVTKKDLVVSAKKENCAEIIRDVKKMKGLDVNARTVDSVLLNGC |
| | | | KHPVETLYIDEAFACHAGTLRALIAIIRPKKAVLCGDPKQCGFFNMMCL |
| | | | KVHFNHEICTQVFHKSISRRCTKSVTSVVSTLFYDKKMRTTNPKETKIV |
| | | | IDTTGSTKPKQDDLILTCFRGWVKQLQIDYKGNEIMTAAASQGLTRKG |
| | | | VYAVRYKVNENPLYAPTSEHVNVLLTRTEDRIVWKTLAGDPWIKTLTA |
| | | | KYPGNFTATIEEWQAEHDAIMRHILERPDPTDVFQNKANVCWAKALV |
| | | | PVLKTAGIDMTTEQWNTVDYFETDKAHSAEIVLNQLCVRFFGLDLDSG |
| | | | LFSAPTVPLSIRNNHWDNSPSPNMYGLNKEVVRQLSRRYPQLPRAVA |
| | | | TGRVYDMNTGTLRNYDPRINLVPVNRRLPHALVLHHNEHPQSDFSSF |
| | | | VSKLKGRTVLVVGEKLSVPGKMVDWLSDRPEATFRARLDLGIPGDVP |
| | | | KYDIIFVNVRTPYKYHHYQQCEDHAIKLSMLTKKACLHLNPGGTCVSIG |
| | | | YGYADRASESIIGAIARQFKFSRVCKPKSSLEETEVLFVFIGYDRKART |
| | | | HNPYKLSSTLTNIYTGSRLHEAGC |
| 5 | VEE non- | Viral RNA | GCACCCUCAUAUCAUGUGGUGCGAGGGGAUAUUGCCACGGCCA |
| | structural | | CCGAAGGAGUGAUUAUAAAUGCUGCUAACAGCAAAGGACAACCU |
| | protein 3 | | GGCGGAGGGGUGUGCGGAGCGCUGUAUAAGAAAUUCCCGGAAA |
| | RNA | | GCUUCGAUUUAGAGCCGAUCGAAGUAGGAAAAGCGCGACUGGUC |
| | | | AAAGGUGCAGCUAAACAUAUCAUUCAUGCCGUAGGACCAAACUU |
| | | | CAACAAAGUUUGGGAGGUUGAAGGUGACAAACAGUUGGCAGAGG |
| | | | CUUAUGAGUCCAUCGCUAAGAUUGUCAACGAUAACAAUUACAAG |
| | | | UCAGUAGCGAUUCCACUGUUGUCCACCGGCAUCUUUUCCGGGAA |
| | | | CAAAGAUCGACUAACCCAAUCAUUGAACCAUUUGCUGACAGCUU |
| | | | UAGACACCACUGAUGCAGAUGUAGCCAUAUACUGCAGGGACAAG |
| | | | AAAUGGGAAAUGACUCUCAAGGAAGCAGUGGCUAGGAGAGAAGC |
| | | | AGUGGAGGAGAUAUGCAUAUCCGACGACUCUUCAGUGACAGAAC |
| | | | CUGAUGCAGAGCUGGUGAGGGUGCAUCCGAAGAGUUCUUUGGC |
| | | | UGGAAGGAAGGGCUACAGCACAAGCGAUGGCAAAACUUUCUCAU |
| | | | AUUUGGAAGGGACCAAGUUUCACCAGGGGGCCAAGGAUAUAGCA |
| | | | GAAAUUAAUGCCAUGUGGCCCGUUGCAACGGAGGCCAAUGAGCA |
| | | | GGUAUGCAUGUAUAUCCUCGGAGAAAGCAUGAGCAGUAUUAGGU |
| | | | CGAAAUGCCCCGUCGAAGAGUCGGAAGCCUCCACACCACCUAGC |
| | | | ACGCUGCCUUGCUUGUGCAUCCAUGCCAUGACUCCAGAAAGAGU |
| | | | ACAGCGCCUAAAAGCCUCACGUCCAGAACAAAUUACUGUGUGCU |
| | | | CAUCCUUUCCAUUGCCGAAGUAUAGAAUCACUGGUGUGCAGAAG |
| | | | AUCCAAUGCUCCCAGCCUAUAUUGUUCUCACCGAAAGUGCCUGC |
| | | | GUAUAUUCAUCCAAGGAAGUAUCUCGUGGAAACACCACCGGUAG |
| | | | ACGAGACUCCGGAGCCAUCGGCAGAGAACCAAUCCACAGAGGGG |
| | | | ACACCUGAACAACCACCAGUUAUAACCGAGGAUGAGACCAGGAC |
| | | | UAGAACGCCUGAGCCGAUCAUCAUCGAAGAGGAAGAAGAGGAUA |
| | | | GCAUAAGUUUGCUGUCAGAUGGCCCGACCCACCAGGUGCUGCAA |
| | | | GUCGAGGCAGACAUUCACGGGCCGCCCUCUGUAUCUAGCUCAU |
| | | | CCUGGUCCAUUCCUCAUGCAUCCGACUUUGAUGUGGACAGUUUA |
| | | | UCCAUACUUGACACCCUGGAGGGAGCUAGCGUGACCAGGGGGG |
| | | | CAACGUCAGCCGAGACUAACUCUUACUUCGCAAAGAGUAUGGAG |
| | | | UUUCUGGCGCGACCGGUGCCUGCGCCUCGAACAGUAUUCAGGA |
| | | | ACCCUCCACAUCCCGCUCCGCGCACAAGAACACCGUCACUUGCA |
| | | | CCCAGCAGGGCCUGCUCGAGAACCAGCCUAGUUUCCACCCCGCC |
| | | | AGGCGUGAAUAGGGUGAUCACUAGAGAGGAGCUCGAGGCGCUU |
| | | | ACCCCGUCACGCACUCCUAGCAGGUCGGUCUCGAGAACCAGCCU |
| | | | GGUCUCCAACCCGCCAGGCGUAAAUAGGGUGAUUACAAGAGAGG |
| | | | AGUUUGAGGCGUUCGUAGCACAACAACAAUGACGGUUUGAUGCG |
| | | | GGUGCA |
| 5A | DNA sequence | | GCACCCTCATATCATGTGGTGCGAGGGGATATTGCCACGGCCACC |
| | corresponding to | | GAAGGAGTGATTATAAATGCTGCTAACAGCAAAGGACAACCTGGC |
| | SEQ ID NO: 5 | | GGAGGGGTGTGCGGAGCGCTGTATAAGAAATTCCCGGAAAGCTTC |
| | | | GATTTACAGCCGATCGAAGTAGGAAAAGCGCGACTGGTCAAAGGT |
| | | | GCAGCTAAACATATCATTCATGCCGTAGGACCAAACTTCAACAAAG |
| | | | TTTCGGAGGTTGAAGGTGACAAACAGTTGGCAGAGGCTTATGAGT |
| | | | CCATCGCTAAGATTGTCAACGATAACAATTACAAGTCAGTAGCGAT |
| | | | TCCACTGTTGTCCACCGGCATCTTTTCCGGGAACAAAGATCGACTA |
| | | | ACCCAATCATTGAACCATTTGCTGACAGCTTTAGACACCACTGATG |
| | | | CAGATGTAGCCATATACTGCAGGGAGAAGAAATGGGAAATGACTC |
| | | | TCAAGGAAGCAGTGGCTAGGAGAGAAGCAGTGGAGGAGATATGC |
| | | | ATATCCGACGACTCTTCAGTGACAGAACCTGATGCAGAGCTGGTG |
| | | | AGGGTGCATCCGAAGAGTTCTTTGGCTGGAAGGAAGGGCTACAGC |
| | | | ACAAGCGATGGCAAAACTTTCTCATATTTGGAAGGGACCAAGTTTC |
| | | | ACCAGGCGGCCAAGGATATAGCAGAAATTAATGCCATGTGGCCCG |
| | | | TTGCAACGGAGGCCAATGAGCAGGTATGCATGTATATCCTCGGAG |
| | | | AAAGCATGAGCAGTATTAGGTCGAAATGCCCCGTCGAAGAGTCGG |
| | | | AAGCCTCCACACCACCTAGCACGCTGCCTTGCTTGTGCATCCATG |
| | | | CCATGACTCCAGAAAGAGTACAGCGCCTAAAAGCCTCACGTCCAG |
| | | | AACAAATTACTGTGTGCTCATCCTTTCCATTGCCGAAGTATAGAATC |
| | | | ACTGGTGTGCAGAAGATCCAATGCTCCCAGCCTATATTGTTCTCAC |
| | | | CGAAAGTGCCTGCGTATATTCATCCAAGGAAGTATCTCGTGGAAAC |
| | | | ACCACCGGTAGACGAGACTCCGGAGCCATCGGCAGAGAACCAAT |
| | | | CCACAGAGGGGACACCTGAACAACCACCACTTATAACCGAGGATG |
| | | | AGACCAGGACTAGAACGCCTGAGCCGATCATCATCGAAGAGGAAG |
| | | | AAGAGGATAGCATAAGTTTGCTGTCAGATGGCCCGACCCACCAGG |
| | | | TGCTGCAAGTCGAGGCAGACATTCACGGGCCGCCCTCTGTATCTA |
| | | | GCTCATCCTGGTCCATTCCTCATGCATCCGACTTTGATGTGGACAG |
| | | | TTTATCCATACTTGACACCCTGGAGGGAGCTAGCGTGACCAGCGG |
| | | | GGCAACGTCAGCCGAGACTAACTCTTACTTCGCAAAGAGTATGGA |
| | | | GTTTCTGGGGGGACCGGTGCCTGCGCCTCGAACAGTATTCAGGAA |
| | | | CCCTCCACATCCCGCTCCGCGCACAAGAACACCGTCACTTGCACC |
| | | | CAGCAGGGCCTGCTCGAGAACCAGCCTAGTTTCCACCCCGCCAG |
| | | | GCGTGAATAGGGTGATCACTAGAGAGGAGCTCGAGGCGCTTACCC |
| | | | CGTCACGCACTCCTAGCAGGTCGGTCTCGAGAACCAGCCTGGTCT |
| | | | CCAACCCGCCAGGCGTAAATAGGGTGATTACAAGAGAGGAGTTTG |
| | | | AGGCGTTCGTAGCACAACAACAATGACGGTTTGATGCGGGTGCA |
| 6 | VEE non- | Viral protein | APSYHVVRGDIATATEGVIINAANSKGQPGGGVCGALYKKFPESFDLQ |
| | structural | | PIEVGKARLVKGAAKHIIHAVGPNFNKVSEVEGDKQLAEAYESIAKIVN |
| | protein 3 | | DNNYKSVAIPLLSTGIFSGNKDRLTQSLNHLLTALDTTDADVAIYCRDK |
| | amino acids | | KWEMTLKEAVARREAVEEICISDDSSVTEPDAELVRVHPKSSLAGRK |
| | | | GYSTSDGKTFSYLEGTKFHQAAKDIAEINAMWPVATEANEQVCMYILG |
| | | | ESMSSIRSKCPVEESEASTPPSTLPCLCIHAMTPERVQRLKASRPEQI |
| | | | TVCSSFPLPKYRITGVQKIQCSQPILFSPKVPAYIHPRKYLVETPPVDET |
| | | | PEPSAENQSTEGTPEQPPLITEDETRTRTPEPIIIEEEEEDSISLLSDGP |
| | | | THQVLQVEADIHGPPSVSSSSWSIPHASDFDVDSLSILDTLEGASVTS |
| | | | GATSAETNSYFAKSMEFLARPVPAPRTVFRNPPHPAPRTRTPSLAPS |
| | | | RACSRTSLVSTPPGVNRVITREELEALTPSRTPSRSVSRTSLVSNPPG |
| | | | VNRVITREEFEAFVAQQQ.RFDAGA |
| 7 | VEE non- | Viral RNA | UACAUCUUUUCCUCCGACACCGGUCAAGGGCAUUUACAACAAAA |
| | structural | | AUCAGUAAGGCAAACGGUGCUAUCCGAAGUGGUGUUGGAGAGG |
| | protein 4 | | ACCGAAUUGGAGAUUUCGUAUGCCCCGCGCCUCGACCAAGAAAA |
| | RNA | | AGAAGAAUUACUACGCAAGAAAUUACAGUUAAAUCCCACACCUGC |
| | | | UAACAGAAGCAGAUACCAGUCCAGGAAGGUGGAGAACAUGAAAG |
| | | | CCAUAACAGCUAGACGUAUUCUGCAAGGCCUAGGGCAUUAUUUG |
| | | | AAGGCAGAAGGAAAAGUGGAGUGCUACCGAACCCUGCAUCCUGU |
| | | | UCCUUUGUAUUCAUCUAGUGUGAACCGUGCCUUUUCAAGCCCCA |
| | | | AGGUCGCAGUGGAAGCCUGUAACGCCAUGUUGAAAGAGAACUUU |
| | | | CCGACUGUGGCUUGUUACUGUAUUAUUCCAGAGUACGAUGCCUA |
| | | | UUUGGACAUGGUUGACGGAGCUUCAUGCUGCUUAGACACUGCC |
| | | | AGUUUUUGCCCUGCAAAGCUGCGCAGCUUUCCAAAGAAACACUC |
| | | | CUAUUUGGAACCCACAAUACGAUCGGCAGUGCCUUCAGCGAUCC |
| | | | AGAACACGCUCCAGAACGUCCUGGCAGCUGCCACAAAAAGAAAU |
| | | | UGCAAUGUCACGCAAAUGAGAGAAUUGCCCGUAUUGGAUUCGGC |
| | | | GGCCUUUAAUGUGGAAUGCUUCAAGAAAUAUGCGUGUAAUAAUG |
| | | | AAUAUUGGGAAACGUUUAAAGAAAACCCCAUCAGGCUUACUGAA |
| | | | GAAAACGUGGUAAAUUACAUUACCAAAUUAAAAGGACCAAAAGCU |
| | | | GCUGCUCUUUUUGCGAAGACACAUAAUUUGAAUAUGUUGCAGGA |
| | | | CAUACCAAUGGACAGGUUUGUAAUGGACUUAAAGAGAGACGUGA |
| | | | AAGUGACUCCAGGAACAAAACAUACUGAAGAACGGCCCAAGGUA |
| | | | CAGGUGAUCCAGGCUGCCGAUCCGCUAGCAACAGCGUAUCUGU |
| | | | GCGGAAUCCACCGAGAGCUGGUUAGGAGAUUAAAUGCGGUCGU |
| | | | GCUUCCGAACAUUCAUACACUGUUUGAUAUGUCGGCUGAAGACU |
| | | | UUGACGCUAUUAUAGCCGAGCACUUCCAGCCUGGGGAUUGUGU |
| | | | UUCUGGAAACUGACAUCGCGUCGUUUGAUAAAAGUGAGGACGACG |
| | | | CCAUGGCUCUGACCGCGUUAAUGAUUCUGGAAGACUUAGGUGU |
| | | | GGACGCAGAGCUGUUGACGCUGAUUGAGGCGGCUUUCGGCGAA |
| | | | AUUUCAUCAAUACAUUUGCCCACUAAAACUAAAUUUAAAUUCGGA |
| | | | GCCAUGAUGAAAUCUGGAAUGUUCCUCACACUGUUUGUGAACAC |
| | | | AGUCAUUAACAUUGUAAUCGCAAGCAGAGUGUUGAGAGAACGGC |
| | | | UAACCGGAUCACCAUGUGCAGCAUUCAUUGGAGAUGACAAUAUC |
| | | | GUGAAAGGAGUCAAAUGGGACAAAUUAAUGGCAGACAGGUGCGC |
| | | | CACCUGGUUGAAUAUGGAAGUCAAGAUUAUAGAUGCUGUGGUGG |
| | | | GCGAGAAAGCGCCUUAUUUCUGUGGAGGGUUUAUUUUGUGUGA |
| | | | CUCCGUGACCGGCACAGCGUGCCGUGUGGCAGACCCCCUAAAAA |
| | | | GGCUGUUUAAGCUUGGCAAACCUCUGGCAGCAGACGAUGAACAU |
| | | | GAUGAUGACAGGAGAAGGGCAUUGCAUGAAGAGUCAACACGCUG |
| | | | GAACCGAGUGGGUAUUCUUUCAGAGCUGUGCAAGGCAGUAGAAU |
| | | | CAAGGUAUGAAACCGUAGGAACUUCCAUCAUAGUUAUGGCCAUG |
| | | | ACUACUCUAGCUAGCAGUGUUAAAUCAUUCAGCUACCUGAGAGG |
| | | | GGCCCCUAUAACUCUCUACGGC |
| 7A | DNA sequence | | TACATCTTTTCCTCCGACACCGGTCAAGGGCATTTACAACAAAAAT |
| | corresponding to | | CAGTAAGGCAAACGGTGCTATCCGAAGTGGTGTTGGAGAGGACCG |
| | SEQ ID NO: 7 | | AATTGGAGATTTCGTATGCCCCGCGCCTCGACCAAGAAAAAGAAG |
| | | | AATTACTACGCAAGAAATTACAGTTAAATCCCACACCTGCTAACAG |
| | | | AAGCAGATACCAGTCCAGGAAGGTGGAGAACATGAAAGCCATAAC |
| | | | AGCTAGACGTATTCTGCAAGGCCTAGGGCATTATTTGAAGGCAGA |
| | | | AGGAAAAGTGGAGTGCTACCGAACCCTGCATCCTGTTCCTTTGTAT |
| | | | TCATCTAGTGTGAACCGTGCCTTTTCAAGCCCCAAGGTCGCAGTG |
| | | | GAAGCCTGTAACGCCATGTTGAAAGAGAACTTTCCGACTGTGGCTT |
| | | | CTTACTGTATTATTCCAGAGTACGATGCCTATTTGGACATGGTTGA |
| | | | CGGAGCTTCATGCTGCTTAGACACTGCCAGTTTTTGCCCTGCAAAG |
| | | | CTGCGCAGCTTTCCAAAGAAACACTCCTATTTGGAACCCACAATAC |
| | | | GATCGGCAGTGCCTTCAGCGATCCAGAACACGCTCCAGAACGTCC |
| | | | TGGCAGCTGCCACAAAAAGAAATTGCAATGTCACGCAAATGAGAG |
| | | | AATTGCCCGTATTGGATTCGGCGGCCTTTAATGTGGAATGCTTCAA |
| | | | GAAATATGCGTGTAATAATGAATATTGGGAAACGTTTAAAGAAAAC |
| | | | CCCATCAGGCTTACTGAAGAAAACGTGGTAAATTACATTACCAAAT |
| | | | TAAAAGGACCAAAAGCTGCTGCTCTTTTTGCGAAGACACATAATTT |
| | | | GAATATGTTGCAGGACATACCAATGGACAGGTTTGTAATGGACTTA |
| | | | AAGAGAGACGTGAAAGTGACTCCAGGAACAAAACATACTGAAGAA |
| | | | CGGCCCAAGGTACAGGTGATCCAGGCTGCCGATCCGCTAGCAAC |
| | | | AGCGTATCTGTGCGGAATCCACCGAGAGCTGGTTAGGAGATTAAA |
| | | | TGCGGTCCTGCTTCCGAACATTCATACACTGTTTGATATGTCGGCT |
| | | | GAAGACTTTGACGCTATTATAGCCGAGCACTTCCAGCCTGGGGAT |
| | | | TGTGTTCTGGAAACTGACATCGCGTCGTTTGATAAAAGTGAGGACG |
| | | | ACGCCATGGCTCTGACCGCGTTAATGATTCTGGAAGACTTAGGTGT |
| | | | GGACGCAGAGCTGTTGACGCTGATTGAGGGGGCTTTCGGCGAAAT |
| | | | TTCATCAATACATTTGCCCACTAAAACTAAATTTAAATTCGGAGCCA |
| | | | TGATGAAATCTGGAATGTTCCTCACACTGTTTGTGAACACAGTCATT |
| | | | AACATTGTAATCGGAAGCAGAGTGTTGAGAGAACGGCTAACCGGA |
| | | | TACCATGTGCAGCATTCATTGGAGATGACAATATCGTGAAAGGAG |
| | | | TCAAATCGGACAAATTAATGGCAGACAGGTGCGCCACCTGGTTGA |
| | | | ATATGGAAGTCAAGATTATAGATGCTGTGGTGGGGGAGAAAGCGC |
| | | | CTTATTTCTGTGGAGGGTTTATTTTGTGTGACTCCGTGACCGGCAC |
| | | | AGCGTGCCGTGTGGCAGACCCCCTAAAAAGGCTGTTTAAGCTTGG |
| | | | CAAACCTCTGGCAGCAGACGATGAACATGATGATGACAGGAGAAG |
| | | | GGCATTGCATGAAGAGTCAACACGCTGGAACCGAGTGGGTATTCT |
| | | | TTCAGAGCTGTGCAAGGCAGTAGAATCAAGGTATGAAACCGTAGG |
| | | | AACTTCCATCATAGTTATGGCCATGACTACTCTAGCTAGCAGTGTT |
| | | | AAATCATTCAGCTACCTGAGAGGGGCCCCTATAACTCTCTACGGC |
| 8 | VEE non- | Viral protein | YIFSSDTGQGHLQQKSVRQTVLSEVVLERTELEISYAPRLDQEKEELL |
| | structural | | RKKLQLNPTPANRSRYQSRKVENMKAITARRILQGLGHYLKAEGKVE |
| | protein 4 | | CYRTLHPVPLYSSSVNRAFSSPKVAVEACNAMLKENFPTVASYCHIPE |
| | amino acids | | YDAYLDMVDGASCCLDTASFCPAKLRSFPKKHSYLEPTIRSAVPSAIQ |
| | | | NTLQNVLAAATKRNCNVTQMRELPVLDSAAFNVECFKKYACNNEYW |
| | | | ETFKENPIRLTEENVVNYITKLKGPKAAALFAKTHNLNMLQDIPMDRFV |
| | | | MDLKRDVKVTPGTKHTEERPKVQVIQAADPLATAYLCGIHRELVRRLN |
| | | | AVLLPNIHTLFDMSAEDFDAIIAEHFQPGDCVLETDIASFDKSEDDAMA |
| | | | LTALMILEDLGVDAELLTLIEAAFGEISSIHLPTKTKFKFGAMMKSGMFL |
| | | | TLFVNTVINIVIASRVLRERLTGSPCAAFIGDDNIVKGVKSDKLMADRCA |
| | | | TWLNMEVKIIDAVVGEKAPYFCGGFILCDSVTGTACRVADPLKRLFKL |
| | | | GKPLAADDEHDDDRRRALHEESTRWNRVGILSELCKAVESRYETVGT |
| | | | SIIVMAMTTLASSVKSFSYLRGAPITLYG |
| 9 | XCL1 DNA | | ATGAGACTTCTCATCCTGGCCCTCCTTGGCATCTGCTCTCTCACTG |
| | | | CATACATTGTGGAAGGTGTAGGGAGTGAAGTCTCAGATAAGAGGA |
| | | | CCTGTGTGAGCCTCACTACCCAGCGACTGCCGGTTAGCAGAATCA |
| | | | AGACCTACACCATCACGGAAGGCTCCTTGAGAGCAGTAATTTTTAT |
| | | | TACCAAACGTGGCCTAAAAGTCTGTGCTGATCCACAAGCCACATG |
| | | | GGTGAGAGACGTGGTCAGGAGCATGGACAGGAAATCCAACACCA |
| | | | GAAATAACATGATCCAGACCAAGCCAACAGGAACCCAGCAATCGA |
| | | | CCAATACAGCTGTGACTCTGACTGGCTAG |
| 9A | RNA sequence | | AUGAGACUUCUCAUCCUGGCCCUCCUUGGCAUCUGCUCUCUCAC |
| | corresponding to | | UGCAUACAUUGUGGAAGGUGUAGGGAGUGAAGUCUCAGAUAAGA |
| | SEQ ID NO: 9 | | GGACCUGUGUGAGCCUCACUACCCAGCGACUGCCGGUUAGCAG |
| | | | AAUCAAGACCUACACCAUCACGGAAGGCUCCUUGAGAGCAGUAA |
| | | | UUUUUAUUACCAAACGUGGCCUAAAAGUCUGUGCUGAUCCACAA |
| | | | GCCACAUGGGUGAGAGACGUGGUCAGGAGCAUGGACAGGAAAU |
| | | | CCAACACCAGAAAUAACAUGAUCCAGACCAAGCCAACAGGAACCC |
| | | | AGCAAUCGACCAAUACAGCUGUGACUCUGACUGGCUAG |
| 10 | XCL1 amino acids | Human protein | MRLLILALLGICSLTAYIVEGVGSEVSDKRTCVSLTTQRLPVSRIK |
| | | | TYTITEGSLRAVIFITKRGLKVCADPQATWVRDVVRSMDRKSNTR |
| | | | NNMIQTKPTGTQQSTNTAVTLTG |
| 11 | XCL2 DNA | | ATGAGACTTCTCATCCTGGCCCTCCTTGGCATCTGCTCTCTCACTG |
| | | | CATACATTGTGGAAGGTGTAGGGAGTGAAGTCTCACATAGGAGGA |
| | | | CCTGTGTGAGCCTCACTACCCAGCGACTGCCAGTTAGCAGAATCA |
| | | | AGACCTACACCATCACGGAAGGCTCCTTGAGAGCAGTAATTTTTAT |
| | | | TACCAAACGTGGCCTAAAAGTGTGTGCTGATCCAGAAGCCACGTG |
| | | | GGTGAGAGACGTGGTCAGGAGCATGGACAGGAAATCCAACACCA |
| | | | GAAATAACATGATCCAGACCAAGCCAACAGGAACCCAGCAATCGA |
| | | | CCAATACAGCTGTGACCCTGACTGGCTAG |
| 11A | RNA | | AUGAGACUUCUCAUCCUGGCCCUCCUUGGCAUCUGCUCUCUCAC |
| | corresponding | | UGCAUACAUUGUGGAAGGUGUAGGGAGUGAAGUCUCACAUAGGA |
| | to SEQ ID | | GGACCUGUGUGAGCCUCACUACCCAGCGACUGCCAGUUAGCAGA |
| | NO: 11 | | AUCAAGACCUACACCAUCACGGAAGGCUCCUUGAGAGCAGUAAU |
| | | | UUUUAUUACCAAACGUGGCCUAAAAGUCUGUGCUGAUCCACAAG |
| | | | CCACGUGGGUGAGAGACGUGGUCAGGAGCAUGGACAGGAAAUC |
| | | | CAACACCAGAAAUAACAUGAUCCAGACCAAGCCAACAGGAACCCA |
| | | | GCAAUCGACCAAUACAGCUGUGACCCUGACUGGCUAG |
| 12 | XCL2 amino acids | Human protein | MRLLILALLGICSLTAYIVEGVGSEVSHRRTCVSLTTQRLPVSRIK |
| | | | TYTITEGSLRAVIFITKRGLKVCADPQATWVRDVVRSMDRKSNTR |
| | | | NNMIQTKPTGTQQSTNTAVTLTG |
| 13 | CCL5 DNA | | ATGAAGGTCTCCGCGGCACGCCTCGCTGTCATCCTCATTGCTACT |
| | | | GCCCTCTGCGCTCCTGCATCTGCCTCCCCATATTCCTCGGACACC |
| | | | ACACCCTGCTGCTTTCCCTACATTGCCCGCCCACTGCCCCGTGCC |
| | | | CACATCAAGGAGTATTTCTACACCAGTGGCAAGTGCTCCAACCCA |
| | | | GCAGTCGTCTTTGTCACCCGAAAGAACCGCCAAGTGTGTGCCAAC |
| | | | CCAGAGAAGAAATGGGTTCGGGAGTACATCAACTCTTTGGAGATG |
| | | | AGCTAG |
| 13A | RNA sequence | | AUGAAGGUCUCCGCGGCACGCCUCGCUGUCAUCCUCAUUGCUA |
| | corresponding to | | CUGCCCUCUGCGCUCCUGCAUCUGCCUCCCCAUAUUCCUCGGAC |
| | SEQ ID NO: 13 | | ACCACACCCUGCUGUUUGCCUACAUUGCCCGCCCACUGCCCCG |
| | | | UGCCCACAUCAAGGAGUAUUUCUACACCAGUGGCAAGUGCUCCA |
| | | | ACCCAGCAGUCGUCUUUGUCACCCGAAAGAACCGCCAAGUGUGU |
| | | | GCCAACCCAGAGAAGAAAUGGGUUCGGGAGUACAUCAACUCUUU |
| | | | GGAGAUGAGCUAG |
| 14 | CCL5 amino acids | Human protein | MKVSAAALAVILIATALCAPASASPYSSDTTPCCFAYIARPLPRAHIKEY |
| | | | FYTSGKCSNPAVVFVTRKNRQVCANPEKKWVREYINSLEMS |
| 15 | CCL4 DNA | Human DNA | ATGAAGCTCTGCGTGACTGTCCTGTCTCTCCTCATGCTAGTAGCTG |
| | | | CCTTCTGCTCTCCAGCGCTCTCAGCACCAATGGGCTCAGACCCTC |
| | | | CCACCGCCTGCTGCTTTTCTTACACCGCGAGGAAGCTTCCTCGCA |
| | | | ACTTTGTGGTAGATTACTATGAGACCAGGAGCCTCTGCTCCCAGCC |
| | | | AGCTGTGGTATTCCAAACCAAAAGAAGCAAGCAAGTCTGTGCTGAT |
| | | | CCCAGTGAATCCTGGGTCCAGGAGTACGTGTATGACCTGGAACTG |
| | | | AACTGA |
| 15A | RNA sequence | | AUGAAGCUCUGCGUGACUGUCCUGUCUCUCCUCAUGCUAGUAG |
| | corresponding to | | CUGCCUUCUGCUCUCCAGCGCUCUCAGCACCAAUGGGCUCAGAC |
| | SEQ ID NO: 15 | | CCUCCCACCGCCUGCUGGUUUUGUUACACCGCGAGGAAGCUUCC |
| | | | UCGCAACUUUGUGGUAGAUUACUAUGAGACCAGCAGCCUCUGCU |
| | | | CCCAGCCAGCUGUGGUAUUCCAAACCAAAAGAAGCAAGCAAGUC |
| | | | UGUGCUGAUCCCAGUGAAUCCUGGGUCCAGGAGUACGUGUAUG |
| | | | ACCUGGAACUGAACUGA |
| 16 | CCL4 amino acids | Human protein | MKLCVTVLSLLMLVAAFCSPALSAPMGSDPPTACCFSYTARKLPRNF |
| | | | VVDYYETSSLCSQPAVVFQTKRSKQVCADPSESWVQEYVYDLELN |
| 17 | sFLT3L DNA | Human DNA | ATGACAGTGCTGGCGCCAGCCTGGAGCCCAACAACCTATCTCCTC |
| | | | CTGCTGCTGCTGCTGAGCTCGGGACTCAGTGGGACCCAGGACTG |
| | | | CTCCTTCCAACACAGCCCCATCTCCTCCGACTTCGCTGTCAAAATC |
| | | | CGTGAGCTGTCTGACTACCTGCTTCAAGATTACCCAGTCACCGTG |
| | | | GCCTCCAACCTGCAGGACGAGGAGCTCTGCGGGGCGCTCTGGCG |
| | | | GCTGGTCCTGGCACAGCGCTGGATGGAGCGGCTCAAGACTGTCG |
| | | | CTGGGTCCAAGATGCAAGGCTTGCTGGAGCGCGTGAACACGGAG |
| | | | ATACACTTTGTCACCAAATGTGCCTTTCAGCCCCCCCCCAGCTGTC |
| | | | TTCGCTTCGTCCAGACCAACATCTCCCGCCTCCTGCAGGAGACCT |
| | | | CCGAGCAGCTGGTGGCGCTGAAGCCCTGGATCACTCGCCAGAAC |
| | | | TTCTCCCGGTGCCTGGAGCTGCAGTGTCAGCCCGACTCCTCAACC |
| | | | CTGCCACCCCCATGGAGTCCCCGGCCCCTGGAGGCCACAGCCCC |
| | | | GACAGCCCCGCAGCCCCCTCTGCTCCTCCTACTGCTGCTGCCCGT |
| | | | GGGCCTCCTGCTGCTGGCCGCTGCCTGGTGCCTGCACTGGCAGA |
| | | | GGACGCGGCGGAGGACACCCCGCCCTGGGGAGCAGGTGCCCCC |
| | | | CGTCCCCAGTCCCCAGGACCTGCTGCTTGTGGAGCACTGA |
| 17A | RNA sequence | | AUGACAGUGCUGGCGCCAGCCUGGAGCCCAACAACCUAUCUCCU |
| | corresponding to | | CCUGCUGCUGCUGCUGAGCUCGGGACUCAGUGGGACCCAGGAC |
| | SEQ ID NO: 17 | | UGCUCCUUCCAACACAGCCCCAUCUCCUCCGACUUCGCUGUCAA |
| | | | AAUCCGUGAGCUGUCUGACUACCUGCUUCAAGAUUACCCAGUCA |
| | | | CCGUGGCCUCCAACCUGCAGGACGAGGAGCUCUGGGGGGGGCU |
| | | | CUGGGGGCUGGUCCUGGCACAGCGCUGGAUGGAGCGGCUCAAG |
| | | | ACUGUCGCUGGGUCCAAGAUGCAAGGCUUGCUGGAGCGCGUGA |
| | | | ACACGGAGAUACACUUUGUCACCAAAUGUGCCUUUCAGCCCCCC |
| | | | CCCAGCUGUCUUCGCUUCGUCCAGACCAACAUCUCCCGCCUCCU |
| | | | GCAGGAGACCUCCGAGCAGCUGGUGGCGCUGAAGCCCUGGAUC |
| | | | ACUCGCCAGAACUUCUCCCGGUGCCUGGAGCUGCAGUGUCAGC |
| | | | CCGACUCCUCAACCCUGCCACCCCCAUGGAGUCCCCGGCCCCUG |
| | | | GAGGCCACAGCCCCGACAGCCCCGCAGCCCCCUCUGCUCCUCC |
| | | | UACUGCUGCUGCCCGUGGGCCUCCUGCUGCUGGCCGCUGCCUG |
| | | | GUGCCUGCACUGGCAGAGGACGCGGCGGAGGACACCCCGCCCU |
| | | | GGGGAGCAGGUGCCCCCCGUCCCCAGUCCCCAGGACCUGCUGC |
| | | | UUGUGGAGCACUGA |
| 18 | sFLT3L amino | Human protein | MTVLAPAWSPTTYLLLLLLLSSGLSGTQDCSFQHSPISSDFAVKIRELS |
| | acids | | DYLLQDYPVTVASNLQDEELCGGLWRLVLAQRWMERLKTVAGSKMQ |
| | | | GLLERVNTEIHFVTKCAFQPPPSCLRFVQTNISRLLQETSEQLVALKP |
| | | | WITRQNFSRCLELQCQPDSSTLPPPWSPRPLEATAPTAPQPPLLLLLL |
| | | | LPVGLLLLAAAWCLHWQRTRRRTPRPGEQVPPVPSPQDLLLVEH |
| 19 | GM-CSF DNA | Human DNA | ATGTGGCTGCAGAGCCTGCTGCTCTTGGGCACTGTGGCCTGCAGC |
| | | | ATCTCTGCACCCGCCCGCTCGCCCAGCCCCAGCACACAGCCCTG |
| | | | GGAGCATGTGAATGCCATCCAGGAGGCCCGGCGTCTCCTGAACCT |
| | | | GAGTAGAGACACTGCTGCTGAGATGAATGAAACAGTAGAAGTCAT |
| | | | CTCAGAAATGTTTGACCTCCAGGAGCCGACCTGCCTACAGACCCG |
| | | | CCTGGAGCTGTACAAGCAGGGCCTGCGGGGCAGCCTCACCAAGC |
| | | | TCAAGGGCCCCTTGACCATGATGGCCAGCCACTACAAACAGCACT |
| | | | GCCCTCCAACCCCGGAAACTTCCTGTGCAACCCAGATTATCACCTT |
| | | | TGAAAGTTTCAAAGAGAACCTGAAGGACTTTCTGCTTGTCATCCCC |
| | | | TTTGACTGCTGGGAGCCAGTCCAGGAGTGA |
| 19A | RNA sequence | | AUGUGGCUGCAGAGCCUGCUGCUCUUGGGCACUGUGGCCUGCA |
| | corresponding to | | GCAUCUCUGCACCCGCCCGCUCGCCCAGCCCCAGCACACAGCCC |
| | SEQ ID NO: 19 | | UGGGAGCAUGUGAAUGCCAUCCAGGAGGCCCGGCGUCUCCUGA |
| | | | ACCUGAGUAGAGACACUGCUGCUGAGAUGAAUGAAACAGUAGAA |
| | | | GUCAUCUCAGAAAUGUUUGACCUCCAGGAGCCGACCUGCCUACA |
| | | | GACCCGCCUGGAGCUGUACAAGCAGGGCCUGGGGGGCAGCCUC |
| | | | ACCAAGCUCAAGGGCCCCUUGACCAUGAUGGCCAGCCACUACAA |
| | | | ACAGCACUGCCCUCCAACCCCGGAAACUUCCUGUGCAACCCAGA |
| | | | UUAUCACCUUUGAAAGUUUCAAAGAGAACCUGAAGGACUUUCUG |
| | | | CUUGUCAUCCCCUUUGACUGCUGGGAGCCAGUCCAGGAGUGA |
| 20 | GM-CSF amino | Human protein | MWLQSLLLLGTVACSISAPARSPSPSTQPWEHVNAIQEARRLLNLSRD |
| | acids | | TAAEMNETVEVISEMFDLQEPTCLQTRLELYKQGLRGSLTKLKGPLTM |
| | | | MASHYKQHCPPTPETSCATQIITFESFKENLKDFLLVIPFDCWEPVQE |
| 21 | CD40L DNA | Human DNA | ATGATCGAAACATACAACCAAACTTCTCCCCGATCTGCGGCCACTG |
| | | | GACTGCCCATCAGCATGAAAATTTTTATGTATTTACTTACTGTTTTT |
| | | | CTTATCACCCAGATGATTGGGTCAGCACTTTTTGCTGTGTATCTTCA |
| | | | TAGAAGGCTGGACAAGATAGAAGATGAAAGGAATCTTCATGAAGAT |
| | | | TTTGTATTCATGAAAACGATACAGAGATGCAACACAGGAGAAAGAT |
| | | | CCTTATCCTTACTGAACTGTGAGGAGATTAAAAGCCAGTTTGAAGG |
| | | | CTTTGTGAAGGATATAATGTTAAACAAAGAGGAGACGAAGAAAGAA |
| | | | AACAGCTTTGAAATGCAAAAAGGTGATCAGAATCCTCAAATTGCGG |
| | | | CACATGTCATAAGTGAGGCCAGCAGTAAAACAACATCTGTGTTACA |
| | | | GTGGGCTGAAAAAGGATACTACACCATGAGCAACAACTTGGTAAC |
| | | | CCTGGAAAATGGGAAACAGCTGACCGTTAAAAGACAAGGACTCTA |
| | | | TTATATCTATGCCCAAGTCACCTTCTGTTCCAATCGGGAAGCTTCG |
| | | | AGTCAAGCTCCATTTATAGCCAGCCTCTGCCTAAAGTCCCCCGGTA |
| | | | GATTCGAGAGAATCTTACTCAGAGCTGCAAATACCCACAGTTCCGC |
| | | | CAAACCTTGCGGGCAACAATCCATTCACTTGGGAGGAGTATTTGAA |
| | | | TTGCAACCAGGTGCTTCGGTGTTTGTCAATGTGACTGATCCAAGCC |
| | | | AAGTGAGCCATGGCACTGGCTTCACGTCCTTTGGCTTACTCAAACT |
| | | | CTGA |
| 21A | RNA sequence | | AUGAUCGAAACAUACAACCAAACUUCUCCCCGAUCUGCGGCCAC |
| | corresponding to | | UGGACUGCCCAUCAGCAUGAAAAUUUUUAUGUAUUUACUUACUG |
| | SEQ ID NO: 21 | | UUUUUCUUAUCACCCAGAUGAUUGGGUCAGCACUUUUUGCUGUG |
| | | | UAUCUUCAUAGAAGGCUGGACAAGAUAGAAGAUGAAAGGAAUCU |
| | | | UCAUGAAGAUUUUGUAUUCAUGAAAACGAUACAGAGAUGCAACA |
| | | | CAGGAGAAAGAUCCUUAUCCUUACUGAACUGUGAGGAGAUUAAA |
| | | | AGCCAGUUUGAAGGCUUUGUGAAGGAUAUAAUGUUAAACAAAGA |
| | | | GGAGACGAAGAAAGAAAACAGCUUUGAAAUGCAAAAAGGUGAUC |
| | | | AGAAUCCUCAAAUUGCGGCACAUGUCAUAAGUGAGGCCAGCAGU |
| | | | AAAACAACAUCUGUGUUACAGUGGGCUGAAAAAGGAUAGUACAC |
| | | | CAUGAGCAACAACUUGGUAACCCUGGAAAAUGGGAAACAGCUGA |
| | | | CCGUUAAAAGACAAGGACUCUAUUAUAUCUAUGCCCAAGUCACC |
| | | | UUCUGUUCCAAUCGGGAAGCUUCGAGUCAAGCUCCAUUUAUAGC |
| | | | CAGCCUCUGCCUAAAGUCCCCCGGUAGAUUCGAGAGAAUCUUAC |
| | | | UCAGAGCUGCAAAUACCCACAGUUCCGCCAAACCUUGCGGGCAA |
| | | | CAAUCCAUUCACUUGGGAGGAGUAUUUGAAUUGCAACCAGGUGC |
| | | | UUCGGUGUUUGUCAAUGUGACUGAUCCAAGCCAAGUGAGCCAUG |
| | | | GCACUGGCUUCACGUCCUUUGGCUUACUCAAACUCUGA |
| 22 | CD40L amino | Human protein | MIETYNQTSPRSAATGLPISMKIFMYLLTVFLITQMIGSALFAVYLHRRL |
| | acids | | DKIEDERNLHEDFVFMKTIQRCNTGERSLSLLNCEEIKSQFEGFVKDIM |
| | | | LNKEETKKENSFEMQKGDQNPQIAAHVISEASSKTTSVLQWAEKGYY |
| | | | TMSNNLVTLENGKQLTVKRQGLYYIYAQVTFCSNREASSQAPFIASLC |
| | | | LKSPGRFERILLRAANTHSSAKPCGQQSIHLGGVFELQPGASVFVNVT |
| | | | DPSQVSHGTGFTSFGLLKL |
| 23 | IL-12 DNA | Human DNA | ATGTGTCCAGCGCGCAGCCTCCTCCTTGTGGCTACCCTGGTCCTC |
| | | | CTGGACCACCTCAGTTTGGCCAGAAACCTCCCCGTGGCCACTCCA |
| | | | GACCCAGGAATGTTCCCATGCCTTCACCACTCCCAAAACCTGCTG |
| | | | AGGGCCGTCAGCAACATGCTCCAGAAGGCCAGAGAAACTCTAGAA |
| | | | TTTTACCCTTGCACTTCTGAAGAGATTGATCATGAAGATATCACAAA |
| | | | AGATAAAACCAGCACAGTGGAGGCCTGTTTACCATTGGAATTAACC |
| | | | AAGAATGAGAGTTGCCTAAATTCCAGAGAGACCTCTTTCATAACTA |
| | | | ATGGGAGTTGCCTGGCCTCCAGAAAGACCTCTTTTATGATGGCCCT |
| | | | GTGCCTTAGTAGTATTTATGAAGACTTGAAGATGTACCAGGTGGAG |
| | | | TTCAAGACCATGAATGCAAAGCTTCTGATGGATCCTAAGAGGCAGA |
| | | | TCTTTCTAGATCAAAACATGCTGGCAGTTATTGATGAGCTGATGCA |
| | | | GGCCCTGAATTTCAACAGTGAGACTGTGCCACAAAAATCCTCCCTT |
| | | | GAAGAACCGGATTTTTATAAAACTAAAATCAAGCTCTGCATACTTCT |
| | | | TCATGCTTTCAGAATTCGGGCAGTGACTATTGACAGAGTGACGAG |
| | | | CTATCTGAATGCTTCCTAA |
| 23A | RNA sequence | | AUGUGUCCAGCGCGCAGCCUCCUCCUUGUGGCUACCCUGGUCC |
| | corresponding to | | UCCUGGACCACCUCAGUUUGGCCAGAAACCUCCCCGUGGCCACU |
| | SEQ ID NO: 23 | | CCAGACCCAGGAAUGUUCCCAUGCCUUCACCACUCCCAAAACCU |
| | | | GCUGAGGGCCGUCAGCAACAUGCUCCAGAAGGCCAGACAAACUC |
| | | | UAGAAUUUUACCCUUGCACUUCUGAAGAGAUUGAUCAUGAAGAU |
| | | | AUCACAAAAGAUAAAACCAGCACAGUGGAGGCCUGUUUACCAUU |
| | | | GGAAUUAACCAAGAAUGAGAGUUGCCUAAAUUCCAGAGAGACCU |
| | | | CUUUCAUAAGUAAUGGGAGUUGCCUGGCCUCCAGAAAGACCUCU |
| | | | UUUAUGAUGGCCCUGUGCCUUAGUAGUAUUUAUGAAGACUUGAA |
| | | | GAUGUACCAGGUGGAGUUCAAGACCAUGAAUGCAAAGCUUCUGA |
| | | | UGGAUCCUAAGAGGCAGAUCUUUCUAGAUCAAAACAUGCUGGCA |
| | | | GUUAUUGAUGAGCUGAUGCAGGCCCUGAAUUUCAAGAGUGAGAC |
| | | | UGUGCCACAAAAAUCCUCCCUUGAAGAACCGGAUUUUUAUAAAA |
| | | | CUAAAAUCAAGCUCUGCAUACUUCUUCAUGCUUUCAGAAUUCGG |
| | | | GCAGUGACUAUUGACAGAGUGACGAGCUAUCUGAAUGCUUCCUAA |
| 24 | IL-12 amino acids | Human protein | MCPARSLLLVATLVLLDHLSLARNLPVATPDPGMFPCLHHSQNLLRAV |
| | | | SNMLQKARQTLEFYPCTSEEIDHEDITKDKTSTVEACLPLELTKNESCL |
| | | | NSRETSFITNGSCLASRKTSFMMALCLSSIYEDLKMYQVEFKTMNAKL |
| | | | LMDPKRQIFLDQNMLAVIDELMQALNFNSETVPQKSSLEEPDFYKTKI |
| | | | KLCILLHAFRIRAVTIDRVMSYLNAS |
| 25 | IL-15 DNA | Human DNA | ATGGCCCCGCGGCGGGCGCGCGGCTGCCGGACCCTCGGTCTCC |
| | | | CGGCGCTGCTACTGCTGCTGCTGCTCCGGCCGCCGGCGACGCGG |
| | | | GGCATCACGTGCCCTCCCCCCATGTCCGTGGAACACGCAGACATC |
| | | | TGGGTCAAGAGCTACAGCTTGTACTCCAGGGAGGGGTACATTTGT |
| | | | AACTCTGGTTTCAAGCGTAAAGCCGGCACGTCCAGCCTGACGGAG |
| | | | TGCGTGTTGAACAAGGCCACGAATGTGGCCCACTGGACAACCCCC |
| | | | AGTCTCAAATGCATTAGAGACCCTGCCCTGGTTCACCAAAGGCCA |
| | | | GCGCCACCCTCCACAGTAACGACGGCAGGGGTGACCCCACAGCC |
| | | | AGAGAGCCTCTCCCCTTCTGGAAAAGAGCCCGCAGCTTCATCTCC |
| | | | CAGCTCAAACAACACAGCGGCCACAACAGCAGCTATTGTCCCGGG |
| | | | CTCCCAGCTGATGCCTTCAAAATCACCTTCCACAGGAACCACAGA |
| | | | GATAAGCAGTCATGAGTCCTCCCACGGCACCCCCTCTCAGACAAC |
| | | | AGCCAAGAACTGGGAACTCACAGCATCCGCCTCCCACCAGCCGCC |
| | | | AGGTGTGTATCCACAGGGCCACAGCGACACCACTGTGGCTATCTC |
| | | | CACGTCCACTGTCCTGCTGTGTGGGCTGAGGGCTGTGTCTCTCCT |
| | | | GGCATGCTACCTCAAGTCAAGGCAAACTCCCCCGCTGGCCAGCGT |
| | | | TGAAATGGAAGCCATGGAGGCTCTGCCGGTGACTTGGGGGACCA |
| | | | GCAGCAGAGATGAAGACTTGGAAAACTGCTCTCACCACCTATGA |
| 25A | RNA sequence | | AUGGCCCCGCGGCGGGCGCGCGGCUGCCGGACCCUCGGUCUCC |
| | corresponding to | | CGGCGCUGCUACUGCUGCUGCUGCUCCGGCCGCCGGCGACGCG |
| | SEQ ID NO: 25 | | GGGCAUCACGUGCCCUCCCCCCAUGUCCGUGGAACACGCAGACA |
| | | | UCUGGGUCAAGAGCUACAGCUUGUACUCCAGGGAGCGGUACAU |
| | | | UUGUAACUCUGGUUUCAAGCGUAAAGCCGGCACGUCCAGCCUGA |
| | | | CGGAGUGCGUGUUGAACAAGGCCACGAAUGUCGCCCACUGGAC |
| | | | AACCCCCAGUCUCAAAUGCAUUAGAGACCCUGCCCUGGUUCACC |
| | | | AAAGGCCAGCGCCACCCUCCACAGUAACGACGGCAGGGGUGACC |
| | | | CCACAGCCAGAGAGCCUCUCCCCUUCUGGAAAAGAGCCCGCAGC |
| | | | UUCAUCUCCCAGCUCAAACAACACAGGGGCCACAACAGCAGCUA |
| | | | UUGUCCCGGGCUCCCAGCUGAUGCCUUCAAAAUCACCUUCCACA |
| | | | GGAACCACAGAGAUAAGCAGUCAUGAGUCCUCCCACGGCACCCC |
| | | | CUCUCAGACAACAGCCAAGAACUGGGAACUCACAGCAUCCGCCU |
| | | | CCCACCAGCCGCCAGGUGUGUAUCCACAGGGCCACAGCGACACC |
| | | | ACUGUGGCUAUCUCCACGUCCACUGUCCUGCUGUGUGGGCUGA |
| | | | GCGCUGUGUCUCUCCUGGCAUGCUACCUCAAGUCAAGGCAAACU |
| | | | CCCCCGCUGGCCAGCGUUGAAAUGGAAGCCAUGGAGGCUCUGC |
| | | | CGGUGACUUGGGGGACCAGCAGCAGAGAUGAAGACUUGGAAAAC |
| | | | UGCUCUCACCACCUAUGA |
| 26 | IL-15 amino acids | Human protein | MAPRRARGCRTLGLPALLLLLLLRPPATRGITCPPPMSVEHADIWVKS |
| | | | YSLYSRERYICNSGFKRKAGTSSLTECVLNKATNVAHWTTPSLKCIRD |
| | | | PALVHQRPAPPSTVTTAGVTPQPESLSPSGKEPAASSPSSNNTAATT |
| | | | AAIVPGSQLMPSKSPSTGTTEISSHESSHGTPSQTTAKNWELTASASH |
| | | | QPPGVYPQGHSDTTVAISTSTVLLCGLSAVSLLACYLKSRQTPPLASV |
| | | | EMEAMEALPVTWGTSSRDEDLENCSHHL |
| 27 | CXCL9 DNA | Human DNA | ATGAAGAAAAGTGGTGTTCTTTTCCTCTTGGGCATCATCTTGCTGG |
| | | | TTCTGATTGGAGTGCAAGGAACCCCAGTAGTGAGAAAGGGTCGCT |
| | | | GTTCCTGCATCAGCACCAACCAAGGGACTATCCACCTACAATCCTT |
| | | | GAAAGACCTTAAACAATTTGCCCCAAGCCCTTCCTGCGAGAAAATT |
| | | | GAAATCATTGCTACACTGAAGAATGGAGTTCAAACATGTCTAAACC |
| | | | CAGATTCAGCAGATGTGAAGGAACTGATTAAAAAGTGGGAGAAAC |
| | | | AGGTCAGCCAAAAGAAAAAGCAAAAGAATGGGAAAAAACATCAAAA |
| | | | AAAGAAAGTTCTGAAAGTTCGAAAATCTCAACGTTCTCGTCAAAAG |
| | | | AAGACTACATAA |
| 27A | RNA sequence | | AUGAAGAAAAGUGGUGUUCUUUUCCUCUUGGGCAUCAUCUUGCU |
| | corresponding to | | GGUUCUGAUUGGAGUGCAAGGAACCCCAGUAGUGAGAAAGGGU |
| | SEQ ID NO: 27 | | CGCUGUUCCUGCAUCAGCACCAACCAAGGGACUAUCCACCUACA |
| | | | AUCCUUGAAAGACCUUAAACAAUUUGCCCCAAGCCCUUCCUGCG |
| | | | AGAAAAUUGAAAUCAUUGCUACACUGAAGAAUGGAGUUCAAACAU |
| | | | GUCUAAACCCAGAUUCAGCAGAUGUGAAGGAACUGAUUAAAAAG |
| | | | UGGGAGAAACAGGUCAGCCAAAAGAAAAAGCAAAAGAAUGGGAAA |
| | | | AAACAUCAAAAAAAGAAAGUUCUGAAAGUUCGAAAAUCUCAACGU |
| | | | UCUCGUCAAAAGAAGAGUACAUAA |
| 28 | CXCL9 amino | Human protein | MKKSGVLFLLGIILLVLIGVQGTPVVRKGRCSCISTNQGTIHLQSLKDLK |
| | acids | | QFAPSPSCEKIEIIATLKNGVQTCLNPDSADVKELIKKWEKQVSQKKKQ |
| | | | KNGKKHQKKKVLKVRKSQRSRQKKTT |
| 29 | CXCL10 DNA | Human DNA | ATGAATCAAACTGCGATTCTGATTTGCTGCCTTATCTTTCTGACTCT |
| | | | AAGTGGCATTCAAGGAGTACCTCTCTCTAGAACCGTACGCTGTACC |
| | | | TGCATCAGCATTAGTAATCAACCTGTTAATCCAAGGTCTTTAGAAAA |
| | | | ACTTGAAATTATTCCTGCAAGCCAATTITGTCCACGTGTTGAGATCA |
| | | | TTGCTACAATGAAAAAGAAGGGTGAGAAGAGATGTCTGAATCCAGA |
| | | | ATCGAAGGCCATCAAGAATTTACTGAAAGCAGTTAGCAAGGAAATG |
| | | | TCTAAAAGATCTCCTTAA |
| 29A | RNA sequence | | AUGAAUCAAACUGCGAUUCUGAUUUGCUGCCUUAUCUUUCUGAC |
| | corresponding to | | UCUAAGUGGCAUUCAAGGAGUACCUCUCUCUAGAACCGUACGCU |
| | SEQ ID NO: 29 | | GUACCUGCAUCAGCAUUAGUAAUCAACCUGUUAAUCCAAGGUCU |
| | | | UUAGAAAAACUUGAAAUUAUUCCUGCAAGCCAAUUUUGUCCAGG |
| | | | UGUUGAGAUCAUUGCUACAAUGAAAAAGAAGGGUGAGAAGAGAU |
| | | | GUCUGAAUCCAGAAUCGAAGGCCAUCAAGAAUUUACUGAAAGCA |
| | | | GUUAGCAAGGAAAUGUCUAAAAGAUCUCCUUAA |
| 30 | CXCL10 amino | Human protein | MNQTAILICCLIFLTLSGIQGVPLSRTVRCTCISISNQPVNPRSLEKLE |
| | acids | | IIPASQFCPRVEIIATMKKKGEKRCLNPESKAIKNLLKAVSKERSKRSP |
| 31 | INF-beta DNA | Human DNA | ATGACCAACAAGTGTCTCCTCCAAATTGCTCTCCTGTTGTGCTTCT |
| | | | CCACTACAGCTCTTTCCATGAGCTACAACTTGCTTGGATTCCTACA |
| | | | AAGAAGCAGCAATTTTCAGTGTCAGAAGCTCCTGTGGCAATTGAAT |
| | | | GGGAGGCTTGAATACTGCCTCAAGGACAGGATGAACTTTGACATC |
| | | | CCTGAGGAGATTAAGCAGCTGCAGCAGTTCCAGAAGGAGGACGC |
| | | | CGCATTGACCATCTATGAGATGCTCCAGAACATCTTTGCTATTTTCA |
| | | | GACAAGATTCATCTAGCACTGGCTGGAATGAGACTATTGTTGAGAA |
| | | | CCTCCTGGCTAATGTCTATCATCAGATAAACCATCTGAAGACAGTC |
| | | | CTGGAAGAAAAACTGGAGAAAGAAGATTTCACCAGGGGAAAACTC |
| | | | ATGAGCAGTCTGCACCTGAAAAGATATTATGGGAGGATTCTGCATT |
| | | | ACCTGAAGGCCAAGGAGTACAGTCACTGTGCCTGGACCATAGTCA |
| | | | GAGTGGAAATCCTAAGGAACTTTTACTTCATTAACAGACTTACAGG |
| | | | TTACCTCCGAAACTGA |
| 31A | RNA sequence | | AUGACCAACAAGUGUCUCCUCCAAAUUGCUCUCCUGUUGUGCUU |
| | corresponding to | | CUCCACUACAGCUCUUUCCAUGAGCUACAACUUGCUUGGAUUCC |
| | SEQ ID NO: 31 | | UACAAAGAAGCAGCAAUUUUCAGUGUCAGAAGCUCCUGUGGCAA |
| | | | UUGAAUGGGAGGCUUGAAUACUGCCUCAAGGACAGGAUGAACUU |
| | | | UGACAUCCCUGAGGAGAUUAAGCAGCUGCAGCAGUUCCAGAAGG |
| | | | AGGACGCCGCAUUGACCAUCUAUGAGAUGCUCCAGAACAUCUUU |
| | | | GCUAUUUUCAGACAAGAUUCAUCUAGCACUGGCUGGAAUGAGAC |
| | | | UAUUGUUGAGAACCUCCUGGCUAAUGUCUAUCAUCAGAUAAACC |
| | | | AUCUGAAGACAGUCCUGGAAGAAAAAGUGGAGAAAGAAGAUUUC |
| | | | ACCAGGGGAAAACUCAUGAGCAGUCUGCACCUGAAAAGAUAUUA |
| | | | UGGGAGGAUUCUGCAUUACCUGAAGGCCAAGGAGUACAGUCACU |
| | | | GUGCCUGGACCAUAGUCAGAGUGGAAAUCCUAAGGAACUUUUAC |
| | | | UUCAUUAACAGACUUACAGGUUACCUCCGAAACUGA |
| 32 | INF-beta amino | Human protein | MTNKCLLQIALLLCFSTTALSMSYNLLGFLQRSSNFQCQKLLWQLNGR |
| | acids | | LEYCLKDRMNFDIPEEIKQLQQFQKEDAALTIYEMLQNIFAIFRQDSSS |
| | | | TGWNETIVENLLANVYHQINHLKTVLEEKLEKEDFTRGKLMSSLHLKR |
| | | | YYGRILHYLKAKEYSHCAWTIVRVEILRNFYFINRLTGYLRN |
| 33 | sFLT3L-furin | Murine RNA | AUGACAGUGCUGGCUCCUGCUUGGAGCCCUAACAGCUCUCUGC |
| | cleavage site- | | UGCUGCUUCUGCUCCUGCUGAGCCCAUGUCUGAGAGGCACCCC |
| | P2A-IL-12-furin | | UGACUGCUACUUCUCUCACAGCCCCAUCAGCAGCAACUUCAAAG |
| | cleavage site- | | UGAAGUUCCGCGAGCUGACCGACCAUCUGCUGAAGGACUACCCU |
| | P2A-XCL1 RNA | | GUGACCGUGGCCGUGAACCUGCAGGAUGAGAAGCACUGCAAGG |
| | | | CCCUGUGGUCCCUGUUCCUGGCUCAGAGAUGGAUCGAGCAGCU |
| | | | GAAAACAGUGGCCGGCAGCAAGAUGCAGACCCUGCUGGAAGAUG |
| | | | UGAACACCGAGAUCCACUUCGUGACCAGCUGCACCUUCCAGCCU |
| | | | CUGCCUGAGUGCCUGAGAUUCGUGCAGACCAACAUCAGCCACCU |
| | | | UCUCAAGGACACAUGCACCCAGCUGCUGGCCCUGAAGCCUUGUA |
| | | | UCGGCAAGGCCUGCCAGAACUUCUCCAGAUGCCUGGAAGUGCA |
| | | | GUGCCAGCCUGACAGCUCUACACUGCUGCCUCCAAGAAGCCCUA |
| | | | UCGCUCUGGAAGCCACAGAGCUGCCUGAGCCUAGACCUAGACAG |
| | | | AGAAGAAAGAGAAGCGGGUCCGGCGCCACCAAGUUCAGCCUGCU |
| | | | UAAACAGGCAGGCGACGUGGAAGAGAACCCCGGACCUAUGUGCC |
| | | | CUCAGAAGCUGACCAUCAGUUGGUUCGCCAUCGUGCUGCUGGU |
| | | | GUCCCCACUGAUGGCUAUGUGGGAACUCGAGAAGGACGUGUAG |
| | | | GUGGUGGAAGUGGACUGGACCCCUGAUGCUCCUGGCGAGACAG |
| | | | UGAACCUGACCUGCGACACACCUGAAGAGGACGACAUCACCUGG |
| | | | ACCAGCGAUCAGAGACACGGCGUGAUCGGCUCUGGCAAGACCCU |
| | | | GACAAUUACCGUGAAAGAGUUCCUGGACGCCGGCCAGUACACCU |
| | | | GUCACAAAGGCGGAGAGACACUGAGCCACUCUCAUCUGCUGCUG |
| | | | CACAAGAAAGAGAACGGCAUCUGGUCCACCGAGAUCCUGAAGAA |
| | | | CUUCAAGAACAAGACCUUCCUGAAGUGCGAGGCCCCUAAGUACA |
| | | | GCGGCAGAUUCACCUGUAGCUGGCUGGUGCAGAGAAACAUGGA |
| | | | CCUGAAGUUCAACAUCAAGUCCUCCAGCAGCAGCCCCGACAGCA |
| | | | GAGCUGUGACAUGUGGCAUGGCUAGCCUGAGCGCCGAGAAAGU |
| | | | GACACUGGACCAGAGAGACUACGAGAAGUACAGCGUGUCCUGCC |
| | | | AAGAGGACGUGACCUGUCCUACCGCCGAGGAAACACUGCCUAUC |
| | | | GAGCUGGCCCUGGAAGCCAGACAGCAGAACAAAUACGAGAACUA |
| | | | CUCUACCAGCUUCUUCAUCCGGGACAUCAUCAAGCCCGAUCCUC |
| | | | CAAAGAACCUGCAGAUGAAGCCUCUGAAGAACAGCCAGGUCGAG |
| | | | GUGUCCUGGGAGUACCCUGACAGCUGGUCUACCCCUCACAGCU |
| | | | ACUUCAGCCUGAAAUUCUUCGUGGGGAUCCAGCGCAAGAAAGAA |
| | | | AAGAUGAAGGAAACCGAGGAAGGCUGCAACCAGAAAGGCGCUUU |
| | | | CCUGGUGGAAAAGACCAGCACCGAGGUGCAGUGCAAAGGCGGC |
| | | | AAUGUCUGUGUGCAGGCCCAGGACCGGUACUACAACAGCAGCUG |
| | | | UAGCAAGUGGGCCUGCGUGCCAUGCAGAGUCAGAUCUGGUGGC |
| | | | GGAGGAUCUGGCGGAGGUGGAAGCGGCGGAGGCGGAUCUAGAG |
| | | | UGAUUCCUGUGUCUGGCCCUGCCAGAUGCCUGAGCCAGUCUAG |
| | | | AAACCUGCUGAAAACCACCGACCGACAUGGUCAAGACCGCCAGAG |
| | | | AGAAGCUGAAGCACUACUCCUGCACAGCCGAGGACAUCGACCAC |
| | | | GAGGAUAUCACCAGGGACCAGACAAGCACCCUGAAAACCUGCCU |
| | | | GCCUCUGGAACUGCAUAAGAACGAGAGCUGCCUGGCCACCAGAG |
| | | | AAACCAGCUCUACCACAAGAGGCAGCUGUCUGCCUCCUCAGAAA |
| | | | ACCAGCCUGAUGAUGACCCUGUGCCUGGGCAGCAUCUACGAGG |
| | | | AUCUGAAGAUGUACCAGACCGAGUUCCAGGCCAUCAACGCCGCU |
| | | | CUGCAGAACCACAACCACCAGCAGAUCAUCCUGGACAAGGGCAU |
| | | | GCUGGUGGCUAUCGACGAGCUGAUGCAGAGCCUGAACCAUAAC |
| | | | GGCGAGACACUGCGGCAGAAGCCUCCAGUUGGAGAGGCCGAUC |
| | | | CUUACAGAGUGAAGAUGAAGCUGUGCAUCCUGCUGCACGCCUUC |
| | | | AGCACCAGAGUGGUCACCAUCAACAGAGUGAUGGGCUACCUGAG |
| | | | CAGCGCCAGGCGGAAGCGGAGCGGGAGCGGCGCUACAAACUUU |
| | | | AGCCUUCUGAAGCAGGGGGGGGAUGUCGAAGAGAAUCCUGGUC |
| | | | CAAUGAGACUGCUGCUGCUGACAUUCCUGGGCGUGUGCUGUCU |
| | | | GACACCCUGGGUUGUCGAAGGCGUGGGAACAGAGGUGGUGGAA |
| | | | GAGUCCAGCUGCGUGAACCUGCAGACCCAGAGACUGCCCGUGC |
| | | | AGAAGAUCAAGACCUACAUCAUCUGGGAGGGCGCCAUGAGAGCC |
| | | | GUGAUCUUCGUGACAAAGAGAGGCCUGAAGAUCUGCGCCGAUCC |
| | | | UGAGGCCAAAUGGGUCAAAGCCGCCAUCAAGACCGUGGACGGCA |
| | | | GAGCCAGCACCAGAAAGAACAUGGCCGAGACAGUGCCUACAGGC |
| | | | GCCCAGAGAUCUACCAGCACAGCCAUCACACUGACCGGCUGA |
| 33A | DNA | | ATGACAGTGCTGGCTCCTGCTTGGAGCCCTAACAGCTCTCTGCTG |
| | corresponding to | | CTGCTTCTGCTCCTGCTGAGCCCATGTGTGAGAGGCACCCCTGAC |
| | SEQ ID NO: 33 | | TGCTACTTCTCTCACAGCCCCATCAGCAGCAACTTCAAAGTGAAGT |
| | | | TCCGCGAGCTGACCGACCATGTGCTGAAGGACTACCGTGTGACCG |
| | | | TGGCCGTGAACCTGCAGGATGAGAAGCACTGCAAGGCCCTGTGG |
| | | | TCCCTGTTCCTGGCTCAGAGATGGATCGAGCAGCTGAAAACAGTG |
| | | | GCCGGCAGCAAGATGCAGACCCTGCTGGAAGATGTGAACACCGA |
| | | | GATCCACTTCGTGACCAGCTGCACCTTCCAGCCTCTGCCTGAGTG |
| | | | CCTGAGATTCGTGCAGACCAACATCAGCCACCTTCTCAAGGACAC |
| | | | ATGCACCCAGCTGCTGGCCCTGAAGCCTTGTATCGGCAAGGCCTG |
| | | | CCAGAACTTCTCCAGATGCCTGGAAGTGCAGTGCCAGCCTGACAG |
| | | | CTCTACACTGCTGCCTCCAAGAAGCCCTATCGCTCTGGAAGCCAC |
| | | | AGAGCTGCCTGAGCCTAGACCTAGACAGAGAAGAAAGAGAAGCG |
| | | | GCTCCGGCGCCACCAACTTCAGCCTGCTTAAACAGGCAGGCGAC |
| | | | GTGGAAGAGAACCCCGGACCTATGTGCCGTCAGAAGCTGACCATC |
| | | | AGTTGGTTCGCCATCGTGCTGCTGGTGTCCCCACTGATGGCTATG |
| | | | TGGGAACTCGAGAAGGACGTGTACGTGGTGGAAGTGGACTGGAC |
| | | | CCCTGATGCTCCTGGCGAGACAGTGAACCTGACCTGCGACACACC |
| | | | TGAAGAGGACGACATCACCTGGACCAGCGATCAGAGACACGGCG |
| | | | TGATCGGCTCTGGCAAGACCCTGACAATTACCGTGAAAGAGTTCC |
| | | | TGGACGCCGGCCAGTACACCTGTCACAAAGGCGGAGAGACACTG |
| | | | AGCCACTCTCATCTGCTGCTGCACAAGAAAGAGAACGGCATCTGG |
| | | | TCCACCGAGATCCTGAAGAACTTCAAGAACAAGACCTTCCTGAAGT |
| | | | GCGAGGCCCCTAACTACAGCGGCAGATTCACCTGTAGCTGGCTGG |
| | | | TGCAGAGAAACATGGACCTGAAGTTCAACATCAAGTCCTCCAGCA |
| | | | GCAGCCCCGACAGCAGAGCTGTGACATGTGGCATGGCTAGCCTG |
| | | | AGCGCCGAGAAAGTGACACTGGACCAGAGAGACTACGAGAAGTAC |
| | | | AGCGTGTCCTGCCAAGAGGACGTGACCTGTCCTACCGCCGAGGA |
| | | | AACACTGCCTATCGAGCTGGCCCTGGAAGCCAGACAGCAGAACAA |
| | | | ATACGAGAACTACTCTACCAGCTTCTTCATCCGGGACATCATCAAG |
| | | | CCCGATCCTCCAAAGAACCTGCAGATGAAGCCTCTGAAGAACAGC |
| | | | CAGGTCGAGGTGTCCTGGGAGTACCCTGACAGCTGGTCTACCCCT |
| | | | CACAGCTACTTCAGCCTGAAATTCTTCGTGCGGATCCAGCGCAAG |
| | | | AAAGAAAAGATGAAGGAAACCGAGGAAGGCTGCAACCAGAAAGGC |
| | | | GCTTTCCTGGTGGAAAAGACCAGCACCGAGGTGCAGTGCAAAGGC |
| | | | GGCAATGTCTGTGTGCAGGCCCAGGACCGGTACTACAACAGCAGC |
| | | | TGTAGCAAGTGGGGCTGCGTGCCATGCAGAGTCAGATCTGGTGGC |
| | | | GGAGGATCTGGCGGAGGTGGAAGCGGGGGAGGCGGATCTAGAGT |
| | | | GATTCCTGTGTCTGGCCCTGCCAGATGCCTGAGCCAGTCTAGAAA |
| | | | CCTGCTGAAAACCACCGACGACATGGTCAAGACCGCCAGAGAGAA |
| | | | GCTGAAGCACTACTCCTGCACAGCCGAGGACATCGACCACGAGGA |
| | | | TATCACCAGGGACCAGACAAGCACCCTGAAAACCTGCCTGCCTCT |
| | | | GGAACTGCATAAGAACGAGAGCTGCCTGGCCACCAGAGAAACCAG |
| | | | CTCTAGCACAAGAGGCAGCTGTCTGCCTCCTCAGAAAACCAGCCT |
| | | | GATGATGACCCTGTGCCTGGGCAGCATCTACGAGGATCTGAAGAT |
| | | | GTACCAGACCGAGTTCCAGGCCATCAACGCCGCTCTGCAGAACCA |
| | | | CAACCACCAGCAGATCATCCTGGACAAGGGCATGCTGGTGGCTAT |
| | | | CGACGAGCTGATGCAGAGCCTGAACCATAACGGCGAGACACTGC |
| | | | GGCAGAAGCCTCCAGTTGGAGAGGCCGATCCTTACAGAGTGAAGA |
| | | | TGAAGCTGTGCATCCTGCTGCACGCCTTCAGCACCAGAGTGGTCA |
| | | | CCATCAACAGAGTGATGGGCTACCTGAGCAGCGCCAGGCGGAAG |
| | | | CGGAGCGGCAGGGGCGCTACAAACTTTAGCCTTCTGAAGCAGGC |
| | | | GGGGGATGTCGAAGAGAATCCTGGTCCAATGAGACTGCTGCTGCT |
| | | | GACATTCCTGGGCGTGTGCTGTCTGACACCCTGGGTTGTCGAAGG |
| | | | CGTGGGAACAGAGGTGCTGGAAGAGTCCAGCTGCGTGAACCTGC |
| | | | AGACCCAGAGACTGCCCGTGCAGAAGATCAAGACCTACATCATCT |
| | | | GGGAGGGCGCCATGAGAGCCGTGATCTTCGTGACAAAGAGAGGC |
| | | | CTGAAGATCTGCGCCGATCCTGAGGCCAAATGGGTCAAAGCCGCC |
| | | | ATCAAGACCGTGGACGGCAGAGCCAGCACCAGAAAGAACATGGC |
| | | | CGAGACAGTGCCTACAGGCGCCCAGAGATCTACCAGCACAGCCAT |
| | | | CACACTGACCGGCTGA |
| 34 | sFLT3L-furin | Murine protein | MTVLAPAWSPNSSLLLLLLLLSPCLRGTPDCYFSHSPISSNFKVKFREL |
| | cleavage site- | | TDHLLKDYPVTVAVNLQDEKHCKALWSLFLAQRWIEQLKTVAGSKMQ |
| | P2A-IL-12-furin | | TLLEDVNTEIHFVTSCTFQPLPECLRFVQTNISHLLKDTCTQLLALKPCI |
| | cleavage site- | | GKACQNFSRCLEVQCQPDSSTLLPPRSPIALEATELPEPRPRQRRKR |
| | P2A-XCL1 amino | | SGSGATNFSLLKQAGDVEENPGPMCPQKLTISWFAIVLLVSPLMAMW |
| | acids | | ELEKDVYVVEVDWTPDAPGETVNLTCDTPEEDDITWTSDQRHGVIGS |
| | | | GKTLTITVKEFLDAGQYTCHKGGETLSHSHLLLHKKENGIWSTEILKNF |
| | | | KNKTFLKCEAPNYSGRFTCSWLVQRNMDLKFNIKSSSSSPDSRAVTC |
| | | | GMASLSAEKVTLDQRDYEKYSVSCQEDVTCPTAEETLPIELALEARQ |
| | | | QNKYENYSTSFFIRDIIKPDPPKNLQMKPLKNSQVEVSWEYPDSWSTP |
| | | | HSYFSLKFFVRIQRKKEKMKETEEGCNQKGAFLVEKTSTEVQCKGGN |
| | | | VCVQAQDRYYNSSCSKWACVPCRVRSGGGGSGGGGSGGGGSRVI |
| | | | PVSGPARCLSQSRNLLKTTDDMVKTAREKLKHYSCTAEDIDHEDITRD |
| | | | QTSTLKTCLPLELHKNESCLATRETSSTTRGSCLPPQKTSLMMTLCLG |
| | | | SIYEDLKMYQTEFQAINAALQNHNHQQIILDKGMLVAIDELMQSLNHN |
| | | | GETLRQKPPVGEADPYRVKMKLCILLHAFSTRVVTINRVMGYLSSARR |
| | | | KRSGSGATNFSLLKQAGDVEENPGPMRLLLLTFLGVCCLTPWVVEGV |
| | | | GTEVLEESSCVNLQTQRLPVQKIKTYIIWEGAMRAVIFVTKRGLKICAD |
| | | | PEAKWVKAAIKTVDGRASTRKNMAETVPTGAQRSTSTAITLTG |
| 35 | IL-36-g DNA | Human DNA | ATGAGAGGCACTCCAGGAGACGCTGATGGTGGAGGAAGGGCCGT |
| | | | CTATCAATCAATGTGTAAACCTATTACTGGGACTATTAATGATTTGA |
| | | | ATCAGCAAGTGTGGACCCTTCAGGGTCAGAACCTTGTGGCAGTTC |
| | | | CACGAAGTGACAGTGTGACCCCAGTCACTGTTGCTGTTATCACATG |
| | | | CAAGTATCCAGAGGCTCTTGAGCAAGGCAGAGGGGATCCCATTTA |
| | | | TTTGGGAATCCAGAATCCAGAAATGTGTTTGTATTGTGAGAAGGTT |
| | | | GGAGAACAGCCCACATTGCAGCTAAAAGAGCAGAAGATCATGGAT |
| | | | CTGTATGGCCAACCCGAGCCCGTGAAACCCTTCCTTTTCTACCGTG |
| | | | CCAAGACTGGTAGGACCTCCACCCTTGAGTCTGTGGCCTTCCCGG |
| | | | ACTGGTTCATTGCCTCCTCCAAGAGAGACCAGCCCATCATTCTGAC |
| | | | TTCAGAACTTGGGAAGTCATACAACACTGCCTTTGAATTAAATATAA |
| | | | ATGACTGA |
| 35A | RNA sequence | | AUGAGAGGCACUCCAGGAGACGCUGAUGGUGGAGGAAGGGCCG |
| | corresponding to | | UCUAUCAAUCAAUGUGUAAACCUAUUACUGGGACUAUUAAUGAU |
| | SEQ ID NO: 35 | | UUGAAUCAGCAAGUGUGGACCCUUCAGGGUCAGAACCUUGUGG |
| | | | CAGUUCCACGAAGUGACAGUGUGACCCCAGUCACUGUUGCUGUU |
| | | | AUCACAUGCAAGUAUCCAGAGGCUCUUGAGCAAGGCAGAGGGGA |
| | | | UCCCAUUUAUUUGGGAAUCCAGAAUCCAGAAAUGUGUUUGUAUU |
| | | | GUGAGAAGGUUGGAGAACAGCCCACAUUGCAGCUAAAAGAGCAG |
| | | | AAGAUCAUGGAUCUGUAUGGCCAACCCGAGCCCGUGAAACCCUU |
| | | | CCUUUUCUACCGUGCCAAGACUGGUAGGACCUCCACCCUUGAGU |
| | | | CUGUGGCCUUCCCGGACUGGUUCAUUGCCUCCUCCAAGAGAGA |
| | | | CCAGCCCAUCAUUCUGACUUCAGAACUUGGGAAGUCAUACAACA |
| | | | CUGCCUUUGAAUUAAAUAUAAAUGACUGA |
| 36 | IL-36-g amino acid | Human protein | MRGTPGDADGGGRAVYQSMCKPITGTINDLNQQVWTLQGQNLVAVP |
| | | | RSDSVTPVTVAVITCKYPEALEQGRGDPIYLGIQNPEMCLYCEKVGEQ |
| | | | PTLQLKEQKIMDLYGQPEPVKPFLFYRAKTGRTSTLESVAFPDWFIAS |
| | | | SKRDQPIILTSELGKSYNTAFELNIND |
| 37 | C3-Tx | synthetic | CTCTTCGgtatcacaggagaatttcagGGAGACATTGATTATTGACTAGT |
| | | | TATTAATAGTAATCAATTACGGGGTCATTAGTTCATAGCCCATATATGGA |
| | | | GTTCCGCGTTACATAACTTACGGTAAATGGCCCGCCTGGCTGACC |
| | | | GCCCAACGACCCCCGCCCATTGACGTCAATAATGACGTATGTTCC |
| | | | CATAGTAACGCCAATAGGGACTTTCCATTGACGTCAATGGGTGGA |
| | | | GTATTTACGGTAAACTGCCCACTTGGCAGTACATCAAGTGTATCAT |
| | | | ATGCCAAGTACGCCCCCTATTGACGTCAATGACGGTAAATGGCCC |
| | | | GCCTGGCATTATGCCCAGTACATGACCTTATGGGACTTTCCTACTT |
| | | | GGCAGTACATCTACGTATTAGTCATCGCTATTACCATGTCGAGGTG |
| | | | AGCCCCACGTTCTGCTTCACTCTCCCCATCTCCCCCCCCTCCCCA |
| | | | CCCCCAATTTTGTATTTATTTATTTTTTAATTATTTTGTGCAGCGATG |
| | | | GGGGCGGGGGGGGGGGGGGGGCGCGCGCCAGGCGGGCGGG |
| | | | GCGGGGCGAGGGGCGGGGCGGGGCGAGGCGGAGAGGTGCGGC |
| | | | GGCAGCCAATCAGAGCGGCGCGCTCCGAAAGTTTCCTTTTATGGC |
| | | | GAGGCGGCGGCGGCGGCGGCCCTATAAAAAGCGAAGCGCGCGG |
| | | | CGGGCGGGAGTCGCTGCGACGCTGCCTTCGCCCCGTGCCCCGCT |
| | | | CCGCCGCCGCCTCGCGCCGCCCGCCCCGGCTCTGACTGACCGCG |
| | | | TTACTCCCACAGGTGAGCGGGCGGGACGGCCCTTCTCCTCCGGG |
| | | | CTGTAATTAGCGCTTGGTTTAATGACGGCTTGTTTCTTTTCTGTGGC |
| | | | TGCGTGAAAGCCTTGAGGGGCTCCGGGAGGGCCCTTTGTGCGGG |
| | | | GGGAGGGGCTCGGGGGGTGCGTGCGTGTGTGTGTGCGTGGGGA |
| | | | GCGCCGCGTGGGGCTCCGCGCTGCCGGGGGGCTGTGAGCGCTG |
| | | | CGGGCGCGGCGCGGGGCTTTGTGCGCTCCGCAGTGTGCGCGAG |
| | | | GGGAGCGCGGCCGGGGGCGGTGCCCCGCGGTGCGGGGGGGGC |
| | | | TGCGAGGGGAACAAAGGCTGCGTGCGGGGTGTGTGCGTGGGGG |
| | | | GGTGAGCAGGGGGTGTGGGCGCGTCGGTCGGGCTGCAACCCCC |
| | | | CCTGCACCCCCCTCCCCGAGTTGCTGAGCACGGCCGGGCTTCGG |
| | | | GTGCGGGGCTCCGTACGGGGCGTGGCGCGGGGCTCGCCGTGCC |
| | | | GGGCGGGGGTGGCGGCAGGTGGGGGTGCCGGGCGGGGCGGG |
| | | | GCCGCCTCGGGCCGGGGAGGGCTCGGGGGAGGGGCGCGGCGG |
| | | | CCCCCGGAGCGCCGGCGGCTGTCGAGGCGCGGCGAGCCGCAGC |
| | | | CATTGCCTTTTATGGTAATCGTGCGAGAGGGCGCAGGGACTTCCT |
| | | | TTGTCCCAAATCTGTGCGGAGCCGAAATCTGGGAGGCGCCGCCG |
| | | | CACCCCCTCTAGCGGGCGCGGGGCGAAGCGGTGCGGCGCCGGC |
| | | | AGGAAGGAAATGGGCGGGGAGGGCCTTCGTGCGTCGCCGCGCC |
| | | | GCCGTCCCCTTCTCCCTCTCCAGCCTCGGGGCTGTCCGCGGGGG |
| | | | GACGGCTGCCTTCGGGGGGGACGGGGCAGGGCGGGGTTCGGCT |
| | | | TCTGGCGTGTGACCGGCGGCTCTAGACAATTGTACTAACCTTCTTC |
| | | | TCTTTCCTCTCCTGACAGGGAGTTTAAACAGATAAGTTTGTACAAAA |
| | | | AAGAGAGGTGCCACCATGACAGTGCTGGCTCCTGCTTGGAGCCCT |
| | | | AACAGCTCTCTGCTGCTGCTTCTGCTCCTGCTGAGCCCATGTCTGA |
| | | | GAGGCACCCCTGACTGCTACTTCTCTCACAGCCCCATCAGCAGCA |
| | | | ACTTCAAAGTGAAGTTCCGCGAGCTGACCGACCATCTGCTGAAGG |
| | | | ACTACCCTGTGACCGTGGCCGTGAACCTGCAGGATGAGAAGCACT |
| | | | GCAAGGCCCTGTGGTCCCTGTTCCTGGCTCAGAGATGGATCGAGC |
| | | | AGCTGAAAACAGTGGCCGGCAGCAAGATGCAGACCCTGCTGGAA |
| | | | GATGTGAACACCGAGATCCACTTCGTGACCAGCTGCACCTTCCAG |
| | | | CCTCTGCCTGAGTGCCTGAGATTCGTGCAGACCAACATCAGCCAC |
| | | | CTTCTCAAGGACACATGCACCCAGCTGCTGGCCCTGAAGCCTTGT |
| | | | ATCGGCAAGGCCTGCCAGAACTTCTCCAGATGCCTGGAAGTGCAG |
| | | | TGCCAGCCTGACAGCTCTACACTGCTGCCTCCAAGAAGCCCTATC |
| | | | GCTCTGGAAGCCACAGAGCTGCCTGAGCCTAGACCTAGACAGTGA |
| | | | GCTTCCACTGGATTGTACAATTACcaacAATAAAATATCTTTATTTTC |
| | | | ATTACATCTGTGTGTTGGTTTTTTGTGTGCGCTactTTCGgtatcacagga |
| | | | gaatttcagGGAGACATTGATTATTGACTAGTTATTAATAGTAATCAATT |
| | | | ACGGGGTCATTAGTTCATAGCCCATATATGGAGTTCCGCGTTACAT |
| | | | AACTTACGGTAAATGGCCCGCCTGGCTGACCGCCCAACGACCCCC |
| | | | GCCCATTGACGTCAATAATGACGTATGTTCCCATAGTAACGCCAAT |
| | | | AGGGACTTTCCATTGACGTCAATGGGTGGAGTATTTACGGTAAACT |
| | | | GCCCACTTGGCAGTACATCAAGTGTATCATATGCCAAGTACGCCC |
| | | | CCTATTGACGTCAATGACGGTAAATGGCCCGCCTGGCATTATGCC |
| | | | CAGTACATGACCTTATGGGACTTTCCTACTTGGCAGTACATCTACG |
| | | | TATTAGTCATCGCTATTACCATGTCGAGGTGAGCCCCACGTTCTGC |
| | | | TTCACTCTCCCCATCTCCCCCCCCTCCCCACCCCCAATTTTGTATT |
| | | | TATTTATTTTTTAATTATTTTGTGCAGCGATGGGGGCGGGGGGGGG |
| | | | GGGGGGGCGCGCGCCAGGCGGGGCGGGGCGGGGCGAGGGGCG |
| | | | GGGCGGGGCGAGGCGGAGAGGTGCGGCGGCAGCCAATCAGAGC |
| | | | GGCGCGCTCCGAAAGTTTCCTTTTATGGCGAGGCGGCGGCGGCG |
| | | | GCGGCCCTATAAAAAGCGAAGCGCGCGGCGGGCGGGAGTCGCTG |
| | | | CGACGCTGCCTTCGCCCCGTGCCCCGCTCCGCCGCCGCCTCGCG |
| | | | CCGCCCGCCCCGGCTCTGACTGACCGCGTTACTCCCACAGGTGA |
| | | | GCGGGCGGGACGGCCCTTCTCCTCCGGGCTGTAATTAGCGCTTG |
| | | | GTTTAATGACGGCTTGTTTCTTTTCTGTGGCTGCGTGAAAGCCTTG |
| | | | AGGGGCTCCGGGAGGGCCCTTTGTGCGGGGGGAGCGGCTCGGG |
| | | | GGGTGCGTGCGTGTGTGTGTGCGTGGGGAGCGCCGCGTGCGGCT |
| | | | CCGCGCTGCCCGGCGGCTGTGAGCGCTGCGGGCGCGGCGCGGG |
| | | | GCTTTGTGCGCTCCGCAGTGTGCGCGAGGGGAGCGCGGCCGGG |
| | | | GGCGGTGCCCCGCGGTGCGGGGGGGGCTGCGAGGGGAACAAAG |
| | | | GCTGCGTGCGGGGTGTGTGCGTGGGGGGGTGAGCAGGGGGTGT |
| | | | GGGCGCGTCGGTCGGGCTGCAACCCCCCCTGCACCCCCCTCCCC |
| | | | GAGTTGCTGAGCACGGCCCGGCTTCGGGTGCGGGGCTCCGTACG |
| | | | GGGCGTGGCGCGGGGCTCGCCGTGCCGGGCGGGGGGTGGCGG |
| | | | CAGGTGGGGGTGCCGGGCGGGGCGGGGCCGCCTCGGGCCGGG |
| | | | GAGGGCTCGGGGGAGGGGCGCGGCGGCCCCCGGAGCGCCGGC |
| | | | GGCTGTCGAGGCGCGGCGAGCCGCAGCCATTGCCTTTTATGGTAA |
| | | | TCGTGCGAGAGGGCGCAGGGACTTCCTTTGTCCCAAATCTGTGCG |
| | | | GAGCCGAAATCTGGGAGGCGCCGCCGCACCCCCTCTAGCGGGCG |
| | | | CGGGGCGAAGCGGTGCGGCGCCGGCAGGAAGGAAATGGGCGGG |
| | | | GAGGGCCTTCGTGCGTCGCCGCGCCGCCGTCCCCTTCTCCCTCT |
| | | | CCAGCCTCGGGGCTGTCCGCGGGGGGACGGCTGCCTTCGGGGG |
| | | | GGACGGGGCAGGGCGGGGTTCGGCTTCTGGCGTGTGACCGGCG |
| | | | GCTCTAGACAATTGTACTAACCTTCTTCTCTTTCCTCTCCTGACAGG |
| | | | GAGTTTAAACAGATAAGTTTGTACAAAAAAGAGAGGTGCCACCATG |
| | | | TGCCCTCAGAAGCTGACCATCAGTTGGTTCGCCATCGTGCTGCTG |
| | | | GTGTCCCCACTGATGGCTATGTGGGAACTCGAGAAGGACGTGTAC |
| | | | GTGGTGGAAGTGGACTGGACCCCTGATGCTCCTGGCGAGACAGT |
| | | | GAACCTGACCTGCGACACACCTGAAGAGGACGACATCACCTGGAC |
| | | | CAGCGATCAGAGACACGGCGTGATCGGCTCTGGCAAGACCCTGA |
| | | | CAATTACCGTGAAAGAGTTCCTGGACGCCGGCCAGTACACCTGTC |
| | | | ACAAAGGCGGAGAGACACTGAGCCACTCTCATCTGCTGCTGCACA |
| | | | AGAAAGAGAACGGCATCTGGTCCACCGAGATCCTGAAGAACTTCA |
| | | | AGAACAAGACCTTCCTGAAGTGCGAGGCCCCTAACTACAGCGGCA |
| | | | GATTCACCTGTAGCTGGCTGGTGCAGAGAAACATGGACCTGAAGT |
| | | | TCAACATCAAGTCCTCCAGCAGCAGCCCCGACAGCAGAGCTGTGA |
| | | | CATGTGGCATGGCTAGCCTGAGCGCCGAGAAAGTGACACTGGAC |
| | | | CAGAGAGACTACGAGAAGTACAGCGTGTCCTGCCAAGAGGACGTG |
| | | | ACCTGTCCTACCGCCGAGGAAACACTGCCTATCGAGCTGGCCCTG |
| | | | GAAGCCAGACAGCAGAACAAATACGAGAACTACTCTACCAGCTTCT |
| | | | TCATCCGGGACATCATCAAGCCCGATCCTCCAAAGAACCTGCAGA |
| | | | TGAAGCCTCTGAAGAACAGCCAGGTCGAGGTGTCCTGGGAGTACC |
| | | | CTGACAGCTGGTCTACCCCTCACAGCTACTTCAGCCTGAAATTCTT |
| | | | CGTGCGGATCCAGCGCAAGAAAGAAAAGATGAAGGAAACCGAGG |
| | | | AAGGCTGCAACCAGAAAGGCGCTTTCCTGGTGGAAAAGACCAGCA |
| | | | CCGAGGTGCAGTGCAAAGGCGGCAATGTCTGTGTGCAGGCCCAG |
| | | | GACCGGTACTACAACAGCAGCTGTAGCAAGTGGGCCTGCGTGCCA |
| | | | TGCAGAGTCAGATCTGGTGGCGGAGGATCTGGCGGAGGTGGAAG |
| | | | CGGCGGAGGCGGATCTAGAGTGATTCCTGTGTCTGGCCCTGCCA |
| | | | GATGCCTGAGCCAGTCTAGAAACCTGCTGAAAACCACCGACGACA |
| | | | TGGTCAAGACCGCCAGAGAGAAGCTGAAGCACTACTCCTGCACAG |
| | | | CCGAGGACATCGACCACGAGGATATCACCAGGGACCAGACAAGC |
| | | | ACCCTGAAAACCTGCCTGCCTCTGGAACTGCATAAGAACGAGAGC |
| | | | TGCCTGGCCACCAGAGAAACCAGCTCTACCACAAGAGGACAGCTGT |
| | | | CTGCCTCCTCAGAAAACCAGCCTGATGATGACCCTGTGCCTGGGC |
| | | | AGCATCTACGAGGATCTGAAGATGTACCAGACCGAGTTCCAGGCC |
| | | | ATCAACGCCGCTCTGCAGAACCACAACCACCAGCAGATCATCCTG |
| | | | GACAAGGGCATGCTGGTGGCTATCGACGAGCTGATGCAGAGCCT |
| | | | GAACCATAACGGCGAGACACTGCGGCAGAAGCCTCCAGTTGGAG |
| | | | AGGCCGATCCTTACAGAGTGAAGATGAAGCTGTGCATCCTGCTGC |
| | | | ACGCCTTCAGCACCAGAGTGGTCACCATCAACAGAGTGATGGGCT |
| | | | ACCTGAGCAGCGCCTGAGCTTCCACTGGATTGTACAATTACcaacAA |
| | | | TAAAATATCTTTATTTTCATTACATCTGTGTGTTGGTTTTTTGTGTGC |
| | | | GCTataTTCGgtatcacaggagaatttcagGGAGACATTGATTATTGACT |
| | | | AGTTATTAATAGTAATCAATTACGGGGTCATTAGTTCATAGCCCATATAT |
| | | | GGAGTTCCGAGTTACATAACTTACGGTAAATGGCCCGCCTGGCTG |
| | | | ACCGCCCAACGACCCCCGCCCATTGACGTCAATAATGACGTATGT |
| | | | TCCCATAGTAACGCCAATAGGGACTTTCCATTGACGTCAATGGGTG |
| | | | GAGTATTTACGGTAAACTGCCCACTTGGCAGTACATCAAGTGTATC |
| | | | ATATGCCAAGTACGCCCCCTATTGACGTCAATGACGGTAAATGGC |
| | | | CCGCCTGGCATTATGCCCAGTACATGACCTTATGGGACTTTCCTAC |
| | | | TTGGCAGTACATCTACGTATTAGTCATCGCTATTACCATGTCGAGG |
| | | | TGAGCCCCACGTTCTGCTTCACTCTCCCCATCTCCCCCCCCTCCC |
| | | | CACCCCCAATTTTGTATTTATTTATTTTTTAATTATTTTGTGCAGCGA |
| | | | TGGGGGCGGGGGGGGGGGGGGGGCGCGCGCCAGGCGGGGCG |
| | | | GGGCGGGGCGAGGGGCGGGGCGGGGCGAGGCGGAGAGGTGCG |
| | | | GCGGCAGCCAATCAGAGCGGCGCGCTCCGAAAGTTTCCTTTTATG |
| | | | GCGAGGCGGCGGCGGCGGCGGCCCTATAAAAAGCGAAGCGCGC |
| | | | GGCGGGCGGGAGTCGCTGCGACGCTGCCTTCGCCCCGTGCCCC |
| | | | GCTCCGCCGCCGCCTCGCGCCGCCCGCCCCGGCTCTGACTGACC |
| | | | GCGTTACTCCCACAGGTGAGCGGGCGGGACGGCCCTTCTCCTCC |
| | | | GGGCTGTAATTAGCGCTTGGTTTAATGACGGCTTGTTTCTTTTCTG |
| | | | TGGCTGCGTGAAAGCCTTGAGGGGCTCCGGGAGGGCCCTTTGTG |
| | | | CGGGGGGAGCGGCTCGGGGGGTGCGTGCGTGTGTGTGTGGCGTG |
| | | | GGGAGCGCCGCGTGCGGCTCCGCGCTGCCCGGCGGCTGTGAGC |
| | | | GCTGCGGGCGCGGCGCGGGGCTTTGTGCGCTCCGCAGTGTGCG |
| | | | CGAGGGGAGCGCGGCCGGGGGCGGTGCCCCGCGGTGCGGGGG |
| | | | GGGCTGCGAGGGGAACAAAGGCTGCGTGCGGGGTGTGTGCGTG |
| | | | GGGGGGTGAGCAGGGGGTGTGGGCGCGTCGGTCGGGCTGCAAC |
| | | | CCCCCCTGCACCCCCCTCCCCGAGTTGCTGAGCACGGCCCGGCT |
| | | | TCGGGTGCGGGGCTCCGTACGGGGCGTGGCGCGGGGCTCGCCG |
| | | | TGCCGGGCGGGGGGTGGCGGCAGGTGGGGGTGCCGGGCGGGG |
| | | | CGGGGCCGCCTCGGGCCGGGGAGGGCTCGGGGGAGGGGCGCG |
| | | | GCGGCCCCCGGAGCGCCGGCGGCTGTCGAGGCGCGGCGAGCCG |
| | | | CAGCCATTGCCTTTTATGGTAATCGTGCGAGAGGGCGCAGGGACT |
| | | | TCCTTTGTCCCAAATCTGTGCGGAGCCGAAATCTGGGAGGCGCCG |
| | | | CCGCACCCCCTCTAGCGGGCGCGGGGCGAAGCGGTGCGGCGCC |
| | | | GGCAGGAAGGAAATGGGCGGGGAGGGCCTTCGTGCGTCGCCGC |
| | | | GCCGCCGTCCCCTTCTCCCTCTCCAGCCTCGGGGCTGTCCGCGG |
| | | | GGGGACGGCTGCCTTCGGGGGGGACGGGGCAGGGCGGGGTTCG |
| | | | GCTTCTGGCGTGTGACCGGCGGCTCTAGACAATTGTACTAACCTT |
| | | | CTTCTCTTTCCTCTCCTGACAGGGAGTTTAAACAGATAAGTTTGTAC |
| | | | AAAAAAGAGAGGTGCCACCATGAGACTGCTGCTGCTGACATTCCT |
| | | | GGGCGTGTGCTGTCTGACACCCTGGGTTGTCGAAGGCGTGGGAA |
| | | | CAGAGGTGCTGGAAGAGTCCAGCTGCGTGAACCTGCAGACCCAG |
| | | | AGACTGCCCGTGCAGAAGATCAAGACCTACATCATCTGGGAGGGC |
| | | | GCCATGAGAGCCGTGATCTTCGTGACAAAGAGAGGCCTGAAGATC |
| | | | TGCGCCGATCCTGAGGCCAAATGGGTCAAAGCCGCCATCAAGACC |
| | | | GTGGACGGCAGAGCCAGCACCAGAAAGAACATGGCCGAGACAGT |
| | | | GCCTACAGGCGCCCAGAGATCTACCAGCACAGCCATCACACTGAC |
| | | | CGGCTGAGCTTCCACTGGATTGTACAATTACcaacAATAAAATATCT |
| | | | TTATTTTCATTACATCTGTGTGTTGGTTTTTTGTGTGCGCTcaaaaaa |

### EXAMPLES

The following are non-limiting examples of methods for generating and testing compositions described above end for treating an individual with such compositions.

### Example 1. Cell-Free Production of Synthetic Circular DNA Vectors, Encoding a Therapeutic Triplet

This example describes the production and characterization of two synthetic circular DNA vectors encoding a therapeutic triplet: (1) a single-transcription unit vector having a single promoter driving expression of all three therapeutic sequences (as shown in FIG. 1); and (2) a multi-transcription unit vector having three promoters, each of which drives expression of one of the three therapeutic sequences (as shown in FIG. 2A). In this example, the therapeutic sequences are sFLT3L, IL-12, and XCL1 (operably linked in a 5′-to-3′ direction).

The following reagents were mixed in 1×Phi29 buffer (New England Biolabs) to prepare the rolling circle amplification (RCA) solution: plasmid DNA containing the therapeutic triplet in either a single-transcription unit or a multi-transcription unit configuration (5 μg/mL final concentration); random primers (50 μM final concentration); NaOH (10 mM final concentration); dNTPs (2 mM final concentration); bovine serum albumin (BSA) (0.2 mg/mL final concentration); Phi29 DNA polymerase (200 U/mL final concentration); and pyrophosphatase stock (New England Biolabs: 0.4 mU/mL final concentration). The RCA solution was continuously mixed for 18 hours at 30° C.

After incubation, the RCA solution was heat-inactivated by raising the temperature to 65° C. for 45 minutes. The temperature of the inactivated RCA solution was then reduced to 37° C.

To produce the Bsal solution, the inactivated RCA solution (0.2 mg DNA/mL final concentration) was added to CUTSMART® buffer (1× final concentration) containing Bsal (2.5 U/μg DNA final concentration). The Bsal solution was continuously mixed for two hours at 37° C. No heat-inactivation was carried out on the Bsal solution. The temperature of the digested Bsal solution was reduced to 25° C.

To produce the ligation solution, the digested Bsal solution (40 μg DNA/mL final concentration) was added to CUTSMART® buffer containing T4 ligase (10 U T4 ligase per μg DNA) and ribo ATP (1 mM final concentration). The ligation solution was incubated for two hours at 25° C.

After incubation, the ligation solution was heat-inactivated by raising the temperature to 65° C. for 45 minutes. The temperature of the inactivated ligation solution was then reduced to 37° C.

To produce the supercoiling solution, the ligation solution was added to gyrase buffer containing DNA gyrase (1.5 U gyrase per μg DNA). Gyrase buffer contains 35 mM Tris-HCl, 24 mM KCl, 4 mM MgCl2, 1 mM ATP, 2 mM DTT, 1.8 mM spermidine, 32% glycerol (w/v), and 100 μg/mL BSA, and 500 μg/mL BSA. The supercoiling solution was continuously mixed for two hours at 37° C. No heat-inactivation was carried out on the supercooling solution.

Next, the supercooling solution was added to potassium acetate buffer (50 mM potassium acetate, final concentration) containing T5 exonuclease (2.5 U T5 exonuclease per μg DNA) to produce the cleanup solution. The cleanup solution was continuously mixed for at least two hours at 37° C. No heat-inactivation was carried out on the cleanup solution.

The cleanup solution was then sterile-filtered through a 0.22 μm filter and diluted 1:1 in a buffer containing 1.5 M NaCl, 100 mM MOPS, 30% isopropyl alcohol (IPA), and 0.3% Triton X-100 (v/v) to achieve a final concentration of 780 mM NaCl, 50 mM MOPS, 15% isopropyl alcohol (IPA), and 0.15% Triton X-100 (v/v). Diluted cleanup solution was added to Qiagen plasmid prep columns, and DNA was eluted with QN buffer. Eluate was diluted with IPA (40% v/v) and centrifuged at 4° C. for 30 minutes at 15,000 g. Pellets were washed with 70% EtOH and centrifuged again at 4° C. for 30 minutes at 15,000 g. After the second centrifugation, pellets were resuspended in PBS at 1.0 mg/mL.

Supercooled monomer was calculated by densitometry analysis of gel electrophoresis preparations using image Lab software (BIO-RAD®). 200 ng synthetic circular DNA vector sample was loaded into tris-acetate-EDTA gels and electrophoresis was run at 100V for 40 minutes prior to staining with 1% EtBr for 20 minutes. For each synthetic circular DNA vector sample, the target band was identified according to its size, and “Band Detection Sensitivity” was set as a “Custom Sensitivity” at a value of 50 in “Detection Settings.”

The methods above yielded 1.7 mg of the single-transcription unit vector of FIG. 1A (4537 bp) from 0.42 mg of plasmid (approximately 4-fold increase), compared to 4.0 mg of the multi-transcription unit vector of FIG. 2A (8035 bp) from 0.75 mg of plasmid (a 5.3-fold increase). Single-transcription unit vector was calculated as 78% supercoiled, as measured by the densitometry analysis discussed above. The multi-transcription unit vector was calculated as 85% supercoiled by the same analysis. By comparison, a sample of synthetic circular DNA vector encoding a single gene (IL-12) driven by a CAG promoter (3466 bp) was produced by the same methods and yielded 2.5 mg of vector from 0.32 mg of plasmid (a 7.8-fold increase). The single gene vector was calculated as having 84% supercoiling, as measured by the densitometry analysis discussed above. This data shows that the cell-free production methods described herein can be utilized for effective production of relatively large synthetic circular DNA vectors containing multiple genes in addition to smaller synthetic circular DNA vectors containing single genes.

### Example 2. Protein Expression for Each of the Three Therapeutic Sequences of a Synthetic Circular DNA Vector

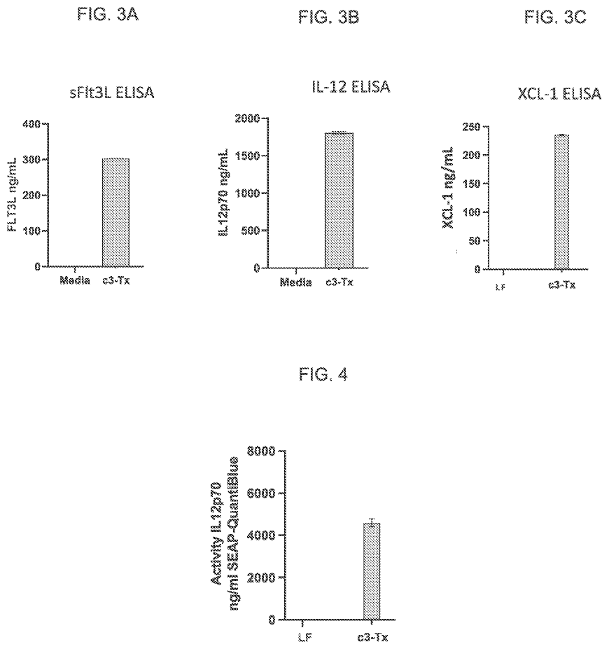

The 8035 bp multi-transcription unit synthetic circular DNA vector of SEQ ID NO: 37 (c3-Tx) was characterized for protein expression in vitro. HEK293T cells were seeded at a density of 200K cell/well in 12-well plates with clear flat bottoms and incubated with vector at a concentration of 1 μg per well+3 μL per well of Lipofetamine3000+2 μL per well of P3000 for 48 hours. Protein expression of sFlt3L, XCL1, and IL-12 by c3-Tx was quantified by ELISA, using media as a negative control. Protein expression was detected for sFlt3L (FIG. 3A), IL-12 (FIG. 3B), and XCL1 (FIG. 3C), demonstrating that c3-Tx leads to protein expression for all three immunomodulatory proteins.

### Example 3. Functional Assays for Proteins Expressed by the Synthetic Circular DNA Vector

Functional assays were performed to determine whether the proteins expressed by the 8035 bp multi-transcription unit synthetic circular DNA vector of FIG. 2A were biologically functional after in vitro incubations as described in Example 2.

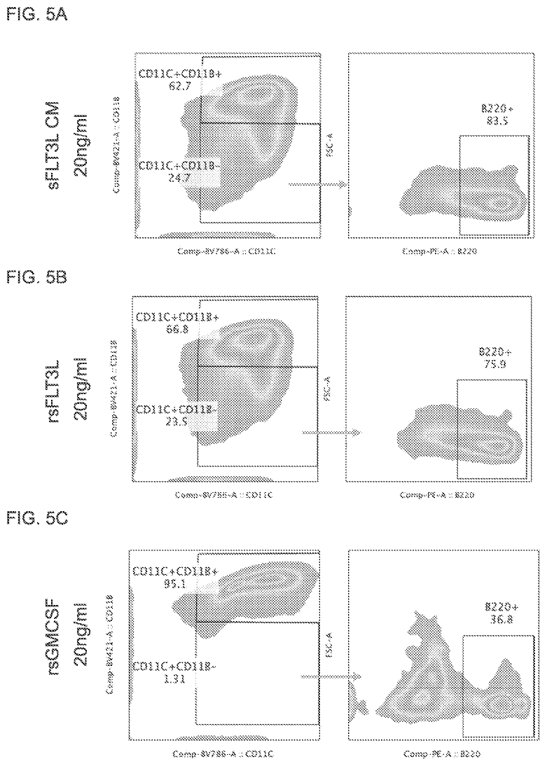

To measure IL-12 activity, a Quanti Blue/secreted embryonic alkaline phosphatase (SEAP) assay was conducted using HEK Blue-IL-12 cells. Briefly, 50,000 HEK-Blue-IL12 cells per well were plated in 96-well plates. Cells were treated with 20 μL/well mulL 12p70 (standard curve), huINFalpha (negative control), or conditioned media from transfected HEK293T cells (diluted 1:100) and incubated for 24 hours at 37° C. 20 μL of HEK-Blue-IL12 cell supernatants from each sample were transferred to wells of 96-well plates, and 180 μL Quanti-Blue solution was added. Samples were incubated for one hour at 37C and absorbance at 630 nm was measured. FIG. 4 shows that IL-12p70 expressed from the 8035 bp multi-transcription unit synthetic circular DNA vector of FIG. 2A was biologically functional.

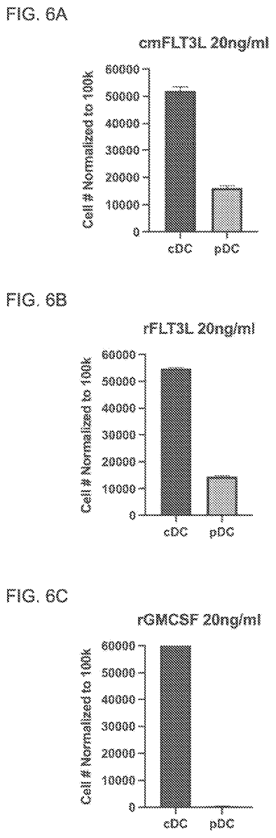

Flt3L activity was measured based on bone marrow derived dendritic cell (BMDC) differentiation by sFlt3L-conditioned media. Briefly, HEK cells were incubated with the 8035 bp multi-transcription unit synthetic circular DNA vector of FIG. 2 to produce conditioned media. Bone marrow progenitor cells were harvested from murine femurs and cultured in the conditioned media (20 ng/mL), media containing recombinant sFLT3L (20 ng/mL) as a positive control, and media containing recombinant GMCSF (20 ng/mL) as a negative control. For each condition, media was replenished at days 3 and 6, and loosely adherent cells were harvested at day 7. Flow cytometry was performed to immunophenotype the cells by gating on CD11c⁺CD11b⁺ cells (conventional DCs; cDCs) and CD11c⁺CD11b⁻B220⁺ cells (plasmacytoid DCs (pDCs)) as shown by the representative plots in FIGS. 5A-5C. FIGS. 6A-6C show the numbers of cDCs and pDCs produced in each condition. sFLT3L conditioned media caused differentiation of BMDCs to a similar cDC/pDC profile as recombinant sFLT3L (FIGS. 6A and 6B), whereas recombinant GMCSF skewed BMDC phenotype away from pDCs toward cDCs (FIG. 6C). Together, these data show that sFLT3L expressed from the 8035 bp multi-transcription unit synthetic circular DNA vector of FIG. 2A was biologically functional.

### Example 4. Improved Intratumoral Expression Persistence of Synthetic Circular DNA Vectors Compared to Plasmid

In this example, synthetic circular DNA vectors were tested for intratumoral expression persistence. Synthetic circular DNA vectors encoding fLuc (c3-fLuc) were prepared using cell-free methods described herein and compared to plasmid DNA vectors encoding fLuc (p-fLuc). BALB/c mice were inoculated with CT-26 tumor cells in the flank eleven days prior to treatment. c3-fLuc or p-fLuc was administered intratumorally (46 uL to 55 uL at a concentration of 1 mg/mL) followed by transmission of electrical energy by plate electrodes positioned to contact the surface of the tumor. Eight 5-ms pulses were administered at 800 V/cm for each tumor to perform electrotransfer of the vector into the tumor cells. Animals were monitored by in-life imaging for 17 days.

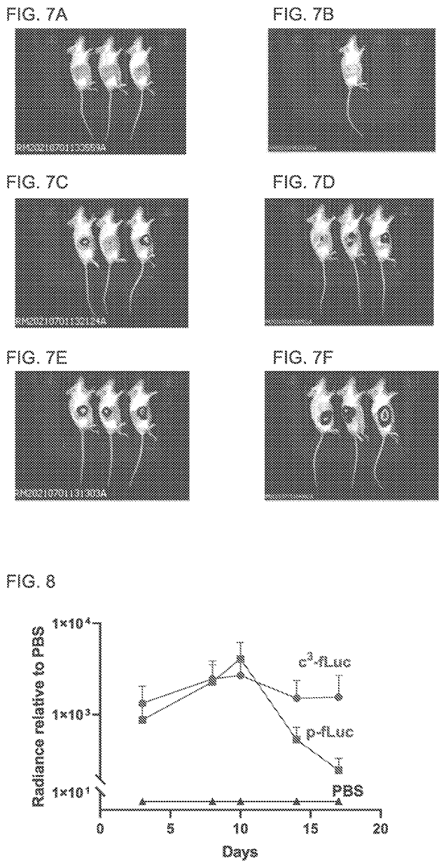

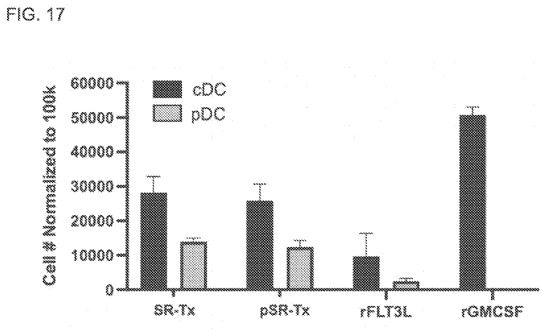

FIGS. 7A-7F are images showing fLuc expression in the mice at 3 days (FIGS. 7A, 7C, and 7E) and 17 days (FIGS. 7B, 7D, and 7F) from treatment with PBS (FIGS. 7A and 7B), p-fLuc (FIGS. 7C and 7D), and c3-fLuc (FIGS. 7E and 7F). A quantification of these data, along with additional timepoints at days 7, 10, and 14, is shown by the graph of FIG. 8. These data show that fLuc radiance was maintained throughout the 17-day time course in animals treated with synthetic circular DNA vectors (c3 fLuc), whereas fLuc expression waned in animals treated with plasmid DNA vectors (p fLuc), suggesting that the synthetic circular DNA vector exhibited enhanced expression persistence compared to plasmid.

### Example 5. Reduction of Tumor Growth in Mice Treated with a Single Dose of Synthetic Therapeutic Circular DNA Vectors

The 8035 bp multi-transcription unit synthetic circular DNA vector of FIG. 2A was tested for tumor reduction in vivo. In this study, BALB/c mice were inoculated with CT-26 tumor cells in the flank 8 days prior to treatment. The synthetic therapeutic circular DNA vector of FIG. 2A (c3-Tx) was prepared using cell-free methods as described herein and formulated as a pharmaceutical composition in PBS at a concentration of 1 mg/mL. A PBS-treated group served as the negative control. A single dose of 50 uL of the synthetic circular DNA vector formulation or PBS was administered into the tumor of each mouse, and electrotransfer was carried out after each administration using a multi-needle electrode array. Six 28-gauge needles were arranged in two rows of three, with 1.5 mm between each needle in each row (3 mm row length) and 2.5 mm between rows. The needle array was inserted intratumorally and eight 5-ms pulses were administered at 800 V/cm (current flowing between rows of needles).

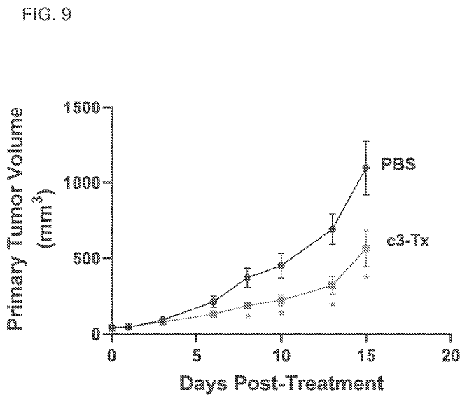

Tumor volumes were monitored over time. As shown in FIG. 9, c3 Tx-treated mice exhibited a significantly lower mean tumor volume at day 7 and beyond, relative to the PBS control group. These data show that electrotransfer of synthetic therapeutic circular DNA vectors in tumor tissue can reduce tumor growth.

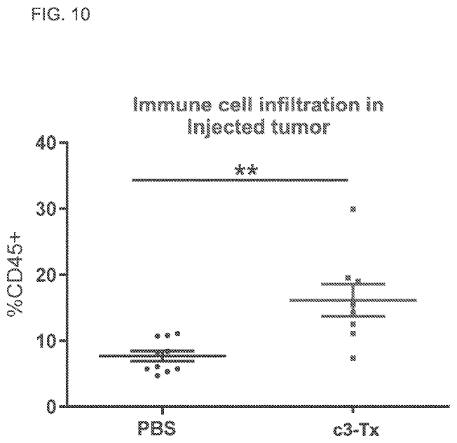

Tumor samples were collected to determine whether the observed effect of c3 Tx was associated with increased tumor infiltration of immune cells. Among tumor cells in PBS-injected tumors, approximately 8% exhibited an immune phenotype, as measured by flow cytometric quantification of CD45′ expression (FIG. 10). In contrast, immune cells accounted for approximately 17% of the cells in c3 Tx-injected tumors. Tumor sample analysis was conducted in lieu of monitoring survival in this single-dose study.

### Example 6. Redosing of Synthetic Therapeutic Circular DNA Vectors Prolonged LED to Reduction of Tumor Growth and Prolonged Survival

The 8035 bp mu ti-transcription unit synthetic circular DNA vector of FIG. 2A was tested for effect on tumor-bearing mouse survival. In this study, BALB/c mice were inoculated with CT-26 tumor cells in the flank 12 days prior to treatment. The synthetic therapeutic circular DNA vector of FIG. 2A (c3-Tx) was prepared using cell-free methods as described herein and formulated as a pharmaceutical composition in PBS at a concentration of 1 mg/mL. Negative controls included PBS group and synthetic circular DNA vector encoding fLuc (c3-fLuc; 1 mg/mL). 50 uL of the synthetic circular DNA vector formulations or PBS were administered into the tumor at days 1 and 4 of the study. Electrotransfer was carried out after each administration of the vector using a bipolar multi-needle electrode array as described in Example 5. Eight 5-ms pulses were administered at 800 V/cm. Tumor volumes were monitored, and mice were euthanized upon reaching tumor burden (2000 mm³).

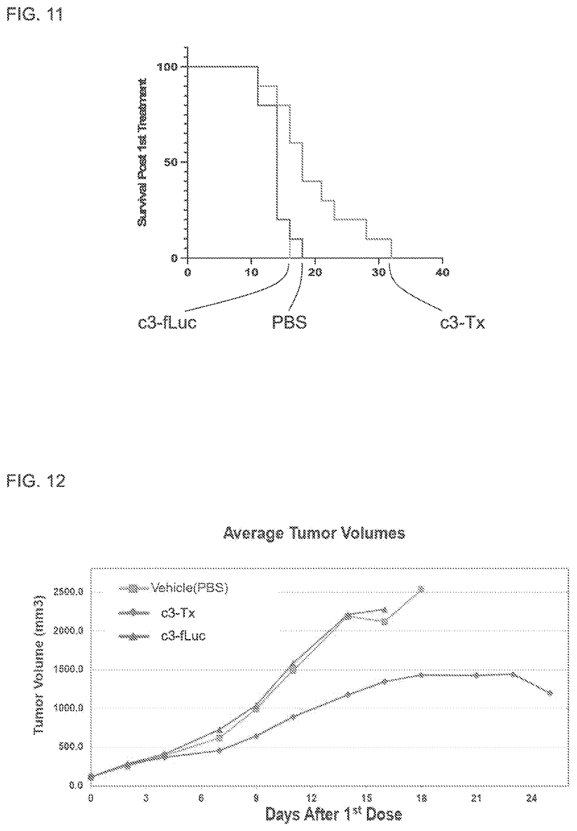

FIG. 11 shows survival curves for each group (n=10). Mice treated with the synthetic therapeutic circular DNA vector survived longer than PBS treated or c3 fLuc controls groups and experienced slower tumor growth, on average, as measured by tumor volume over time (FIG. 12). This data confirms the observations in Example 6. Moreover, these data indicate that the therapeutic effect of the synthetic circular DNA vector is dependent on is therapeutic sequence, rather than a non-specific immune response to the DNA vector itself, as shown by the lack of efficacy of c3 fLuc.

### Example 7. Intratumoral Electrotransfer of mRNA Vectors

To determine the feasibility of expressing mRNA in tumor tissue by electrotransfer, 50 uL naked mRNA (1 mg/mL) encoding GFP was injected into CT-26 flank tumors on BALB/c mice (N=12) at day 1 of the study. Synthetic circular DNA vector encoding GFP (c3-GFP) was used as a positive control, and PBS was used as a negative control. Immediately after nucleic acid injection, electrotransfer was performed using a bipolar multi-needle array as described in Example 5. Eight 5-ms pulses were administered at 800 V/cm. Tumors were collected at terminal endpoints (days, 2, 5, and 8) for GFP protein expression in the tumor.

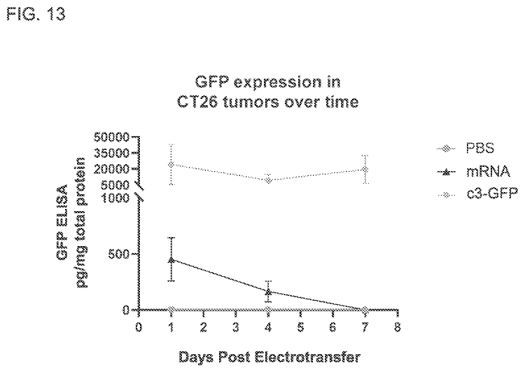

FIG. 13 shows GFP expression over time. In tumors injected with synthetic circular DNA vectors, GFP was highly expressed at each timepoint. Despite lower expression relative to the synthetic circular DNA vector group, tumors injected with naked mRNA showed detectable expression at days 2 and 5.

### Example 8. Protein Expression for Each of the Three Therapeutic Sequences of a Tricistronic Self-Replicating RNA Molecule and Plasmid Encoding a Tricistronic Self-Replicating RNA Molecule

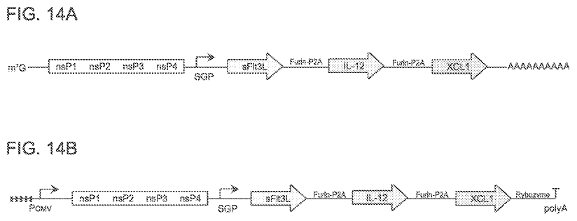

A self-replicating RNA molecule encoding sFLT3L, IL-12, and XCL1 having a length of 10,429 bp (SR-Tx; FIG. 14A) was produced and characterized for protein expression in vitro. For comparison, a plasmid encoding the self-replicating RNA molecule encoding sFLT3L, IL-12, and XCL1 having a length of 13,978 pm (pSR-Tx; FIG. 14B) was also produced and tested. Transfection control (TC) is also shown.

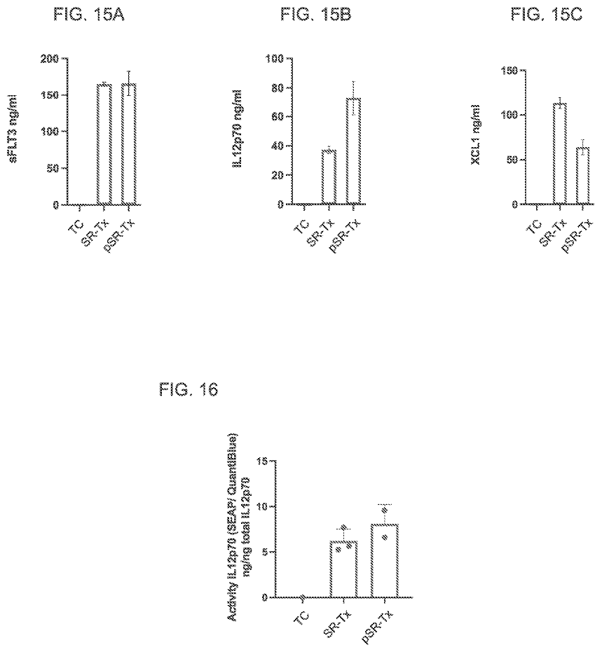

HEK293T cells were seeded at a density of 200K cell/well in 12-well plates with clear flat bottoms and incubated with vector at a concentration of 1 μg per well+3 μL per well of Lipofetamine3000+2 μL per well of P3000, or 3 of P3000 per well of lipofectamine messenger max, for 48 hours. Protein expression of sFlt3L, XCL1, and IL-12 by c3-Tx was quantified by ELISA. Protein expression was detected for sFlt3L (FIG. 15A), IL-12 (FIG. 15B), and XCL1 (FIG. 15C), for both self-replicating RNA (SR-Tx) and plasmid encoding self-replicating RNA (pSR-Tx).

### Example 9. Functional Assay for Proteins Expressed by the Tricistronic Self-Replicating RNA Molecule and Plasmid Encoding the Tricistronic Self-Replicating RNA Molecule

Functional assays were performed to determine whether the proteins expressed by the tricistronic self-replicating RNA molecule and plasmid encoding the tricistronic self-replicating RNA molecule were biologically functional after in vitro incubations as described in Example 2.

To measure IL-12 activity, a Quanti Blue/SEAP assay was conducted as described in Example 3 above. FIG. 16 shows that IL-12p70 expressed from the tricistronic self-replicating RNA molecule and plasmid encoding the tricistronic self-replicating RNA molecule were biologically functional.

Flt3L activity was measured based on BMDC differentiation by sFlt3L-conditioned media, as described in Example 3, above. Flow cytometry was performed to immunophenotype the cells by gating on CD11c⁺CD11b⁺ cells (conventional DCs; cDCs) and CD11c⁺CD11b⁺B220⁺ cells (plasmacytoid DCs (pDCs)). FIG. 17 show the numbers of cDCs and pDCs produced in each condition. sFLT3L-conditioned media from both test constructs caused differentiation of BMDCs into both pDCs and cDCs, similar to recombinant sFLT3L, whereas recombinant GMCSF skewed BMDC phenotype away from pDCs toward cDCs. Together, these data show that sFLT3L expressed from the tricistronic self-replicating RNA molecule and plasmid encoding the tricistronic self-replicating RNA molecule were biologically functional.

### Example 10. Cell-Free Production of a Circular DNA Vector Encoding a Tricistronic Self-Replicating RNA Molecule

Described herein is an exemplary method of synthesizing a DNA vector encoding a tricistronic self-replicating RNA molecule for immunomodulation. A person of ordinary skill in the art will recognize that other methods may be used to arrive at a similar self-replicating RNA molecule or a self-replicating RNA molecule having other sequences encompassed by the present description.

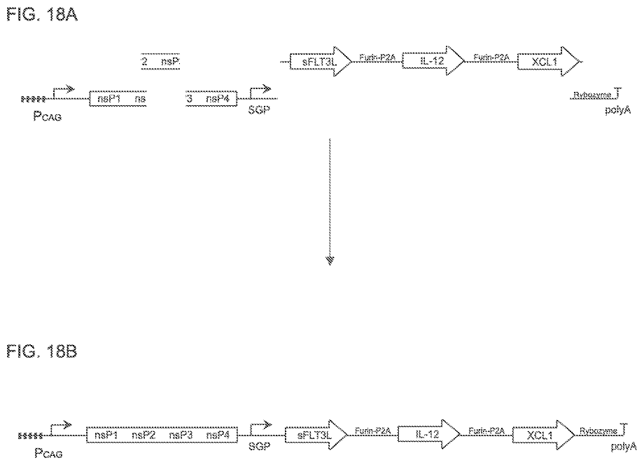

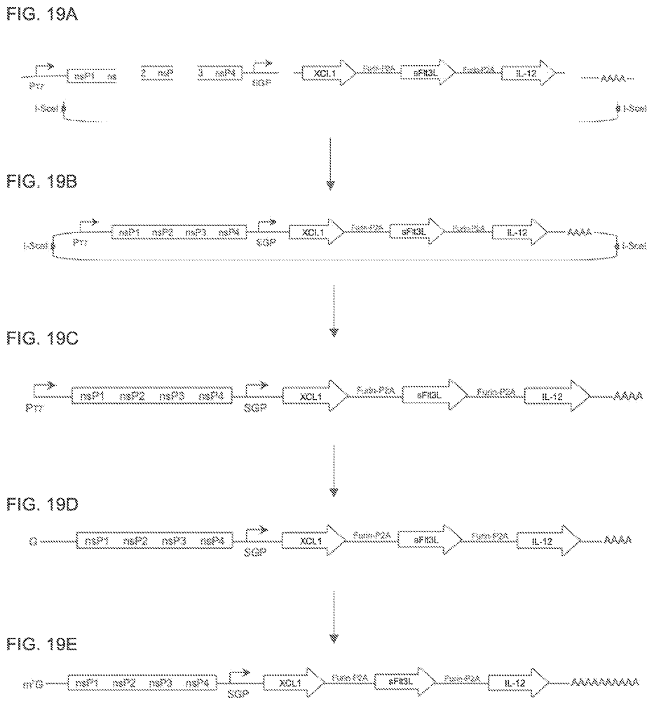

First, individual DNA fragments encoding 5′ UTR nonstructural proteins 1-4 (snP1-rMP4) and three immunomodulatory genes (represented here as sFlt3L, IL-12, and XCL1) are assembled with a 3′ UTR polyadenylation sequence and a plasmid backbone through a golden-gate assembly process (FIGS. 18A and 18B). The product is a circular plasmid having a size of about 13 kb (FIG. 188). This circular plasmid undergoes rolling circle amplification using phi29 polymerase and digestion with a restriction enzyme. The DNA is ligated to form a circular DNA vector.

### Example 11. Treatment of Cancer Using Pulsed Electric Field-Mediated Administration of a Circular DNA Vector

Described herein is an exemplary method of administering a circular DNA vector of the invention as part of a cancer treatment.

In this example, the patient being treated is an individual who has been diagnosed with a nodular melanoma characterized by the presence of a tumor having a diameter of about 10 mm. Treatment of the melanoma includes administration of a pharmaceutical composition containing an immunomodulatory circular DNA vector in conjunction with transmission of a pulsed electric field therapy to the tumor. Treatment is performed in an outpatient setting.

A circular DNA vector encoding three immunomodulatory proteins (i.e., XCL1, sFlt3L, and IL-12, as shown in FIG. 1A or 2A) is synthesized according to methods described herein and in international Patent Publication WO 2019/178500. The composition containing the circular DNA vector is provided as a lyophilized powder in a single-use glass vial containing pharmaceutically acceptable salts as buffering agent. The quantity of DNA in the lyophilized composition is about 200 μg. A clinical professional adds 200 μL sterile water to the vial to resuspend the circular DNA vector, thereby preparing a liquid pharmaceutical composition of 1.0 mg/mL for administration. The circular DNA vector-containing pharmaceutical composition is loaded into a sterile syringe equipped with a 30-gauge needle.

Using the 30-gauge needle, the physician administers the circular DNA vector-containing pharmaceutical composition directly into the tumor under direct visual and/or computed tomographic (CT) guidance to avoid major blood vessels.

Within thirty minutes of intratumoral injection of the pharmaceutical composition, the physician inserts a needle electrode at or near the injection site and transmits an intratumoral pulsed electric field. The electric field is transmitted by generating a series of eight square waveforms of 50 ms in duration each and 500 V in amplitude. Pulses are delivered about every one second. The treatment is complete upon transmission of the pulsed electric field.

The patient is monitored weekly after treatment for tumor progression by CT. A reduction in tumor diameter (i.e., less than 10 mm) confirms that the treatment is effective.

### Example 12. Synthesis of a Tricistronic Self-Replicating RNA Molecule

Described herein is an exemplary method of synthesizing a tricistronic self-replicating RNA molecule for immunomodulation. A person of ordinary skill in the art will recognize that other methods may be used to arrive at a similar self-replicating RNA molecule or a self-replicating RNA molecule having other sequences encompassed by the present description.

First, individual DNA fragments encoding 5′ UTR nonstructural proteins 1-4 (snP1-nsP4) and three immunomodulatory genes (represented here as XCL1, sFlt3L, and IL-12) are assembled with a 3′ UTR polyadenylation sequence and a plasmid backbone through a golden-gate assembly process (FIGS. 14A and 148). The product is a circular plasmid having a size of about 13.5 kb (FIG. 148). Next, the circular plasmid is cut by digestion with a restriction enzyme, 1-Scel, to produce a linearized piece of DNA with a 5′ 17 promoter (P₁₇) and a polyadenylated 3′ end. FIG. 14C shows the resulting linear DNA molecule, which has a size of about 10.5 kb. The linear DNA is transcribed using in-vitro transcription into a linear, self-replicating RNA molecule (FIG. 14D). Next, the 5′ end is capped (m⁷G) by mRNA Cap 2′-O-Methyltransferase, and the poly(A) tail is extended by E. coli Poly(A) Polymerase, thereby completing the synthesis of the self-replicating RNA molecule. 5′ cap analogues can be used to alter the characteristics of synthesized mRNA, including half-life.

FIG. 14E illustrates the final product—a 5′ capped self-replicating RNA molecule having an elongated polyadenylation sequence, a replicase-encoding sequence (nsP1, nsP2, nsP3, and nsP4) that transcribes RNA from the self-replicating RNA molecule, and a tricistronic immunomodulatory sequence having a dendritic cell chemoattractant-encoding gene (i.e., XCL1), a dendritic cell growth factor or activator-encoding gene (i.e., sFlt3L), and a lymphocyte signaling protein-encoding gene (i.e., IL-12).

### Example 13. Treatment of Cancer Using Pulsed Electric Field-Mediated Administration of a Self-Replicating RNA Molecule

Described herein is an exemplary method of administering a self-replicating RNA molecule of the invention as part of a cancer treatment.

In this example, the patient being treated is an individual who has been diagnosed with a squamous cell carcinoma of the head and neck (HNSCC) characterized by the presence of a nasopharyngeal tumor having a volume of about 10 cm³. Treatment of the squamous cell carcinoma includes administration of a pharmaceutical composition of a self-replicating RNA molecule in conjunction with transmission of a pulsed electric field therapy to the nasopharyngeal tumor. Treatment Is performed in an outpatient setting.

A self-replicating RNA molecule encoding three immunomodulatory genes (i.e., XCL1, sFlt3L, and IL-12) is synthesized according to the method of Example 1 and provided as a lyophilized powder in a single-use glass vial containing pharmaceutically acceptable salts as buffering agent. The quantity of self-replicating RNA in the lyophilized composition is about 200 μg. A clinical professional adds 200 μL sterile water to the vial to resuspend the self-replicating RNA, thereby preparing a liquid pharmaceutical composition of 1.0 mg/mL for administration. The self-replicating RNA-containing pharmaceutical composition is loaded into a sterile syringe equipped with a 30-gauge needle.

Using the 30-gauge needle, the physician administers the self-replicating RNA-containing pharmaceutical composition directly into the tumor under direct visual and/or computed tomographic (CT) guidance to avoid major blood vessels.

Within thirty minutes of intratumoral injection of the self-replicating RNA-containing pharmaceutical composition, the physician inserts a needle electrode at or near the injection site and transmits an intratumoral pulsed electric field. The electric field is transmitted by generating a series of eight square waveforms of 50 ms in duration each and 500 V in amplitude. Pulses are delivered about every one second. The treatment is complete upon transmission of the pulsed electric field.

The patient is monitored weekly after treatment for tumor progression by CT. A reduction in tumor volume (i.e., less than 10 cm³) confirms that the treatment is effective

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following. In general, the principles of the invention and including such departures from the present disclosure that come within known or customary practice within the art to which the invention pertains and may be applied to the essential features hereinbefore set forth, and follows in the scope of the claims.

### Numerated Embodiments

Some embodiments of the technology described herein can be defined according to any of the following numbered paragraphs:
1. A nucleic acid vector comprising a dendritic cell chemoattractant-encoding gene, a dendritic cell growth factor or activator-encoding gene, and a lymphocyte signaling protein-encoding gene.
2. The nucleic acid vector of paragraph 1, wherein the dendritic cell chemoattractant-encoding gene is XCL1, XCL2, CCL5, or CCL4.
3. The nucleic acid vector of paragraph 1 or 2, wherein the dendritic cell growth factor or activator is FLT3L, GMCSF, or CD40L
4. The nucleic acid vector of any one of paragraphs 1-3, wherein the lymphocyte-signaling protein is IL-12. IL-15, CXCL9, or CXCL10.
5. The nucleic acid vector of any one of paragraphs 1-4, wherein the nucleic acid vector comprises a first, second, and third promoter driving expression of the dendritic cell chemoattractant-encoding gene, the dendritic cell growth factor or activator-encoding gene, and the lymphocyte signaling protein-encoding gene, respectively.
8. The nucleic acid vector of any one of paragraphs 1-4, wherein the nucleic acid vector comprises a single promoter driving expression of the dendritic cell chemoattractant-encoding gene, the dendritic cell growth factor or activator-encoding gene, and the lymphocyte signaling protein-encoding gene.
7. A nucleic acid vector comprising an XCL1-encoding gene, a FLT3L-encoding gene, and an IL-12-encoding gene.
8. A nucleic acid vector comprising an XCL1-encoding gene, a FLT3L-encoding gene, and an IL-15-encoding gene.
9. A nucleic acid vector comprising an XCL1-encoding gene, a GMCSF-encoding gene, and an IL-15-encoding gene.
10. The nucleic acid vector of any one of paragraphs 1-9, which is a DNA vector.
11. The nucleic acid vector of paragraph 10, which is a circular DNA vector.
12. The nucleic acid vector of paragraph 11, wherein the circular DNA vector lacks a bacterial origin of replication and/or a drug resistance gene.
13. The nucleic acid vector of any one of paragraphs 1-12, wherein the nucleic acid vector is 2.5 kb to 20 kb in length.
14. The nucleic acid vector or circular DNA vector of paragraph 13, wherein the nucleic acid vector is 3.5 kb to 10 kb in length.
15. A circular DNA vector comprising, in a 5′ to 3′ direction:
(a) a promoter;
(b) a self-replicating RNA molecule-encoding sequence comprising (i) a replicase-encoding sequence that transcribes RNA from the self-replicating RNA molecule and (ii) one or more heterologous protein-encoding genes; and
(c) a polyadenylation sequence, wherein the circular DNA vector lacks a bacterial origin of replication and/or a drug resistance gene.
16. The circular DNA vector of paragraph 15, wherein the one or more heterologous protein-encoding genes comprise one or more immunomodulatory protein-encoding genes.
17. The circular DNA vector of paragraph 16, wherein the one or more immunomodulatory protein-encoding genes comprises a dendritic cell chemoattractant-encoding gene, a dendritic cell growth factor or activator-encoding gene, and/or a lymphocyte signaling protein-encoding gene.
18. The circular DNA vector of paragraph 17, wherein the dendritic cell chemoattractant-encoding gene is XCL1, XCL2, CCL5, or CCL4.
19. The circular DNA vector of paragraph 17 or 18, wherein the dendritic cell growth factor or activator is FLT3L, GMCSF, or CD40L.
20. The circular DNA vector of any one of paragraphs 17-19, wherein the lymphocyte-signaling protein is IL-12, IL-15, CXCL9, or CXC110.
21. The circular DNA of any one of paragraphs 15-20, wherein the nucleic acid vector is 10 kb to 13 kb in length.
22. The circular DNA vector of paragraph 21, wherein the nucleic acid vector is about 11.5 kb in length.
23. A pharmaceutical composition comprising: (a) the nucleic acid vector of any one of paragraphs 1-14 or the circular DNA vector of any one of paragraphs 15-22 and (b) a pharmaceutically acceptable carrier.
24. A method of treating a cancer in an individual, the method comprising administering the nucleic acid vector of any one of paragraphs 1-14, the circular DNA vector of any one of paragraphs 15-22, or the pharmaceutical composition of paragraph 23 to the individual in an effective amount to treat the cancer.
25. A method of modulating a tumor microenvironment in an individual in need thereof, the method comprising:
(a) administering the nucleic acid vector of any one of paragraphs 1-14, the circular DNA vector of any one of paragraphs 15-22, or the pharmaceutical composition of paragraph 23 to the individual; and
(b) transmitting an electric field into the tumor microenvironment, wherein the electric field promotes transfer of the nucleic acid molecule or the circular DNA vector into a tumor cell, thereby delivering a modulatory protein-encoding gene to the tumor cell to modulate the tumor microenvironment in the individual.
26. The method of paragraph 25, wherein step (b) comprises transmitting a pulsed electric field.
27. The method of paragraph 26, wherein the pulsed electric field is transmitted through an intratumorally positioned electrode.
28. The method of any one of paragraphs 24-27, wherein the nucleic acid vector, the circular DNA vector, or the pharmaceutical composition is administered intratumorally.
29. The method of any one of paragraphs 24-27, wherein the nucleic acid vector, the circular DNA vector, or the pharmaceutical composition is administered systemically.
30. The method of any one of paragraphs 24-29, wherein the nucleic acid vector, the circular DNA vector, or the pharmaceutical composition is administered in combination with an additional anti-cancer therapy.
31. A method of modulating a tumor microenvironment in an individual in need thereof, the method comprising:
(a) administering a non-viral nucleic acid vector comprising a dendritic cell chemoattractant-encoding gene; and
(b) transmitting an electric field into the tumor microenvironment, wherein the electric field promotes transfer of the non-viral nucleic acid vector into a tumor cell, thereby delivering the dendritic cell chemoattractant-encoding gene to the tumor cell to modulate the tumor microenvironment in the individual.
32. The method of paragraph 31, wherein step (b) comprises transmitting a pulsed electric field.
33. The method of paragraph 32, wherein the pulsed electric field is transmitted through an intratumorally positioned electrode.
34. The method of any one of paragraphs 31-33, wherein the non-viral nucleic acid vector is administered intratumorally.
35. The method of any one of paragraphs 31-33, wherein the non-viral nucleic acid vector is administered systemically.
36. The method of any one of paragraphs 31-35, wherein the non-viral nucleic acid vector is administered in combination with an additional anti-cancer therapy.

Other embodiments of the technology described herein can be defined according to any of the following numbered paragraphs:
1. An immunomodulatory self-replicating RNA molecule comprising (a) a replicase-encoding sequence that transcribes RNA from the self-replicating RNA molecule and (b) a polycistronic immunomodulatory sequence comprising a dendritic cell chemoattractant-encoding gene and one or more immunomodulatory protein-encoding genes.
2. The immunomodulatory self-replicating RNA molecule of paragraph 1, wherein the one or more additional immunomodulatory protein-encoding genes comprises a dendritic cell growth factor or activator-encoding gene.
3. The immunomodulatory self-replicating RNA molecule of paragraph 1 or 2, wherein the one or more additional immunomodulatory protein-encoding genes comprises a lymphocyte signaling protein-encoding gene.
4. The immunomodulatory self-replicating RNA molecule of paragraph 1, wherein the immunomodulatory sequence comprises a dendritic cell chemoattractant-encoding gene, a dendritic cell growth factor or activator-encoding gene, and a lymphocyte signaling protein-encoding gene.
5. The immunomodulatory self-replicating RNA molecule of any one of paragraphs 1-4, wherein the dendritic cell chemoattractant-encoding gene is XCL1, XCL2, CCL5, or CCL4.
6. The immunomodulatory self-replicating RNA molecule of paragraph 2 or 4, wherein the dendritic cell growth factor or activator is FLT3L, GMCSF, or CD40L.
7. The immunomodulatory self-replicating RNA molecule of paragraph 3 or 4, wherein the lymphocyte-signaling protein is IL-12, IL-15, CXCL9, or CXCL10.
8. An immunomodulatory self-replicating RNA molecule comprising (a) a replicase that transcribes RNA from the self-replicating RNA molecule; and (b) a tricistronic immunomodulatory sequence comprising an XCL1-encoding gene, a FLT3L-encoding gene, and an IL-12-encoding gene.
9. An immunomodulatory self-replicating RNA molecule comprising (a) a replicase that transcribes RNA from the self-replicating RNA molecule; and (b) a tricistronic immunomodulatory sequence comprising an XCL1-encoding gene, a FLT3L-encoding gene, and an IL-15-encoding gene.
10. An immunomodulatory self-replicating RNA molecule comprising (a) a replicase that transcribes RNA from the self-replicating RNA molecule; and (b) a tricistronic immunomodulatory sequence comprising an XCL1-encoding gene, a GMCSF-encoding gene, and an IL-15-encoding gene.
11. The immunomodulatory self-replicating RNA molecule of any one of paragraphs 1-10, wherein the self-replicating RNA molecule is 5 kb to 20 kb in length.
12. The immunomodulatory self-replicating RNA molecule of paragraph 11, wherein the self-replicating RNA molecule is 9 kb to 12 kb in length.
13. The immunomodulatory self-replicating RNA molecule of any one of paragraphs 1-12, wherein the self-replicating RNA molecule comprises a 5 cap.
14. The immunomodulatory self-replicating RNA molecule of any one of paragraphs 1-13, wherein the self-replicating RNA molecule comprises an elongated polyadenylation sequence.
15. A pharmaceutical composition comprising: (a) the self-replicating RNA molecule of any one of paragraphs 1-14 and (b) a pharmaceutically acceptable carrier.
16. A method of treating a cancer in an individual, the method comprising administering the immunomodulatory self-replicating RNA molecule of any one of paragraphs 1-14 or the pharmaceutical composition of paragraph 15 to the individual in an effective amount to treat the cancer.
17. The method of paragraph 16, wherein the immunomodulatory self-replicating RNA molecule or the pharmaceutical composition is administered intratumorally.
18. The method of paragraph 16, wherein the immunomodulatory self-replicating RNA molecule or the pharmaceutical composition is administered systemically.
19. The method of any one of paragraphs 16-18, wherein the immunomodulatory self-replicating RNA molecule is administered in combination with a pulsed electric field therapy.
20. The method of paragraph 19, wherein the immunomodulatory self-replicating RNA molecule is administered in combination with an additional anti-cancer therapy.
21. A method of modulating a tumor microenvironment in an individual in need thereof, the method comprising:
(a) administering a self-replicating RNA molecule to the individual, wherein the self-replicating RNA molecule comprises: (i) a replicase-encoding sequence that transcribes RNA from the self-replicating RNA molecule; and (ii) a heterologous modulatory gene; and
(b) transmitting an electric field into the tumor microenvironment, wherein the electric field promotes transfer of the self-replicating RNA molecule into a tumor cell, thereby delivering the heterologous modulatory gene to the tumor cell to modulate the tumor microenvironment in the individual.
22. The method of paragraph 21, wherein the heterologous modulatory gene is an immunomodulatory protein-encoding gene.
23. The method of paragraph 22, wherein the immunomodulatory protein is a chemoattractant.
24. The method of paragraph 23, wherein the chemoattractant is a dendritic cell chemoattractant.
25. The method of paragraph 24, wherein the dendritic cell chemoattractant is XCL1, XCL2, CCL5, or CCL4.
26. The method of any one of paragraphs 21-25, wherein the self-replicating RNA molecule further comprises one or more additional immunomodulatory protein-encoding genes.
27. The method of paragraph 26, wherein the one or more additional immunomodulatory proteins is a dendritic cell growth factor or activator.
28. The method of paragraph 26 or 27, wherein the one or more additional immunomodulatory proteins is a lymphocyte signaling protein.
29. The method of paragraph 28, wherein the self-replicating RNA molecule comprises an XCL1-encoding gene, a dendritic cell growth factor or activator-encoding gene, and a lymphocyte signaling protein-encoding gene.
30. The method of paragraph 29, wherein the dendritic cell growth factor or activator is FLT3L, GMCSF, or CD40L.
31. The method of paragraph 29 or 30, wherein the lymphocyte signaling protein is IL-12, IL-15, CXCL9, or CXCL10.
32. The method of paragraph 31, wherein the self-replicating RNA molecule comprises an XCL1-encoding gene, a FLT3L-encoding gene, and an IL-12-encoding gene.
33. The method of paragraph 31, wherein the self-replicating RNA molecule comprises an XCL1-encoding gene, a FLT3L-encoding gene, and an IL-15-encoding gene.
34. The method of paragraph 31, wherein the self-replicating RNA molecule comprises an XCL1-encoding gene, a GMCSF-encoding gene, and an IL-12-encoding gene.
35. The method of any one of paragraphs 21-34, wherein step (a) comprises intratumoral administration.
36. The method of any one of paragraphs 21-34, wherein step (a) comprises systemic administration.
37. The method of any one of paragraphs 21-36, wherein step (b) comprises transmitting a pulsed electric field.
38. The method of paragraph 37, wherein the pulsed electric field is transmitted through an intratumorally positioned electrode.

Other embodiments are within the claims.

What is claimed is:

1. A nucleic acid vector comprising a dendritic cell chemoattractant-encoding gene, a dendritic cell growth factor or activator-encoding gene, and a lymphocyte signaling protein-encoding gene.

2. The nucleic add vector of claim 1, wherein the dendritic cell chemoattractant-encoding gene is XCL1, XCL2, CCL5, or CCL4.

3. The nucleic acid vector of claim 1 or 2, wherein the dendritic cell growth factor or activator is FLT3L, GMCSF, or CD40.

4. The nucleic acid vector of any one of claims 1-3, wherein the lymphocyte-signaling protein is IL-12, IL-15, CXCL9, or CXCL10.

5. The nucleic acid vector of any one of claims 1-4, wherein the nucleic acid vector comprises a first, second, and third promoter driving expression of the dendritic cell chemoattractant-encoding gene, the dendritic cell growth factor or activator-encoding gene, and the lymphocyte signaling protein-encoding gene, respectively.

6. The nucleic acid vector of any one of claims 1-4, wherein the nucleic acid vector comprises a single promoter driving expression of the dendritic cell chemoattractant-encoding gene, the dendritic cell growth factor or activator-encoding gene, and the lymphocyte signaling protein-encoding gene.

7. A nucleic acid vector comprising an XCL1-encoding gene, a FLT3L-encoding gene, and an IL-12-encoding gene.

8. A nucleic acid vector comprising an XCL1-encoding gene, a FLT3L-encoding gene, and an IL-15-encoding gene.

9. A nucleic acid vector comprising an XCL1-encoding gene, a GMCSF-encoding gene, and an IL-15-encoding gene.

10. The nucleic acid vector of any one of claims 1-9, which is a DNA vector.

11. The nucleic acid vector of claim 10, which is a circular DNA vector.

12. The nucleic acid vector of claim 11, wherein the circular DNA vector is a synthetic circular DNA vector which lacks a bacterial origin of replication, a drug resistance gene, and a recombination site.

13. The nucleic acid vector of any one of claims 1-12, wherein the nucleic acid vector is 2.5 kb to 20 kb in length.

14. The nucleic acid vector of claim 13, wherein the nucleic acid vector is 3.5 kb to 10 kb in length.

15. The nucleic acid vector of any one of claims 1-14, comprising, in a 5′ to 3′ direction, the dendritic cell growth factor or activator-encoding gene, the lymphocyte signaling protein-encoding gene, and the dendritic cell chemoattractant-encoding gene.

16. A circular DNA vector comprising, in a 5′ to 3′ direction:
(a) a promoter;
(b) a self-replicating RNA molecule-encoding sequence comprising (i) a replicase-encoding sequence that transcribes RNA from the self-replicating RNA molecule and (ii) one or more heterologous protein-encoding sequences; and
(c) a polyadenylation sequence,
wherein the circular DNA vector lacks a bacterial origin of replication and/or a drug resistance gene.

17. The circular DNA vector of claim 16, wherein the circular DNA vector is a synthetic circular DNA vector lacking a recombination site.

18. The circular DNA vector of claim 16 or 17, wherein the one or more heterologous protein-encoding sequences comprise one or more immunomodulatory protein-encoding genes.

19. The circular DNA vector of claim 18, wherein the one or more immunomodulatory protein-encoding genes comprises a dendritic cell chemoattractant-encoding gene, a dendritic cell growth factor or activator-encoding gene, and/or a lymphocyte signaling protein-encoding gene.

20. The circular DNA vector of claim 19, wherein the dendritic cell chemoattractant-encoding gene is XCL1, XCL2, CCL5, or CCL4.

21. The circular DNA vector of claim 19 or 20, wherein the dendritic cell growth factor or activator is FLT3L, GMCSF, or CD40.

22. The circular DNA vector of any one of claims 19-21, wherein the lymphocyte-signaling protein is IL-12, IL-15, CXCL9, or CXCL10.

23. The circular DNA of any one of claims 16-22, wherein the nucleic acid vector is 10 kb to 13 kb in length.

24. The circular DNA vector of claim 23, wherein the nucleic acid vector is about 11.5 kb in length.

25. The circular DNA vector of any one of claims 16-24, comprising, in a 5′ to 3′ direction, the dendritic cell growth factor or activator-encoding gene, the lymphocyte signaling protein-encoding gene, and the dendritic cell chemoattractant-encoding gene.

26. A pharmaceutical composition comprising: (a) the nucleic acid vector of any one of claims 1-15 or the circular DNA vector of any one of claims 18-25 and (b) a pharmaceutically acceptable carrier.

27. A method of treating a cancer in an individual, the method comprising administering the nucleic acid vector of any one of claims 1-15, the circular DNA vector of any one of claims 16-25, or the pharmaceutical composition of claim 26 to the individual in an effective amount to treat the cancer.

28. A method of modulating a tumor microenvironment in an individual in need thereof, the method comprising:
(a) administering the nucleic acid vector of any one of claims 1-15, the circular DNA vector of any one of claims 16-25, or the pharmaceutical composition of claim 26 to the individual; and
(b) transmitting an electric field into the tumor microenvironment, wherein the electric field promotes transfer of the nucleic acid molecule or the circular DNA vector into a tumor cell, thereby delivering a modulatory protein-encoding gene to the tumor cell to modulate the tumor microenvironment in the individual.

29. The method of claim 28, wherein step (b) comprises transmitting a pulsed electric field.

30. The method of claim 29, wherein the pulsed electric field is transmitted through an intratumorally positioned electrode.

31. The method of any one of claims 27-30, wherein the nucleic acid vector, the circular DNA vector, or the pharmaceutical composition is administered intratumorally.

32. The method of any one of claims 27-31, wherein the nucleic acid vector, the circular DNA vector, or the pharmaceutical composition is administered systemically.

33. The method of any one of claims 27-32, wherein the nucleic acid vector, the circular DNA vector, or the pharmaceutical composition is administered in combination with an additional anti-cancer therapy.

34. A method of modulating a tumor microenvironment in an individual in need thereof, the method comprising:
(a) administering a non-viral nucleic acid vector comprising a dendritic cell chemoattractant-encoding gene; and
(b) transmitting an electric field into the tumor microenvironment, wherein the electric field promotes transfer of the non-viral nucleic acid vector into a tumor cell, thereby delivering the dendritic cell chemoattractant-encoding gene to the tumor cell to modulate the tumor microenvironment in the individual.

35. The method of claim 34, wherein step (b) comprises transmitting a pulsed electric field.

36. The method of claim 35, wherein the pulsed electric field is transmitted through an intratumorally positioned electrode.

37. The method of any one of claims 34-36, wherein the non-viral nucleic acid vector is administered intratumorally.

38. The method of any one of claims 34-36, wherein the non-viral nucleic acid vector is administered systemically.

39. The method of any one of claims 34-38, wherein the non-viral nucleic acid vector is administered in combination with an additional anti-cancer therapy.

40. A method of expressing a protein in a tumor microenvironment in an individual in need thereof, the method comprising:
(a) administering a synthetic circular DNA vector encoding the protein; and
(b) transmitting an electric field into the tumor microenvironment, wherein the electric field promotes transfer of the synthetic circular DNA vector into a tumor cell, thereby delivering the protein-encoding sequence to the tumor cell to be expressed in the tumor microenvironment in the individual.

41. The method of claim 40, wherein step (b) comprises transmitting a pulsed electric field.

42. The method of claim 41, wherein the pulsed electric field is transmitted through an intratumorally positioned electrode.

43. The method of any one of claims 40-42, wherein the synthetic circular DNA vector is administered intratumorally.

44. The method of any one of claims 40-43, wherein the synthetic circular DNA vector is administered systemically.

45. The method of any one of claims 40-44, wherein the synthetic circular DNA vector is administered in combination with an additional anti-cancer therapy.

46. The method of any one of claims 40-45, wherein the protein is selected from the group consisting of a dendritic cell chemoattractant-encoding gene, a dendritic cell growth factor or activator-encoding gene, and a lymphocyte signaling protein-encoding gene.

47. The method of any one of claims 40-45, wherein the synthetic circular DNA vector encodes a dendritic cell chemoattractant-encoding gene, a dendritic cell growth factor or activator-encoding gene, and a lymphocyte signaling protein-encoding gene.

48. The method of any one of claims 40-47, wherein the synthetic circular DNA vector does not comprise an origin of replication, a drug resistance gene, or a recombination site.

49. The method of any one of claims 40-48, wherein the synthetic circular DNA vector does not comprise a bacterial backbone.


SEQUENCE LISTING (56 sequences)

SEQ ID NO: 1 - RNA, 1605 nt, Venezuelan equine encephalitis virus
auggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcucgccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgagaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggcc

SEQ ID NO: 2 - protein, 535 aa, Venezuelan equine encephalitis virus
MEKVHVDIEEDSPFLRALQRSFPQFEVEAKQVTDNDHANARAFSHLASKLIETEVDPSDTILDIGSAPARRMYSKHKYHCICPMRCAEDPDRLYKYATKLKKNCKEITDKELDKKMKELAAVMSDPDLETETMCLHDDESCRYEGQVAVYQDVYAVDGPTSLYHQANKGVRVAYWIGFDTTPFMFKNLAGAYPSYSTNWADETVLTARNIGLCSSDVMERSRRGMSILRKKYLKPSNNVLFSVGSTIYHEKRDLLRSWHLPSVFHLRGKQNYTCRCETIVSCDGYVVKRIAISPGLYGKPSGYAATMHREGFLCCKVTDTLNGERVSFPVCTYVPATLCDQMTGILATDVSADDAQKLLVGLNQRIVVNGRTQRNTNTMKNYLLPVVAQAFARWAKEYKEDQEDERPLGLRDRQLVMGCCWAFRRHKITSIYKRPDTQTIIKVNSDFHSFVLPRIGSNTLEIGLRTRIRKMLEEHKEPSPLITAEDVQEAKCAADEAKEVREAEELRAALPPLAADVEEPTLEADVDLMLQEAGA

SEQ ID NO: 3 - RNA, 2382 nt, Venezuelan equine encephalitis virus
ggcucaguggagacaccucguggcuugauaaagguuaccagcuacgcuggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagaacuagucacugggcuagggcucacaggcgagcugguggauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaagcuuuugcuugucaugcagguacucucagagcgcucauagccauuauaagaccuaaaaaggcagugcucugcggggaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacuggaagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuuagcauguugaccaagaaagcuugucugcaucugaaucccggcggaaccugugucagcauagguuaugguuacgcugacagggccagcgaaagcaucauuggugcuauagcgcggcaguucaaguuuucccggguaugcaaaccgaaauccucacuugaagagacggaaguucuguuuguauucauuggguacgaucgcaaggcccguacgcacaauccuuacaagcuuucaucaaccuugaccaacauuuauacagguuccagacuccacgaagccggaugu

SEQ ID NO: 4 - protein, 794 aa, Venezuelan equine encephalitis virus
GSVETPRGLIKVTSYAGEDKIGSYAVLSPQAVLKSEKLSCIHPLAEQVIVITHSGRKGRYAVEPYHGKVVVPEGHAIPVQDFQALSESATIVYNEREFVNRYLHHIATHGGALNTDEEYYKTVKPSEHDGEYLYDIDRKQCVKKELVTGLGLTGELVDPPFHEFAYESLRTRPAAPYQVPTIGVYGVPGSGKSGIIKSAVTKKDLVVSAKKENCAEIIRDVKKMKGLDVNARTVDSVLLNGCKHPVETLYIDEAFACHAGTLRALIAIIRPKKAVLCGDPKQCGFFNMMCLKVHFNHEICTQVFHKSISRRCTKSVTSVVSTLFYDKKMRTTNPKETKIVIDTTGSTKPKQDDLILTCFRGWVKQLQIDYKGNEIMTAAASQGLTRKGVYAVRYKVNENPLYAPTSEHVNVLLTRTEDRIVWKTLAGDPWIKTLTAKYPGNFTATIEEWQAEHDAIMRHILERPDPTDVFQNKANVCWAKALVPVLKTAGIDMTTEQWNTVDYFETDKAHSAEIVLNQLCVRFFGLDLDSGLFSAPTVPLSIRNNHWDNSPSPNMYGLNKEVVRQLSRRYPQLPRAVATGRVYDMNTGTLRNYDPRINLVPVNRRLPHALVLHHNEHPQSDFSSFVSKLKGRTVLVVGEKLSVPGKMVDWLSDRPEATFRARLDLGIPGDVPKYDIIFVNVRTPYKYHHYQQCEDHAIKLSMLTKKACLHLNPGGTCVSIGYGYADRASESIIGAIARQFKFSRVCKPKSSLEETEVLFVFIGYDRKARTHNPYKLSSTLTNIYTGSRLHEAGC

SEQ ID NO: 5 - RNA, 1671 nt, Venezuelan equine encephalitis virus
gcacccucauaucauguggugcgaggggauauugccacggccaccgaaggagugauuauaaaugcugcuaacagcaaaggacaaccuggcggaggggugugcggagcgcuguauaagaaauucccggaaagcuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgacucuucagugacagaaccugaugcagagcuggugagggugcauccgaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaagagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaggaagaagaggauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcucgagaaccagccuaguuuccaccccgccaggcgugaauagggugaucacuagagaggagcucgaggcgcuuaccccgucacgcacuccuagcaggucggucucgagaaccagccuggucuccaacccgccaggcguaaauagggugauuacaagagaggaguuugaggcguucguagcacaacaacaaugacgguuugaugcgggugca

SEQ ID NO: 6 - protein, 556 aa, Venezuelan equine encephalitis virus
APSYHVVRGDIATATEGVIINAANSKGQPGGGVCGALYKKFPESFDLQPIEVGKARLVKGAAKHIIHAVGPNFNKVSEVEGDKQLAEAYESIAKIVNDNNYKSVAIPLLSTGIFSGNKDRLTQSLNHLLTALDTTDADVAIYCRDKKWEMTLKEAVARREAVEEICISDDSSVTEPDAELVRVHPKSSLAGRKGYSTSDGKTFSYLEGTKFHQAAKDIAEINAMWPVATEANEQVCMYILGESMSSIRSKCPVEESEASTPPSTLPCLCIHAMTPERVQRLKASRPEQITVCSSFPLPKYRITGVQKIQCSQPILFSPKVPAYIHPRKYLVETPPVDETPEPSAENQSTEGTPEQPPLITEDETRTRTPEPIIIEEEEEDSISLLSDGPTHQVLQVEADIHGPPSVSSSSWSIPHASDFDVDSLSILDTLEGASVTSGATSAETNSYFAKSMEFLARPVPAPRTVFRNPPHPAPRTRTPSLAPSRACSRTSLVSTPPGVNRVITREELEALTPSRTPSRSVSRTSLVSNPPGVNRVITREEFEAFVAQQQRFDAGA

SEQ ID NO: 7 - RNA, 1821 nt, Venezuelan equine encephalitis virus
uacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuuggcaaaccucuggcagcagacgaugaacaugaugaugacaggagaagggcauugcaugaagagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggc

SEQ ID NO: 8 - protein, 607 aa, Venezuelan equine encephalitis virus
YIFSSDTGQGHLQQKSVRQTVLSEVVLERTELEISYAPRLDQEKEELLRKKLQLNPTPANRSRYQSRKVENMKAITARRILQGLGHYLKAEGKVECYRTLHPVPLYSSSVNRAFSSPKVAVEACNAMLKENFPTVASYCIIPEYDAYLDMVDGASCCLDTASFCPAKLRSFPKKHSYLEPTIRSAVPSAIQNTLQNVLAAATKRNCNVTQMRELPVLDSAAFNVECFKKYACNNEYWETFKENPIRLTEENVVNYITKLKGPKAAALFAKTHNLNMLQDIPMDRFVMDLKRDVKVTPGTKHTEERPKVQVIQAADPLATAYLCGIHRELVRRLNAVLLPNIHTLFDMSAEDFDAIIAEHFQPGDCVLETDIASFDKSEDDAMALTALMILEDLGVDAELLTLIEAAFGEISSIHLPTKTKFKFGAMMKSGMFLTLFVNTVINIVIASRVLRERLTGSPCAAFIGDDNIVKGVKSDKLMADRCATWLNMEVKIIDAVVGEKAPYFCGGFILCDSVTGTACRVADPLKRLFKLGKPLAADDEHDDDRRRALHEESTRWNRVGILSELCKAVESRYETVGTSIIVMAMTTLASSVKSFSYLRGAPITLYG

SEQ ID NO: 9 - DNA, 345 bp, Homo sapiens
atgagacttctcatcctggccctccttggcatctgctctctcactgcatacattgtggaaggtgtagggagtgaagtctcagataagaggacctgtgtgagcctcactacccagcgactgccggttagcagaatcaagacctacaccatcacggaaggctccttgagagcagtaatttttattaccaaacgtggcctaaaagtctgtgctgatccacaagccacatgggtgagagacgtggtcaggagcatggacaggaaatccaacaccagaaataacatgatccagaccaagccaacaggaacccagcaatcgaccaatacagctgtgactctgactggctag

SEQ ID NO: 10 - protein, 114 aa, Homo sapiens
MRLLILALLGICSLTAYIVEGVGSEVSDKRTCVSLTTQRLPVSRIKTYTITEGSLRAVIFITKRGLKVCADPQATWVRDVVRSMDRKSNTRNNMIQTKPTGTQQSTNTAVTLTG

SEQ ID NO: 11 - DNA, 345 bp, Homo sapiens
atgagacttctcatcctggccctccttggcatctgctctctcactgcatacattgtggaaggtgtagggagtgaagtctcacataggaggacctgtgtgagcctcactacccagcgactgccagttagcagaatcaagacctacaccatcacggaaggctccttgagagcagtaatttttattaccaaacgtggcctaaaagtctgtgctgatccacaagccacgtgggtgagagacgtggtcaggagcatggacaggaaatccaacaccagaaataacatgatccagaccaagccaacaggaacccagcaatcgaccaatacagctgtgaccctgactggctag

SEQ ID NO: 12 - protein, 114 aa, Homo sapiens
MRLLILALLGICSLTAYIVEGVGSEVSHRRTCVSLTTQRLPVSRIKTYTITEGSLRAVIFITKRGLKVCADPQATWVRDVVRSMDRKSNTRNNMIQTKPTGTQQSTNTAVTLTG

SEQ ID NO: 13 - DNA, 276 bp, Homo sapiens
atgaaggtctccgcggcacgcctcgctgtcatcctcattgctactgccctctgcgctcctgcatctgcctccccatattcctcggacaccacaccctgctgctttgcctacattgcccgcccactgccccgtgcccacatcaaggagtatttctacaccagtggcaagtgctccaacccagcagtcgtctttgtcacccgaaagaaccgccaagtgtgtgccaacccagagaagaaatgggttcgggagtacatcaactctttggagatgagctag

SEQ ID NO: 14 - protein, 91 aa, Homo sapiens
MKVSAAALAVILIATALCAPASASPYSSDTTPCCFAYIARPLPRAHIKEYFYTSGKCSNPAVVFVTRKNRQVCANPEKKWVREYINSLEMS

SEQ ID NO: 15 - DNA, 279 bp, Homo sapiens
atgaagctctgcgtgactgtcctgtctctcctcatgctagtagctgccttctgctctccagcgctctcagcaccaatgggctcagaccctcccaccgcctgctgcttttcttacaccgcgaggaagcttcctcgcaactttgtggtagattactatgagaccagcagcctctgctcccagccagctgtggtattccaaaccaaaagaagcaagcaagtctgtgctgatcccagtgaatcctgggtccaggagtacgtgtatgacctggaactgaactga

SEQ ID NO: 16 - protein, 92 aa, Homo sapiens
MKLCVTVLSLLMLVAAFCSPALSAPMGSDPPTACCFSYTARKLPRNFVVDYYETSSLCSQPAVVFQTKRSKQVCADPSESWVQEYVYDLELN

SEQ ID NO: 17 - DNA, 708 bp, Homo sapiens
atgacagtgctggcgccagcctggagcccaacaacctatctcctcctgctgctgctgctgagctcgggactcagtgggacccaggactgctccttccaacacagccccatctcctccgacttcgctgtcaaaatccgtgagctgtctgactacctgcttcaagattacccagtcaccgtggcctccaacctgcaggacgaggagctctgcggggcgctctggcggctggtcctggcacagcgctggatggagcggctcaagactgtcgctgggtccaagatgcaaggcttgctggagcgcgtgaacacggagatacactttgtcaccaaatgtgcctttcagcccccccccagctgtcttcgcttcgtccagaccaacatctcccgcctcctgcaggagacctccgagcagctggtggcgctgaagccctggatcactcgccagaacttctcccggtgcctggagctgcagtgtcagcccgactcctcaaccctgccacccccatggagtccccggcccctggaggccacagccccgacagccccgcagccccctctgctcctcctactgctgctgcccgtgggcctcctgctgctggccgctgcctggtgcctgcactggcagaggacgcggcggaggacaccccgccctggggagcaggtgccccccgtccccagtccccaggacctgctgcttgtggagcactga

SEQ ID NO: 18 - protein, 235 aa, Homo sapiens
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 19 - DNA, 435 bp, Homo sapiens
atgtggctgcagagcctgctgctcttgggcactgtggcctgcagcatctctgcacccgcccgctcgcccagccccagcacacagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaaacagcactgccctccaaccccggaaacttcctgtgcaacccagattatcacctttgaaagtttcaaagagaacctgaaggactttctgcttgtcatcccctttgactgctgggagccagtccaggagtga

SEQ ID NO: 20 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 21 - DNA, 786 bp, Homo sapiens
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggctggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 22 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 23 - DNA, 660 bp, Homo sapiens
atgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgacagagtgacgagctatctgaatgcttcctaa

SEQ ID NO: 24 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 25 - DNA, 804 bp, Homo sapiens
atggccccgcggcgggcgcgcggctgccggaccctcggtctcccggcgctgctactgctgctgctgctccggccgccggcgacgcggggcatcacgtgccctccccccatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaccctgccctggttcaccaaaggccagcgccaccctccacagtaacgacggcaggggtgaccccacagccagagagcctctccccttctggaaaagagcccgcagcttcatctcccagctcaaacaacacagcggccacaacagcagctattgtcccgggctcccagctgatgccttcaaaatcaccttccacaggaaccacagagataagcagtcatgagtcctcccacggcaccccctctcagacaacagccaagaactgggaactcacagcatccgcctcccaccagccgccaggtgtgtatccacagggccacagcgacaccactgtggctatctccacgtccactgtcctgctgtgtgggctgagcgctgtgtctctcctggcatgctacctcaagtcaaggcaaactcccccgctggccagcgttgaaatggaagccatggaggctctgccggtgacttgggggaccagcagcagagatgaagacttggaaaactgctctcaccacctatga

SEQ ID NO: 26 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 27 - DNA, 378 bp, Homo sapiens
atgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataa

SEQ ID NO: 28 - protein, 125 aa, Homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 29 - DNA, 297 bp, Homo sapiens
atgaatcaaactgcgattctgatttgctgccttatctttctgactctaagtggcattcaaggagtacctctctctagaaccgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaatgtctaaaagatctccttaa

SEQ ID NO: 30 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 31 - DNA, 564 bp, Homo sapiens
atgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactga

SEQ ID NO: 32 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 33 - RNA, 2703 nt, Artificial Sequence
  Description of Artificial Sequence Synthetic polynucleotide
augacagugcuggcuccugcuuggagcccuaacagcucucugcugcugcuucugcuccugcugagcccaugucugagaggcaccccugacugcuacuucucucacagccccaucagcagcaacuucaaagugaaguuccgcgagcugaccgaccaucugcugaaggacuacccugugaccguggccgugaaccugcaggaugagaagcacugcaaggcccuguggucccuguuccuggcucagagauggaucgagcagcugaaaacaguggccggcagcaagaugcagacccugcuggaagaugugaacaccgagauccacuucgugaccagcugcaccuuccagccucugccugagugccugagauucgugcagaccaacaucagccaccuucucaaggacacaugcacccagcugcuggcccugaagccuuguaucggcaaggccugccagaacuucuccagaugccuggaagugcagugccagccugacagcucuacacugcugccuccaagaagcccuaucgcucuggaagccacagagcugccugagccuagaccuagacagagaagaaagagaagcggcuccggcgccaccaacuucagccugcuuaaacaggcaggcgacguggaagagaaccccggaccuaugugcccucagaagcugaccaucaguugguucgccaucgugcugcugguguccccacugauggcuaugugggaacucgagaaggacguguacgugguggaaguggacuggaccccugaugcuccuggcgagacagugaaccugaccugcgacacaccugaagaggacgacaucaccuggaccagcgaucagagacacggcgugaucggcucuggcaagacccugacaauuaccgugaaagaguuccuggacgccggccaguacaccugucacaaaggcggagagacacugagccacucucaucugcugcugcacaagaaagagaacggcaucugguccaccgagauccugaagaacuucaagaacaagaccuuccugaagugcgaggccccuaacuacagcggcagauucaccuguagcuggcuggugcagagaaacauggaccugaaguucaacaucaaguccuccagcagcagccccgacagcagagcugugacauguggcauggcuagccugagcgccgagaaagugacacuggaccagagagacuacgagaaguacagcguguccugccaagaggacgugaccuguccuaccgccgaggaaacacugccuaucgagcuggcccuggaagccagacagcagaacaaauacgagaacuacucuaccagcuucuucauccgggacaucaucaagcccgauccuccaaagaaccugcagaugaagccucugaagaacagccaggucgagguguccugggaguacccugacagcuggucuaccccucacagcuacuucagccugaaauucuucgugcggauccagcgcaagaaagaaaagaugaaggaaaccgaggaaggcugcaaccagaaaggcgcuuuccugguggaaaagaccagcaccgaggugcagugcaaaggcggcaaugucugugugcaggcccaggaccgguacuacaacagcagcuguagcaagugggccugcgugccaugcagagucagaucugguggcggaggaucuggcggagguggaagcggcggaggcggaucuagagugauuccugugucuggcccugccagaugccugagccagucuagaaaccugcugaaaaccaccgacgacauggucaagaccgccagagagaagcugaagcacuacuccugcacagccgaggacaucgaccacgaggauaucaccagggaccagacaagcacccugaaaaccugccugccucuggaacugcauaagaacgagagcugccuggccaccagagaaaccagcucuaccacaagaggcagcugucugccuccucagaaaaccagccugaugaugacccugugccugggcagcaucuacgaggaucugaagauguaccagaccgaguuccaggccaucaacgccgcucugcagaaccacaaccaccagcagaucauccuggacaagggcaugcugguggcuaucgacgagcugaugcagagccugaaccauaacggcgagacacugcggcagaagccuccaguuggagaggccgauccuuacagagugaagaugaagcugugcauccugcugcacgccuucagcaccagaguggucaccaucaacagagugaugggcuaccugagcagcgccaggcggaagcggagcggcagcggcgcuacaaacuuuagccuucugaagcaggccggggaugucgaagagaauccugguccaaugagacugcugcugcugacauuccugggcgugugcugucugacacccuggguugucgaaggcgugggaacagaggugcuggaagaguccagcugcgugaaccugcagacccagagacugcccgugcagaagaucaagaccuacaucaucugggagggcgccaugagagccgugaucuucgugacaaagagaggccugaagaucugcgccgauccugaggccaaaugggucaaagccgccaucaagaccguggacggcagagccagcaccagaaagaacauggccgagacagugccuacaggcgcccagagaucuaccagcacagccaucacacugaccggcuga

SEQ ID NO: 34 - protein, 900 aa, Artificial Sequence
  Description of Artificial Sequence Synthetic polypepide
MTVLAPAWSPNSSLLLLLLLLSPCLRGTPDCYFSHSPISSNFKVKFRELTDHLLKDYPVTVAVNLQDEKHCKALWSLFLAQRWIEQLKTVAGSKMQTLLEDVNTEIHFVTSCTFQPLPECLRFVQTNISHLLKDTCTQLLALKPCIGKACQNFSRCLEVQCQPDSSTLLPPRSPIALEATELPEPRPRQRRKRSGSGATNFSLLKQAGDVEENPGPMCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSARRKRSGSGATNFSLLKQAGDVEENPGPMRLLLLTFLGVCCLTPWVVEGVGTEVLEESSCVNLQTQRLPVQKIKTYIIWEGAMRAVIFVTKRGLKICADPEAKWVKAAIKTVDGRASTRKNMAETVPTGAQRSTSTAITLTG

SEQ ID NO: 35 - DNA, 510 bp, Homo sapiens
atgagaggcactccaggagacgctgatggtggaggaagggccgtctatcaatcaatgtgtaaacctattactgggactattaatgatttgaatcagcaagtgtggacccttcagggtcagaaccttgtggcagttccacgaagtgacagtgtgaccccagtcactgttgctgttatcacatgcaagtatccagaggctcttgagcaaggcagaggggatcccatttatttgggaatccagaatccagaaatgtgtttgtattgtgagaaggttggagaacagcccacattgcagctaaaagagcagaagatcatggatctgtatggccaacccgagcccgtgaaacccttccttttctaccgtgccaagactggtaggacctccacccttgagtctgtggccttcccggactggttcattgcctcctccaagagagaccagcccatcattctgacttcagaacttgggaagtcatacaacactgcctttgaattaaatataaatgactga

SEQ ID NO: 36 - protein, 169 aa, Homo sapiens
MRGTPGDADGGGRAVYQSMCKPITGTINDLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGKSYNTAFELNIND

SEQ ID NO: 37 - DNA, 8035 bp, Artificial Sequence
  Description of Artificial Sequence Synthetic polynucleotide
ctcttcggtatcacaggagaatttcagggagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacagggagtttaaacagataagtttgtacaaaaaagagaggtgccaccatgacagtgctggctcctgcttggagccctaacagctctctgctgctgcttctgctcctgctgagcccatgtctgagaggcacccctgactgctacttctctcacagccccatcagcagcaacttcaaagtgaagttccgcgagctgaccgaccatctgctgaaggactaccctgtgaccgtggccgtgaacctgcaggatgagaagcactgcaaggccctgtggtccctgttcctggctcagagatggatcgagcagctgaaaacagtggccggcagcaagatgcagaccctgctggaagatgtgaacaccgagatccacttcgtgaccagctgcaccttccagcctctgcctgagtgcctgagattcgtgcagaccaacatcagccaccttctcaaggacacatgcacccagctgctggccctgaagccttgtatcggcaaggcctgccagaacttctccagatgcctggaagtgcagtgccagcctgacagctctacactgctgcctccaagaagccctatcgctctggaagccacagagctgcctgagcctagacctagacagtgagcttccactggattgtacaattaccaacaataaaatatctttattttcattacatctgtgtgttggttttttgtgtgcgctactttcggtatcacaggagaatttcagggagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacagggagtttaaacagataagtttgtacaaaaaagagaggtgccaccatgtgccctcagaagctgaccatcagttggttcgccatcgtgctgctggtgtccccactgatggctatgtgggaactcgagaaggacgtgtacgtggtggaagtggactggacccctgatgctcctggcgagacagtgaacctgacctgcgacacacctgaagaggacgacatcacctggaccagcgatcagagacacggcgtgatcggctctggcaagaccctgacaattaccgtgaaagagttcctggacgccggccagtacacctgtcacaaaggcggagagacactgagccactctcatctgctgctgcacaagaaagagaacggcatctggtccaccgagatcctgaagaacttcaagaacaagaccttcctgaagtgcgaggcccctaactacagcggcagattcacctgtagctggctggtgcagagaaacatggacctgaagttcaacatcaagtcctccagcagcagccccgacagcagagctgtgacatgtggcatggctagcctgagcgccgagaaagtgacactggaccagagagactacgagaagtacagcgtgtcctgccaagaggacgtgacctgtcctaccgccgaggaaacactgcctatcgagctggccctggaagccagacagcagaacaaatacgagaactactctaccagcttcttcatccgggacatcatcaagcccgatcctccaaagaacctgcagatgaagcctctgaagaacagccaggtcgaggtgtcctgggagtaccctgacagctggtctacccctcacagctacttcagcctgaaattcttcgtgcggatccagcgcaagaaagaaaagatgaaggaaaccgaggaaggctgcaaccagaaaggcgctttcctggtggaaaagaccagcaccgaggtgcagtgcaaaggcggcaatgtctgtgtgcaggcccaggaccggtactacaacagcagctgtagcaagtgggcctgcgtgccatgcagagtcagatctggtggcggaggatctggcggaggtggaagcggcggaggcggatctagagtgattcctgtgtctggccctgccagatgcctgagccagtctagaaacctgctgaaaaccaccgacgacatggtcaagaccgccagagagaagctgaagcactactcctgcacagccgaggacatcgaccacgaggatatcaccagggaccagacaagcaccctgaaaacctgcctgcctctggaactgcataagaacgagagctgcctggccaccagagaaaccagctctaccacaagaggcagctgtctgcctcctcagaaaaccagcctgatgatgaccctgtgcctgggcagcatctacgaggatctgaagatgtaccagaccgagttccaggccatcaacgccgctctgcagaaccacaaccaccagcagatcatcctggacaagggcatgctggtggctatcgacgagctgatgcagagcctgaaccataacggcgagacactgcggcagaagcctccagttggagaggccgatccttacagagtgaagatgaagctgtgcatcctgctgcacgccttcagcaccagagtggtcaccatcaacagagtgatgggctacctgagcagcgcctgagcttccactggattgtacaattaccaacaataaaatatctttattttcattacatctgtgtgttggttttttgtgtgcgctatattcggtatcacaggagaatttcagggagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagacaattgtactaaccttcttctctttcctctcctgacagggagtttaaacagataagtttgtacaaaaaagagaggtgccaccatgagactgctgctgctgacattcctgggcgtgtgctgtctgacaccctgggttgtcgaaggcgtgggaacagaggtgctggaagagtccagctgcgtgaacctgcagacccagagactgcccgtgcagaagatcaagacctacatcatctgggagggcgccatgagagccgtgatcttcgtgacaaagagaggcctgaagatctgcgccgatcctgaggccaaatgggtcaaagccgccatcaagaccgtggacggcagagccagcaccagaaagaacatggccgagacagtgcctacaggcgcccagagatctaccagcacagccatcacactgaccggctgagcttccactggattgtacaattaccaacaataaaatatctttattttcattacatctgtgtgttggttttttgtgtgcgctcaaaaaa

SEQ ID NO: 38 - DNA, 1605 bp, Venezuelan equine encephalitis virus
atggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggcc

SEQ ID NO: 39 - DNA, 2382 bp, Venezuelan equine encephalitis virus
ggctcagtggagacacctcgtggcttgataaaggttaccagctacgctggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgt

SEQ ID NO: 40 - DNA, 1671 bp, Venezuelan equine encephalitis virus
gcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgca

SEQ ID NO: 41 - DNA, 1821 bp, Venezuelan equine encephalitis virus
tacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggc

SEQ ID NO: 42 - RNA, 345 nt, Homo sapiens
augagacuucucauccuggcccuccuuggcaucugcucucucacugcauacauuguggaagguguagggagugaagucucagauaagaggaccugugugagccucacuacccagcgacugccgguuagcagaaucaagaccuacaccaucacggaaggcuccuugagagcaguaauuuuuauuaccaaacguggccuaaaagucugugcugauccacaagccacaugggugagagacguggucaggagcauggacaggaaauccaacaccagaaauaacaugauccagaccaagccaacaggaacccagcaaucgaccaauacagcugugacucugacuggcuag

SEQ ID NO: 43 - RNA, 345 nt, Homo sapiens
augagacuucucauccuggcccuccuuggcaucugcucucucacugcauacauuguggaagguguagggagugaagucucacauaggaggaccugugugagccucacuacccagcgacugccaguuagcagaaucaagaccuacaccaucacggaaggcuccuugagagcaguaauuuuuauuaccaaacguggccuaaaagucugugcugauccacaagccacgugggugagagacguggucaggagcauggacaggaaauccaacaccagaaauaacaugauccagaccaagccaacaggaacccagcaaucgaccaauacagcugugacccugacuggcuag

SEQ ID NO: 44 - RNA, 276 nt, Homo sapiens
augaaggucuccgcggcacgccucgcugucauccucauugcuacugcccucugcgcuccugcaucugccuccccauauuccucggacaccacacccugcugcuuugccuacauugcccgcccacugccccgugcccacaucaaggaguauuucuacaccaguggcaagugcuccaacccagcagucgucuuugucacccgaaagaaccgccaagugugugccaacccagagaagaaauggguucgggaguacaucaacucuuuggagaugagcuag

SEQ ID NO: 45 - RNA, 279 nt, Homo sapiens
augaagcucugcgugacuguccugucucuccucaugcuaguagcugccuucugcucuccagcgcucucagcaccaaugggcucagacccucccaccgccugcugcuuuucuuacaccgcgaggaagcuuccucgcaacuuugugguagauuacuaugagaccagcagccucugcucccagccagcugugguauuccaaaccaaaagaagcaagcaagucugugcugaucccagugaauccuggguccaggaguacguguaugaccuggaacugaacuga

SEQ ID NO: 46 - RNA, 708 nt, Homo sapiens
augacagugcuggcgccagccuggagcccaacaaccuaucuccuccugcugcugcugcugagcucgggacucagugggacccaggacugcuccuuccaacacagccccaucuccuccgacuucgcugucaaaauccgugagcugucugacuaccugcuucaagauuacccagucaccguggccuccaaccugcaggacgaggagcucugcggggcgcucuggcggcugguccuggcacagcgcuggauggagcggcucaagacugucgcuggguccaagaugcaaggcuugcuggagcgcgugaacacggagauacacuuugucaccaaaugugccuuucagcccccccccagcugucuucgcuucguccagaccaacaucucccgccuccugcaggagaccuccgagcagcugguggcgcugaagcccuggaucacucgccagaacuucucccggugccuggagcugcagugucagcccgacuccucaacccugccacccccauggaguccccggccccuggaggccacagccccgacagccccgcagcccccucugcuccuccuacugcugcugcccgugggccuccugcugcuggccgcugccuggugccugcacuggcagaggacgcggcggaggacaccccgcccuggggagcaggugccccccguccccaguccccaggaccugcugcuuguggagcacuga

SEQ ID NO: 47 - RNA, 435 nt, Homo sapiens
auguggcugcagagccugcugcucuugggcacuguggccugcagcaucucugcacccgcccgcucgcccagccccagcacacagcccugggagcaugugaaugccauccaggaggcccggcgucuccugaaccugaguagagacacugcugcugagaugaaugaaacaguagaagucaucucagaaauguuugaccuccaggagccgaccugccuacagacccgccuggagcuguacaagcagggccugcggggcagccucaccaagcucaagggccccuugaccaugauggccagccacuacaaacagcacugcccuccaaccccggaaacuuccugugcaacccagauuaucaccuuugaaaguuucaaagagaaccugaaggacuuucugcuugucauccccuuugacugcugggagccaguccaggaguga

SEQ ID NO: 48 - RNA, 786 nt, Homo sapiens
augaucgaaacauacaaccaaacuucuccccgaucugcggccacuggacugcccaucagcaugaaaauuuuuauguauuuacuuacuguuuuucuuaucacccagaugauugggucagcacuuuuugcuguguaucuucauagaaggcuggacaagauagaagaugaaaggaaucuucaugaagauuuuguauucaugaaaacgauacagagaugcaacacaggagaaagauccuuauccuuacugaacugugaggagauuaaaagccaguuugaaggcuuugugaaggauauaauguuaaacaaagaggagacgaagaaagaaaacagcuuugaaaugcaaaaaggugaucagaauccucaaauugcggcacaugucauaagugaggccagcaguaaaacaacaucuguguuacagugggcugaaaaaggauacuacaccaugagcaacaacuugguaacccuggaaaaugggaaacagcugaccguuaaaagacaaggacucuauuauaucuaugcccaagucaccuucuguuccaaucgggaagcuucgagucaagcuccauuuauagccagccucugccuaaagucccccgguagauucgagagaaucuuacucagagcugcaaauacccacaguuccgccaaaccuugcgggcaacaauccauucacuugggaggaguauuugaauugcaaccaggugcuucgguguuugucaaugugacugauccaagccaagugagccauggcacuggcuucacguccuuuggcuuacucaaacucuga

SEQ ID NO: 49 - RNA, 660 nt, Homo sapiens
auguguccagcgcgcagccuccuccuuguggcuacccugguccuccuggaccaccucaguuuggccagaaaccuccccguggccacuccagacccaggaauguucccaugccuucaccacucccaaaaccugcugagggccgucagcaacaugcuccagaaggccagacaaacucuagaauuuuacccuugcacuucugaagagauugaucaugaagauaucacaaaagauaaaaccagcacaguggaggccuguuuaccauuggaauuaaccaagaaugagaguugccuaaauuccagagagaccucuuucauaacuaaugggaguugccuggccuccagaaagaccucuuuuaugauggcccugugccuuaguaguauuuaugaagacuugaagauguaccagguggaguucaagaccaugaaugcaaagcuucugauggauccuaagaggcagaucuuucuagaucaaaacaugcuggcaguuauugaugagcugaugcaggcccugaauuucaacagugagacugugccacaaaaauccucccuugaagaaccggauuuuuauaaaacuaaaaucaagcucugcauacuucuucaugcuuucagaauucgggcagugacuauugacagagugacgagcuaucugaaugcuuccuaa

SEQ ID NO: 50 - RNA, 804 nt, Homo sapiens
auggccccgcggcgggcgcgcggcugccggacccucggucucccggcgcugcuacugcugcugcugcuccggccgccggcgacgcggggcaucacgugcccuccccccauguccguggaacacgcagacaucugggucaagagcuacagcuuguacuccagggagcgguacauuuguaacucugguuucaagcguaaagccggcacguccagccugacggagugcguguugaacaaggccacgaaugucgcccacuggacaacccccagucucaaaugcauuagagacccugcccugguucaccaaaggccagcgccacccuccacaguaacgacggcaggggugaccccacagccagagagccucuccccuucuggaaaagagcccgcagcuucaucucccagcucaaacaacacagcggccacaacagcagcuauugucccgggcucccagcugaugccuucaaaaucaccuuccacaggaaccacagagauaagcagucaugaguccucccacggcacccccucucagacaacagccaagaacugggaacucacagcauccgccucccaccagccgccagguguguauccacagggccacagcgacaccacuguggcuaucuccacguccacuguccugcugugugggcugagcgcugugucucuccuggcaugcuaccucaagucaaggcaaacucccccgcuggccagcguugaaauggaagccauggaggcucugccggugacuugggggaccagcagcagagaugaagacuuggaaaacugcucucaccaccuauga

SEQ ID NO: 51 - RNA, 378 nt, Homo sapiens
augaagaaaagugguguucuuuuccucuugggcaucaucuugcugguucugauuggagugcaaggaaccccaguagugagaaagggucgcuguuccugcaucagcaccaaccaagggacuauccaccuacaauccuugaaagaccuuaaacaauuugccccaagcccuuccugcgagaaaauugaaaucauugcuacacugaagaauggaguucaaacaugucuaaacccagauucagcagaugugaaggaacugauuaaaaagugggagaaacaggucagccaaaagaaaaagcaaaagaaugggaaaaaacaucaaaaaaagaaaguucugaaaguucgaaaaucucaacguucucgucaaaagaagacuacauaa

SEQ ID NO: 52 - RNA, 297 nt, Homo sapiens
augaaucaaacugcgauucugauuugcugccuuaucuuucugacucuaaguggcauucaaggaguaccucucucuagaaccguacgcuguaccugcaucagcauuaguaaucaaccuguuaauccaaggucuuuagaaaaacuugaaauuauuccugcaagccaauuuuguccacguguugagaucauugcuacaaugaaaaagaagggugagaagagaugucugaauccagaaucgaaggccaucaagaauuuacugaaagcaguuagcaaggaaaugucuaaaagaucuccuuaa

SEQ ID NO: 53 - RNA, 564 nt, Homo sapiens
augaccaacaagugucuccuccaaauugcucuccuguugugcuucuccacuacagcucuuuccaugagcuacaacuugcuuggauuccuacaaagaagcagcaauuuucagugucagaagcuccuguggcaauugaaugggaggcuugaauacugccucaaggacaggaugaacuuugacaucccugaggagauuaagcagcugcagcaguuccagaaggaggacgccgcauugaccaucuaugagaugcuccagaacaucuuugcuauuuucagacaagauucaucuagcacuggcuggaaugagacuauuguugagaaccuccuggcuaaugucuaucaucagauaaaccaucugaagacaguccuggaagaaaaacuggagaaagaagauuucaccaggggaaaacucaugagcagucugcaccugaaaagauauuaugggaggauucugcauuaccugaaggccaaggaguacagucacugugccuggaccauagucagaguggaaauccuaaggaacuuuuacuucauuaacagacuuacagguuaccuccgaaacuga

SEQ ID NO: 54 - DNA, 2703 bp, Artificial Sequence
  Description of Artificial Sequence Synthetic polynucleotide
atgacagtgctggctcctgcttggagccctaacagctctctgctgctgcttctgctcctgctgagcccatgtctgagaggcacccctgactgctacttctctcacagccccatcagcagcaacttcaaagtgaagttccgcgagctgaccgaccatctgctgaaggactaccctgtgaccgtggccgtgaacctgcaggatgagaagcactgcaaggccctgtggtccctgttcctggctcagagatggatcgagcagctgaaaacagtggccggcagcaagatgcagaccctgctggaagatgtgaacaccgagatccacttcgtgaccagctgcaccttccagcctctgcctgagtgcctgagattcgtgcagaccaacatcagccaccttctcaaggacacatgcacccagctgctggccctgaagccttgtatcggcaaggcctgccagaacttctccagatgcctggaagtgcagtgccagcctgacagctctacactgctgcctccaagaagccctatcgctctggaagccacagagctgcctgagcctagacctagacagagaagaaagagaagcggctccggcgccaccaacttcagcctgcttaaacaggcaggcgacgtggaagagaaccccggacctatgtgccctcagaagctgaccatcagttggttcgccatcgtgctgctggtgtccccactgatggctatgtgggaactcgagaaggacgtgtacgtggtggaagtggactggacccctgatgctcctggcgagacagtgaacctgacctgcgacacacctgaagaggacgacatcacctggaccagcgatcagagacacggcgtgatcggctctggcaagaccctgacaattaccgtgaaagagttcctggacgccggccagtacacctgtcacaaaggcggagagacactgagccactctcatctgctgctgcacaagaaagagaacggcatctggtccaccgagatcctgaagaacttcaagaacaagaccttcctgaagtgcgaggcccctaactacagcggcagattcacctgtagctggctggtgcagagaaacatggacctgaagttcaacatcaagtcctccagcagcagccccgacagcagagctgtgacatgtggcatggctagcctgagcgccgagaaagtgacactggaccagagagactacgagaagtacagcgtgtcctgccaagaggacgtgacctgtcctaccgccgaggaaacactgcctatcgagctggccctggaagccagacagcagaacaaatacgagaactactctaccagcttcttcatccgggacatcatcaagcccgatcctccaaagaacctgcagatgaagcctctgaagaacagccaggtcgaggtgtcctgggagtaccctgacagctggtctacccctcacagctacttcagcctgaaattcttcgtgcggatccagcgcaagaaagaaaagatgaaggaaaccgaggaaggctgcaaccagaaaggcgctttcctggtggaaaagaccagcaccgaggtgcagtgcaaaggcggcaatgtctgtgtgcaggcccaggaccggtactacaacagcagctgtagcaagtgggcctgcgtgccatgcagagtcagatctggtggcggaggatctggcggaggtggaagcggcggaggcggatctagagtgattcctgtgtctggccctgccagatgcctgagccagtctagaaacctgctgaaaaccaccgacgacatggtcaagaccgccagagagaagctgaagcactactcctgcacagccgaggacatcgaccacgaggatatcaccagggaccagacaagcaccctgaaaacctgcctgcctctggaactgcataagaacgagagctgcctggccaccagagaaaccagctctaccacaagaggcagctgtctgcctcctcagaaaaccagcctgatgatgaccctgtgcctgggcagcatctacgaggatctgaagatgtaccagaccgagttccaggccatcaacgccgctctgcagaaccacaaccaccagcagatcatcctggacaagggcatgctggtggctatcgacgagctgatgcagagcctgaaccataacggcgagacactgcggcagaagcctccagttggagaggccgatccttacagagtgaagatgaagctgtgcatcctgctgcacgccttcagcaccagagtggtcaccatcaacagagtgatgggctacctgagcagcgccaggcggaagcggagcggcagcggcgctacaaactttagccttctgaagcaggccggggatgtcgaagagaatcctggtccaatgagactgctgctgctgacattcctgggcgtgtgctgtctgacaccctgggttgtcgaaggcgtgggaacagaggtgctggaagagtccagctgcgtgaacctgcagacccagagactgcccgtgcagaagatcaagacctacatcatctgggagggcgccatgagagccgtgatcttcgtgacaaagagaggcctgaagatctgcgccgatcctgaggccaaatgggtcaaagccgccatcaagaccgtggacggcagagccagcaccagaaagaacatggccgagacagtgcctacaggcgcccagagatctaccagcacagccatcacactgaccggctga

SEQ ID NO: 55 - RNA, 510 nt, Homo sapiens
augagaggcacuccaggagacgcugaugguggaggaagggccgucuaucaaucaauguguaaaccuauuacugggacuauuaaugauuugaaucagcaaguguggacccuucagggucagaaccuuguggcaguuccacgaagugacagugugaccccagucacuguugcuguuaucacaugcaaguauccagaggcucuugagcaaggcagaggggaucccauuuauuugggaauccagaauccagaaauguguuuguauugugagaagguuggagaacagcccacauugcagcuaaaagagcagaagaucauggaucuguauggccaacccgagcccgugaaacccuuccuuuucuaccgugccaagacugguaggaccuccacccuugagucuguggccuucccggacugguucauugccuccuccaagagagaccagcccaucauucugacuucagaacuugggaagucauacaacacugccuuugaauuaaauauaaaugacuga

SEQ ID NO: 56 - RNA, 10 nt, Artificial Sequence
  Description of Artificial Sequence Synthetic oligonucleotide
aaaaaaaaaa